(19) United States
(12) Patent Application Publication
(10) Pub. No.: US 2025/0027099 A1
(43) Pub. Date: Jan. 23, 2025

(54) PHOSPHITE DEHYDROGENASE AS A SELECTABLE MARKER FOR MITOCHONDRIAL TRANSFORMATION

(71) Applicant: Napigen, Inc., Wilmington, DE (US)

(72) Inventors: Narendra Yadav, Wilmington, DE (US); Hajime Sakai, Newark, DE (US); Dilbag Multani, Urbandale, IA (US); Cheryl Caster, Landenberg, PA (US); Emil Orozco, Cochranville, PA (US); Ganesh Kishore, Creve Coeur, MO (US)

(21) Appl. No.: 18/716,877

(22) PCT Filed: Dec. 5, 2022
(86) PCT No.: PCT/US22/80942

§ 371 Date: Jun. 5, 2024

Related U.S. Application Data
(60) Provisional application No. 63/286,398, filed on Dec. 6, 2021.

(51) Int. Cl. C12N 15/82; A01N 59/26; A01P 13/02; C12N 5/04; C12N 9/02

(52) U.S. Cl. CPC C12N 15/8214; A01N 59/26; A01P 13/02; C12N 5/04; C12N 9/0004; C12Y 120/01001

(57) ABSTRACT

The present disclosure relates to genetically modified cells containing mitochondria that have been transformed with a polynucleotide encoding a phosphite dehydrogenase enzyme, such that the cells can utilize phosphite as a phosphorus source.

### CROSS-REFERENCE

This application claims benefit of U.S. Provisional Patent Application No. 63/286,398, filed on Dec. 6, 2021, which application is incorporated herein by reference in its entirety.

### GOVERNMENT RIGHTS

This invention was made with government support under Grant Number 2020-33610-31806 awarded by from the Small Business Innovation Research Program (SBIR) of the United States Department of Agriculture National Institute of Food and Agriculture (USDA-NIFA). The government has certain rights in the invention.

### SEQUENCE LISTING INCORPORATION BY REFERENCE

The application herein contains a Sequence Listing which has been submitted electronically in XML file format and is hereby incorporated by reference in its entirety. Said XML copy, created on Dec. 2, 2022, is named 51090-704_601_SL.xml.xml and is 197,307 bytes in size.

### BACKGROUND

Modification of mitochondrial genomes is of immense importance for basic and applied research. Transgenic plants with stably modified mitochondrial genomes can have new traits such as herbicide tolerance, insect resistance, and/or accumulation of valuable proteins such as pharmaceutical proteins and industrial enzymes.

### SUMMARY OF THE INVENTION

Aspects disclosed herein provide a cell comprising an edited mitochondrial genome, wherein the edited mitochondrial genome comprises an exogenous polynucleotide encoding a phosphite dehydrogenase or a biologically active fragment thereof. In some embodiments, the cell is a eukaryotic cell. In some embodiments, the eukaryotic cell is selected from the group consisting of a protist cell, a yeast cell, an algal cell, a plant cell, an insect cell, a non-human animal cell, an isolated and purified human cell, and a mammalian tissue culture cell. In some embodiments, the eukaryotic cell is a plant cell. In some embodiments, the plant cell is selected from the group consisting of: a wheat cell, a maize cell, a rice cell, a barley cell, a sorghum cell, a rye cell, a canola cell, a broccoli cell, a cauliflower cell, and a soybean cell. In some embodiments, the cell described herein can be an engineered non naturally occurring cell. In some embodiments, the edited mitochondrial genome comprises introduction of replacement, substitution, deletion or insertion of at least one nucleotide. In some embodiments, the cell comprises a transformed mitochondrion, wherein the transformed mitochondrion comprises the edited mitochondrial genome. In some embodiments, a nucleic acid sequence of the exogenous polynucleotide encoding the phosphite dehydrogenase or a biologically active fragment thereof comprises at least 80%, 85%, 90%, 95%, 96%, 97%, 98% or 99% sequence identity to SEQ ID NO: 28. In some embodiments, the nucleic acid sequence of the exogenous polynucleotide encoding the phosphite dehydrogenase or a biologically active fragment thereof comprises SEQ ID NO: 28. In some embodiments, an amino acid sequence of the phosphite dehydrogenase or a biologically active fragment thereof encoded by the exogenous polynucleotide comprises at least 80%, 85%, 90%, 95%, 96%, 97%, 98% or 99% sequence identity to SEQ ID NO: 29, 48, 49, 50, 51, 52, 53, 54, 55, 56, 57, 58, 59, or 60. In some embodiments, the amino acid sequence of the phosphite dehydrogenase or a biologically active fragment thereof comprises SEQ ID NO: 29. In some embodiments, a sequence encoding a start codon of the exogenous polynucleotide is replaced with a sequence encoding a mitochondrial RNA editing site. In some embodiments, the mitochondrial RNA editing site is from a mitochondrial nad4L gene or a mitochondrial cox2 gene. In some embodiments, the sequence encoding the mitochondrial RNA editing site comprises SEQ ID NO: 46. In some embodiments, the exogenous polynucleotide encoding the phosphite dehydrogenase or a biologically active fragment thereof comprises SEQ ID NO: 47. In some embodiments, the edited mitochondrial genome further comprises a second polynucleotide encoding a polypeptide or a functional RNA, or both, wherein the polypeptide and the functional RNA are exogenous to the mitochondria. In some embodiments, the cell comprises the second polynucleotide. In some embodiments, the second polynucleotide comprises a cytoplasmic male sterility (CMS) coding region. In some embodiments, the CMS coding region is orf79. In some embodiments, the cell is a rice cell. In some embodiments, the CMS coding region is orf256 or is orf279. In some embodiments, the cell is a wheat cell. In some embodiments, the cell further comprises a third exogenous polynucleotide in a nucleus of the cell, wherein the third exogenous polynucleotide encodes a selectable marker polypeptide that provides the cell with tolerance to a selective agent. In some embodiments, the selectable marker polypeptide is hygromycin phosphotransferase (HPT). In some embodiments, the selective agent is hygromycin. In some embodiments, the cell comprises a plurality of mitochondrial genomes wherein at least 50%, 60%, 70%, 80%, 90%, or 100% of the plurality of mitochondrial genomes comprise the edited mitochondrial genome. In some embodiments, the cell is homoplasmic for the edited mitochondrial genome. In some embodiments, the cell expresses the phosphite dehydrogenase or the biologically active fragment thereof encoded by the exogenous polynucleotide. In some embodiments, the cell grows in a medium wherein phosphite is present. In some embodiments, the cell grows when phosphite is present as a primary phosphorus source and wherein phosphate is present at less than 3 mg/liter. In some embodiments, the cell grows in a medium wherein the phosphite is present at 50 mM or greater. In some embodiments, the cell grows in a medium wherein the phosphite is present at 100 mM or greater. Aspects disclosed herein provide transgenic plant or parts thereof comprising the cells disclosed herein. In some embodiments, the transgenic plant or parts thereof comprises a cell, a tissue, a propagation material, a seed, a pollen, a progeny, or any combination thereof. In some embodiments, the transgenic plant or parts thereof is grown in a temperature-controlled incubator. In some embodiments, the temperature-controlled incubator further comprises a light-dark cycle. In some embodiments, a food product comprises the cell described herein. In some embodiments, a field comprises the cell described herein. In some embodiments, a kit comprising the cell described herein or the transgenic plant or parts thereof described herein.

Another aspect of the present disclosure provides a method comprising introducing into a mitochondrion of a cell, a first polynucleotide encoding a first polypeptide, wherein the first polypeptide comprises a phosphite dehydrogenase or a biologically active fragment thereof. In some embodiments, the method further comprises growing the cell under conditions in which the phosphite dehydrogenase or a biologically active fragment thereof is produced. In some embodiments, the method further comprises growing the cell in a medium wherein a phosphite is present. In some embodiments, the method further comprises selecting an edited mitochondrial genome comprising the first polynucleotide. In some embodiments, the method further comprises introducing into the mitochondrion of the cell a donor DNA, wherein the donor DNA comprises: (a) a second polynucleotide encoding a second polypeptide or a functional RNA, or both, wherein the second polypeptide and the functional RNA are exogenous to the mitochondrion; (b) a third polynucleotide at one end; and (c) a fourth polynucleotide at the other end; wherein the third polynucleotide and the fourth polynucleotide each comprises a sequence capable of homologous recombination with an endogenous mitochondrial DNA sequence, wherein homologous recombination of all or part of the third polynucleotide, the fourth polynucleotide, or both the third polynucleotide and the fourth polynucleotide, with the endogenous mitochondrial DNA sequence results in integration of the second polynucleotide into the endogenous mitochondrial DNA sequence; and selecting a cell with the edited mitochondrial genome, wherein the edited mitochondrial genome comprises the second polynucleotide. In some embodiments, the donor DNA further comprises the first polynucleotide. In some embodiments, the edited mitochondrial genome comprises both the first polynucleotide and the second polynucleotide. In some embodiments, the second polynucleotide comprises a cytoplasmic male sterility (CMS) coding region. In some embodiments, the CMS coding region comprises orf79. In some cases the orf79 is from a rice cell. In some embodiments, the CMS coding region comprises orf256 or orf279. In some embodiments, the orf256 or the orf279 is from a wheat cell. In some embodiments, the sequence capable of homologous recombination in the third polynucleotide has a size of 25-75 nucleotides, 25-100 nucleotides, 25-150 nucleotides, 25-200 nucleotides, 25-300 nucleotides, 25-400 nucleotides, 25-500 nucleotides, 25-1000 nucleotides, 25-1500 nucleotides, or 25-2000 nucleotides. In some embodiments, the sequence capable of homologous recombination in the fourth polynucleotide has a size of 25-75 nucleotides, 25-100 nucleotides, 25-150 nucleotides, 25-200 nucleotides, 25-300 nucleotides, 25-400 nucleotides, 25-500 nucleotides, 25-1000 nucleotides, 25-1500 nucleotides, or 25-2000 nucleotides. In some embodiments, the first polynucleotide, the second polynucleotide, the third polynucleotide and the fourth polynucleotide are all introduced into the mitochondrion as components of a single recombinant DNA construct. In some embodiments, at least one selected from the group consisting of: the first polynucleotide, the second polynucleotide, the third polynucleotide, the fourth polynucleotide, and any combination thereof, is introduced into the cell via microinjection, meristem transformation, electroporation, Agrobacterium-mediated transformation, viral based gene transfer, transfection, vacuum infiltration, biolistic particle bombardment or any combination thereof. In some embodiments, at least one selected from the group consisting of: the first polynucleotide, the second polynucleotide, the third polynucleotide, the fourth polynucleotide, and any combination thereof, is introduced into the cell as a peptide-polynucleotide complex, wherein the peptide-polynucleotide complex comprises at least one peptide. In some embodiments, at least one peptide of the peptide-polynucleotide complex comprises at least one selected from the group consisting of: a cell penetrating peptide (CPP), an organellar targeting peptide, a mitochondrial targeting peptide, a histidine-rich peptide, a lysine-rich peptide, and any combination thereof. In some embodiments, the method further comprises: (a) introducing into the mitochondrion of the cell a recombinant DNA construct comprising: (i) a first additional polynucleotide encoding at least one guide polynucleotide, wherein the at least one guide polynucleotide directs a polynucleotide guided polypeptide to cleave at least one target sequence present in an organelle genome; and (ii) a second additional polynucleotide encoding the polynucleotide guided polypeptide, wherein the polynucleotide guided polypeptide, when associated with the guide polynucleotide, cleaves the at least one target sequence. In some embodiments, the method further comprises: (a) introducing into a nucleus of the cell: (i) a first additional polynucleotide encoding a modified polynucleotide guided polypeptide, wherein the modified polynucleotide guided polypeptide comprises a polynucleotide guided polypeptide operably linked to a mitochondrial targeting peptide, wherein the polynucleotide guided polypeptide when associated with a guide RNA, cleaves at least one target sequence present in the mitochondrial genome; and (ii) a second additional polynucleotide encoding at least one guide RNA, wherein the at least one guide RNA directs the polynucleotide guided polypeptide to cleave the at least one target sequence present in the mitochondrial genome. In some embodiments, the method further comprises: (a) introducing into a nucleus of the cell: (i) a first additional polynucleotide encoding a modified polynucleotide guided polypeptide, wherein the modified polynucleotide guided polypeptide comprises a polynucleotide guided polypeptide operably linked to a mitochondrial targeting peptide, wherein the polynucleotide guided polypeptide when associated with a guide RNA, cleaves at least one target sequence present in the mitochondrial genome; and (b) introducing into the mitochondrion of the cell: (i) a second additional polynucleotide encoding at least one guide RNA, wherein the at least one guide RNA directs the polynucleotide guided polypeptide to cleave the at least one target sequence present in the mitochondrial genome. In some embodiments, the polynucleotide guided polypeptide is at least one selected from the group consisting of: a Cas9 protein, a Cas3 protein, a MAD2 protein, a MAD7 protein, a CRISPR nuclease, a nuclease domain of a Cas protein, a Cpf1 protein, an Argonaute, modified versions thereof, a biologically active fragment thereof, and any combination thereof. In some embodiments, homologous recombination of all or part of the third polynucleotide, or all or part of the fourth polynucleotide, or both, with endogenous mitochondrial DNA sequence results in an edited mitochondrial genome lacking the at least one target sequence. In some embodiments, the method further comprises: (a) introducing into a nucleus of the cell: (i) the second additional polynucleotide, wherein the second additional polynucleotide encodes a modified site-directed nuclease, wherein the modified site-directed nuclease comprises a site-directed nuclease operably linked to a mitochondrial targeting peptide, wherein the site-directed nuclease cleaves at least one target sequence present in the mitochondrial genome. In some embodiments, the site-directed nuclease is at least one selected from the group consisting of: a TALEN, a Zinc-Finger Nuclease, a Meganuclease, a restriction enzyme, and any combination thereof. In some embodiments, the method further comprises: (a) introducing into a nucleus of the cell: (i) a third additional polynucleotide encoding a selectable marker polypeptide that provides tolerance to a selective agent; and (b) selecting a cell that grows in the presence of the selective agent. In some embodiments, the first polynucleotide encoding the phosphite dehydrogenase or a biologically active fragment thereof further comprises a T7 RNA polymerase promoter, wherein expression of the phosphite dehydrogenase or a biologically active fragment thereof is under control of the T7 RNA polymerase promoter. In some embodiments, the method further comprising: (a) introducing into a nucleus of the cell: (i) a fourth additional polynucleotide encoding a modified T7 RNA polymerase, wherein the modified T7 RNA polymerase comprises a T7 RNA polymerase operably linked to a mitochondrial targeting peptide. In some embodiments, the mitochondrial targeting peptide is encoded by SEQ ID NO: 38. In some embodiments, the phosphite dehydrogenase or a biologically active fragment thereof comprises an amino acid sequence with at least 70%, 80%, 85%, 90%, 91%, 92%, 93%, 94%, 95% or 99% sequence identity to SEQ ID NO: 29, 48, 49, 50, 51, 52, 53, 54, 55, 56, 57, 58, 59, or 60. In some embodiments, the first polynucleotide encoding the phosphite dehydrogenase or a biologically active fragment thereof further comprises SEQ ID NO: 44 or SEQ ID NO: 45. In some embodiments, a sequence encoding a start codon of the phosphite dehydrogenase or a biologically active fragment thereof is replaced with a sequence encoding a mitochondrial RNA editing site. In some embodiments, the mitochondrial RNA editing site is from a mitochondrial nad4L gene or a mitochondrial cox2 gene. In some embodiments, the sequence encoding the mitochondrial RNA editing site comprises SEQ ID NO: 46. In some embodiments, the first polynucleotide encoding the phosphite dehydrogenase or the biologically active fragment thereof comprises SEQ ID NO: 47. In some embodiments, the cell is grown simultaneously in a presence of a selective agent and in a presence of a phosphite as a primary phosphorus source, wherein phosphate is present at less than 3 mg/liter. In some embodiments, the cell is grown sequentially first in a presence of a selective agent and subsequently in a presence of a phosphite as a primary phosphorus source, wherein phosphate is present at less than 3 mg/liter. In some embodiments, the selectable marker polypeptide is hygromycin phosphotransferase (HPT) and the selective agent is hygromycin. In some embodiments, the method further comprises removing the first polynucleotide encoding the phosphite dehydrogenase or a biologically active fragment thereof after inserting the second polypeptide. In some embodiments, the method further comprises selecting a cell that comprises a plurality of mitochondrial genomes, wherein at least 50%, 60%, 70%, 80%, 90%, or 100% of the plurality of mitochondrial genomes comprise the edited mitochondrial genome. In some embodiments, the method further comprises selecting a cell that is homoplasmic for the edited mitochondrial genome. In some embodiments, the cell is a yeast cell, an algal cell, a plant cell, an insect cell, a non-human animal cell, an isolated and purified human cell, or a mammalian tissue culture cell. In some embodiments, the cell described herein can be an engineered non naturally occurring cell. In some embodiments, the cell is a plant cell. In some embodiment, a plant, cell, tissue, propagation material, seed, root, leaf, flower, fruit, pollen, progeny, or part thereof, produced from the plant cell described herein, wherein the plant, cell, tissue, propagation material, seed, root, leaf, flower, fruit, pollen, progeny, or part thereof comprises the edited mitochondrial genome. In some embodiments, the method of using the cells described herein, or the method described herein for growing a plant.

Another aspect of the present disclosure provides a method of controlling weeds, the method comprising (a) growing a plurality of plants in a presence of a phosphite, wherein at least one plant of the plurality of plants comprises a mitochondrion having an exogenous polynucleotide that encodes phosphite dehydrogenase or a biologically active fragment thereof, wherein the presence of the phosphite is sufficient to selectively promote growth of the at least one plant of the plurality of plants, resulting in an increased growth of the at least one plant of the plurality of plants relative to plants lacking phosphite dehydrogenase or a biologically active fragment thereof. In some embodiments, the method further comprises applying phosphite to the plant, the plurality of plants, soil adjacent to the plant, or any combination thereof. In some embodiments, the phosphite is applied as a foliar fertilizer. In some cases the phosphite is applied as a soil amendment. In some embodiments, the at least one plant of the plurality of plants is selected from the group consisting of: wheat, maize, rice, barley, sorghum, rye, sugarcane, potato, tomato, canola, broccoli, cauliflower, and soybean. In some embodiments, a plant lacking phosphite dehydrogenase or a biologically active fragment thereof is a weed. In some embodiments, the phosphite dehydrogenase or a biologically active fragment thereof comprises an amino acid sequence with at least 80%, 85%, 90%, 91%, 92%, 93%, 94%, or 95% sequence identity to SEQ ID NO: 29, 48, 49, 50, 51, 52, 53, 54, 55, 56, 57, 58, 59, or 60. In some embodiments, the phosphite dehydrogenase or a biologically active fragment thereof comprises an amino acid sequence of SEQ ID NO: 29. In some embodiments, the method of using the cells described herein or the methods described herein for growing a plant. In some embodiments, a field or a greenhouse comprises the plant described herein. In some embodiments, a food product comprises the cell described herein. In some embodiments, a field comprises the cell described herein. In some embodiments, a kit comprising the cell described herein or the transgenic plant or parts thereof described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows yeast transformed with constructs containing a ptxD gene, grown on a medium containing phosphite as a sole phosphorus source. FIG. 1A shows a Wild-type strain (CUY563) transformed with pNY101, a nuclear construct expressing a PtxD protein targeting to mitochondria (pTEF::MTS:PtxD); FIG. 1B shows a wild-type strain (CUY563) transformed with an empty vector, pYES2; FIG. 1C shows a wild-type strain (CUY563) transformed with pNY104, a mitochondrial plasmid expressing a PtxD protein in a mitochondria.

FIG. 2 shows a map of plasmid pNAP256. The plasmid contains a sequence encoding a fusion protein comprising a mitochondrial targeting sequence of an rps10 gene, a ptxD gene optimized for expression in a rice nucleus, a PVAT linker (SEQ ID NO: 72), and a fluorescent reporter eGFP. The coding region is under the control of a maize UBI-1 promoter and intron and a nos terminator. The plasmid also contains a coding region for hygromycin phosphotransferase (HPT) under the control of a 35S promoter and a CaMV 3′-UTR.

FIG. 3 shows growth of rice callus cells in a phosphite medium, wherein the rice callus cells were transformed with pNAP256, a nuclear construct expressing a PtxD enzyme fused with a mitochondrial targeting peptide. FIG. 3A: tissue from a slow growing event; FIG. 3B: tissue from a faster growing event.

FIG. 4 shows a map of plasmid pNAP250 that contains a coding region for a fusion protein consisting of PtxD and eGFP. The PtxD and eGFP proteins are connected with a PVAT linker (SEQ ID NO: 72). The PtxD-eGFP coding region is linked to a rice mitochondrial ATP1 promoter and a rice mitochondrial ATP1 terminator. The plasmid also contains a rice autonomous B4 element.

FIG. 5 shows a map of plasmid pNAP233 that contains a coding region for a fusion protein consisting of PtxD and eGFP; the two enzymes are connected with a GGGGS linker (SEQ ID NO: 84). The fusion protein coding region is linked to a T7 promoter at a 5′ end and to a T7 terminator and a truncated fragment of a rice ATP1 terminator at a 3′ end. The plasmid also contains a rice autonomous B4 element.

FIG. 6 shows a map of plasmid pNAP160 that contains a coding region for a fusion protein consisting of a mitochondrial targeting sequence (MTS) and a T7 RNA polymerase. The MTS-T7 RNA Polymerase coding region is linked to a maize ubiquitin-1 (UBI-1) promoter and intron and an Agrobacterium tumefaciens nopaline synthase (NOS) terminator. The plasmid also contains a coding region for hygromycin phosphotransferase (HPT) under control of a 35S promoter and a CaMV terminator.

FIG. 7 shows growth of transformed rice callus on phosphite medium. Events were selected on hygromycin-containing medium having phosphite as a sole phosphorus source for three weeks and subcultured on the same medium for two weeks. Events were transformed with the following expression units on the indicated plasmid DNAs in FIG. 7A-D. FIG. 7A shows pATP1::ptxD-eGFP (pNAP250).

FIG. 7B shows pATP1::RNAed-ptxD-eGFP (pNAP251). FIG. 7C shows pT7::ptxD-eGFP (pNAP233).

FIG. 7D shows pT7::RNAed-ptxD-eGFP (pNAP246). All mitochondrial constructs were co-transformed with nuclear constructs containing a HPT hygromycin resistance gene. pATP1: promoter for the rice mitochondrial ATP1 gene. RNAed: ATG was replaced with a mitochondrial RNA editing site as described in Example 9 (see FIG. 8). A bar indicating 1 mm in size is shown.

FIG. 8 shows a diagrammatic illustration of the strategy employed for mitochondria-specific gene expression using a naturally occurring mitochondrial RNA editing site. The sequence (SEQ ID NO: 110, RICE NAD4L) surrounding the start codon of the endogenous rice mitochondrial NAD4L gene is shown; the RNA editing site is shown in italics. The initial amino acids (SEQ ID NO: 111) encoded by NAD4L are shown below this sequence. The sequence (SEQ ID NO: 112, pATP1-ptxD) surrounding the ATG start codon of ptxD in the pATP1-ptxD expression unit is shown. The initial amino acids (SEQ ID NO: 113) encoded by pATP1-ptxD are shown below this sequence. The ATG codon of pATP1-ptxD was replaced with the RNA editing site of NAD4L and the modified sequence (SEQ ID NO: 114, pATP1-RNAed-ptxD) is shown. Upon transcription and subsequent RNA processing in the mitochondria, an ACG sequence in the primary transcript is edited to be AUG, i.e., the mRNA start codon. The edited mRNA sequence is shown (SEQ ID NO: 115, mRNA). The initial amino acids (SEQ ID NO: 116) encoded by the edited mRNA sequence are shown below the sequence.

FIG. 9 shows a map of plasmid pNAP251. Plasmid pNAP251 encodes a fusion protein of PtxD and eGFP protein, joined by a PVAT linker (SEQ ID NO: 72). The coding region has a rice mitochondrial RNA editing site at the 5′ end to provide the start codon (see FIG. 8). The fusion protein coding sequence is linked to the rice ATP1 promoter and the rice ATP1 terminator. The plasmid also contains the rice autonomous B4 element.

FIG. 10 shows a map of plasmid pNAP246 that contains a coding region for a fusion protein consisting of PtxD and eGFP linked together with a PVAT linker (SEQ ID NO: 72). The ptxD-eGFP coding region also contains a rice mitochondrial RNA editing sequence at the translation initiation codon (see FIG. 8). The ptxD-eGFP coding region is linked to a T7 promoter at the 5′ end and to a T7 terminator and a truncated fragment of a rice ATP1 terminator at the 3′ end. The plasmid also contains the rice autonomous B4 element.

FIG. 11 shows a diagrammatic illustration of where a Donor DNA is targeted to a mitochondrial genome. In FIG. 11 the Donor DNA contains two regions of homology (HR) with the mitochondrial genome. The Donor DNA also has modified gRNA1 and gRNA2 sites, where the modified sequence is no longer a substrate for MAD7. Within the Donor DNA are sequences encoding a CMS gene, an ORF79, and a fluorescent protein TagRFP. The position of targeted integration into the mitochondrial genome at the end of the atp6 gene is shown. Alternative Donor DNAs use gRNA2 and gRNA4 instead of gRNA1 and gRNA3.

FIG. 12 shows a map of Edit Plasmid pNAP294. This plasmid contains a Donor DNA targeted to gRNA1 and gRNA3 sites. Also present on the plasmid is an expression unit encoding a selectable marker fusion protein ptxD-eGFP. The fusion protein has a rice mitochondrial RNA editing site at a 5′ end to provide an AUG start codon in a corresponding mRNA. The expression unit also contains a multigene cassette encoding trnP-gRNA1-trnE-gRNA3-trnK. The expression unit contains a T7 promoter at a 5′ end and a T7 terminator at a 3′ end. Also present on the Edit Plasmid is a rice autonomous B4 element for mitochondrial DNA replication.

FIG. 13 shows a map of plasmid pNAP255. One expression unit encodes a fusion protein having a mitochondrial targeting sequence (MTS) fused to T7 RNA polymerase. This expression unit is under control of a maize UBI-1 promoter and an Agrobacterium tumefaciens octopine synthase (OCS) terminator. A second expression unit encodes fusion protein having a mitochondrial targeting sequence (MTS) fused to MAD7. This expression unit is under control of a rice actin-1 promoter and an NOS terminator. A third expression unit encodes an HPT selectable marker. This expression unit is under control of a 35S promoter and a CaMV terminator.

FIG. 14 shows a PCR analysis of Donor DNA integration at gRNA1 & gRNA2 sites. The integration site was amplified with a primer set, one from a mitochondrial genomic region near a cleavage site and another from within the Donor DNA. The position of an expected junction fragment of 484 bp is indicated with an arrow. Lanes #1 & 30: Molecular size standards; Lanes #2-16: Independent events transformed with pNAP291 expressing gRNA2 & gRNA4; Lanes #17-24 and lanes #26-28: Independent events transformed with pNAP294 expressing gRNA1 & gRNA3. In each construct, gRNAs were expressed from a T7 promoter. Lanes #25 & 29: Negative controls without DNA samples.

FIG. 15 shows DNA sequences of fragments obtained from PCR amplification of integration sites using primer ATP6-1 (SEQ ID NO: 106) and primer 79-2 (SEQ ID NO: 107). FIG. 15A shows a sequence (SEQ ID NO: 117) integrated at the gRNA1 site of multiple independent events. FIG. 15B shows a sequence (SEQ ID NO: 118) integrated at the gRNA2 site of two independent events. In both FIG. 15A and FIG. 15B, the break points of homologous recombination were found directly downstream of the gRNA sites. In FIG. 15A and FIG. 15B the fragment sequence identical to wild-type mtDNA genomic sequence is shown in roman font (i.e., not italics); the single nucleotide residue at the 5′ end of the homologous region of the Donor DNA is both underlined and in bold font; the gRNA sequence within the homologous region of the Donor DNA is shown in bold font; the sequence corresponding to the window of recombination is shown as underlined; and the non-homologous Donor DNA sequence is shown in italics.

FIG. 16A and FIG. 16B show the PCR analysis of Donor DNA integration at the gRNA1 & gRNA4 sites for MAD7. Each integration site was amplified with a primer set, one primer specific to the mitochondrial genomic region outside of the homologous region in the Donor DNA and the other primer specific to a unique region within the Donor DNA. FIG. 16A shows the position of the expected 5′ junction fragment of 1.8 kb is indicated with an arrow. FIG. 16B shows the position of the expected 3′ junction fragment of 1.4 kb is indicated with an arrow. Lanes M: Molecular size standards; Lanes #1-7: Independent events transformed with gel-purified Donor DNA fragments; Lanes C: Control reaction with no DNA.

FIG. 17 shows the RT-PCR analysis for expression of mOsPtxD. Lanes M: Molecular size standards; Lanes wt: Control with wild-type (non-transformed) callus DNA; Lane #1: DNA from an event derived from co-transformation with pNAP420 (mitochondrial expression construct; nad4L_long RNA editing sequence; ATP1+T7 promoter) and pNAP255 (nuclear expression construct); Lane #2: DNA from an event derived from co-transformation with pNAP391 (mitochondrial expression construct; nad4L_short RNA editing sequence; ATP1 promoter) and pNAP199 (nuclear expression construct); Lane #3: DNA from an event derived from co-transformation of pNAP422 (mitochondrial expression construct; cox2 RNA editing sequence; ATP1+T7 promoter) and pNAP255 (nuclear expression construct); Lanes dH2O: Control reactions with no DNA. Left half: RT-PCR reactions using Act1 primers produced the expected 346 bp product derived from Act1 mRNA (shown with arrow) without any 460 bp product derived from intron-containing genomic Act1 DNA. Right half: RT-PCR reactions using mOsPtxD primers produced the expected 417 bp fragment derived from mOsPtxD mRNA (shown with arrow).

### DETAILED DESCRIPTION OF THE INVENTION

In some cases, mitochondrial genome editing can be more difficult than nuclear genome or plastid genome editing. In some cases, a new selectable marker gene can be used to generate and identify a cell comprising an edited mitochondrial genome. In some cases, a new selectable marker gene can be needed to edit mitochondrial genome of a plant.

Disclosed herein in some embodiments, are methods and compositions for making and using organisms having a polynucleotide in an edited mitochondrial genome. In some embodiments, a transformed mitochondrion may comprise the edited mitochondrial genome. In some embodiments, a polynucleotide can encode an enzyme having phosphite dehydrogenase (NAD:phosphite oxidoreductase) or a biologically active fragment thereof activity. In some embodiments, an enzyme can be of bacterial origin. In some embodiments, an enzyme can be a PtxD polypeptide or a biologically active fragment thereof of Pseudomonas stutzeri. In some embodiments, a phosphite dehydrogenase enzyme or a biologically active fragment thereof in a mitochondria can enable metabolism of phosphite as a source of phosphorus which can allow for its use as a selectable marker. In some embodiments, a polypeptide disclosed herein can comprise a sequence listed in Table 1. In some embodiments, a polypeptide disclosed herein can comprise at least about 80%, 81%, 82%, 83%, 84%, 85%, 86%, 87%, 88%, 89%, 90%, 91%, 92%, 93%, 94%, 95%, 96%, 97%, 98%, 99% or 100% homology to a sequence listed in Table 1. In some embodiments, a polypeptide disclosed herein can comprise at least about 80% homology to a sequence listed in Table 1.

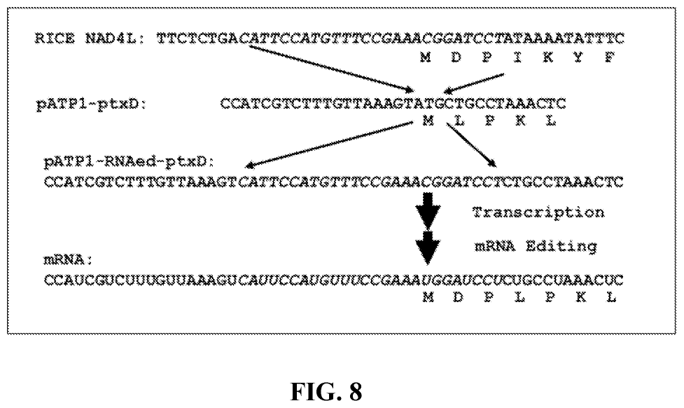

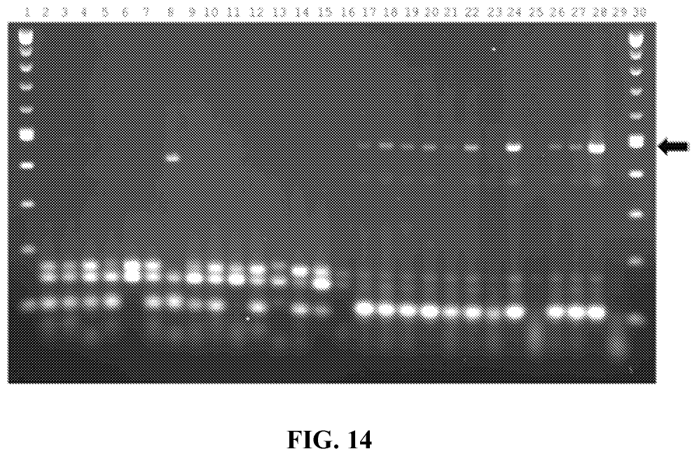

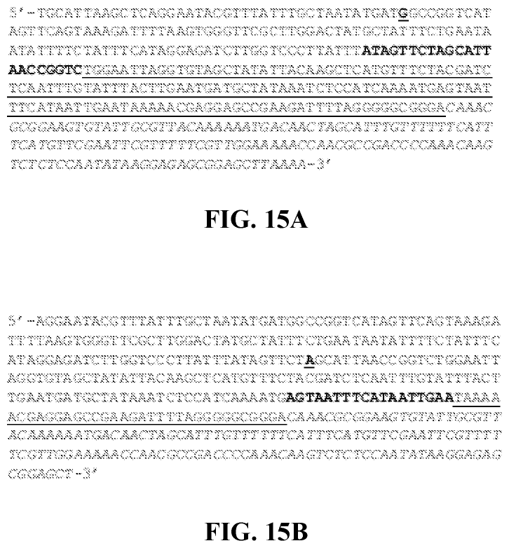

TABLE 1

| | Brief Description | |
|---|---|---|
| SEQ | of the | |
| ID | Sequence | Sequence |
| 1 | Amino acid sequence of | MDKKYSIGLDIGTNSVGWAVITDEYKVPSK |
| | SpCas9, the Cas9 from | KFKVLGNTDRHSIKKNLIGALLFDSGETAE |
| | Streptococcus pyogenes | ATRLKRTARRRYTRRKNRICYLQEIFSNEM |
| | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKLESEFVYGDYKVYDVRK |
| | | MIAKSEQEIGKATAKYFFYSNIMNFFKTEI |
| | | TLANGEIRKRPLIETNGETGEIVWDKGRDF |
| | | ATVRKVLSMPQVNIVKKTEVQTGGFSKESI |
| | | LPKRNSDKLIARKKDWDPKKYGGFDSPTVA |
| | | YSVLVVAKVEKGKSKKLKSVKELLGITIME |
| | | RSSFEKNPIDFLEAKGYKEVKKDLIIKLPK |
| | | YSLFELENGRKRMLASAGELQKGNELALPS |
| | | KYVNFLYLASHYEKLKGSPEDNEQKQLFVE |
| | | QHKHYLDEIIEQISEFSKRVILADANLDKV |
| | | LSAYNKHRDKPIREQAENIIHLFTLTNLGA |
| | | PAAFKYFDTTIDRKRYTSTKEVLDATLIHQ |
| | | SITGLYETRIDLSQLGGD |
| 2 | Amino acid sequence for | MSSLTKFTNKYSKQLTIKNELIPVGKTLEN |
| | MAD2 | IKENGLIDGDEQLNENYQKAKIIVDDFLRD |
| | | FINKALNNTQIGNWRELADALNKEDEDNIE |
| | | KLQDKIRGIIVSKFETFDLFSSYSIKKDEK |
| | | IIDDDNDVEEEELDLGKKTSSFKYIFKKNL |
| | | FKLVLPSYLKTTNQDKLKIISSFDNFSTYF |
| | | RGFFENRKNIFTKKPISTSIAYRIVHDNFP |
| | | KFLDNIRCFNVWQTECPQLIVKADNYLKSK |
| | | NVIAKDKSLANYFTVGAYDYFLSQNGIDFY |
| | | NNIIGGLPAFAGHEKIQGLNEFINQECQKD |
| | | SELKSKLKNRHAFKMAVLFKQILSDREKSF |
| | | VIDEFESDAQVIDAVKNFYAEQCKDNNVIF |
| | | NLLNLIKNIAFLSDDELDGIFIEGKYLSSV |
| | | SQKLYSDWSKLRNDIEDSANSKQGNKELAK |
| | | KIKTNKGDVEKAISKYEFSLSELNSIVHDN |
| | | TKFSDLLSCTLHKVASEKLVKVNEGDWPKH |
| | | LKNNEEKQKIKEPLDALLEIYNTLLIFNCK |
| | | SFNKNGNFYVDYDRCINELSSVVYLYNKTR |
| | | NYCTKKPYNTDKFKLNFNSPQLGEGFSKSK |
| | | ENDCLTLLFKKDDNYYVGIIRKGAKINFDD |
| | | TQAIADNTDNCIFKMNYFLLKDAKKFIPKC |
| | | SIQLKEVKAHFKKSEDDYILSDKEKFASPL |
| | | VIKKSTFLLATAHVKGKKGNIKKFQKEYSK |
| | | ENPTEYRNSLNEWIAFCKEFLKTYKAATIF |
| | | DITTLKKAEEYADIVEFYKDVDNLCYKLEF |
| | | CPIKTSFIENLIDNGDLYLFRINNKDFSSK |
| | | STGTKNLHTLYLQAIFDERNLNNPTIMLNG |
| | | GAELFYRKESIEQKNRITHKAGSILVNKVC |
| | | KDGTSLDDKIRNEIYQYENKFIDTLSDEAK |
| | | KVLPNVIKKEATHDITKDKRFTSDKFFFHC |
| | | PLTINYKEGDTKQFNNEVLSFLRGNPDINI |
| | | IGIDRGERNLIYVTVINQKGEILDSVSFNT |
| | | VTNKSSKIEQTVDYEEKLAVREKERIEAKR |
| | | SWDSISKIATLKEGYLSAIVHEICLLMIKH |
| | | NAIVVLENLNAGFKRIRGGLSEKSVYQKFE |
| | | KMLINKLNYFVSKKESDWNKPSGLLNGLQL |
| | | SDQFESFEKLGIQSGFIFYVPAAYTSKIDP |
| | | TTGFANVLNLSKVRNVDAIKSFFSNFNEIS |
| | | YSKKEALFKFSFDLDSLSKKGFSSFVKFSK |
| | | SKWNVYTFGERIIKPKNKQGYREDKRINLT |
| | | FEMKKLLNEYKVSFDLENNLIPNLTSANLK |
| | | DTFWKELFFIFKTTLQLRNSVTNGKEDVLI |
| | | SPVKNAKGEFFVSGTHNKTLPQDCDANGAY |
| | | HIALKGLMILERNNLVREEKDTKKIMAISN |
| | | VDWFEYVQKRRGVL |
| 3 | Amino acid sequence for | MNNGTNNFQNFIGISSLQKTLRNALIPTET |
| | MAD7 | TQQFIVKNGIIKEDELRGENRQILKDIMDD |
| | | YYRGFISETLSSIDDIDWTSLFEKMEIQLK |
| | | NGDNKDTLIKEQTEYRKAIHKKFANDDRFK |
| | | NMFSAKLISDILPEFVIHNNNYSASEKEEK |
| | | TQVIKLFSRFATSFKDYFKNRANCFSADDI |
| | | SSSSCHRIVNDNAEIFFSNALVYRRIVKSL |
| | | SNDDINKISGDMKDSLKEMSLEEIYSYEKY |
| | | GEFITQEGISFYNDICGKVNSFMNLYCQKN |
| | | KENKNLYKLQKLHKQILCIADTSYEVPYKF |
| | | ESDEEVYQSVNGFLDNISSKHIVERLRKIG |
| | | DNYNGYNLDKIYIVSKFYESVSQKTYRDWE |
| | | TINTALEIHYNNILPGNGKSKADKVKKAVK |
| | | NDLQKSITEINELVSNYKLCSDDNIKAETY |
| | | IHEISHILNNFEAQELKYNPEIHLVESELK |
| | | ASELKNVLDVIMNAFHWCSVFMTEELVDKD |
| | | NNFYAELEEIYDEIYPVISLYNLVRNYVTQ |
| | | KPYSTKKIKLNFGIPTLADGWSKSKEYSNN |
| | | AIILMRDNLYYLGIFNAKNKPDKKIIEGNT |
| | | SENKGDYKKMIYNLLPGPNKMIPKVFLSSK |
| | | TGVETYKPSAYILEGYKQNKHIKSSKDFDI |
| | | TFCHDLIDYFKNCIAIHPEWKNFGFDFSDT |
| | | STYEDISGFYREVELQGYKIDWTYISEKDI |
| | | DLLQEKGQLYLFQIYNKDFSKKSTGNDNLH |
| | | TMYLKNLFSEENLKDIVLKLNGEAEIFFRK |
| | | SSIKNPIIHKKGSILVNRTYEAEEKDQFGN |
| | | IQIVRKNIPENIYQELYKYFNDKSDKELSD |
| | | EAAKLKNVVGHHEAATNIVKDYRYTYDKYF |
| | | LHMPITINFKANKTGFINDRILQYIAKEKD |
| | | LHVIGIDRGERNLIYVSVIDTCGNIVEQKS |
| | | FNIVNGYDYQIKLKQQEGARQIARKEWKEI |
| | | GKIKEIKEGYLSLVIHEISKMVIKYNAIIA |
| | | MEDLSYGFKKGRFKVERQVYQKFETMLINK |
| | | LNYLVFKDISITENGGLLKGYQLTYIPDKL |
| | | KNVGHQCGCIFYVPAAYTSKIDPTTGFVNI |
| | | FKFKDLTVDAKREFIKKFDSIRYDSEKNLF |
| | | CFTFDYNNFITQNTVMSKSSWSVYTYGVRI |
| | | KRRFVNGRFSNESDTIDITKDMEKTLEMTD |
| | | INWRDGHDLRQDIIDYEIVQHIFEIFRLTV |
| | | QMRNSLSELEDRDYDRLISPVLNENNIFYD |
| | | SAKAGDALPKDADANGAYCIALKGLYEIKQ |
| | | ITENWKEDGKFSRDKLKISNKDWFDFIQNK |
| | | RYL |
| 4 | Amino acid sequence | LAGLIDADG |
| | LAGLIDADG, a | |
| | conserved sequence | |
| | motif for one of the four | |
| | Meganuclease families | |
| 5 | Amino acid sequence of | DEVDFQGPCNDSSDPLVVAASIIGILHLIL |
| | a hydrophobic | WILDRL |
| | quenching peptide that | |
| | tetramerizes GFP and | |
| | prevents maturation of | |
| | the chromophore | |
| 6 | Amino acid sequence of | DEVD |
| | a caspase recognition | |
| | sequence | |
| 7 | Nucleotide sequence for | ACUUUUGACAGUUAUACGAUUCCAGAA |
| | a candidate RNA editing | |
| | sequence present in the | |
| | wheat mitochondrial | |
| | cox2 gene at position | |
| | 449 of the gene | |
| 8 | Nucleotide sequence for | UGGGCUGUACCUUCCUCAGGUGUCAAA |
| | a candidate RNA editing | |
| | sequences present in the | |
| | wheat mitochondrial | |
| | cox2 gene at position | |
| | 587 of the gene | |
| 9 | Nucleotide sequence for | GCUGUACCUGGUCGUUCAAAUCUUACC |
| | a candidate RNA editing | |
| | sequence present in the | |
| | wheat mitochondrial | |
| | cox2 gene at position | |
| | 620 of the gene | |
| 10 | Amino acid sequence for | RQIKIWFQNRRMKWKK |
| | a permeant peptide | |
| | derived from the third | |
| | alpha helix of | |
| | Drosophila melanogaster | |
| | transcription factor | |
| | Antennapaedia, referred | |
| | to as penetratin | |
| 11 | Amino acid sequence for | KHKHKHKHKHKHKHKHKHMFAFQYLLVM |
| | KH-AtOEP34 | |
| 12 | Amino acid sequence for | RKKRRQRRR |
| | TAT | |
| 13 | Amino acid sequence for | RRRRRRRRR |
| | R9 | |
| 14 | Amino acid sequence for | KETWWETWWTEWSQPKKKRKV |
| | Pep-1 | |
| 15 | Amino acid sequence for | GALFLGFLGAAGSTMGAWSQPKKKRKV |
| | MPG | |
| 16 | Amino acid sequence for | VRLPPP |
| | gamma-ZEIN | |
| 17 | Amino acid sequence for | GWTLNSAGYLLGKINLKALAALAKKIL |
| | Transportan | |
| 18 | Amino acid sequence for | KLALKLALKALKAALKLA |
| | MAP | |
| 19 | Amino acid sequence for | PLILLRLLRGQF |
| | Pept 1 | |
| 20 | Amino acid sequence for | PLIYLRLLRGQF |
| | Pept 2 | |
| 21 | Amino acid sequence for | KLWMRWYSPTTRRYG |
| | IVV-14 | |
| 22 | Amino acid sequence for | MGLGLHLLVLAAALQGAKKKRKV |
| | Ig(v) | |
| 23 | Amino acid sequence for | KLALKLALKALKAALKLA |
| | Amphiphilic model | |
| | peptide | |
| 24 | Amino acid sequence for | LLIILRRRIRKQAHAHSK |
| | pVEC | |
| 25 | Amino acid sequence for | RRIPNRRPRR |
| | HRSV | |
| 26 | Amino acid sequence for | KKLFKKILKYL |
| | Bp100 | |
| 27 | Amino acid sequence for | RKKRRQRRRRKKRRQRRR |
| | TAT2, a dimer of the | |
| | HIV-1 Tat basic domain | |
| 28 | Nucleotide sequence of | ATGCTGCCIAAACTCGTTATAACTCACCGA |
| | the PtxD CDS optimized | GTACAtGATGAGATCCTGCAACTGCTGGCG |
| | for rice mitochondria | CCACATTGCGAGCTGATGACCAACCAaACC |
| | (mOsPtxD) | GACAGCACaCTGACaCGCGAGGAAATTCTG |
| | | CGtCGaTGTCGtGATGCTCAaGCGATGATG |
| | | GCGTTCATGCCCGATCGaGTCGATGCAGAC |
| | | TTTCTTCAAGCCTGCCCTGAGCTGCGTGTA |
| | | GTCGGCTGCGCGCTCAAGGGCTTCGACAAT |
| | | TTCGATGTGGACGCCTGTACTGCCCGtGGG |
| | | GTCTGGCTGACCTTCGTGCCTGATCTGTTG |
| | | ACaGTCCCaACTGCCGAGCTGGCGATCGGA |
| | | CTGGCGGTGGGGCTGGGGCGaCATCTGCGa |
| | | GCAGCAGATGCGTTCGTCCGtTCTGGCGAG |
| | | TTCCAaGGCTGGCAACCACAaTTCTAtGGC |
| | | ACaGGGCTGGATAACGCTACaGTCGGCATC |
| | | CTTGGCATGGGCGCCATCGGACTGGCCATG |
| | | GCTGATCGaTTGCAaGGATGGGGCGCGACC |
| | | CTGCAaTAtCACGAGGCGAAGGCTCTGGAT |
| | | ACACAAACCGAGCAACGaCTCGGCCTGCGt |
| | | CAaGTGGCGTGCAGCGAACTCTTCGCCAGC |
| | | TCGGACTTCATCCTGCTGGCGCTTCCCTTG |
| | | AATGCCGATACCCAaCATCTGGTCAACGCC |
| | | GAGCTGCTTGCCCTCGTACGaCCaGGCGCT |
| | | CTGCTTGTAAACCCCTGTCGTGGTTCGGTA |
| | | GTGGATGAAGCCGCCGTGCTCGCGGCGCTT |
| | | GAGCGAGGCCAaCTCGGCGGGTATGCGGCG |
| | | GATGTATTCGAAATGGAAGACTGGGCTCGt |
| | | GCGGACCGaCCaCGaCTGATCGATCCTGCG |
| | | CTGCTCGCGCATCCIAATACaCTGTTCACT |
| | | CCaCAtATAGGGTCGGCAGTGCGtGCGGTG |
| | | CGtCTGGAGATTGAACGTTGTGCAGCGCAa |
| | | AACATCATCCAaGTATTGGCAGGTGCGCGt |
| | | CCAATCAACGCTGCGAACCGTCTGCCCAAG |
| | | GCCGAGCCTGCCGCATGTTGA |
| 29 | Amino acid sequence of | MLPKLVITHRVHDEILQLLAPHCELMTNQT |
| | mOsPtxD that is | DSTLTREEILRRCRDAQAMMAFMPDRVDAD |
| | encoded by SEQ ID NO: | FLQACPELRVVGCALKGFDNFDVDACTARG |
| | 28 and is 100% identical | VWLTFVPDLLTVPTAELAIGLAVGLGRHLR |
| | to the PtxD protein | AADAFVRSGEFQGWQPQFYGTGLDNATVGI |
| | (GenBank: | LGMGAIGLAMADRLQGWGATLQYHEAKALD |
| | AAC71709.1) of | TQTEQRLGLRQVACSELFASSDFILLALPL |
| | Pseudomonas stutzeri | NADTQHLVNAELLALVRPGALLVNPCRGSV |
| | WM 88 | VDEAAVLAALERGQLGGYAADVFEMEDWAR |
| | | ADRPRLIDPALLAHPNTLFTPHIGSAVRAV |
| | | RLEIERCAAQNIIQVLAGARPINAANRLPK |
| | | AEPAAC |
| 30 | Nucleotide sequence of | TCTGCTTGAAAGCCTGCAGAGTCCAATTTT |
| | the putative promoter | GAGTATTTTCAGTTAGAATCTAGAGTCAGC |
| | sequence of the ATP1 | CTATTCAGTTCTTAGCCCTTAAGGGTAAGG |
| | gene that is encoded in | CAGGGGGTAATATGGATAGTCTCTGTCCCT |
| | the rice mitochondrial | GTATTCACATTCCACCTTCAACAAAGTGTT |
| | DNA (accession number | GATTTCCCGTAAAGCTAACTGTAGTCCTTT |
| | NC_011033) | AAGTAAGTAGATATCTTAGGCAAGTTAGCA |
| | | ATCTCGTTATATTACCAAGGCCTTCCCTTC |
| | | TATTGTAGAAAGAGTTCTCAGCCATCTAAT |
| | | TGCAGTGCCAGTTGCCAGCTATCCAGTTTC |
| | | ATTTGAAGTTGCTGGGGGTCCAAACGAGCT |
| | | AGTTGCTTTTATTCGTCCTATAAGTCCTTC |
| | | CACAAGCGAGTCAATAGGGTGCTGGCTAGT |
| | | TGTAGTTGTTGGCGTGCCTTTCCTTTCATC |
| | | TTGAATATTAATAAATATTTGGATAAATTA |
| | | CTTTAGAATAAGAAGTTCATGTTTTATAGA |
| | | CTAGTAAGATAACTAACTAGTTAAGTAATA |
| | | CGAATCCATACTAGGAAAATGAAAATGTGA |
| | | GTCCTAGGCACTGGAATTGGTTCTCTTCTC |
| | | CCTAATCCCTATAAGCCAGAAAGGGTAATA |
| | | GGCTTCAGTGTAAGCATTTCCTTCAAGCAA |
| | | GTCATCTCAAGTTTTAAATTCTAGAGAATA |
| | | GCTCCGATCAACCCATTTTAGTTTGGTTCT |
| | | GCAATTCATTCGCATAAATGAAAAAAAAAG |
| | | CGAGATGTGCACGAAAGAAGATCATAGTTC |
| | | AGCTTTAAAATGGTGGTGTCCCTGTGTTAG |
| | | TAAGTGGTTGAAATAGCTCATGGGAGTGTC |
| | | TGCCCCATTCGATAATGGCATTTATGATCT |
| | | AGTGGAGTGAGTGATTGTGTGGTGTTCAGT |
| | | CTAAGGCTTTTTGAAAAGCGGATTTCTCCC |
| | | TTCTCTCATCCATCGTCTTTGTTAAAGT |
| 31 | Nucleotide sequence of | ATAGACCTTTTTATTTTTCGTCATTCGATC |
| | the terminator region of | ACGAAAACAGGGATTCTGGAACGGCCAAGA |
| | the rice ATPI gene | ATCCCAGCGGTTGTTCGGGTCGAAAAACCG |
| | | AGAACAAGACATGCCACAAAGTGGCAGATG |
| | | AAGGCAGGGGGGAGAGCCTAGTCCTCAACC |
| | | TCTTCTTCCCCAAAAGGTAGTTATGAACGT |
| | | GCCAAACTTATTGGATTTATTCTTGGAATG |
| | | CTCATAACCACCTTTACTCTTTTTTTCATT |
| | | CTTTACTCAGAGGAAGCCATGCCGTTTGGA |
| | | GAAGAGCACCAAGTGGGGGGAGTGTGGAGT |
| | | CCCCGAAAGAGGAGCTTTCTAAAGGCAAGA |
| | | GAAAAGCTCCGATGGAGCCCTTGGAGCTAC |
| | | AGGGACCACCAACCCTTCGCAGCTTGGACG |
| | | ATTTGATTCTTGTGCCACTCAGCCCTGAGG |
| | | AGGGCGCCTGCTCGACCCAGTCAGGTACTA |
| | | CTCCGCCGCCGGCCCCGAGTAACTCTGCGG |
| | | GGGTCGGTGCAGCCCTTTCTTCTATTCCGG |
| | | AGTGCATAAACAAGGATCCTCAAAAAGCGA |
| | | AATCATTTCGTTAATGGCATTTCAGAAATG |
| | | AGTCATAGGCGCCTGTACAATGACAGAATA |
| | | GAGAGTCCTTTTTTTCCAGAATGAATCATT |
| | | CTATTCAAATCTCACAAGTTCTCTTTACGC |
| | | GTCTTCTAGGGGCATTGTTGAACGCAATCT |
| | | GCAGGAACAAGAAATGATTCTTTCTTATTT |
| | | TGAAACAGAATTCAAAATAAAGGAGGATTT |
| | | AATTCGGTTGCTTTATGAAGGCCGACGCCG |
| | | TGCCGATAGATACGTTATACACGAAACGAA |
| | | AATAGCCAGTACGGTGGACGCGTTCCTTTC |
| | | CAAAAAGGGATTATCAGGAGCTCCCAGTGC |
| | | CG |
| 32 | Nucleotide sequence of | AAGGTCTCGAATTCAATGGGCCCTTAGCTC |
| | a multiple-cloning site | GAG |
| | for the 5′ end of an | |
| | expression cassette | |
| 33 | Nucleotide sequence of | GAGCTCGGTACCAAAGGCGCGCCAAACAAT |
| | a multiple-cloning site | TGTCGAC |
| | for the 3′ end of an | |
| | expression cassette | |
| 34 | Nucleotide sequence of | TTCAATGGGCCCCTGTTTTCGAAGCTATAG |
| | pNAP76, a pBR322- | CATAGGTTTAGTGAGGTTTAGGTACTTTGA |
| | derived plasmid DNA | CACCTAAACCGATTTGAAATGCGAAATATC |
| | containing an eGFP | GCATTTCTGTAAGCATAACTAGTATTGTCC |
| | expression cassette | CTCGCCGTGCACGCGCACGTGCACGCGCGG |
| | under the control of the | GTGATGTGCGCATGCGTATGCGCACGTGAT |
| | COBI promoter and | CTATTGGTGCGCGCATGCGCACGCGTTCTA |
| | terminator of rice | TTACGCGCGAGCGCACACGCGGATGCGTGT |
| | mitochondria and a B4 | GCGCGTGTATTCCCTCCCTTACTTGAATGA |
| | autonomous sequence of | TCCCCCCCTCAAGGGGTATCATCCCAGTAA |
| | rice mitochondria | GCTCGGGTCTTTCATAGGAAGGGAGCAGGC |
| | | CCCCGCTCCCTTCCGTCCATCAGTGATTTA |
| | | TTCAAAACCCGAAATCGAGTTGAGTTGAAT |
| | | CGGGTTCAAGTCAACTAGATGAAGGGTTTC |
| | | TTTGTAGTGAGGGAACGAGTTACTGAACAC |
| | | AGTGAAATATCACGTTTTGTCATGTCATGC |
| | | ACATGTGTCTTTCCCTCGATCCCGAACCTT |
| | | GACTGGACGTATAGGTATGCGGTATGCCAA |
| | | TGACAGTTATCGAAGCTGCCATCAGTTATG |
| | | CATTTATGGATTCGGGTCTAGTGAATAGGG |
| | | TATGCCTAAGCGCCCACCCGAGATTGGACT |
| | | CGAGGGTGGGTAAGACCCGGAGCGAGGTTG |
| | | TCCACGAGCGGAGGCCCCTCGAAGAGGCGA |
| | | GCCCGGAAACCACTCGTTTTTTTAGTACCC |
| | | AAAACCCTAGTGTTTTAAAGTGAGAGGGAT |
| | | TCTACCTTTGGGGAAGGTAGGATTCCTCGG |
| | | AGCAAAGGAAAACTAATGTTGAATGTTTTC |
| | | GTGGAAGTGAGATAAGTACTTCCTTGGGAA |
| | | GGGAGTACTTATCGGAGTAAAGGAAACTGC |
| | | GGAGGTTCTTGTTGTAAGGAGGCTAGTCCC |
| | | GTTGTTAGGAAGAGTTAGCGGTGTTTACAC |
| | | CGGTGTCACGTGTACGGGGATACGTGTATT |
| | | GAGAAAAAAGGCCGAGAAGGTCGAGGGGGT |
| | | CATCCCATTGGCCAGACTGAGCATCAAGCC |
| | | AGCCAAGAAGTAAAAGCTGAGAAGGAGTGA |
| | | CTCGCATGAGTCAACACTTACTACTCAGGT |
| | | CCGGTAGAGCAATCTCAAATTATCATATAG |
| | | AAATGTTAATGTTATGATTTCGGTATTGAT |
| | | CAAAAGGTGCTGGGACCTTAGGGCATACAT |
| | | TAGTGCCATGCCCTATTGCGGAACGGTCGT |
| | | ATCCTGGTAACCTAGCCCCCGTAAGAGCTC |
| | | TACCTAATCGTCGGGGTAGAAGGCTGTGCT |
| | | TATTCTCGGCAAATAGCTAAGTCGACACCC |
| | | CGAGGGAGCAACTCAACTCTTCGTAGATCA |
| | | AAACAAGTGTTCACTGGAAAGTGGATCAAA |
| | | GAAAAAAACTTCTTCGTTTCGTTGGAAAAA |
| | | CCGACGCCAATATCATATTGACTCTCTCTC |
| | | GTCCAATAAGAGTTTCCGAGAGTTACTTTA |
| | | TTCAAATTCTCTCCTTTCCAAAGCTCCACA |
| | | AGGCAGGCAAAAAGAGTAATAGGACAACAA |
| | | GCAATCTTGTCTTTCATTTATTTGGAGTTC |
| | | TTTCTTTGTTGAGATGGAAATCGACGTTCT |
| | | TTTGAAAAGGGCTAGGTAGTTTGCACGCAG |
| | | GCAAAACTTCTTCATGAAAGGTAATAAATA |
| | | GACTTTTTTTTCATGGGTTTCTTAATGACT |
| | | AGTCGTTCGTTTGAAGCCTTAAGAAACCGG |
| | | CAGTTTTTTTTCCGAATGACCTTATTTCGA |
| | | GAATCAACTAACCGACAAATCCGTAGCCCA |
| | | GGTGATTCGCTGCCTCCCTCTCGCCAAAAT |
| | | GGGATGAATCTTCTCATGCAGCTTTTTTCT |
| | | TGTTCAGGGCGCAGCGAAGCCAATTTCCAT |
| | | CAAGGCAAGGGGGTAAATAAGGGGGAAGAG |
| | | GAGTTGTCACGATAGAAAAGAGAAACTTTT |
| | | GACAGTTATACGATTCCAGAAGTGAGTAAG |
| | | GGAGAGGAGCTGTTCACCGGGGTGGTGCCT |
| | | ATCCTGGTCGAGCTGGATGGTGATGTAAAC |
| | | GGTCATAAATTCAGTGTGTCCGGTGAAGGT |
| | | GAAGGTGATGCCACCTATGGTAAGCTGACC |
| | | CTTAAGTTCATCTGTACCACCGGAAAGCTG |
| | | CCTGTGCCTTGGCCTACCCTCGTGACCACC |
| | | CTGACATATGGAGTGCAATGTTTCAGTCGT |
| | | TATCCTGATCATATGAAGCAACATGATTTC |
| | | TTTAAATCCGCCATGCCTGAAGGTTATGTC |
| | | CAAGAGCGTACCATATTCTTTAAAGATGAT |
| | | GGTAACTATAAGACCCGTGCCGAGGTGAAG |
| | | TTCGAGGGTGATACCCTGGTGAACCGTATT |
| | | GAGCTTAAGGGTATCGATTTCAAGGAGGAT |
| | | GGAAACATCCTGGGGCATAAGCTGGAGTAT |
| | | AACTATAACAGTCATAACGTCTATATCATG |
| | | GCCGATAAGCAAAAGAACGGTATCAAGGTG |
| | | AACTTCAAGATCCGTCATAATATCGAAGAT |
| | | GGAAGTGTGCAACTCGCCGATCATTATCAA |
| | | CAAAACACCCCTATCGGTGATGGTCCTGTG |
| | | CTGCTGCCTGATAACCATTATCTGAGTACC |
| | | CAATCCGCCCTGAGTAAAGATCCTAACGAG |
| | | AAGCGTGATCAAATGGTACTGCTTGAGTTC |
| | | GTTACCGCCGCCGGGATCACTCTCGGTATG |
| | | GATGAGCTGTATAAGTAATAGACGGATGAG |
| | | ACTGATCACACCTGATCAGTGATCAATTCT |
| | | GGCACAATGAATTTACGAGTTATTTTACAC |
| | | AATGAATTTACAAGCAGATGAGTTTGCAAC |
| | | GGTAGACCTATCTCCTGAAAAGAGTTCAGT |
| | | AAACAAGGGAACGAAGCGACCGATAACGTC |
| | | CCCTCGGGGAGGAGTGTTTTGGATCCGTAA |
| | | CCATGGCTTTGTCGACCGATGCCCTTGAGA |
| | | GCCTTCAACCCAGTCAGCTCCTTCCGGTGG |
| | | GCGCGGGGCATGACTATCGTCGCCGCACTT |
| | | ATGACTGTCTTCTTTATCATGCAACTCGTA |
| | | GGACAGGTGCCGGCAGCGCTCTGGGTCATT |
| | | TTCGGCGAGGACCGCTTTCGCTGGAGCGCG |
| | | ACGATGATCGGCCTGTCGCTTGCGGTATTC |
| | | GGAATCTTGCACGCCCTCGCTCAAGCCTTC |
| | | GTCACTGGTCCCGCCACCAAACGTTTCGGC |
| | | GAGAAGCAGGCCATTATCGCCGGCATGGCG |
| | | GCCGACGCGCTGGGCTACGTCTTGCTGGCG |
| | | TTCGCGACGCGAGGCTGGATGGCCTTCCCC |
| | | ATTATGATTCTTCTCGCTTCCGGCGGCATC |
| | | GGGATGCCCGCGTTGCAGGCCATGCTGTCC |
| | | AGGCAGGTAGATGACGACCATCAGGGACAG |
| | | CTTCAAGGATCGCTCGCGGCTCTTACCAGC |
| | | CTAACTTCGATCACTGGACCGCTGATCGTC |
| | | ACGGCGATTTATGCCGCCTCGGCGAGCACA |
| | | TGGAACGGGTTGGCATGGATTGTAGGCGCC |
| | | GCCCTATACCTTGTCTGCCTCCCCGCGTTG |
| | | CGTCGCGGTGCATGGAGCCGGGCCACCTCG |
| | | ACCTGAATGGAAGCCGGCGGCACCTCGCTA |
| | | ACGGATTCACCACTCCAAGAATTGGAGCCA |
| | | ATCAATTCTTGCGGAGAACTGTGAATGCGC |
| | | AAACCAACCCTTGGCAGAACATATCCATCG |
| | | CGTCCGCCATCTCCAGCAGCCGCACGCGGC |
| | | GCATCTCGGGCAGCGTTGGGTCCTGGCCAC |
| | | GGGTGCGCATGATCGTGCTCCTGTCGTTGA |
| | | GGACCCGGCTAGGCTGGCGGGGTTGCCTTA |
| | | CTGGTTAGCAGAATGAATCACCGATACGCG |
| | | AGCGAACGTGAAGCGACTGCTGCTGCAAAA |
| | | CGTCTGCGACCTGAGCAACAACATGAATGG |
| | | TCTTCGGTTTCCGTGTTTCGTAAAGTCTGG |
| | | AAACGCGGAAGTCAGCGCCCTGCACCATTA |
| | | TGTTCCGGATCTGCATCGCAGGATGCTGCT |
| | | GGCTACCCTGTGGAACACCTACATCTGTAT |
| | | TAACGAAGCGCTGGCATTGACCCTGAGTGA |
| | | TTTTTCTCTGGTCCCGCCGCATCCATACCG |
| | | CCAGTTGTTTACCCTCACAACGTTCCAGTA |
| | | ACCGGGCATGTTCATCATCAGTAACCCGTA |
| | | TCGTGAGCATCCTCTCTCGTTTCATCGGTA |
| | | TCATTACCCCCATGAACAGAAATCCCCCTT |
| | | ACACGGAGGCATCAGTGACCAAACAGGAAA |
| | | AAACCGCCCTTAACATGGCCCGCTTTATCA |
| | | GAAGCCAGACATTAACGCTTCTGGAGAAAC |
| | | TCAACGAGCTGGACGCGGATGAACAGGCAG |
| | | ACATCTGTGAATCGCTTCACGACCACGCTG |
| | | ATGAGCTTTACCGCAGCTGCCTCGCGCGTT |
| | | TCGGTGATGACGGTGAAAACCTCTGACACA |
| | | TGCAGCTCCCGGAGACGGTCACAGCTTGTC |
| | | TGTAAGCGGATGCCGGGAGCAGACAAGCCC |
| | | GTCAGGGCGCGTCAGCGGGTGTTGGCGGGT |
| | | GTCGGGGCGCAGCCATGACCCAGTCACGTA |
| | | GCGATAGCGGAGTGTATACTGGCTTAACTA |
| | | TGCGGCATCAGAGCAGATTGTACTGAGAGT |
| | | GCACCATATGCGGTGTGAAATACCGCACAG |
| | | ATGCGTAAGGAGAAAATACCGCATCAGGCG |
| | | CTCTTCCGCTTCCTCGCTCACTGACTCGCT |
| | | GCGCTCGGTCGTTCGGCTGCGGCGAGCGGT |
| | | ATCAGCTCACTCAAAGGCGGTAATACGGTT |
| | | ATCCACAGAATCAGGGGATAACGCAGGAAA |
| | | GAACATGTGAGCAAAAGGCCAGCAAAAGGC |
| | | CAGGAACCGTAAAAAGGCCGCGTTGCTGGC |
| | | GTTTTTCCATAGGCTCCGCCCCCCTGACGA |
| | | GCATCACAAAAATCGACGCTCAAGTCAGAG |
| | | GTGGCGAAACCCGACAGGACTATAAAGATA |
| | | CCAGGCGTTTCCCCCTGGAAGCTCCCTCGT |
| | | GCGCTCTCCTGTTCCGACCCTGCCGCTTAC |
| | | CGGATACCTGTCCGCCTTTCTCCCTTCGGG |
| | | AAGCGTGGCGCTTTCTCATAGCTCACGCTG |
| | | TAGGTATCTCAGTTCGGTGTAGGTCGTTCG |
| | | CTCCAAGCTGGGCTGTGTGCACGAACCCCC |
| | | CGTTCAGCCCGACCGCTGCGCCTTATCCGG |
| | | TAACTATCGTCTTGAGTCCAACCCGGTAAG |
| | | ACACGACTTATCGCCACTGGCAGCAGCCAC |
| | | TGGTAACAGGATTAGCAGAGCGAGGTATGT |
| | | AGGCGGTGCTACAGAGTTCTTGAAGTGGTG |
| | | GCCTAACTACGGCTACACTAGAAGGACAGT |
| | | ATTTGGTATCTGCGCTCTGCTGAAGCCAGT |
| | | TACCTTCGGAAAAAGAGTTGGTAGCTCTTG |
| | | ATCCGGCAAACAAACCACCGCTGGTAGCGG |
| | | TGGTTTTTTTGTTTGCAAGCAGCAGATTAC |
| | | GCGCAGAAAAAAAGGATCTCAAGAAGATCC |
| | | TTTGATCTTTTCTACGGGGTCTGACGCTCA |
| | | GTGGAACGAAAACTCACGTTAAGGGATTTT |
| | | GGTCATGAGATTATCAAAAAGGATCTTCAC |
| | | CTAGATCCTTTTAAATTAAAAATGAAGTTT |
| | | TAAATCAATCTAAAGTATATATGAGTAAAC |
| | | TTGGTCTGACAGTTACCAATGCTTAATCAG |
| | | TGAGGCACCTATCTCAGCGATCTGTCTATT |
| | | TCGTTCATCCATAGTTGCCTGACTCCCCGT |
| | | CGTGTAGATAACTACGATACGGGAGGGCTT |
| | | ACCATCTGGCCCCAGTGCTGCAATGATACC |
| | | GCGAGACCCACGCTCACCGGCTCCAGATTT |
| | | ATCAGCAATAAACCAGCCAGCCGGAAGGGC |
| | | CGAGCGCAGAAGTGGTCCTGCAACTTTATC |
| | | CGCCTCCATCCAGTCTATTAATTGTTGCCG |
| | | GGAAGCTAGAGTAAGTAGTTCGCCAGTTAA |
| | | TAGTTTGCGCAACGTTGTTGCCATTGCTGC |
| | | AGGCATCGTGGTGTCACGCTCGTCGTTTGG |
| | | TATGGCTTCATTCAGCTCCGGTTCCCAACG |
| | | ATCAAGGCGAGTTACATGATCCCCCATGTT |
| | | GTGCAAAAAAGCGGTTAGCTCCTTCGGTCC |
| | | TCCGATCGTTGTCAGAAGTAAGTTGGCCGC |
| | | AGTGTTATCACTCATGGTTATGGCAGCACT |
| | | GCATAATTCTCTTACTGTCATGCCATCCGT |
| | | AAGATGCTTTTCTGTGACTGGTGAGTACTC |
| | | AACCAAGTCATTCTGAGAATAGTGTATGCG |
| | | GCGACCGAGTTGCTCTTGCCCGGCGTCAAC |
| | | ACGGGATAATACCGCGCCACATAGCAGAAC |
| | | TTTAAAAGTGCTCATCATTGGAAAACGTTC |
| | | TTCGGGGCGAAAACTCTCAAGGATCTTACC |
| | | GCTGTTGAGATCCAGTTCGATGTAACCCAC |
| | | TCGTGCACCCAACTGATCTTCAGCATCTTT |
| | | TACTTTCACCAGCGTTTCTGGGTGAGCAAA |
| | | AACAGGAAGGCAAAATGCCGCAAAAAAGGG |
| | | AATAAGGGCGACACGGAAATGTTGAATACT |
| | | CATACTCTTCCTTTTTCAATATTATTGAAG |
| | | CATTTATCAGGGTTATTGTCTCATGAGCGG |
| | | ATACATATTTGAATGTATTTAGAAAAATAA |
| | | ACAAATAGGGGTTCCGCGCACATTTCCCCG |
| | | AAAAGTGCCACCTGACGTCTAAGAAACCAT |
| | | TATTATCATGACATTAACCTATAAAAATAG |
| | | GCGTATCACGAGGCCCTTTCGTCTTCAAGA |
| | | A |
| 35 | Nucleotide sequence | ATGAAAAAGCCTGAACTCACCGCGACGTCT |
| | encoding a hygromycin | GTCGAGAAGTTTCTGATCGAAAAGTTCGAC |
| | phosphotransferase | AGCGTCTCCGACCTGATGCAGCTCTCGGAG |
| | (HPT) that confers | GGCGAAGAATCTCGTGCTTTCAGCTTCGAT |
| | resistance to the | GTAGGAGGGCGTGGATATGTCCTGCGGGTA |
| | antibiotic hygromycin | AATAGCTGCGCCGATGGTTTCTACAAAGAT |
| | | CGTTATGTTTATCGGCACTTTGCATCGGCC |
| | | GCGCTCCCGATTCCGGAAGTGCTTGACATT |
| | | GGGGAGTTTAGCGAGAGCCTGACCTATTGC |
| | | ATCTCCCGCCGTGCACAGGGTGTCACGTTG |
| | | CAAGACCTGCCTGAAACCGAACTGCCCGCT |
| | | GTTCTACAACCGGTCGCGGAGGCTATGGAT |
| | | GCGATCGCTGCGGCCGATCTTAGCCAGACG |
| | | AGCGGGTTCGGCCCATTCGGACCGCAAGGA |
| | | ATCGGTCAATACACTACATGGCGTGATTTC |
| | | ATATGCGCGATTGCTGATCCCCATGTGTAT |
| | | CACTGGCAAACTGTGATGGACGACACCGTC |
| | | AGTGCGTCCGTCGCGCAGGCTCTCGATGAG |
| | | CTGATGCTTTGGGCCGAGGACTGCCCCGAA |
| | | GTCCGGCACCTCGTGCACGCGGATTTCGGC |
| | | TCCAACAATGTCCTGACGGACAATGGCCGC |
| | | ATAACAGCGGTCATTGACTGGAGCGAGGCG |
| | | ATGTTCGGGGATTCCCAATACGAGGTCGCC |
| | | AACATCTTCTTCTGGAGGCCGTGGTTGGCT |
| | | TGTATGGAGCAGCAGACGCGCTACTTCGAG |
| | | CGGAGGCATCCGGAGCTTGCAGGATCGCCA |
| | | CGACTCCGGGCGTATATGCTCCGCATTGGT |
| | | CTTGACCAACTCTATCAGAGCTTGGTTGAC |
| | | GGCAATTTCGATGATGCAGCTTGGGCGCAG |
| | | GGTCGATGCGACGCAATCGTCCGATCCGGA |
| | | GCCGGGACTGTCGGGCGTACACAAATCGCC |
| | | CGCAGAAGCGCGGCCGTCTGGACCGATGGC |
| | | TGTGTAGAAGTACTCGCCGATAGTGGAAAC |
| | | CGACGCCCCAGCACTCGTCCGAGGGCAAAG |
| | | AAATAG |
| 36 | Nucleotide sequence of | GATCGTTCAAACATTTGGCAATAAAGTTTC |
| | the CaMV 35S promoter | TTAAGATTGAATCCTGTTGCCGGTCTTGCG |
| | sequence | ATGATTATCATATAATTTCTGTTGAATTAC |
| | | GTTAAGCATGTAATAATTAACATGTAATGC |
| | | ATGACGTTATTTATGAGATGGGTTTTTATG |
| | | ATTAGAGTCCCGCAATTATACATTTAATAC |
| | | GCGATAGAAAACAAAATATAGCGCGCAAAC |
| | | TAGGATAAATTATCGCGCGCGGTGTCATCT |
| | | ATGTTACTAGATCCCGGCGCGCCAACATGG |
| | | TGGAGCACGACACTCTCGTCTACTCCAAGA |
| | | ATATCAAAGATACAGTCTCAGAAGACCAAA |
| | | GGGCTATTGAGACTTTTCAACAAAGGGTAA |
| | | TATCGGGAAACCTCCTCGGATTCCATTGCC |
| | | CAGCTATCTGTCACTTCATCAAAAGGACAG |
| | | TAGAAAAGGAAGGTGGCACCTACAAATGCC |
| | | ATCATTGCGATAAAGGAAAGGCTATCGTTC |
| | | AAGATGCCTCTGCCGACAGTGGTCCCAAAG |
| | | ATGGACCCCCACCCACGAGGAGCATCGTGG |
| | | AAAAAGAAGACGTTCCAACCACGTCTTCAA |
| | | AGCAAGTGGATTGATGTGATAACATGGTGG |
| | | AGCACGACACTCTCGTCTACTCCAAGAATA |
| | | TCAAAGATACAGTCTCAGAAGACCAAAGGG |
| | | CTATTGAGACTTTTCAACAAAGGGTAATAT |
| | | CGGGAAACCTCCTCGGATTCCATTGCCCAG |
| | | CTATCTGTCACTTCATCAAAAGGACAGTAG |
| | | AAAAGGAAGGTGGCACCTACAAATGCCATC |
| | | ATTGCGATAAAGGAAAGGCTATCGTTCAAG |
| | | ATGCCTCTGCCGACAGTGGTCCCAAAGATG |
| | | GACCCCCACCCACGAGGAGCATCGTGGAAA |
| | | AAGAAGACGTTCCAACCACGTCTTCAAAGC |
| | | AAGTGGATTGATGTGATATCTCCACTGACG |
| | | TAAGGGATGACGCACAATCCCACTATCCTT |
| | | CGCAAGACCTTCCTCTATATAAGGAAGTTC |
| | | ATTTCATTTGGAGAGGACACGCTGAAATCA |
| | | CCAGTCTCTCTCTACAAATCTATCTCTCTC |
| | | GAGCTTTCGCAGATCCCGGGGGGCAATGAG |
| | | AT |
| 37 | Nucleotide sequence of | GATCTGTCGATCGACAAGCTCGAGTTTCTC |
| | a CaMV 3′ UTR that | CATAATAATGTGTGAGTAGTTCCCAGATAA |
| | carries a poly(A) signal | GGGAATTAGGGTTCCTATAGGGTTTCGCTC |
| | | ATGTGTTGAGCATATAAGAAACCCTTAGTA |
| | | TGTATTTGTATTTGTAAAATACTTCTATCA |
| | | ATAAAATTTCTAATTCCTAAAACCAAAATC |
| | | CAGTACTAAAATCCAGATCCCCCGAATTA |
| 38 | Nucleotide sequence of | ATGTTTAAACAAGCTTCTCGTCTCCTCTCC |
| | the mitochondrial | CGATCTGTCGCCGCCGCATCTTCCAAATCG |
| | targeting sequence | GTGACGACTCGTGCCTTTTCAACGGAACTT |
| | (MTS) of the | CCATCGACGCTCGATTCC |
| | Arabidopsis gene | |
| | At5g47030 | |
| 39 | Nucleotide sequence of | CTGCAGTGCAGCGTGACCCGGTCGTGCCCC |
| | the maize ubiquitin 1 | TCTCTAGAGATAATGAGCATTGCATGTCTA |
| | promoter with the first | AGTTATAAAAAATTACCACATATTTTTTTT |
| | intron | GTCACACTTGTTTGAAGTGCAGTTTATCTA |
| | | TCTTTATACATATATTTAAACTTTACTCTA |
| | | CGAATAATATAATCTATAGTACTACAATAA |
| | | TATCAGTGTTTTAGAGAATCATATAAATGA |
| | | ACAGTTAGACATGGTCTAAAGGACAATTGA |
| | | GTATTTTGACAACAGGACTCTACAGTTTTA |
| | | TCTTTTTAGTGTGCATGTGTTCTCCTTTTT |
| | | TTTTGCAAATAGCTTCACCTATATAATACT |
| | | TCATCCATTTTATTAGTACATCCATTTAGG |
| | | GTTTAGGGTTAATGGTTTTTATAGACTAAT |
| | | TTTTTTAGTACATCTATTTTATTCTATTTT |
| | | AGCCTCTAAATTAAGAAAACTAAAACTCTA |
| | | TTTTAGTTTTTTTATTTAATAATTTAGATA |
| | | TAAAATAGAATAAAATAAAGTGACTAAAAA |
| | | TTAAACAAATACCCTTTAAGAAATTAAAAA |
| | | AACTAAGGAAACATTTTTCTTGTTTCGAGT |
| | | AGATAATGCCAGCCTGTTAAACGCCGTCGA |
| | | CGAGTCTAACGGACACCAACCAGCGAACCA |
| | | GCAGCGTCGCGTCGGGCCAAGCGAAGCAGA |
| | | CGGCACGGCATCTCTGTCGCTGCCTCTGGA |
| | | CCCCTCTCGAGAGTTCCGCTCCACCGTTGG |
| | | ACTTGCTCCGCTGTCGGCATCCAGAAATTG |
| | | CGTGGCGGAGCGGCAGACGTGAGCCGGCAC |
| | | GGCAGGCGGCCTCCTCCTCCTCTCACGGCA |
| | | CCGGCAGCTACGGGGGATTCCTTTCCCACC |
| | | GCTCCTTCGCTTTCCCTTCCTCGCCCGCCG |
| | | TAATAAATAGACACCCCCTCCACACCCTCT |
| | | TTCCCCAACCTCGTGTTGTTCGGAGCGCAC |
| | | ACACACACAACCAGATCTCCCCCAAATCCA |
| | | CCCGTCGGCACCTCCGCTTCAAGGTACGCC |
| | | GCTCGTCCTCCCCCCCCCCCCCTCTCTACC |
| | | TTCTCTAGATCGGCGTTCCGGTCCATGGTT |
| | | AGGGCCCGGTAGTTCTACTTCTGTTCATGT |
| | | TTGTGTTAGATCCGTGTTTGTGTTAGATCC |
| | | GTGCTGCTAGCGTTCGTACACGGATGCGAC |
| | | CTGTACGTCAGACACGTTCTGATTGCTAAC |
| | | TTGCCAGTGTTTCTCTTTGGGGAATCCTGG |
| | | GATGGCTCTAGCCGTTCCGCAGACGGGATC |
| | | GATTTCATGATTTTTTTTGTTTCGTTGCAT |
| | | AGGGTTTGGTTTGCCCTTTTCCTTTATTTC |
| | | AATATATGCCGTGCACTTGTTTGTCGGGTC |
| | | ATCTTTTCATGCTTTTTTTTGTCTTGGTTG |
| | | TGATGATGTGGTCTGGTTGGGCGGTCGTTC |
| | | TAGATCGGAGTAGAATTAATTCTGTTTCAA |
| | | ACTACCTGGTGGATTTATTAATTTTGGATC |
| | | TGTATGTGTGTGCCATACATATTCATAGTT |
| | | ACGAATTGAAGATGATGGATGGAAATATCG |
| | | ATCTAGGATAGGTATACATGTTGATGCGGG |
| | | TTTTACTGATGCATATACAGAGATGCTTTT |
| | | TGTTCGCTTGGTTGTGATGATGTGGTGTGG |
| | | TTGGGCGGTCGTTCATTCGTTCTAGATCGG |
| | | AGTAGAATACTGTTTCAAACTACCTGGTGT |
| | | ATTTATTAATTTTGGAACTGTATGTGTGTG |
| | | TCATACATCTTCATAGTTACGAGTTTAAGA |
| | | TGGATGGAAATATCGATCTAGGATAGGTAT |
| | | ACATGTTGATGTGGGTTTTACTGATGCATA |
| | | TACATGATGGCATATGCAGCATCTATTCAT |
| | | ATGCTCTAACCTTGAGTACCTATCTATTAT |
| | | AATAAACAAGTATGTTTTATAATTATTTTG |
| | | ATCTTGATATACTTGGATGATGGCATATGC |
| | | AGCAGCTATATGTGGATTTTTTTAGCCCTG |
| | | CCTTCATACGCTATTTATTTGCTTGGTACT |
| | | GTTTCTTTTGTCGATGCTCACCCTGTTGTT |
| | | TGGTGTTACTTCTGCAGGTCGACTCTAGAG |
| | | GATCC |
| 40 | Nucleotide sequence of | GATCGTTCAAACATTTGGCAATAAAGTTTC |
| | the Nos terminator | TTAAGATTGAATCCTGTTGCCGGTCTTGCG |
| | | ATGATTATCATATAATTTCTGTTGAATTAC |
| | | GTTAAGCATGTAATAATTAACATGTAATGC |
| | | ATGACGTTATTTATGAGATGGGTTTTTATG |
| | | ATTAGAGTCCCGCAATTATACATTTAATAC |
| | | GCGATAGAAAACAAAATATAGCGCGCAAAC |
| | | TAGGATAAATTATCGCGCGCGGTGTCATCT |
| | | ATGTTACTAGATC |
| 41 | Nucleotide sequence of | CTGCAGTGCAGCGTGACCCGGTCGTGCCCC |
| | the entire expression | TCTCTAGAGATAATGAGCATTGCATGTCTA |
| | cassette encoding the | AGTTATAAAAAATTACCACATATTTTTTTT |
| | MTS-T7 RNA | GTCACACTTGTTTGAAGTGCAGTTTATCTA |
| | polymerase fusion | TCTTTATACATATATTTAAACTTTACTCTA |
| | protein | CGAATAATATAATCTATAGTACTACAATAA |
| | | TATCAGTGTTTTAGAGAATCATATAAATGA |
| | | ACAGTTAGACATGGTCTAAAGGACAATTGA |
| | | GTATTTTGACAACAGGACTCTACAGTTTTA |
| | | TCTTTTTAGTGTGCATGTGTTCTCCTTTTT |
| | | TTTTGCAAATAGCTTCACCTATATAATACT |
| | | TCATCCATTTTATTAGTACATCCATTTAGG |
| | | GTTTAGGGTTAATGGTTTTTATAGACTAAT |
| | | TTTTTTAGTACATCTATTTTATTCTATTTT |
| | | AGCCTCTAAATTAAGAAAACTAAAACTCTA |
| | | TTTTAGTTTTTTTATTTAATAATTTAGATA |
| | | TAAAATAGAATAAAATAAAGTGACTAAAAA |
| | | TTAAACAAATACCCTTTAAGAAATTAAAAA |
| | | AACTAAGGAAACATTTTTCTTGTTTCGAGT |
| | | AGATAATGCCAGCCTGTTAAACGCCGTCGA |
| | | CGAGTCTAACGGACACCAACCAGCGAACCA |
| | | GCAGCGTCGCGTCGGGCCAAGCGAAGCAGA |
| | | CGGCACGGCATCTCTGTCGCTGCCTCTGGA |
| | | CCCCTCTCGAGAGTTCCGCTCCACCGTTGG |
| | | ACTTGCTCCGCTGTCGGCATCCAGAAATTG |
| | | CGTGGCGGAGCGGCAGACGTGAGCCGGCAC |
| | | GGCAGGCGGCCTCCTCCTCCTCTCACGGCA |
| | | CCGGCAGCTACGGGGGATTCCTTTCCCACC |
| | | GCTCCTTCGCTTTCCCTTCCTCGCCCGCCG |
| | | TAATAAATAGACACCCCCTCCACACCCTCT |
| | | TTCCCCAACCTCGTGTTGTTCGGAGCGCAC |
| | | ACACACACAACCAGATCTCCCCCAAATCCA |
| | | CCCGTCGGCACCTCCGCTTCAAGGTACGCC |
| | | GCTCGTCCTCCCCCCCCCCCCCTCTCTACC |
| | | TTCTCTAGATCGGCGTTCCGGTCCATGGTT |
| | | AGGGCCCGGTAGTTCTACTTCTGTTCATGT |
| | | TTGTGTTAGATCCGTGTTTGTGTTAGATCC |
| | | GTGCTGCTAGCGTTCGTACACGGATGCGAC |
| | | CTGTACGTCAGACACGTTCTGATTGCTAAC |
| | | TTGCCAGTGTTTCTCTTTGGGGAATCCTGG |
| | | GATGGCTCTAGCCGTTCCGCAGACGGGATC |
| | | GATTTCATGATTTTTTTTGTTTCGTTGCAT |
| | | AGGGTTTGGTTTGCCCTTTTCCTTTATTTC |
| | | AATATATGCCGTGCACTTGTTTGTCGGGTC |
| | | ATCTTTTCATGCTTTTTTTTGTCTTGGTTG |
| | | TGATGATGTGGTCTGGTTGGGCGGTCGTTC |
| | | TAGATCGGAGTAGAATTAATTCTGTTTCAA |
| | | ACTACCTGGTGGATTTATTAATTTTGGATC |
| | | TGTATGTGTGTGCCATACATATTCATAGTT |
| | | ACGAATTGAAGATGATGGATGGAAATATCG |
| | | ATCTAGGATAGGTATACATGTTGATGCGGG |
| | | TTTTACTGATGCATATACAGAGATGCTTTT |
| | | TGTTCGCTTGGTTGTGATGATGTGGTGTGG |
| | | TTGGGCGGTCGTTCATTCGTTCTAGATCGG |
| | | AGTAGAATACTGTTTCAAACTACCTGGTGT |
| | | ATTTATTAATTTTGGAACTGTATGTGTGTG |
| | | TCATACATCTTCATAGTTACGAGTTTAAGA |
| | | TGGATGGAAATATCGATCTAGGATAGGTAT |
| | | ACATGTTGATGTGGGTTTTACTGATGCATA |
| | | TACATGATGGCATATGCAGCATCTATTCAT |
| | | ATGCTCTAACCTTGAGTACCTATCTATTAT |
| | | AATAAACAAGTATGTTTTATAATTATTTTG |
| | | ATCTTGATATACTTGGATGATGGCATATGC |
| | | AGCAGCTATATGTGGATTTTTTTAGCCCTG |
| | | CCTTCATACGCTATTTATTTGCTTGGTACT |
| | | GTTTCTTTTGTCGATGCTCACCCTGTTGTT |
| | | TGGTGTTACTTCTGCAGGTCGACTCTAGAG |
| | | GATCCATGTTTAAACAAGCTTCTCGTCTCC |
| | | TCTCCCGATCTGTCGCCGCCGCATCTTCCA |
| | | AATCGGTGACGACTCGTGCCTTTTCAACGG |
| | | AACTTCCATCGACGCTCGATTCCAACACGA |
| | | TTAACATCGCTAAGAACGACTTCTCTGACA |
| | | TCGAACTGGCTGCTATCCCGTTCAACACTC |
| | | TGGCTGACCATTACGGTGAGCGTTTAGCTC |
| | | GCGAACAGTTGGCCCTTGAGCATGAGTCTT |
| | | ACGAGATGGGTGAAGCACGCTTCCGCAAGA |
| | | TGTTTGAGCGTCAACTTAAAGCTGGTGAGG |
| | | TTGCGGATAACGCTGCCGCCAAGCCTCTCA |
| | | TCACTACCCTACTCCCTAAGATGATTGCAC |
| | | GCATCAACGACTGGTTTGAGGAAGTGAAAG |
| | | CTAAGCGCGGCAAGCGCCCGACAGCCTTCC |
| | | AGTTCCTGCAAGAAATCAAGCCGGAAGCCG |
| | | TAGCGTACATCACCATTAAGACCACTCTGG |
| | | CTTGCCTAACCAGTGCTGACAATACAACCG |
| | | TTCAGGCTGTAGCAAGCGCAATCGGTCGGG |
| | | CCATTGAGGACGAGGCTCGCTTCGGTCGTA |
| | | TCCGTGACCTTGAAGCTAAGCACTTCAAGA |
| | | AAAACGTTGAGGAACAACTCAACAAGCGCG |
| | | TAGGGCACGTCTACAAGAAAGCATTTATGC |
| | | AAGTTGTCGAGGCTGACATGCTCTCTAAGG |
| | | GTCTACTCGGTGGCGAGGCGTGGTCTTCGT |
| | | GGCATAAGGAAGACTCTATTCATGTAGGAG |
| | | TACGCTGCATCGAGATGCTCATTGAGTCAA |
| | | CCGGAATGGTTAGCTTACACCGCCAAAATG |
| | | CTGGCGTAGTAGGTCAAGACTCTGAGACTA |
| | | TCGAACTCGCACCTGAATACGCTGAGGCTA |
| | | TCGCAACCCGTGCAGGTGCGCTGGCTGGCA |
| | | TCTCTCCGATGTTCCAACCTTGCGTAGTTC |
| | | CTCCTAAGCCGTGGACTGGCATTACTGGTG |
| | | GTGGCTATTGGGCTAACGGTCGTCGTCCTC |
| | | TGGCGCTGGTGCGTACTCACAGTAAGAAAG |
| | | CACTGATGCGCTACGAAGACGTTTACATGC |
| | | CTGAGGTGTACAAAGCGATTAACATTGCGC |
| | | AAAACACCGCATGGAAAATCAACAAGAAAG |
| | | TCCTAGCGGTCGCCAACGTAATCACCAAGT |
| | | GGAAGCATTGTCCGGTCGAGGACATCCCTG |
| | | CGATTGAGCGTGAAGAACTCCCGATGAAAC |
| | | CGGAAGACATCGACATGAATCCTGAGGCTC |
| | | TCACCGCGTGGAAACGTGCTGCCGCTGCTG |
| | | TGTACCGCAAGGACAAGGCTCGCAAGTCTC |
| | | GCCGTATCAGCCTTGAGTTCATGCTTGAGC |
| | | AAGCCAATAAGTTTGCTAACCATAAGGCCA |
| | | TCTGGTTCCCTTACAACATGGACTGGCGCG |
| | | GTCGTGTTTACGCTGTGTCAATGTTCAACC |
| | | CGCAAGGTAACGATATGACCAAAGGACTGC |
| | | TTACGCTGGCGAAAGGTAAACCAATCGGTA |
| | | AGGAAGGTTACTACTGGCTGAAAATCCACG |
| | | GTGCAAACTGTGCGGGTGTCGATAAGGTTC |
| | | CGTTCCCTGAGCGCATCAAGTTCATTGAGG |
| | | AAAACCACGAGAACATCATGGCTTGCGCTA |
| | | AGTCTCCACTGGAGAACACTTGGTGGGCTG |
| | | AGCAAGATTCTCCGTTCTGCTTCCTTGCGT |
| | | TCTGCTTTGAGTACGCTGGGGTACAGCACC |
| | | ACGGCCTGAGCTATAACTGCTCCCTTCCGC |
| | | TGGCGTTTGACGGGTCTTGCTCTGGCATCC |
| | | AGCACTTCTCCGCGATGCTCCGAGATGAGG |
| | | TAGGTGGTCGCGCGGTTAACTTGCTTCCTA |
| | | GTGAAACCGTTCAGGACATCTACGGGATTG |
| | | TTGCTAAGAAAGTCAACGAGATTCTACAAG |
| | | CAGACGCAATCAATGGGACCGATAACGAAG |
| | | TAGTTACCGTGACCGATGAGAACACTGGTG |
| | | AAATCTCTGAGAAAGTCAAGCTGGGCACTA |
| | | AGGCACTGGCTGGTCAATGGCTGGCTTACG |
| | | GTGTTACTCGCAGTGTGACTAAGCGTTCAG |
| | | TCATGACGCTGGCTTACGGGTCCAAAGAGT |
| | | TCGGCTTCCGTCAACAAGTGCTGGAAGATA |
| | | CCATTCAGCCAGCTATTGATTCCGGCAAGG |
| | | GTCTGATGTTCACTCAGCCGAATCAGGCTG |
| | | CTGGATACATGGCTAAGCTGATTTGGGAAT |
| | | CTGTGAGCGTGACGGTGGTAGCTGCGGTTG |
| | | AAGCAATGAACTGGCTTAAGTCTGCTGCTA |
| | | AGCTGCTGGCTGCTGAGGTCAAAGATAAGA |
| | | AGACTGGAGAGATTCTTCGCAAGCGTTGCG |
| | | CTGTGCATTGGGTAACTCCTGATGGTTTCC |
| | | CTGTGTGGCAGGAATACAAGAAGCCTATTC |
| | | AGACGCGCTTGAACCTGATGTTCCTCGGTC |
| | | AGTTCCGCTTACAGCCTACCATTAACACCA |
| | | ACAAAGATAGCGAGATTGATGCACACAAAC |
| | | AGGAGTCTGGTATCGCTCCTAACTTTGTAC |
| | | ACAGCCAAGACGGTAGCCACCTTCGTAAGA |
| | | CTGTAGTGTGGGCACACGAGAAGTACGGAA |
| | | TCGAATCTTTTGCACTGATTCACGACTCCT |
| | | TCGGTACCATTCCGGCTGACGCTGCGAACC |
| | | TGTTCAAAGCAGTGCGCGAAACTATGGTTG |
| | | ACACATATGAGTCTTGTGATGTACTGGCTG |
| | | ATTTCTACGACCAGTTCGCTGACCAGTTGC |
| | | ACGAGTCTCAATTGGACAAAATGCCAGCAC |
| | | TTCCGGCTAAAGGTAACTTGAACCTCCGTG |
| | | ACATCTTAGAGTCGGACTTCGCGTTCGCGT |
| | | AAGCATGCTGAAGCGGCCGGGTACCGAGCT |
| | | CGAATTTCCCCGATCGTTCAAACATTTGGC |
| | | AATAAAGTTTCTTAAGATTGAATCCTGTTG |
| | | CCGGTCTTGCGATGATTATCATATAATTTC |
| | | TGTTGAATTACGTTAAGCATGTAATAATTA |
| | | ACATGTAATGCATGACGTTATTTATGAGAT |
| | | GGGTTTTTATGATTAGAGTCCCGCAATTAT |
| | | ACATTTAATACGCGATAGAAAACAAAATAT |
| | | AGCGCGCAAACTAGGATAAATTATCGCGCG |
| | | CGGTGTCATCTATGTTACTAGATC |
| 42 | Nucleotide sequence of | TAATACGACTCACTATAG |
| | the promoter of the T7 | |
| | RNA | |
| 43 | Nucleotide sequence of | AACCCCTTGGGGCCTCTAAACGGGTCTTGA |
| | the terminator of the T7 | GGGGTTTTTTG |
| | RNA Polymerase | |
| 44 | Nucleotide sequence of | TCTGCTTGAAAGCCTGCAGAGTCCAATTTT |
| | the hybrid promoter in | GAGTATTTTCAGTTAGAATCTAGAGTCAGC |
| | which the T7 promoter | CTATTCAGTTCTTAGCCCTTAAGGGTAAGG |
| | is inserted upstream of | CAGGGGGTAATATGGATAGTCTCTGTCCCT |
| | the first transcription | GTATTCACATTCCACCTTCAACAAAGTGTT |
| | start site of the | GATTTCCCGTAAAGCTAACTGTAGTCCTTT |
| | mitochondrial ATP1 | AAGTAAGTAGATATCTTAGGCAAGTTAGCA |
| | gene | ATCTCGTTATATTACCAAGGCCTTCCCTTC |
| | | TATTGTAGAAAGAGTTCTCAGCCATCTAAT |
| | | TGCAGTGCCAGTTGCCAGCTATCCAGTTTC |
| | | ATTTGAAGTTGCTGGGGGTCCAAACGAGCT |
| | | AGTTGCTTTTATTCGTCCTATAAGTCCTTC |
| | | CACAAGCGAGTCAATAGGGTGCTGGCTAGT |
| | | TGTAGTTGTTGGCGTGCCTTTCCTTTCATC |
| | | TTGAATATTAATAAATATTTGGATAAATTA |
| | | CTTTAGAATAAGAAGTTCATGTTTTAATAC |
| | | GACTCACTATAGTAAGTAATACGAATCCAT |
| | | ACTAGGAAAATGAAAATGTGAGTCCTAGGC |
| | | ACTGGAATTGGTTCTCTTCTCCCTAATCCC |
| | | TATAAGCCAGAAAGGGTAATAGGCTTCAGT |
| | | GTAAGCATTTCCTTCAAGCAAGTCATCTCA |
| | | AGTTTTAAATTCTAGAGAATAGCTCCGATC |
| | | AACCCATTTTAGTTTGGTTCTGCAATTCAT |
| | | TCGCATAAATGAAAAAAAAAGCGAGATGTG |
| | | CACGAAAGAAGATCATAGTTCAGCTTTAAA |
| | | ATGGTGGTGTCCCTGTGTTAGTAAGTGGTT |
| | | GAAATAGCTCATGGGAGTGTCTGCCCCATT |
| | | CGATAATGGCATTTATGATCTAGTGGAGTG |
| | | AGTGATTGTGTGGTGTTCAGTCTAAGGCTT |
| | | TTTGAAAAGCGGATTTCTCCCTTCTCTCAT |
| | | CCATCGTCTTTGTTAAAGT |
| 45 | Nucleotide sequence of | TCTGCTTGAAAGCCTGCAGAGTCCAATTTT |
| | the hybrid promoter in | GAGTATTTTCAGTTAGAATCTAGAGTCAGC |
| | which the T7 promoter | CTATTCAGTTCTTAGCCCTTAAGGGTAAGG |
| | is inserted upstream of | CAGGGGGTAATATGGATAGTCTCTGTCCCT |
| | the third transcription | GTATTCACATTCCACCTTCAACAAAGTGTT |
| | start site of the | GATTTCCCGTAAAGCTAACTGTAGTCCTTT |
| | mitochondrial ATP1 | AAGTAAGTAGATATCTTAGGCAAGTTAGCA |
| | gene | ATCTCGTTATATTACCAAGGCCTTCCCTTC |
| | | TATTGTAGAAAGAGTTCTCAGCCATCTAAT |
| | | TGCAGTGCCAGTTGCCAGCTATCCAGTTTC |
| | | ATTTGAAGTTGCTGGGGGTCCAAACGAGCT |
| | | AGTTGCTTTTATTCGTCCTATAAGTCCTTC |
| | | CACAAGCGAGTCAATAGGGTGCTGGCTAGT |
| | | TGTAGTTGTTGGCGTGCCTTTCCTTTCATC |
| | | TTGAATATTAATAAATATTTGGATAAATTA |
| | | CTTTAGAATAAGAAGTTCATGTTTTATAGA |
| | | CTAGTAAGATAACTAACTAGTTAAGTAATA |
| | | CGAATCCATACTAGGAAAATGAAAATGTGA |
| | | GTCCTAGGCACTGGAATTGGTTCTCTTCTC |
| | | CCTAATCCCTATAAGCCAGAAAGGGTAATA |
| | | GGCTTCAGTGTAAGCATTTCCTTCAAGCAA |
| | | GTCATCTCAAGTTTTAAATTCTAGAGAATA |
| | | GCTCCGATCAACCCATTTTAGTTTGGTTCT |
| | | GCAATTCATTCGCATAAATGAAAAAAAAAG |
| | | CGAGATGTGCACGAAAGAAGATCATAGTTC |
| | | AGCTTTAAAATGGTGGTGTCCCTGTGTTAG |
| | | TAAGTGGTTGAAATAGCTCATGGGAGTGTC |
| | | TGCCCCATTCGATAATGGCATAATACGACT |
| | | CACTATAGTTTATGATCTAGTGGAGTGAGT |
| | | GATTGTGTGGTGTTCAGTCTAAGGCTTTTT |
| | | GAAAAGCGGATTTCTCCCTTCTCTCATCCA |
| | | TCGTCTTTGTTAAAGT |
| 46 | Nucleotide sequence of | ACTTTTGACAGTTATAcGATTCCAGAA |
| | a mitochondrial RNA | |
| | editing site useful to | |
| | create an AUG | |
| | translation initiation | |
| | codon in an mRNA | |
| 47 | Nucleotide sequence of | ACTTTTGACAGTTATACGATTCCAGAACTG |
| | a PtxD CDS lacking an | CCTAAACTCGTTATAACTCACCGAGTACAT |
| | AUG initiation codon | GATGAGATCCTGCAACTGCTGGCGCCACAT |
| | but having the RNA | TGCGAGCTGATGACCAACCAAACCGACAGC |
| | editing site of SEQ ID | ACACTGACACGCGAGGAAATTCTGCGTCGA |
| | NO: 46 fused to the 5′ | TGTCGTGATGCTCAAGCGATGATGGCGTTC |
| | end | ATGCCCGATCGAGTCGATGCAGACTTTCTT |
| | | CAAGCCTGCCCTGAGCTGCGTGTAGTCGGC |
| | | TGCGCGCTCAAGGGCTTCGACAATTTCGAT |
| | | GTGGACGCCTGTACTGCCCGTGGGGTCTGG |
| | | CTGACCTTCGTGCCTGATCTGTTGACAGTC |
| | | CCAACTGCCGAGCTGGCGATCGGACTGGCG |
| | | GTGGGGCTGGGGCGACATCTGCGAGCAGCA |
| | | GATGCGTTCGTCCGTTCTGGCGAGTTCCAA |
| | | GGCTGGCAACCACAATTCTATGGCACAGGG |
| | | CTGGATAACGCTACAGTCGGCATCCTTGGC |
| | | ATGGGCGCCATCGGACTGGCCATGGCTGAT |
| | | CGATTGCAAGGATGGGGCGCGACCCTGCAA |
| | | TATCACGAGGCGAAGGCTCTGGATACACAA |
| | | ACCGAGCAACGACTCGGCCTGCGTCAAGTG |
| | | GCGTGCAGCGAACTCTTCGCCAGCTCGGAC |
| | | TTCATCCTGCTGGCGCTTCCCTTGAATGCC |
| | | GATACCCAACATCTGGTCAACGCCGAGCTG |
| | | CTTGCCCTCGTACGACCAGGCGCTCTGCTT |
| | | GTAAACCCCTGTCGTGGTTCGGTAGTGGAT |
| | | GAAGCCGCCGTGCTCGCGGCGCTTGAGCGA |
| | | GGCCAACTCGGCGGGTATGCGGCGGATGTA |
| | | TTCGAAATGGAAGACTGGGCTCGTGCGGAC |
| | | CGACCACGACTGATCGATCCTGCGCTGCTC |
| | | GCGCATCCTAATACACTGTTCACTCCACAT |
| | | ATAGGGTCGGCAGTGCGTGCGGTGCGTCTG |
| | | GAGATTGAACGTTGTGCAGCGCAAAACATC |
| | | ATCCAAGTATTGGCAGGTGCGCGTCCAATC |
| | | AACGCTGCGAACCGTCTGCCCAAGGCCGAG |
| | | CCTGCCGCATGTTGA |
| 48 | Amino acid sequence of | MKQKIVLTHWVHPEIIDYLQSVADVVPNMT |
| | a PtxD homolog from | RDTMSRAELLERAKDADALMVFMPDSIDDD |
| | Acinetobacter | FLASCPKLKIVSAALKGYDNFDVDACTRRG |
| | radioresistens SK82 | IWFSIVPDLLTIPTAELTIGLLLGLTRHLA |
| | (GenBank EET83888.1) | EGDRRIRTHGENGWRPELYGTGLTGRTLGI |
| | | IGMGAVGRAIAKRLSSFDMRVLYCDDIALN |
| | | QEQEKAWNARQVSLDELLSSSDFVVPMLPM |
| | | TPQTLHLLNAETIGTMRTGSYLINACRGSV |
| | | VDELAVAEALESGKLAGYAADVFELEEWIR |
| | | VDRPTAIPQELLTNTAQTFFTPHLGSAVDD |
| | | VRFEIEQLAANNILQALTGQRPSDAINNPI |
| | | LEGVN |
| 49 | Amino acid sequence of | MKPRIVTTHRIHPDTLALLETAAEVISNQS |
| | a PtxD homolog from | DSTMSREEVLLRTNDADGMMVFMPDSIDAD |
| | Alcaligenes faecalis | FLSACPNLKVIGAALKGYDNFDVEACTRHG |
| | (GenBank AAT12779.1) | IWFTIVPDLLTSPTAELTIGLLLSITRNML |
| | | QGDNYIRSRQFNGWTPRFYGTGLTGKTAGI |
| | | IGTGAVGRAVAKRLAAFDMQIQYTDPQPLP |
| | | QESERAWNASRTSLDQLLATSDFIIPMLPM |
| | | SSDTHHTINARALDRMKPGAYLVNACRGSI |
| | | VDERAVVHALRTGHLGGYAADVFEMEEWAR |
| | | PDRPHSIPDELLDPALPTFFTPHLGSAVKS |
| | | VRMEIEREAALSILEALQGRIPRGAVNHVG |
| | | AGR |
| 50 | Amino acid sequence of | MKQKIVLTHWVHPEIIDYLQSVADVVPNMT |
| | a PtxD homolog from | RDTMSRAELLERAKDADALMVFMPDSIDDD |
| | Cyanothece sp. | FLASCPKLKIVSAALKGYDNFDVDACTRRG |
| | CCY0110 (GenBank | IWFSIVPDLLTIPTAELTIGLLLGLTRHLA |
| | EAZ89932.1) | EGDRRIRTHGFNGWRPELYGTGLTGRTLGI |
| | | IGMGAVGRAIAKRLSSFDMRVLYCDDIALN |
| | | QEQEKAWNARQVSLDELLSSSDFVVPMLPM |
| | | TPQTLHLLNAETIGTMRTGSYLINACRGSV |
| | | VDELAVAEALESGKLAGYAADVFELEEWIR |
| | | VDRPTAIPQELLTNTAQTFFTPHLGSAVDD |
| | | VRFEIEQLAANNILQALTGQRPSDAINNPI |
| | | LEGVN |
| 51 | Amino acid sequence of | MKPKIVITSWVHPQTLDMLRPHCDVVANET |
| | a PtxD homolog from | RERLSREEIIKRCSDAVAVMTFMPDSIDDA |
| | Gallionella ferruginea | FLAECPQLRLVACALKGYDNYDVAACTRRG |
| | (GenBank EES62080.1) | VRITNVPDLLTIPTAELTVGLLIGLTRKVL |
| | | QGDRFVRSGQFTGWRPMLYGAGLTGRTLGI |
| | | IGMGAVGRAIAARLQGYEMELLYTDPQPLP |
| | | PELEARLGLRKVGLVQLLAESDYVVPMVPY |
| | | TQDTLHMINAASLSIMKPGAYLVNTCRGSV |
| | | VDEKAVADALDSGKLAGYAADVFELEEWMR |
| | | PDRPESISERLLSNTELTLFTPHIGSAVDT |
| | | VRLAIEMEAATNILQVLKGQIPQGAINHPL |
| | | DKVAV |
| 52 | Amino acid sequence of | MKPKIVITHWVHPEIVEMLSSVAEVVINDT |
| | a PtxD homolog from | LETLPREELLRRSKDADAVMAFMPDSVDDS |
| | Janthinobacterium sp. | FLAACPKLKIVFAALKGYDNFDVDACTKRG |
| | Marseille (GenBank | VWFGIVPDLLTVPTAELTVGLLLGLTRHVM |
| | ABR91484.1) | AGDDHVRSGTFHGWRPKLYGAGLAGSTIGI |
| | | IGMGRVGKAIAKRLSGFEMNAVYCDSVPLN |
| | | PVDEQAWNARQVSFDELLTCSDFVVPMLPM |
| | | TSDTFHLIDAHAISKMRRGSYLLNTSRGSV |
| | | VDENAVVEALNQGHLAGYAADVFEMEEWAR |
| | | PDRPLTVPQALLNNRTQTLFTPHVGSGVKK |
| | | VRLEIERYSAHSILQALAGQRPDGALNEPL |
| | | KASVAA |
| 53 | Amino acid sequence of | MLPKLVITHRVHDEILQLLAPHCELVTNQT |
| | a PtxD homolog from | DSTLTREEILRRCRDAQAMMAFMPDRVDAD |
| | Klebsiella pneumoniae | FLQACPELRVVGCALKGFDNFDVDACTARG |
| | (GenBank ABR80271.1) | VWLTFVPDLLTVPTAELAIGLAVGLGRHLR |
| | | AADAFVRSGEFQGWQPQFYGTGLDNATVGI |
| | | LGMGAIGLAMAERLQGWGATLQYHEAKALD |
| | | TQTEQRLGLRQVACSELFASSDFILLALPL |
| | | NADTQHLVNAELLALVRPGALLVNPCRGSV |
| | | VDEAAVLAALERGQLGGYAADVFEMEDWAR |
| | | ADRPRLIDPALLAHPNTLFTPHIGSAVRAV |
| | | RLEIERCAAQNIIQVLAGARPINAANRLPK |
| | | AEPAAC |
| 54 | Amino acid sequence of | MKPRVVITHRVHDSILASLEPHCELITNQS |
| | a PtxD homolog from | AVTLPPDSVRARAATADAMMAFMPDRVSEE |
| | Marinobacter algicola | FLVACPDLKVIGAALKGFDNFDVDACTRHG |
| | (GenBank | VWLTFVPDLLTVPTAELTVGLTIGLIRQIR |
| | EDM49754.1) | PADQFVRSGEFQGWQPQFYGLGIEGSTIGI |
| | | VGMGAIGKAVATRLQGWGARVLYSQPESLP |
| | | AAEEGALGLSRSELDDLLAESDIVILALAL |
| | | NEHTLHTLNADRLRQMKRGSFLINPCRGSV |
| | | VDEAAVLQSLTYGHLSGYAADVFEMEDWAR |
| | | PDRPQRIDPALLAHPNTLFTAHTGSAVRDV |
| | | RFAIELRAADNILQALRGHQPQDAVNSPLE |
| | | PKGTVC |
| 55 | Amino acid sequence of | MRFKVVVTNPVFPETREILEGLCDVDINPG |
| | a PtxD homolog from | PEPWPAAEVRARCSDADALLAFMTDCVDAG |
| | Methylobacterium | FLEACPRLKVVACALKGWDNFDVEACTRSG |
| | extorquens | IWLTAVPDLLTEPTAELAVGLAIGLCRNVV |
| | (NCBI | AGDRAVRAGFDGWRPRLYGSGLYGSVVGVA |
| | YP_003066079.1) | GMGKVGRAITRRLKGFGARELLYFDEQALP |
| | | ASAEAELGACRVSWDTLVGRSDVLILALPL |
| | | TPDTRHMLDAAALAAASPGLRIVNAGRGSV |
| | | VDEAAVAEALAEGRLGGYAADVFEMEDWAL |
| | | DDRPRRIAPGLLTVEDRTLFTPHLGSGVVD |
| | | TRRRIEAAAAHNLLDALKGLVPADSINHPE |
| | | SLRGFDGAN |
| 56 | Amino acid sequence of | MKPKVVITNWVHPEVIELLKPSCEVIANPS |
| | a PtxD homolog from | KEALSREEILQRAKDAEALMVFMPDTIDEA |
| | Nostoc sp. PCC 7120 | FLRECPKLKIIAAALKGYDNFDVAACTHRG |
| | (GenBank BAB77417.1) | IWFTIVPSLLSAPTAEITIGLLIGLGRQML |
| | | EGDRFIRTGKFTGWRPQFYSLGLANRTLGI |
| | | VGMGALGKAIAGRLAGFEMQLLYSDPVALP |
| | | PEQEATGNISRVPFETLIESSDFVVLVVPL |
| | | QPATLHLINANTLAKMKPGSFLINPCRGSV |
| | | VDEQAVCKALESGHLAGYAADVFEMEDWYR |
| | | SDRPHNIPQPLLENTKQTFFTPHIGSAVDE |
| | | LRHNIALEAAQNILQALQGQKPQGAVNYLR |
| | | ES |
| 57 | Amino acid sequence of | MNKQKVVLTHWVHPEIVEMLQEKTDVVANL |
| | a PtxD homolog from | SRKTFTRDELLERAAAADALMAFMPDCIDE |
| | Oxalobacter formigenes | DFLKACPKLKVIGAALKGYDNFDVKACTER |
| | (NCBI ZP_04579760.1) | GVWLTIAPDLLTIPTAELTVGLVLAITRNM |
| | | LEGDRHIRSGQFNGWRPELYGLGLHKRTAG |
| | | IIGMGFVGKAVAERLKGFGMDILYADQSPL |
| | | SQEEERELGLTRTGLPQLMHSSDVVIPLLP |
| | | LTEQTFHLFDKDILGQMKQGSYLVNACRGS |
| | | VVDEKAVVHSLKTGQLAGYAADVFEMEDWI |
| | | RSDRPREIPQELLDNTAQTFFTPHLGSAVD |
| | | EIRIEIERYCATSILQALAGDIPDGRVNDI |
| | | R |
| 58 | Amino acid sequence of | MVTHWIHPEVVDYLRRFCDPVVPVETEVLG |
| | a PtxD homolog from | RRQCLELAADADALIMCMADRVDDDFLAQC |
| | Streptomyces sviceus | PRLRVISTVVKGYDNFDAEACARRGVWLTV |
| | (GenBank EDY59675.1) | LPDLLTAPTAELAVTLAVALGRRIREGDAL |
| | | MRSGRYDGWRPVLYGTGLYRSRVGVVGMGR |
| | | LGRAVARRLSGFEPSEVLYYDKQPLGASEE |
| | | RRLGVRAAGLEELMGRCQVVLSLLPLAMDT |
| | | RHLIGSDAIAAARPGQLLVNVGRGSVVDED |
| | | AVAAALDCGPLGGYAADVFGCEDLTAPGHL |
| | | REVPRRLLTHPRTLLTPHLGSAVDVIRRDM |
| | | EIAAAHQVEQALSGRVPDHEVTAGLLRE |
| 59 | Amino acid sequence of | MLPKLVITHRVHDEILQLLAPHCELMTNQT |
| | a PtxD homolog from | DSTLPREEILRRCRDAQAMMAFMPDRVDAD |
| | Thioalkalivibrio sp. HL- | FLQACPELRVVGCALKGFDNFDVDACTARG |
| | EbGR7 (GenBank | VWLTFVPDLLTVPTAELAIGLAVGLGRHLR |
| | ACL72000.1) | AADAFVRSGEFQGWQPQFYGTGLDNATVGI |
| | | LGMGAIGLAMADRLQGWGATLQYHEAKALD |
| | | TQTEQRLGLRRVACSELFASSDFILLALPL |
| | | NADTQHLVNAELLALVRPGALLVNPCRGSV |
| | | VDEAAVLAALERGQLGGYAADVFEMEDWAR |
| | | ADRPRLIDPALLTHPNTLFTPHIGSAVRAV |
| | | RLEIERCAAQNIIQVLAGARPINAANRLPK |
| | | AEPAAC |
| 60 | Amino acid sequence of | MARKTIVTNWVHPEVLDLLSTRGPAEANTT |
| | a PtxD homolog from | REPWPRDEIIRRAHGADAMLAFMTDHVDAA |
| | Xanthobacter flavus | FLDACPELKIVACALKGADNFDMEACRARK |
| | (GenBank ABG73582.1) | VAVTIVPDLLTAPTAELAVGLMITLGRNLL |
| | | AGDRLIRERPFAGWRPVLYGTGLDGAEVGI |
| | | VGMGAVGQAIAHRLRPFRCRLSYCDARPLS |
| | | PAAEDAQGLLRRDLADLVARSDYLVLALPL |
| | | TPASRHLIDAAALAGMKPGALLINPARGSL |
| | | VDEAAVADALEAGHLGGYAADVFETEDWAR |
| | | PDRPAAIEARLLAHPRTVLTPHIGSAVDSV |
| | | RRDIALAAARDILRHLDGLQQDPPSRDRSA |
| | | A |
| 61 | Amino acid sequence of | VGILGMGAIG |
| | an NAD-binding motif | |
| 62 | Amino acid sequence of | XPGALLVNPCRGSVVD |
| | a conserved signature | |
| | sequence for the D- | |
| | isomer specific 2- | |
| | hydroxyacid family | |
| 63 | Amino acid sequence of | RGSVVD |
| | a shorter consensus | |
| | sequence within SEQ ID | |
| | NO: 62 | |
| 64 | Amino acid sequence of | GWQPQFYGTGL |
| | a consensus sequence | |
| | for a motif that may | |
| | enable hydrogenases to | |
| | use phosphite as a | |
| | substrate | |
| 65 | Amino acid sequence of | GWXPXXYXXGL |
| | a more generic | |
| | consensus sequence of a | |
| | motif that may enable | |
| | hydrogenases to use | |
| | phosphite as a substrate. | |
| 66 | Nucleotide sequence for | ATGttgCCAAAGTTGGTCATCACCCATaga |
| | a ptxD protein coding | GTCCAtgatgaaATCTTGcaaTTGttaGCT |
| | region with codons | CCAcatTGCgaattaATGactAATcaaact |
| | optimized for good gene | gatTCTacattaactAGAgaagaaATCTTG |
| | expression in the | AGAAGATGTAGAGATGCTCAAGCTATGATG |
| | nucleus of yeast, | GCCTTCATGCCAGATAGAGTTgatGCTgat |
| | Saccharomyces | TTCTTGCAAGCATGTCCAGAATTGagaGTC |
| | cerevisiae | GTTGGTTGCGCCttgAAGGGTTTCgataat |
| | | TTCgatGTCgatGCTTGTactGCTAGAGGT |
| | | GTCTGGttgacaTTCGTCCCAGATTTGTTG |
| | | actGTCCCAactGCTGAATTGGCTATTGGT |
| | | TTGGCCGTCGGTCTTggtAGAcatTTGAGA |
| | | GCCGCTgatGCTTTCGTTAGATCTggtGAG |
| | | TTTCAAGGTTGGcaaCCACAATTCTACggt |
| | | actggtTTGgatAACGCCactGTTGGTATC |
| | | TTGGGTATGggtGCCATCGGTTTGGCTATG |
| | | GCCgatAGATTGCAAggtTGGGGTGCTact |
| | | TTGcaaTACcatGAAGCTAAGGCCTTGGAT |
| | | ACCCAAactGAAcaaagattaGGTTTGaga |
| | | CAAGTTGCTTGTtctgaaCTTTTTGCCTCT |
| | | tcagatTTCATCTTGTTGGCCTTGCCTttg |
| | | AACGCCgatactcaacatttgGTCAATGCC |
| | | gaattaTTGGCCTTGGTCAGAccaGGTGCC |
| | | TTGCTTGTCAACCCTTGTAGAggttctGTT |
| | | GTTgatGAAGCTGCCGTTttgGCCGCTCTT |
| | | GAAagaGGTcaattaGGTGGTTATGCCGCC |
| | | gatGTTTTCGAAATGGAGGATTGGGCTaga |
| | | GCTGATAGGCCAAGATTGATCgatCCAGCT |
| | | TTGttaGCTcatCCTAACACCTTGTTCact |
| | | CCAcatATCggtTCTGCTGTTagaGCTGTT |
| | | AGACTTgaaATTGAGagaTGCGCCGCCCAG |
| | | AACATCATCCAAGTCTTGGCTGGTGCCAGA |
| | | CCTATTAACGCCGCCAATagaTTGCCAAAG |
| | | GCTGAACCAGCTGCTTGTTAA |
| 67 | Nucleotide sequence | ATGttaTCAttgagaCAATCTATAAGATTT |
| | encoding the | TTCAAGCCAGCCACAAGAACTTTGTGTtca |
| | mitochondrial targeting | TCTAGATATttgtta |
| | sequence (MTS) of the | |
| | yeast COX4 gene | |
| 68 | Nucleotide sequence | ATGttaTCAttgagaCAATCTATAAGATTT |
| | encoding a chimeric | TTCAAGCCAGCCACAAGAACTTTGTGTtca |
| | MTS(COX4)-ptxD | TCTAGATATttgttaATGttgCCAAAGTTG |
| | fusion protein | GTCATCACCCATagaGTCCAtgatgaaATC |
| | | TTGcaaTTGttaGCTCCAcatTGCgaatta |
| | | ATGactAATcaaactgatTCTacattaact |
| | | AGAgaagaaATCTTGAGAAGATGTAGAGAT |
| | | GCTCAAGCTATGATGGCCTTCATGCCAGAT |
| | | AGAGTTgatGCTgatTTCTTGCAAGCATGT |
| | | CCAGAATTGagaGTCGTTGGTTGCGCCttg |
| | | AAGGGTTTCgataatTTCgatGTCgatGCT |
| | | TGTactGCTAGAGGTGTCTGGttgacaTTC |
| | | GTCCCAGATTTGTTGactGTCCCAactGCT |
| | | GAATTGGCTATTGGTTTGGCCGTCGGTCTT |
| | | ggtAGAcatTTGAGAGCCGCTgatGCTTTC |
| | | GTTAGATCTggtGAGTTTCAAGGTTGGcaa |
| | | CCACAATTCTACggtactggtTTGgatAAC |
| | | GCCactGTTGGTATCTTGGGTATGggtGCC |
| | | ATCGGTTTGGCTATGGCCgatAGATTGCAA |
| | | ggtTGGGGTGCTactTTGcaaTACcatGAA |
| | | GCTAAGGCCTTGGATACCCAAactGAAcaa |
| | | agattaGGTTTGagaCAAGTTGCTTGTtct |
| | | gaaCTTTTTGCCTCTtcagatTTCATCTTG |
| | | TTGGCCTTGCCTttgAACGCCgatactcaa |
| | | catttgGTCAATGCCgaattaTTGGCCTTG |
| | | GTCAGAccaGGTGCCTTGCTTGTCAACCCT |
| | | TGTAGAggttctGTTGTTgatGAAGCTGCC |
| | | GTTttgGCCGCTCTTGAAagaGGTcaatta |
| | | GGTGGTTATGCCGCCgatGTTTTCGAAATG |
| | | GAGGATTGGGCTagaGCTGATAGGCCAAGA |
| | | TTGATCgatCCAGCTTTGttaGCTcatCCT |
| | | AACACCTTGTTCactCCAcatATCggtTCT |
| | | GCTGTTagaGCTGTTAGACTTgaaATTGAG |
| | | agaTGCGCCGCCCAGAACATCATCCAAGTC |
| | | TTGGCTGGTGCCAGACCTATTAACGCCGCC |
| | | AATagaTTGCCAAAGGCTGAACCAGCTGCT |
| | | TGTtaa |
| 69 | Nucleotide sequence for | CATAGCTTCAAAATGTTTCTACTCCTTTTT |
| | the strong constitutive | TACTCTTCCAGATTTTCTCGGACTCCGCGC |
| | TEF1 promoter present | ATCGCCGTACCACTTCAAAACACCCAAGCA |
| | in plasmid pNY101 | CAGCATACTAAATTTCCCCTCTTTCTTCCT |
| | | CTAGGGTGTCGTTAATTACCCGTACTAAAG |
| | | GTTTGGAAAAGAAAAAAGAGACCGCCTCGT |
| | | TTCTTTTTCTTCGTCGAAAAAGGCAATAAA |
| | | AATTTTTATCACGTTTCTTTTTCTTGAAAA |
| | | TTTTTTTTTTTGATTTTTTTCTCTTTCGAT |
| | | GACCTCCCATTGATATTTAAGTTAATAAAC |
| | | GGTCTTCAATTTCTCAAGTTTCAGTTTCAT |
| | | TTTTCTTGTTCTATTACAACTTTTTTTACT |
| | | TCTTGCTCATTAGAAAGAAAGCATAGCAAT |
| | | CTAATCTAAG |
| 70 | Nucleotide sequence for | ATGCTGCCGAAACTCGTTATcACTCACCGg |
| | a ptxD protein coding | GTgCACGATGAGATCCTGCAACTGCTGGCG |
| | region with codons | CCACATTGCGAGCTGATGACCAACCAGACC |
| | optimized for good gene | GACAGCACGCTGACGCGCGAGGAAATTCTG |
| | expression in the | CGCCGCTGcCGCGATGCTCAGGCGATGATG |
| | nucleus of rice, Oryza | GCGTTCATGCCCGATCGGGTCGATGCAGAC |
| | sativa | TTTCTTCAAGCCTGCCCTGAGCTGCGcGTg |
| | | GTCGGCTGCGCGCTCAAGGGCTTCGACAAT |
| | | TTCGATGTGGACGCCTGcACTGCCCGCGGG |
| | | GTCTGGCTGACCTTCGTGCCTGATCTGTTG |
| | | ACGGTCCCGACTGCCGAGCTGGCGATCGGA |
| | | CTGGCGGTGGGGCTGGGGCGGCATCTGCGG |
| | | GCAGCAGATGCGTTCGTCCGCTCTGGCGAG |
| | | TTCCAGGGCTGGCAACCACAGTTCTACGGC |
| | | ACGGGGCTGGATAACGCTACGGTCGGCATC |
| | | CTTGGCATGGGCGCCATCGGACTGGCCATG |
| | | GCTGATCGCTTGCAGGGATGGGGCGCGACC |
| | | CTGCAGTACCACGAGGCGAAGGCTCTGGAT |
| | | ACACAAACCGAGCAACGGCTCGGCCTGCGC |
| | | CAGGTGGCGTGCAGCGAACTCTTCGCCAGC |
| | | TCGGACTTCATCCTGCTGGCGCTTCCCTTG |
| | | AATGCCGATACCCAGCATCTGGTCAACGCC |
| | | GAGCTGCTTGCCCTCGTgCGGCCGGGCGCT |
| | | CTGCTTGTgAACCCCTGcCGcGGTTCGGTg |
| | | GTGGATGAAGCCGCCGTGCTCGCGGCGCTT |
| | | GAGCGgGGCCAGCTCGGCGGGTATGCGGCG |
| | | GATGTgTTCGAAATGGAAGACTGGGCTCGC |
| | | GCGGACCGGCCGCGGCTGATCGATCCTGCG |
| | | CTGCTCGCGCATCCGAATACGCTGTTCACT |
| | | CCGCACATcGGGTCGGCAGTGCGCGCGGTG |
| | | CGCCTGGAGATTGAACGcTGcGCAGCGCAG |
| | | AACATCATCCAGGTgTTGGCAGGTGCGCGC |
| | | CCAATCAACGCTGCGAACCGcCTGCCCAAG |
| | | GCCGAGCCTGCCGCATGcTGA |
| 71 | Nucleotide sequence | ATGGCCGCCAAGATcCGCATcGTGATGAAA |
| | encoding the | TCTTTTATGAGCCAAGCTAACAAAGTTGAA |
| | mitochondrial targeting | GGGGTTATTCCATACGCGCAGAAGGTTGGA |
| | sequence (MTS) of the | TTGCCTGAATCACGATCCTTGTATACCGTG |
| | rice RPS10 gene | CTACGATCGCCTCACATcGACAAGAAGTCG |
| | | AGGGAGCAGTTCTCGATG |
| 72 | Amino acid sequence of | PVAT |
| | the PVAT linker that | |
| | connects the ptxD and | |
| | eGFP proteins in the | |
| | fusion protein encoded | |
| | by pNAP256 | |
| 73 | Nucleotide sequence for | ATGttaCCaAAAttaGTTATtACTCAtaga |
| | a ptxD protein coding | GTACAtGATGAaATtttaCAAttattaGCa |
| | region with codons | CCACATTGtGAattaATGACtAACCAaACt |
| | optimized for gene | GAtAGtACattaACaagaGAaGAAATTtta |
| | expression in the | agaagaTGTagaGATGCTCAaGCaATGATG |
| | mitochondria of yeast, | GCaTTCATGCCtGATagaGTtGATGCAGAt |
| | Saccharomyces | TTcttaCAAGCtTGtCCTGAattaagaGTA |
| | cerevisiae, e.g., by | GTtGGtTGtGCattaAAaGGtTTCGAtAAT |
| | changing tryptophan | TTCGATGTaGAtGCtTGTACTGCtagaGGa |
| | codons to UGA, which | GTtTGattaACtTTCGTaCCTGATttaTTa |
| | is recognized as a stop | ACaGTtCCaACTGCtGAattaGCaATtGGA |
| | codon in the cytoplasm | ttaGCaGTaGGattaGGaagaCATttaaga |
| | but as a tryptophan | GCAGCAGATGCaTTCGTtagaTCTGGtGAa |
| | codon in mitochondria | TTCCAaGGtTGaCAACCACAaTTCTAtGGt |
| | | ACaGGattaGATAAtGCTACaGTtGGtATt |
| | | ttaGGtATGGGtGCtATtGGAttaGCtATG |
| | | GCTGATagaTTaCAaGGATGaGGtGCaACt |
| | | ttaCAaTAtCAtGAaGCaAAaGCTttaGAT |
| | | ACACAAACtGAaCAAagattaGGtttaaga |
| | | CAaGTaGCaTGtAGtGAAttaTTCGCtAGt |
| | | TCaGAtTTCATtttattaGCattaCCtTTa |
| | | AATGCtGATACtCAaCATttaGTtAAtGCt |
| | | GAattattaGCtttaGTAagaCCaGGtGCT |
| | | ttattaGTAAAtCCtTGTagaGGTTCaGTA |
| | | GTaGATGAAGCtGCtGTattaGCaGCatta |
| | | GAaagaGGtCAattaGGtGGaTATGCaGCa |
| | | GATGTATTCGAAATGGAAGAtTGaGCTaga |
| | | GCaGAtagaCCaagattaATtGATCCTGCa |
| | | ttattaGCaCATCCaAATACattaTTCACT |
| | | CCaCAtATtGGaTCaGCAGTaagaGCaGTa |
| | | agattaGAaATTGAAagaTGTGCAGCaCAa |
| | | AACATtATtCAaGTATTaGCAGGTGCaaga |
| | | CCAATtAACGCTGCaAACagattaCCtAAa |
| | | GCtGAaCCTGCtGCATGTTaA |
| 74 | Nucleotide sequence for | TATTGTGTTACCTTATTTATAAAGGTATGA |
| | the yeast mitochondrial | AGCAAAGGTGTTATTATTTATTATTATTAT |
| | COX2 promoter present | TATTATTATTAATATAATATATATATATAT |
| | in plasmid pNY104 | ATATGATATGAATATTATTAGTTTTCGGGA |
| | | AGCGGGAATCCCGTAAGGAGTGAGGGACCC |
| | | TCCCTATACTAAGGGAGGGGGACCGAACCC |
| | | CGAAGGAGTTTTATTTTTAGTATTTTATAA |
| | | AATATATATTTATATGATTAATAATATTAT |
| | | ATATATTATTTATAAAAATAATATATAATT |
| | | TTAATTATTTTTAATAAAAAAAGGTGGGGT |
| | | TTGGTAATATAATATTTTTATTTTATTTAT |
| | | AATATATAATAATAAATTATAAATAAATTT |
| | | TAATTAAAAGTAGTATTAACATATTATAAA |
| | | TAGACAAAAGAGTCTAAAGGTTAAGATTTA |
| | | TTAAA |
| 75 | Nucleotide sequence for | ttaatatttttaattattaaaaataataat |
| | the yeast mitochondrial | aataataataattataataatattcttaaa |
| | COX2 terminator present | tataataaagatatagatttatattctatt |
| | in plasmid pNY104 | caatcaccttat |
| 76 | Nucleotide sequence of | atagacctttttatttttcgtcattcgatc |
| | the 863 bp-long region | acgaaaacagggattctggaacggccaaga |
| | downstream of the rice | atcccagcggttgttcgggtcgaaaaaccg |
| | mitochondrial ATPl stop | agaacaagacatgccacaaagtggcagatg |
| | codon, a putative | aaggcaggggggagagcctagtcctcaacc |
| | terminator sequence | tcttcttccccaaaaggtagttatgaacgt |
| | | gccaaacttattggatttattcttggaatg |
| | | ctcataaccacctttactctttttttcatt |
| | | ctttactcagaggaagccatgccgtttgga |
| | | gaagagcaccaagtggggggagtgtggagt |
| | | ccccgaaagaggagctttctaaaggcaaga |
| | | gaaaagctccgatggagcccttggagctac |
| | | agggaccaccaacccttcgcagcttggacg |
| | | atttgattcttgtgccactcagccctgagg |
| | | agggcgcctgctcgacccagtcaggtacta |
| | | ctccgccgccggccccgagtaactctgcgg |
| | | gggtcggtgcagccctttcttctattccgg |
| | | agtgcataaacaaggatcctcaaaaagcga |
| | | aatcatttcgttaatggcatttcagaaatg |
| | | agtcataggcgcctgtacaatgacagaata |
| | | gagagtcctttttttccagaatgaatcatt |
| | | ctattcaaatctcacaagttctctttacgc |
| | | gtcttctaggggcattgttgaacgcaatct |
| | | gcaggaacaagaaatgattctttcttattt |
| | | tgaaacagaattcaaaataaaggaggattt |
| | | aattcggttgctttatgaaggccgacgccg |
| | | tgccgatagatacgttatacacgaaacgaa |
| | | aatagccagtacggtggacgcgttcctttc |
| | | caaaaagggattatcagGAGCTC |
| 77 | Nucleotide sequence of | gtctgccccattcgataatggcaTAATACG |
| | a synthetic promoter of | ACTCACTATAGtttatgatctagtggagtg |
| | 139 nucleotides | agtgattgtgtggtgttcagtctaaggctt |
| | consisting of the T7 | tttgaaaagcggatttctcccttctctcat |
| | promoter inserted | ccatcgtctttgttaaagt |
| | upstream of the nearest | |
| | rice mitochondrial ATP1 | |
| | transcription start site | |
| 78 | Nucleotide sequence of | ggtaccaagcgatcgcaaacctaggaaaag |
| | a synthetic terminator | atctaaaaagcttaagcggccgcaaaAACC |
| | consisting of the T7 | CCTTGGGGCCTCTAAACGGGTCTTGAGGGG |
| | terminator inserted | TTTTTTGtagatagacctttttatttttcg |
| | upstream of a short AT- | tcattcgatcacgaaaaggccggccaaacc |
| | rich 40 nucleotide | tcgaggaaaggtaccaaaggcgcgcc |
| | sequence from the rice | |
| | mitochondrial ATP1 | |
| | terminator | |
| 79 | Nucleotide sequence | ATGGCCGCCAAGATcCGCATcGTGATGAAA |
| | encoding a fusion | TCTTTTATGAGCCAAGCTAACAAAGTTGAA |
| | protein in which the | GGGGTTATTCCATACGCGCAGAAGGTTGGA |
| | mitochondrial targeting | TTGCCTGAATCACGATCCTTGTATACCGTG |
| | sequence of the RPS10 | CTACGATCGCCTCACATcGACAAGAAGTCG |
| | protein is fused to the | AGGGAGCAGTTCTCGATGaacacgattaac |
| | amino-terminus of the | atcgctaagaacgacttctctgacatcgaa |
| | T7 RNA polymerase | ctggctgctatcccgttcaacactctggct |
| | | gaccattacggtgagcgtttagctcgcgaa |
| | | cagttggcccttgagcatgagtcttacgag |
| | | atgggtgaagcacgcttccgcaagatgttt |
| | | gagcgtcaacttaaagctggtgaggttgcg |
| | | gataacgctgccgccaagcctctcatcact |
| | | accctactccctaagatgattgcacgcatc |
| | | aacgactggtttgaggaagtgaaagctaag |
| | | cgcggcaagcgcccgacagccttccagttc |
| | | ctgcaagaaatcaagccggaagccgtagcg |
| | | tacatcaccattaagaccactctggcttgc |
| | | ctaaccagtgctgacaatacaaccgttcag |
| | | gctgtagcaagcgcaatcggtcgggccatt |
| | | gaggacgaggctcgcttcggtcgtatccgt |
| | | gaccttgaagctaagcacttcaagaaaaac |
| | | gttgaggaacaactcaacaagcgcgtaggg |
| | | cacgtctacaagaaagcatttatgcaagtt |
| | | gtcgaggctgacatgctctctaagggtcta |
| | | ctcggtggcgaggcgtggtcttcgtggcat |
| | | aaggaagactctattcatgtaggagtacgc |
| | | tgcatcgagatgctcattgagtcaaccgga |
| | | atggttagcttacaccgccaaaatgctggc |
| | | gtagtaggtcaagactctgagactatcgaa |
| | | ctcgcacctgaatacgctgaggctatcgca |
| | | acccgtgcaggtgcgctggctggcatctct |
| | | ccgatgttccaaccttgcgtagttcctcct |
| | | aagccgtggactggcattactggtggtggc |
| | | tattgggctaacggtcgtcgtcctctggcg |
| | | ctggtgcgtactcacagtaagaaagcactg |
| | | atgcgctacgaagacgtttacatgcctgag |
| | | gtgtacaaagcgattaacattgcgcaaaac |
| | | accgcatggaaaatcaacaagaaagtccta |
| | | gcggtcgccaacgtaatcaccaagtggaag |
| | | cattgtccggtcgaggacatccctgcgatt |
| | | gagcgtgaagaactcccgatgaaaccggaa |
| | | gacatcgacatgaatcctgaggctctcacc |
| | | gcgtggaaacgtgctgccgctgctgtgtac |
| | | cgcaaggacaaggctcgcaagtctcgccgt |
| | | atcagccttgagttcatgcttgagcaagcc |
| | | aataagtttgctaaccataaggccatctgg |
| | | ttcccttacaacatggactggcgcggtcgt |
| | | gtttacgctgtgtcaatgttcaacccgcaa |
| | | ggtaacgatatgaccaaaggactgcttacg |
| | | ctggcgaaaggtaaaccaatcggtaaggaa |
| | | ggttactactggctgaaaatccacggtgca |
| | | aactgtgcgggtgtcgataaggttccgttc |
| | | cctgagcgcatcaagttcattgaggaaaac |
| | | cacgagaacatcatggcttgcgctaagtct |
| | | ccactggagaacacttggtgggctgagcaa |
| | | gattctccgttctgcttccttgcgttctgc |
| | | tttgagtacgctggggtacagcaccacggc |
| | | ctgagctataactgctcccttccgctggcg |
| | | tttgacgggtcttgctctggcatccagcac |
| | | ttctccgcgatgctccgagatgaggtaggt |
| | | ggtcgcgcggttaacttgcttcctagtgaa |
| | | accgttcaggacatctacgggattgttgct |
| | | aagaaagtcaacgagattctacaagcagac |
| | | gcaatcaatgggaccgataacgaagtagtt |
| | | accgtgaccgatgagaacactggtgaaatc |
| | | tctgagaaagtcaagctgggcactaaggca |
| | | ctggctggtcaatggctggcttacggtgtt |
| | | actcgcagtgtgactaagcgttcagtcatg |
| | | acgctggcttacgggtccaaagagttcggc |
| | | ttccgtcaacaagtgctggaagataccatt |
| | | cagccagctattgattccggcaagggtctg |
| | | atgttcactcagccgaatcaggctgctgga |
| | | tacatggctaagctgatttgggaatctgtg |
| | | agcgtgacggtggtagctgcggttgaagca |
| | | atgaactggcttaagtctgctgctaagctg |
| | | ctggctgctgaggtcaaagataagaagact |
| | | ggagagattcttcgcaagcgttgcgctgtg |
| | | cattgggtaactcctgatggtttccctgtg |
| | | tggcaggaatacaagaagcctattcagacg |
| | | cgcttgaacctgatgttcctcggtcagttc |
| | | cgcttacagcctaccattaacaccaacaaa |
| | | gatagcgagattgatgcacacaaacaggag |
| | | tctggtatcgctcctaactttgtacacagc |
| | | caagacggtagccaccttcgtaagactgta |
| | | gtgtgggcacacgagaagtacggaatcgaa |
| | | tcttttgcactgattcacgactccttcggt |
| | | accattccggctgacgctgcgaacctgttc |
| | | aaagcagtgcgcgaaactatggttgacaca |
| | | tatgagtcttgtgatgtactggctgatttc |
| | | tacgaccagttcgctgaccagttgcacgag |
| | | tctcaattggacaaaatgccagcacttccg |
| | | gctaaaggtaacttgaacctccgtgacatc |
| | | ttagagtcggacttcgcgttcgcgtaaGCA |
| | | TGCTGA |
| 80 | Nucleotide sequence | ATGCTGCCAAACTCGTTATAACTCACCGAG |
| | encoding a ptxD-eGFP | TACAtGATGAGATCCTGCAACTGCTGGCGC |
| | fusion protein in which | CACATTGCGAGCTGATGACCAACCAaACCG |
| | the ptxD protein is fused | ACAGCACaCTGACaCGCGAGGAAATTCTGC |
| | to the eGFP protein by | GtCGaTGTCGtGATGCTCAaGCGATGATGG |
| | use of a PVAT linker | CGTTCATGCCCGATCGaGTCGATGCAGACT |
| | | TTCTTCAAGCCTGCCCTGAGCTGCGTGTAG |
| | | TCGGCTGCGCGCTCAAGGGCTTCGACAATT |
| | | TCGATGTGGACGCCTGTACTGCCCGtGGGG |
| | | TCTGGCTGACCTTCGTGCCTGATCTGTTGA |
| | | CaGTCCCaACTGCCGAGCTGGCGATCGGAC |
| | | TGGCGGTGGGGCTGGGGCGaCATCTGCGaG |
| | | CAGCAGATGCGTTCGTCCGtTCTGGCGAGT |
| | | TCCAaGGCTGGCAACCACAaTTCTAtGGCA |
| | | CaGGGCTGGATAACGCTACaGTCGGCATCC |
| | | TTGGCATGGGCGCCATCGGACTGGCCATGG |
| | | CTGATCGaTTGCAaGGATGGGGCGCGACCC |
| | | TGCAaTAtCACGAGGCGAAGGCTCTGGATA |
| | | CACAAACCGAGCAACGaCTCGGCCTGCGtC |
| | | AaGTGGCGTGCAGCGAACTCTTCGCCAGCT |
| | | CGGACTTCATCCTGCTGGCGCTTCCCTTGA |
| | | ATGCCGATACCCAaCATCTGGTCAACGCCG |
| | | AGCTGCTTGCCCTCGTACGaCCaGGCGCTC |
| | | TGCTTGTAAACCCCTGTCGTGGTTCGGTAG |
| | | TGGATGAAGCCGCCGTGCTCGCGGCGCTTG |
| | | AGCGAGGCCAaCTCGGCGGGTATGCGGCGG |
| | | ATGTATTCGAAATGGAAGACTGGGCTCGtG |
| | | CGGACCGaCCaCGaCTGATCGATCCTGCGC |
| | | TGCTCGCGCATCCtAATACaCTGTTCACTC |
| | | CaCAtATAGGGTCGGCAGTGCGtGCGGTGC |
| | | GtCTGGAGATTGAACGTTGTGCAGCGCAaA |
| | | ACATCATCCAaGTATTGGCAGGTGCGCGtC |
| | | CAATCAACGCTGCGAACCGTCTGCCCAAGG |
| | | CCGAGCCTGCCGCATGTccagttgctactA |
| | | TGGTGAGTAAGGGAGAGGAGCTGTTCACCG |
| | | GGGTGGTGCCTATCCTGGTCGAGCTGGATG |
| | | GTGATGTAAACGGTCATAAATTCAGTGTGT |
| | | CCGGTGAAGGTGAAGGTGATGCCACCTATG |
| | | GTAAGCTGACCCTTAAGTTCATCTGTACCA |
| | | CCGGAAAGCTGCCTGTGCCTTGGCCTACCC |
| | | TCGTGACCACCCTGACATATGGAGTGCAAT |
| | | GTTTCAGTCGTTATCCTGATCATATGAAGC |
| | | AACATGATTTCTTTAAATCCGCCATGCCTG |
| | | AAGGTTATGTCCAAGAGCGTACCATATTCT |
| | | TTAAAGATGATGGTAACTATAAGACCCGTG |
| | | CCGAGGTGAAGTTCGAGGGTGATACCCTGG |
| | | TGAACCGTATTGAGCTTAAGGGTATCGATT |
| | | TCAAGGAGGATGGAAACATCCTGGGGCATA |
| | | AGCTGGAGTATAACTATAACAGTCATAACG |
| | | TCTATATCATGGCCGATAAGCAAAAGAACG |
| | | GTATCAAGGTGAACTTCAAGATCCGTCATA |
| | | ATATCGAAGATGGAAGTGTGCAACTCGCCG |
| | | ATCATTATCAACAAAACACCCCTATCGGTG |
| | | ATGGTCCTGTGCTGCTGCCTGATAACCATT |
| | | ATCTGAGTACCCAATCCGCCCTGAGTAAAG |
| | | ATCCTAACGAGAAGCGTGATCAAATGGTAC |
| | | TGCTTGAGTTCGTTACCGCCGCCGGGATCA |
| | | CTCTCGGTATGGATGAGCTGTATAAGTAA |
| 81 | Amino acid sequence of | MLPKLVITHRVHDEILQLLAPHCELMTNQT |
| | the ptxD-eGFP fusion | DSTLTREEILRRCRDAQAMMAFMPDRVDAD |
| | protein encoded by SEQ | FLQACPELRVVGCALKGFDNFDVDACTARG |
| | ID NO: 80 | VWLTFVPDLLTVPTAELAIGLAVGLGRHLR |
| | | AADAFVRSGEFQGWQPQFYGTGLDNATVGI |
| | | LGMGAIGLAMADRLQGWGATLQYHEAKALD |
| | | TQTEQRLGLRQVACSELFASSDFILLALPL |
| | | NADTQHLVNAELLALVRPGALLVNPCRGSV |
| | | VDEAAVLAALERGQLGGYAADVFEMEDWAR |
| | | ADRPRLIDPALLAHPNTLFTPHIGSAVRAV |
| | | RLEIERCAAQNIIQVLAGARPINAANRLPK |
| | | AEPAACPVATMVSKGEELFTGVVPILVELD |
| | | GDVNGHKFSVSGEGEGDATYGKLTLKFICT |
| | | TGKLPVPWPTLVTTLTYGVQCFSRYPDHMK |
| | | QHDFFKSAMPEGYVQERTIFFKDDGNYKTR |
| | | AEVKFEGDTLVNRIELKGIDFKEDGNILGH |
| | | KLEYNYNSHNVYIMADKQKNGIKVNFKIRH |
| | | NIEDGSVQLADHYQQNTPIGDGPVLLPDNH |
| | | YLSTQSALSKDPNEKRDQMVLLEFVTAAGI |
| | | TLGMDELYK |
| 82 | Nucleotide sequence | ATGCTGCCIAAACTCGTTATAACTCACCGA |
| | encoding a ptxD-eGFP | GTACAtGATGAGATCCTGCAACTGCTGGCG |
| | fusion protein in which | CCACATTGCGAGCTGATGACCAACCAaACC |
| | the ptxD protein is fused | GACAGCACaCTGACaCGCGAGGAAATTCTG |
| | to the eGFP protein by | CGtCGaTGTCGtGATGCTCAaGCGATGATG |
| | use of a GGGGS linker | GCGTTCATGCCCGATCGaGTCGATGCAGAC |
| | | TTTCTTCAAGCCTGCCCTGAGCTGCGTGTA |
| | | GTCGGCTGCGCGCTCAAGGGCTTCGACAAT |
| | | TTCGATGTGGACGCCTGTACTGCCCGtGGG |
| | | GTCTGGCTGACCTTCGTGCCTGATCTGTTG |
| | | ACaGTCCCaACTGCCGAGCTGGCGATCGGA |
| | | CTGGCGGTGGGGCTGGGGCGaCATCTGCGa |
| | | GCAGCAGATGCGTTCGTCCGtTCTGGCGAG |
| | | TTCCAaGGCTGGCAACCACAaTTCTAtGGC |
| | | ACaGGGCTGGATAACGCTACaGTCGGCATC |
| | | CTTGGCATGGGCGCCATCGGACTGGCCATG |
| | | GCTGATCGaTTGCAaGGATGGGGCGCGACC |
| | | CTGCAaTAtCACGAGGCGAAGGCTCTGGAT |
| | | ACACAAACCGAGCAACGaCTCGGCCTGCGt |
| | | CAaGTGGCGTGCAGCGAACTCTTCGCCAGC |
| | | TCGGACTTCATCCTGCTGGCGCTTCCCTTG |
| | | AATGCCGATACCCAaCATCTGGTCAACGCC |
| | | GAGCTGCTTGCCCTCGTACGaCCaGGCGCT |
| | | CTGCTTGTAAACCCCTGTCGTGGTTCGGTA |
| | | GTGGATGAAGCCGCCGTGCTCGCGGCGCTT |
| | | GAGCGAGGCCAaCTCGGCGGGTATGCGGCG |
| | | GATGTATTCGAAATGGAAGACTGGGCTCGt |
| | | GCGGACCGaCCaCGaCTGATCGATCCTGCG |
| | | CTGCTCGCGCATCCtAATACaCTGTTCACT |
| | | CCaCAtATAGGGTCGGCAGTGCGtGCGGTG |
| | | CGtCTGGAGATTGAACGTTGTGCAGCGCAa |
| | | AACATCATCCAaGTATTGGCAGGTGCGCGt |
| | | CCAATCAACGCTGCGAACCGTCTGCCCAAG |
| | | GCCGAGCCTGCCGCATGTggaggtggaggt |
| | | tcaGTGAGTAAGGGAGAGGAGCTGTTCACC |
| | | GGGGTGGTGCCTATCCTGGTCGAGCTGGAT |
| | | GGTGATGTAAACGGTCATAAATTCAGTGTG |
| | | TCCGGTGAAGGTGAAGGTGATGCCACCTAT |
| | | GGTAAGCTGACCCTTAAGTTCATCTGTACC |
| | | ACCGGAAAGCTGCCTGTGCCTTGGCCTACC |
| | | CTCGTGACCACCCTGACATATGGAGTGCAA |
| | | TGTTTCAGTCGTTATCCTGATCATATGAAG |
| | | CAACATGATTTCTTTAAATCCGCCATGCCT |
| | | GAAGGTTATGTCCAAGAGCGTACCATATTC |
| | | TTTAAAGATGATGGTAACTATAAGACCCGT |
| | | GCCGAGGTGAAGTTCGAGGGTGATACCCTG |
| | | GTGAACCGTATTGAGCTTAAGGGTATCGAT |
| | | TTCAAGGAGGATGGAAACATCCTGGGGCAT |
| | | AAGCTGGAGTATAACTATAACAGTCATAAC |
| | | GTCTATATCATGGCCGATAAGCAAAAGAAC |
| | | GGTATCAAGGTGAACTTCAAGATCCGTCAT |
| | | AATATCGAAGATGGAAGTGTGCAACTCGCC |
| | | GATCATTATCAACAAAACACCCCTATCGGT |
| | | GATGGTCCTGTGCTGCTGCCTGATAACCAT |
| | | TATCTGAGTACCCAATCCGCCCTGAGTAAA |
| | | GATCCTAACGAGAAGCGTGATCAAATGGTA |
| | | CTGCTTGAGTTCGTTACCGCCGCCGGGATC |
| | | ACTCTCGGTATGGATGAGCTGTATAAGTAA |
| 83 | Amino acid sequence of | MLPKLVITHRVHDEILQLLAPHCELMTNQT |
| | the ptxD-eGFP fusion | DSTLTREEILRRCRDAQAMMAFMPDRVDAD |
| | protein encoded by SEQ | FLQACPELRVVGCALKGFDNFDVDACTARG |
| | ID NO: 82 | VWLTFVPDLLTVPTAELAIGLAVGLGRHLR |
| | | AADAFVRSGEFQGWQPQFYGTGLDNATVGI |
| | | LGMGAIGLAMADRLQGWGATLQYHEAKALD |
| | | TQTEQRLGLRQVACSELFASSDFILLALPL |
| | | NADTQHLVNAELLALVRPGALLVNPCRGSV |
| | | VDEAAVLAALERGQLGGYAADVFEMEDWAR |
| | | ADRPRLIDPALLAHPNTLFTPHIGSAVRAV |
| | | RLEIERCAAQNIIQVLAGARPINAANRLPK |
| | | AEPAACGGGGSVSKGEELFTGVVPILVELD |
| | | GDVNGHKFSVSGEGEGDATYGKLTLKFICT |
| | | TGKLPVPWPTLVTTLTYGVQCFSRYPDHMK |
| | | QHDFFKSAMPEGYVQERTIFFKDDGNYKTR |
| | | AEVKFEGDTLVNRIELKGIDFKEDGNILGH |
| | | KLEYNYNSHNVYIMADKQKNGIKVNFKIRH |
| | | NIEDGSVQLADHYQQNTPIGDGPVLLPDNH |
| | | YLSTQSALSKDPNEKRDQMVLLEFVTAAGI |
| | | TLGMDELYK |
| 84 | Amino acid sequence of | GGGGS |
| | the GGGGS linker | |
| 85 | Nucleotide sequence | cattccatgtttccgaaacggatcctCTGC |
| | encoding a RNAed- | CtAAACTCGTTATAACTCACCGAGTACAtG |
| | ptxD-eGFP fusion | ATGAGATCCTGCAACTGCTGGCGCCACATT |
| | protein in which the | GCGAGCTGATGACCAACCAaACCGACAGCA |
| | ptxD and eGFP enzymes | CaCTGACaCGCGAGGAAATTCTGCGtCGaT |
| | are connected with a | GTCGtGATGCTCAaGCGATGATGGCGTTCA |
| | PVAT-linker, and in | TGCCCGATCGaGTCGATGCAGACTTTCTTC |
| | which the ATG start | AAGCCTGCCCTGAGCTGCGTGTAGTCGGCT |
| | codon has been replaced | GCGCGCTCAAGGGCTTCGACAATTTCGATG |
| | with a mitochondrial | TGGACGCCTGTACTGCCCGtGGGGTCTGGC |
| | RNA-editing site | TGACCTTCGTGCCTGATCTGTTGACaGTCC |
| | | CaACTGCCGAGCTGGCGATCGGACTGGCGG |
| | | TGGGGCTGGGGCGaCATCTGCGaGCAGCAG |
| | | ATGCGTTCGTCCGtTCTGGCGAGTTCCAaG |
| | | GCTGGCAACCACAaTTCTAtGGCACaGGGC |
| | | TGGATAACGCTACaGTCGGCATCCTTGGCA |
| | | TGGGCGCCATCGGACTGGCCATGGCTGATC |
| | | GaTTGCAaGGATGGGGCGCGACCCTGCAaT |
| | | AtCACGAGGCGAAGGCTCTGGATACACAAA |
| | | CCGAGCAACGaCTCGGCCTGCGtCAaGTGG |
| | | CGTGCAGCGAACTCTTCGCCAGCTCGGACT |
| | | TCATCCTGCTGGCGCTTCCCTTGAATGCCG |
| | | ATACCCAaCATCTGGTCAACGCCGAGCTGC |
| | | TTGCCCTCGTACGaCCaGGCGCTCTGCTTG |
| | | TAAACCCCTGTCGTGGTTCGGTAGTGGATG |
| | | AAGCCGCCGTGCTCGCGGCGCTTGAGCGAG |
| | | GCCAaCTCGGCGGGTATGCGGCGGATGTAT |
| | | TCGAAATGGAAGACTGGGCTCGtGCGGACC |
| | | GaCCaCGaCTGATCGATCCTGCGCTGCTCG |
| | | CGCATCCtAATACaCTGTTCACTCCaCAtA |
| | | TAGGGTCGGCAGTGCGtGCGGTGCGtCTGG |
| | | AGATTGAACGTTGTGCAGCGCAaAACATCA |
| | | TCCAaGTATTGGCAGGTGCGCGtCCAATCA |
| | | ACGCTGCGAACCGTCTGCCCAAGGCCGAGC |
| | | CTGCCGCATGTccagttgctactATGGTGA |
| | | GTAAGGGAGAGGAGCTGTTCACCGGGGTGG |
| | | TGCCTATCCTGGTCGAGCTGGATGGTGATG |
| | | TAAACGGTCATAAATTCAGTGTGTCCGGTG |
| | | AAGGTGAAGGTGATGCCACCTATGGTAAGC |
| | | TGACCCTTAAGTTCATCTGTACCACCGGAA |
| | | AGCTGCCTGTGCCTTGGCCTACCCTCGTGA |
| | | CCACCCTGACATATGGAGTGCAATGTTTCA |
| | | GTCGTTATCCTGATCATATGAAGCAACATG |
| | | ATTTCTTTAAATCCGCCATGCCTGAAGGTT |
| | | ATGTCCAAGAGCGTACCATATTCTTTAAAG |
| | | ATGATGGTAACTATAAGACCCGTGCCGAGG |
| | | TGAAGTTCGAGGGTGATACCCTGGTGAACC |
| | | GTATTGAGCTTAAGGGTATCGATTTCAAGG |
| | | AGGATGGAAACATCCTGGGGCATAAGCTGG |
| | | AGTATAACTATAACAGTCATAACGTCTATA |
| | | TCATGGCCGATAAGCAAAAGAACGGTATCA |
| | | AGGTGAACTTCAAGATCCGTCATAATATCG |
| | | AAGATGGAAGTGTGCAACTCGCCGATCATT |
| | | ATCAACAAAACACCCCTATCGGTGATGGTC |
| | | CTGTGCTGCTGCCTGATAACCATTATCTGA |
| | | GTACCCAATCCGCCCTGAGTAAAGATCCTA |
| | | ACGAGAAGCGTGATCAAATGGTACTGCTTG |
| | | AGTTCGTTACCGCCGCCGGGATCACTCTCG |
| | | GTATGGATGAGCTGTATAAGTAA |
| 86 | Amino acid sequence of | MDPLPKLVITHRVHDEILQLLAPHCELMTN |
| | the RNAed-ptxD-eGFP | QTDSTLTREEILRRCRDAQAMMAFMPDRVD |
| | fusion protein encoded | ADFLQACPELRVVGCALKGFDNFDVDACTA |
| | by SEQ ID NO: 85 | RGVWLTFVPDLLTVPTAELAIGLAVGLGRH |
| | | LRAADAFVRSGEFQGWQPQFYGTGLDNATV |
| | | GILGMGAIGLAMADRLQGWGATLQYHEAKA |
| | | LDTQTEQRLGLRQVACSELFASSDFILLAL |
| | | PLNADTQHLVNAELLALVRPGALLVNPCRG |
| | | SVVDEAAVLAALERGQLGGYAADVFEMEDW |
| | | ARADRPRLIDPALLAHPNTLFTPHIGSAVR |
| | | AVRLEIERCAAQNIIQVLAGARPINAANRL |
| | | PKAEPAACPVATMVSKGEELFTGVVPILVE |
| | | LDGDVNGHKFSVSGEGEGDATYGKLTLKFI |
| | | CTTGKLPVPWPTLVTTLTYGVQCFSRYPDH |
| | | MKQHDFFKSAMPEGYVQERTIFFKDDGNYK |
| | | TRAEVKFEGDTLVNRIELKGIDFKEDGNIL |
| | | GHKLEYNYNSHNVYIMADKQKNGIKVNFKI |
| | | RHNIEDGSVQLADHYQQNTPIGDGPVLLPD |
| | | NHYLSTQSALSKDPNEKRDQMVLLEFVTAA |
| | | GITLGMDELYK |
| 87 | Nucleotide sequence of | ATGGCAAATCTGGTCCGATGGCTCTTCTCC |
| | the rice CMS gene, | ACTACCCGAGGGACTAACGGTCTTCCATAT |
| | orf79, that is present in | TTCATCTTCGGTGTCGTTGTAGGAGGCGCC |
| | the rice CMS line Boro | CTGTTGTTTGCTTTGCTAAAGTATCAGGCC |
| | II Taichung | CCTCTGTACGACCCGGCTTTAATGGAAAAA |
| | | ATCATAGATCATAATATAAAAGCCGGGCAC |
| | | CCTATAGAGGTTGACTATTCGTGGTGGGGC |
| | | ACCTCTATTCGTGTAGTCTTTCCTAAGTAA |
| 88 | Nucleotide sequence | ATGGCCGCCAAGATcCGCATcGTGATGAAA |
| | encoding an | TCTTTTATGAGCCAAGCTAACAAAGTTGAA |
| | MTS(RPS10)-MAD7 | GGGGTTATTCCATACGCGCAGAAGGTTGGA |
| | fusion protein in which | TTGCCTGAATCACGATCCTTGTATACCGTG |
| | the MAD7 enzyme is | CTACGATCGCCTCACATcGACAAGAAGTCG |
| | fused at the amino | AGGGAGCAGTTCTCGATGAACAACGGTACA |
| | terminus with the | AATAATTTCCAAAACTTCATCGGGATCTCA |
| | mitochondrial targeting | AGTTTGCAAAAGACACTGCGTAATGCTCTG |
| | sequence of the rice | ATCCCTACTGAAACCACTCAACAATTCAT |
| | RPS10 protein | CGTCAAGAACGGAATAATTAAAGAAGATGA |
| | | GTTACGTGGTGAGAACCGACAAATTCTGAA |
| | | AGATATCATGGATGATTATTATCGTGGATT |
| | | CATCTCTGAGACTCTGAGTTCTATTGATGA |
| | | TATAGATTGGACTAGTCTGTTCGAAAAGAT |
| | | GGAAATTCAACTGAAGAATGGTGATAATAA |
| | | AGATACCTTGATTAAGGAACAAACAGAGTA |
| | | TCGTAAAGCAATCCATAAGAAATTTGCTAA |
| | | CGATGATCGATTTAAGAACATGTTTAGTGC |
| | | CAAACTGATTAGTGATATATTACCTGAGTT |
| | | CGTCATCCATAACAATAATTATTCGGCATC |
| | | AGAGAAAGAGGAAAAGACCCAAGTGATAAA |
| | | GTTGTTCTCGCGATTTGCAACTAGTTTCAA |
| | | GGATTATTTCAAGAACCGTGCAAATTGTTT |
| | | CTCAGCAGATGATATTTCATCAAGTAGTTG |
| | | TCATCGTATCGTCAACGATAATGCAGAGAT |
| | | ATTCTTCTCAAATGCACTGGTCTATCGTCG |
| | | AATCGTAAAGTCGCTGAGTAATGATGATAT |
| | | CAACAAAATTTCGGGTGATATGAAAGATTC |
| | | ATTAAAAGAAATGAGTCTGGAAGAAATATA |
| | | TTCTTATGAGAAGTATGGTGAATTTATTAC |
| | | TCAAGAAGGTATTAGTTTCTATAATGATAT |
| | | CTGTGGTAAAGTAAATTCATTCATGAACCT |
| | | GTATTGTCAAAAGAATAAGGAAAACAAGAA |
| | | TTTATATAAACTTCAAAAACTTCATAAACA |
| | | AATTCTATGTATTGCAGATACTAGTTATGA |
| | | GGTCCCATATAAGTTTGAAAGTGATGAGGA |
| | | AGTGTATCAATCAGTTAACGGTTTCCTTGA |
| | | TAACATTAGTAGTAAACATATAGTCGAAAG |
| | | ATTACGTAAAATCGGTGATAACTATAACGG |
| | | TTATAACCTGGATAAAATTTATATCGTGTC |
| | | CAAATTCTATGAGAGTGTTAGTCAAAAGAC |
| | | CTATCGTGATTGGGAAACAATTAATACCGC |
| | | CCTCGAAATTCATTATAATAATATCTTGCC |
| | | TGGTAACGGTAAAAGTAAAGCCGATAAAGT |
| | | AAAGAAAGCAGTTAAGAATGATTTACAAAA |
| | | GTCCATCACCGAAATAAATGAACTAGTGTC |
| | | AAACTATAAGCTGTGTAGTGATGATAACAT |
| | | CAAAGCAGAGACTTATATACATGAGATTAG |
| | | TCATATCTTGAATAACTTTGAAGCACAAGA |
| | | ATTGAAATATAATCCAGAAATTCATCTAGT |
| | | TGAATCCGAGCTTAAAGCAAGTGAGCTTAA |
| | | AAACGTGCTGGATGTGATCATGAATGCCTT |
| | | TCATTGGTGTTCGGTTTTTATGACTGAGGA |
| | | ACTTGTTGATAAAGATAACAATTTTTATGC |
| | | AGAACTGGAGGAGATTTATGATGAAATTTA |
| | | TCCAGTAATTAGTCTGTATAACCTGGTTCG |
| | | TAACTATGTTACCCAAAAACCATATAGTAC |
| | | TAAGAAGATTAAATTGAACTTTGGAATACC |
| | | AACATTAGCAGATGGTTGGTCAAAGTCCAA |
| | | AGAGTATTCTAATAACGCTATCATACTGAT |
| | | GCGTGATAATCTGTATTATCTGGGTATCTT |
| | | TAATGCAAAGAATAAACCAGATAAGAAGAT |
| | | TATCGAGGGTAATACATCAGAAAATAAGGG |
| | | TGATTATAAGAAGATGATTTATAATTTGCT |
| | | CCCTGGTCCTAACAAAATGATCCCAAAAGT |
| | | TTTCTTGAGTAGTAAGACAGGGGTGGAAAC |
| | | CTATAAACCAAGTGCCTATATCCTAGAGGG |
| | | GTATAAACAAAATAAACATATCAAGTCTTC |
| | | AAAAGATTTCGATATCACTTTCTGTCATGA |
| | | TCTGATCGATTATTTCAAAAACTGTATTGC |
| | | AATTCATCCTGAGTGGAAGAACTTCGGGTT |
| | | TGATTTCAGTGATACCAGTACTTATGAAGA |
| | | TATTTCCGGGTTCTATCGTGAGGTAGAGTT |
| | | ACAAGGTTATAAGATTGATTGGACATATAT |
| | | TAGTGAAAAGGATATTGATCTGCTGCAAGA |
| | | AAAGGGTCAACTGTATCTGTTCCAAATATA |
| | | TAACAAAGATTTCTCGAAGAAATCAACCGG |
| | | GAATGATAACCTTCATACCATGTATCTGAA |
| | | AAATCTTTTCTCAGAAGAAAATCTTAAGGA |
| | | TATCGTCCTGAAACTTAACGGTGAAGCTGA |
| | | AATATTCTTCAGAAAGAGTAGTATAAAGAA |
| | | CCCAATCATTCATAAGAAAGGTTCGATATT |
| | | AGTCAACCGTACCTATGAAGCAGAAGAAAA |
| | | GGATCAATTTGGTAACATTCAAATTGTGCG |
| | | TAAGAATATTCCAGAGAACATTTATCAAGA |
| | | GCTGTATAAATATTTCAACGATAAGTCAGA |
| | | TAAAGAGCTGTCTGATGAAGCAGCCAAACT |
| | | GAAGAATGTAGTGGGACATCATGAGGCAGC |
| | | AACAAATATAGTCAAGGATTATCGTTATAC |
| | | ATATGATAAATATTTCCTTCATATGCCTAT |
| | | TACAATCAATTTCAAAGCCAATAAAACTGG |
| | | TTTCATTAATGATAGAATCTTACAATATAT |
| | | CGCTAAAGAAAAGGATTTACATGTTATCGG |
| | | TATTGATCGAGGTGAGCGTAACCTGATCTA |
| | | TGTGTCAGTGATTGATACTTGTGGTAATAT |
| | | AGTTGAACAAAAGAGTTTTAACATTGTAAA |
| | | CGGTTATGATTATCAAATAAAACTGAAACA |
| | | ACAAGAGGGTGCTAGACAAATTGCTCGAAA |
| | | GGAATGGAAAGAAATTGGTAAGATTAAAGA |
| | | GATCAAAGAGGGTTATCTGAGTTTAGTAAT |
| | | CCATGAGATCTCTAAGATGGTAATCAAATA |
| | | TAATGCAATTATAGCAATGGAGGATTTGTC |
| | | TTATGGTTTCAAGAAAGGGCGTTTCAAGGT |
| | | CGAACGACAAGTTTATCAAAAGTTCGAAAC |
| | | TATGCTCATCAATAAACTCAACTATCTGGT |
| | | ATTCAAAGATATTTCGATTACCGAGAATGG |
| | | TGGACTCCTGAAAGGTTATCAACTGACATA |
| | | TATTCCTGATAAACTTAAGAACGTGGGTCA |
| | | TCAATGTGGTTGTATTTTCTATGTGCCTGC |
| | | TGCATATACAAGTAAGATTGATCCAACCAC |
| | | CGGTTTCGTGAATATCTTCAAGTTCAAAGA |
| | | TCTGACAGTGGATGCAAAGCGTGAGTTCAT |
| | | TAAGAAGTTCGATTCAATTCGTTATGATAG |
| | | TGAAAAGAATCTGTTCTGTTTCACATTCGA |
| | | TTATAATAACTTCATTACACAAAACACAGT |
| | | CATGAGTAAGTCATCGTGGAGTGTGTATAC |
| | | ATATGGTGTGCGTATCAAACGTCGATTTGT |
| | | GAACGGTCGTTTCTCAAACGAAAGTGATAC |
| | | CATTGATATAACCAAAGATATGGAGAAGAC |
| | | ATTGGAAATGACAGATATTAACTGGCGTGA |
| | | TGGTCATGATCTTCGTCAAGATATTATAGA |
| | | TTATGAAATTGTTCAACATATATTCGAAAT |
| | | TTTCCGTCTAACAGTGCAAATGCGTAACTC |
| | | CTTGTCTGAACTGGAGGATCGTGATTATGA |
| | | TCGTCTCATTTCACCTGTACTGAACGAAAA |
| | | TAACATATTCTATGATAGTGCAAAGGCTGG |
| | | AGATGCACTTCCTAAGGATGCCGATGCAAA |
| | | TGGTGCATATTGTATTGCATTAAAAGGGTT |
| | | ATATGAAATTAAACAAATTACCGAAAATTG |
| | | GAAAGAAGATGGTAAATTTTCGCGTGATAA |
| | | ACTCAAAATCAGTAATAAAGATTGGTTCGA |
| | | TTTCATCCAAAATAAGCGTTATCTCTAA |
| 89 | Amino acid sequence of | MAAKIRIVMKSFMSQANKVEGVIPYAQKVG |
| | the MTS(RPS10)- | LPESRSLYTVLRSPHIDKKSREQFSMNNGT |
| | MAD7 fusion protein | NNFQNFIGISSLQKTLRNALIPTETTQQFI |
| | encoded by SEQ ID NO: | VKNGIIKEDELRGENRQILKDIMDDYYRGF |
| | 88 | ISETLSSIDDIDWTSLFEKMEIQLKNGDNK |
| | | DTLIKEQTEYRKAIHKKFANDDRFKNMFSA |
| | | KLISDILPEFVIHNNNYSASEKEEKTQVIK |
| | | LFSRFATSFKDYFKNRANCFSADDISSSSC |
| | | HRIVNDNAEIFFSNALVYRRIVKSLSNDDI |
| | | NKISGDMKDSLKEMSLEEIYSYEKYGEFIT |
| | | QEGISFYNDICGKVNSFMNLYCQKNKENKN |
| | | LYKLQKLHKQILCIADTSYEVPYKFESDEE |
| | | VYQSVNGFLDNISSKHIVERLRKIGDNYNG |
| | | YNLDKIYIVSKFYESVSQKTYRDWETINTA |
| | | LEIHYNNILPGNGKSKADKVKKAVKNDLQK |
| | | SITEINELVSNYKLCSDDNIKAETYIHEIS |
| | | HILNNFEAQELKYNPEIHLVESELKASELK |
| | | NVLDVIMNAFHWCSVFMTEELVDKDNNFYA |
| | | ELEEIYDEIYPVISLYNLVRNYVTQKPYST |
| | | KKIKLNFGIPTLADGWSKSKEYSNNAIILM |
| | | RDNLYYLGIFNAKNKPDKKIIEGNTSENKG |
| | | DYKKMIYNLLPGPNKMIPKVFLSSKTGVET |
| | | YKPSAYILEGYKQNKHIKSSKDFDITFCHD |
| | | LIDYFKNCIAIHPEWKNFGFDFSDTSTYED |
| | | ISGFYREVELQGYKIDWTYISEKDIDLLQE |
| | | KGQLYLFQIYNKDFSKKSTGNDNLHTMYLK |
| | | NLFSEENLKDIVLKLNGEAEIFFRKSSIKN |
| | | PIIHKKGSILVNRTYEAEEKDQFGNIQIVR |
| | | KNIPENIYQELYKYFNDKSDKELSDEAAKL |
| | | KNVVGHHEAATNIVKDYRYTYDKYFLHMPI |
| | | TINFKANKTGFINDRILQYIAKEKDLHVIG |
| | | IDRGERNLIYVSVIDTCGNIVEQKSFNIVN |
| | | GYDYQIKLKQQEGARQIARKEWKEIGKIKE |
| | | IKEGYLSLVIHEISKMVIKYNAIIAMEDLS |
| | | YGFKKGRFKVERQVYQKFETMLINKLNYLV |
| | | FKDISITENGGLLKGYQLTYIPDKLKNVGH |
| | | QCGCIFYVPAAYTSKIDPTTGFVNIFKFKD |
| | | LTVDAKREFIKKFDSIRYDSEKNLFCFTFD |
| | | YNNFITQNTVMSKSSWSVYTYGVRIKRRFV |
| | | NGRFSNESDTIDITKDMEKTLEMTDINWRD |
| | | GHDLRQDIIDYEIVQHIFEIFRLTVQMRNS |
| | | LSELEDRDYDRLISPVLNENNIFYDSAKAG |
| | | DALPKDADANGAYCIALKGLYEIKQITENW |
| | | KEDGKFSRDKLKISNKDWFDFIQNKRYL |
| 90 | Amino acid sequence of | MAAKIRIVMKSFMSQANKVEGVIPYAQKVG |
| | the mitochondrial | LPESRSLYTVLRSPHIDKKSREQFSM |
| | targeting sequence of the | |
| | rice RPS10 protein | |
| 91 | Amino acid sequence of | MAAKIRIVMKSFMSQANKVEGVIPYAQKVG |
| | the fusion protein | LPESRSLYTVLRSPHIDKKSREQFSMNTIN |
| | encoded by SEQ ID NO: | IAKNDFSDIELAAIPFNTLADHYGERLARE |
| | 38, in which the | QLALEHESYEMGEARFRKMFERQLKAGEVA |
| | mitochondrial targeting | DNAAAKPLITTLLPKMIARINDWFEEVKAK |
| | sequence of the | RGKRPTAFQFLQEIKPEAVAYITIKTTLAC |
| | At5G47030 protein from | LTSADNTTVQAVASAIGRAIEDEARFGRIR |
| | Arabidopsis thaliana | DLEAKHFKKNVEEQLNKRVGHVYKKAFMQV |
| | is fused to the amino- | VEADMLSKGLLGGEAWSSWHKEDSIHVGVR |
| | terminus of the T7 RNA | CIEMLIESTGMVSLHRQNAGVVGQDSETIE |
| | polymerase | LAPEYAEAIATRAGALAGISPMFQPCVVPP |
| | | KPWTGITGGGYWANGRRPLALVRTHSKKAL |
| | | MRYEDVYMPEVYKAINIAQNTAWKINKKVL |
| | | AVANVITKWKHCPVEDIPAIEREELPMKPE |
| | | DIDMNPEALTAWKRAAAAVYRKDKARKSRR |
| | | ISLEFMLEQANKFANHKAIWFPYNMDWRGR |
| | | VYAVSMFNPQGNDMTKGLLTLAKGKPIGKE |
| | | GYYWLKIHGANCAGVDKVPFPERIKFIEEN |
| | | HENIMACAKSPLENTWWAEQDSPFCFLAFC |
| | | FEYAGVQHHGLSYNCSLPLAFDGSCSGIQH |
| | | FSAMLRDEVGGRAVNLLPSETVQDIYGIVA |
| | | KKVNEILQADAINGTDNEVVTVTDENTGEI |
| | | SEKVKLGTKALAGQWLAYGVTRSVTKRSVM |
| | | TLAYGSKEFGFRQQVLEDTIQPAIDSGKGL |
| | | MFTQPNQAAGYMAKLIWESVSVTVVAAVEA |
| | | MNWLKSAAKLLAAEVKDKKTGEILRKRCAV |
| | | HWVTPDGFPVWQEYKKPIQTRLNLMFLGQF |
| | | RLQPTINTNKDSEIDAHKQESGIAPNFVHS |
| | | QDGSHLRKTVVWAHEKYGIESFALIHDSFG |
| | | TIPADAANLFKAVRETMVDTYESCDVLADF |
| | | YDQFADQLHESQLDKMPALPAKGNLNLRDI |
| | | LESDFAFA |
| 92 | Amino acid sequence of | MFKQASRLLSRSVAAASSKSVTTRAFSTEL |
| | the mitochondrial | PSTLDS |
| | targeting sequence of the | |
| | Arabidopsis thaliana | |
| | At5G47030 protein | |
| 93 | Nucleotide sequence of | tttatagttctagcattaaccggtc |
| | the gRNA1 target site in | |
| | the rice mitochondrial | |
| | genome. The first four | |
| | nucleotides correspond | |
| | to the MAD7 PAM | |
| | sequence | |
| 94 | Nucleotide sequence of | tttattcaattatgaaattactcat |
| | the gRNA2 target site in | |
| | the rice mitochondrial | |
| | genome. The first four | |
| | nucleotides correspond | |
| | to the MAD7 PAM | |
| | sequence | |
| 95 | Nucleotide sequence of | cttccggatatagaaacattaaagc |
| | the gRNA3 target site in | |
| | the rice mitochondrial | |
| | genome. The first four | |
| | nucleotides correspond | |
| | to the MAD7 PAM | |
| | sequence | |
| 96 | Nucleotide sequence of | tttagtcacatttctaccggtgcac |
| | the gRNA4 target site in | |
| | the rice mitochondrial | |
| | genome. The first four | |
| | nucleotides correspond | |
| | to the MAD7 PAM | |
| | sequence | |
| 97 | Nucleotide sequence of | ttCatTgtATtGgcTCTTacTggAT |
| | the modified gRNA1 | |
| | target site in the Donor | |
| | DNA targeted to gRNA1 | |
| | and gRNA3 sites, such | |
| | that it is no longer a | |
| | target for MAD7 | |
| 98 | Nucleotide sequence of | tttattcattactcat |
| | the modified gRNA2 | |
| | target site in the Donor | |
| | DNA targeted to gRNA2 | |
| | and gRNA4 sites, such | |
| | that it is no longer a | |
| | target for MAD7 | |
| 99 | Nucleotide sequence of | GtGAcggatatagaaacattaaagc |
| | the modified gRNA3 | |
| | target site in the Donor | |
| | DNA targeted to gRNA1 | |
| | and gRNA3 sites, such | |
| | that it is no longer a | |
| | target for MAD7 | |
| 100 | Nucleotide sequence | GTCTGGCCCCAAATTCTAATTTCTACTGTT |
| | encoding gRNA1 | GTAGATtagttctagcattaaccggtc |
| 101 | Nucleotide sequence | GTCTGGCCCCAAATTCTAATTTCTACTGTT |
| | encoding gRNA2 | GTAGATttcaattatgaaattactcat |
| 102 | Nucleotide sequence | GTCTGGCCCCAAATTCTAATTTCTACTGTT |
| | encoding gRNA3 | GTAGATcggatatagaaacattaaagc |
| 103 | Nucleotide sequence | GTCTGGCCCCAAATTCTAATTTCTACTGTT |
| | encoding gRNA4 | GTAGATgtcacatttctaccggtgcac |
| 104 | Nucleotide sequence of | GGGCCCgccggtcatagttcagtaaagatt |
| | the Donor DNA that is | ttaagtgggttcgcttggactatgctattt |
| | targeted to the rice | ctgaataatattttctatttcataggagat |
| | mitochondrial gRNA1 | cttggtcccttactttaatgtttctatatc |
| | and gRNA3 sites, with | cgtggaattaggtgtagctatattacaagc |
| | PspOMI sites at each to | tcatgtttctacgatctcaatttgtattta |
| | facilitate cloning | cttgaatgatgctataaatctccatcaaaa |
| | | tgagtaatttcataattgaataaaaacgag |
| | | gagccgaagattttagggggcgggaCAAAC |
| | | GCGGAAGTGTATTGCGTTACAAAAAATGAC |
| | | AACTAGCATTTGTTTTTTCATTTCATGTTC |
| | | GAATTCGTTTTTCGTTGGAAAAACCAACGC |
| | | CGACCCCAAACAAGTCTCTCCAATATAAGG |
| | | AGAGCGGAGCTTAAAAATATTATTTTATTG |
| | | TGCTATGGCAAATCTGGTCCGATGGCTCTT |
| | | CTCCACTACCCGAGGGACTAACGGTCTTCC |
| | | ATATTTCATCTTCGGTGTCGTTGTAGGAGG |
| | | CGCCCTGTTGTTTGCTTTGCTAAAGTATCA |
| | | GGCCCCTCTGTACGACCCGGCTTTAATGGA |
| | | AAAAATCATAGATCATAATATAAAAGCCGG |
| | | GCACCCTATAGAGGTTGACTATTCGTGGTG |
| | | GGGCACCTCTATTCGTGTAGTCTTTCCTAA |
| | | GggaggtggaggttcaAGTGAGCTGATTAA |
| | | GGAGAACATGCATATGAAGCTGTATATGGA |
| | | GGGTACCGTGAACAACCATCATTTCAAGTG |
| | | TACATCCGAGGGTGAAGGTAAGCCTTATGA |
| | | GGGTACCCAAACCATGAGAATCAAGGTGGT |
| | | CGAGGGTGGTCCTCTCCCTTTCGCTTTCGA |
| | | TATTCTGGCTACCAGTTTCATGTATGGTAG |
| | | TAGAACCTTCATCAACCATACTCAAGGTAT |
| | | CCCTGATTTCTTTAAGCAATCCTTCCCTGA |
| | | GGGTTTCACATGGGAGAGAGTCACCACATA |
| | | TGAAGATGGGGGTGTGCTGACCGCTACCCA |
| | | AGATACCAGTCTCCAAGATGGTTGTCTCAT |
| | | CTATAACGTCAAGATCAGAGGGGTGAACTT |
| | | CCCATCCAACGGTCCTGTGATGCAAAAGAA |
| | | AACACTCGGTTGGGAGGCCAACACCGAGAT |
| | | GCTGTATCCTGCTGATGGTGGTCTGGAAGG |
| | | TAGAAGTGATATGGCCCTGAAGCTCGTGGG |
| | | TGGGGGTCATCTGATCTGTAACTTCAAGAC |
| | | CACATATAGATCCAAGAAACCAGCTAAGAA |
| | | CCTCAAGATGCCTGGTGTCTATTATGTGGA |
| | | TCATAGACTGGAAAGAATCAAGGAGGCCGA |
| | | TAAAGAGACTTATGTCGAGCAACATGAGGT |
| | | GGCTGTGGCTAGATATTGTGATCTCCCTAG |
| | | TAAACTGGGGCATAAGTAAGAAAGACAGGA |
| | | CAGTGGTGGTTTGCTCATACTTTCATTACA |
| | | AAACCATACTATGGAATgctttaatgtttc |
| | | tatatccgTCaCtagggaatctcgtgcttg |
| | | catatctaaatctaagttttgagacagacc |
| | | tttcatgggttcaaagaaaagaagagtacg |
| | | agtgggtgatgtgattgaGGGCCC |
| 105 | Nucleotide sequence of | GGGCCCgcattaacTggtctggaattaggt |
| | the Donor DNA that is | gtagctatattacaagctcatgtttctacg |
| | targeted to the rice | atctcaatttgtatttacttgaatgatgct |
| | mitochondrial gRNA2 | ataaatctccatcaaaatgagtaatgaata |
| | and gRNA4 sites, with | aaaacgaggagccgaagattttaggggggg |
| | PspOMI sites at each to | gaCAAACGCGGAAGTGTATTGCGTTACAAA |
| | facilitate cloning | AAATGACAACTAGCATTTGTTTTTTCATTT |
| | | CATGTTCGAATTCGTTTTTCGTTGGAAAAA |
| | | CCAACGCCGACCCCAAACAAGTCTCTCCAA |
| | | TATAAGGAGAGCGGAGCTTAAAAATATTAT |
| | | TTTATTGTGCTATGGCAAATCTGGTCCGAT |
| | | GGCTCTTCTCCACTACCCGAGGGACTAACG |
| | | GTCTTCCATATTTCATCTTCGGTGTCGTTG |
| | | TAGGAGGCGCCCTGTTGTTTGCTTTGCTAA |
| | | AGTATCAGGCCCCTCTGTACGACCCGGCTT |
| | | TAATGGAAAAAATCATAGATCATAATATAA |
| | | AAGCCGGGCACCCTATAGAGGTTGACTATT |
| | | CGTGGTGGGGCACCTCTATTCGTGTAGTCT |
| | | TTCCTAAGggaggtggaggttcaAGTGAGC |
| | | TGATTAAGGAGAACATGCATATGAAGCTGT |
| | | ATATGGAGGGTACCGTGAACAACCATCATT |
| | | TCAAGTGTACATCCGAGGGTGAAGGTAAGC |
| | | CTTATGAGGGTACCCAAACCATGAGAATCA |
| | | AGGTGGTCGAGGGTGGTCCTCTCCCTTTCG |
| | | CTTTCGATATTCTGGCTACCAGTTTCATGT |
| | | ATGGTAGTAGAACCTTCATCAACCATACTC |
| | | AAGGTATCCCTGATTTCTTTAAGCAATCCT |
| | | TCCCTGAGGGTTTCACATGGGAGAGAGTCA |
| | | CCACATATGAAGATGGGGGTGTGCTGACCG |
| | | CTACCCAAGATACCAGTCTCCAAGATGGTT |
| | | GTCTCATCTATAACGTCAAGATCAGAGGGG |
| | | TGAACTTCCCATCCAACGGTCCTGTGATGC |
| | | AAAAGAAAACACTCGGTTGGGAGGCCAACA |
| | | CCGAGATGCTGTATCCTGCTGATGGTGGTC |
| | | TGGAAGGTAGAAGTGATATGGCCCTGAAGC |
| | | TCGTGGGTGGGGGTCATCTGATCTGTAACT |
| | | TCAAGACCACATATAGATCCAAGAAACCAG |
| | | CTAAGAACCTCAAGATGCCTGGTGTCTATT |
| | | ATGTGGATCATAGACTGGAAAGAATCAAGG |
| | | AGGCCGATAAAGAGACTTATGTCGAGCAAC |
| | | ATGAGGTGGCTGTGGCTAGATATTGTGATC |
| | | TCCCTAGTAAACTGGGGCATAAGTAAGAAA |
| | | GACAGGACAGTGGTGGTTTGCTCATACTTT |
| | | CATTACAAAACCATACTATGGAATTCactt |
| | | ctcatgaaataattccctttccaaggaaag |
| | | gaaaacaagaactcgaatactcgtaatagc |
| | | gatcccgatccacctacttttttctattct |
| | | ttgattcgGGGCCC |
| 106 | Nucleotide sequence of | cgtgcattaagctcaggaatacgt |
| | the ATP6-1 primer used | |
| | for PCR amplification of | |
| | the junction region of | |
| | Donor DNA integration | |
| | into rice mitochondrial | |
| | DNA (FIG. 14) | |
| 107 | Nucleotide sequence of | CATCGGACCAGATTTGCCATAGCA |
| | the 79-2 primer used for | |
| | PCR amplification of the | |
| | junction region of Donor | |
| | DNA integration into | |
| | rice mitochondrial DNA | |
| | (FIG. 14) | |
| 108 | Nucleotide sequence | ATGGCCGCCAAGATcCGCATcGTGATGAAA |
| | encoding the | TCTTTTATGAGCCAAGCTAACAAAGTTGAA |
| | MTS(RPS10)-ptxD- | GGGGTTATTCCATACGCGCAGAAGGTTGGA |
| | eGFP fusion protein in | TTGCCTGAATCACGATCCTTGTATACCGTG |
| | plasmid pNAP256 | CTACGATCGCCTCACATcGACAAGAAGTCG |
| | | AGGGAGCAGTTCTCGATGCTGCCGAAACTC |
| | | GTTATcACTCACCGgGTgCACGATGAGATC |
| | | CTGCAACTGCTGGCGCCACATTGCGAGCTG |
| | | ATGACCAACCAGACCGACAGCACGCTGACG |
| | | CGCGAGGAAATTCTGCGCCGCTGcCGCGAT |
| | | GCTCAGGCGATGATGGCGTTCATGCCCGAT |
| | | CGGGTCGATGCAGACTTTCTTCAAGCCTGC |
| | | CCTGAGCTGCGcGTgGTCGGCTGCGCGCTC |
| | | AAGGGCTTCGACAATTTCGATGTGGACGCC |
| | | TGcACTGCCCGCGGGGTCTGGCTGACCTTC |
| | | GTGCCTGATCTGTTGACGGTCCCGACTGCC |
| | | GAGCTGGCGATCGGACTGGCGGTGGGGCTG |
| | | GGGCGGCATCTGCGGGCAGCAGATGCGTTC |
| | | GTCCGCTCTGGCGAGTTCCAGGGCTGGCAA |
| | | CCACAGTTCTACGGCACGGGGCTGGATAAC |
| | | GCTACGGTCGGCATCCTTGGCATGGGCGCC |
| | | ATCGGACTGGCCATGGCTGATCGCTTGCAG |
| | | GGATGGGGCGCGACCCTGCAGTACCACGAG |
| | | GCGAAGGCTCTGGATACACAAACCGAGCAA |
| | | CGGCTCGGCCTGCGCCAGGTGGCGTGCAGC |
| | | GAACTCTTCGCCAGCTCGGACTTCATCCTG |
| | | CTGGCGCTTCCCTTGAATGCCGATACCCAG |
| | | CATCTGGTCAACGCCGAGCTGCTTGCCCTC |
| | | GTgCGGCCGGGCGCTCTGCTTGTgAACCCC |
| | | TGcCGcGGTTCGGTgGTGGATGAAGCCGCC |
| | | GTGCTCGCGGCGCTTGAGCGgGGCCAGCTC |
| | | GGCGGGTATGCGGCGGATGTgTTCGAAATG |
| | | GAAGACTGGGCTCGCGCGGACCGGCCGCGG |
| | | CTGATCGATCCTGCGCTGCTCGCGCATCCG |
| | | AATACGCTGTTCACTCCGCACATcGGGTCG |
| | | GCAGTGCGCGCGGTGCGCCTGGAGATTGAA |
| | | CGcTGcGCAGCGCAGAACATCATCCAGGTg |
| | | TTGGCAGGTGCGCGCCCAATCAACGCTGCG |
| | | AACCGcCTGCCCAAGGCCGAGCCTGCCGCA |
| | | TGcccagttgctactATGGTGAGTAAGGGA |
| | | GAGGAGCTGTTCACCGGGGTGGTGCCTATC |
| | | CTGGTCGAGCTGGATGGTGATGTAAACGGT |
| | | CATAAATTCAGTGTGTCCGGTGAAGGTGAA |
| | | GGTGATGCCACCTATGGTAAGCTGACCCTT |
| | | AAGTTCATCTGTACCACCGGAAAGCTGCCT |
| | | GTGCCTTGGCCTACCCTCGTGACCACCCTG |
| | | ACATATGGAGTGCAATGTTTCAGTCGTTAT |
| | | CCTGATCATATGAAGCAACATGATTTCTTT |
| | | AAATCCGCCATGCCTGAAGGTTATGTCCAA |
| | | GAGCGTACCATATTCTTTAAAGATGATGGT |
| | | AACTATAAGACCCGTGCCGAGGTGAAGTTC |
| | | GAGGGTGATACCCTGGTGAACCGTATTGAG |
| | | CTTAAGGGTATCGATTTCAAGGAGGATGGA |
| | | AACATCCTGGGGCATAAGCTGGAGTATAAC |
| | | TATAACAGTCATAACGTCTATATCATGGCC |
| | | GATAAGCAAAAGAACGGTATCAAGGTGAAC |
| | | TTCAAGATCCGTCATAATATCGAAGATGGA |
| | | AGTGTGCAACTCGCCGATCATTATCAACAA |
| | | AACACCCCTATCGGTGATGGTCCTGTGCTG |
| | | CTGCCTGATAACCATTATCTGAGTACCCAA |
| | | TCCGCCCTGAGTAAAGATCCTAACGAGAAG |
| | | CGTGATCAAATGGTACTGCTTGAGTTCGTT |
| | | ACCGCCGCCGGGATCACTCTCGGTATGGAT |
| | | GAGCTGTATAAGTAA |
| 109 | Amino acid sequence of | MAAKIRIVMKSFMSQANKVEGVIPYAQKVG |
| | the MTS(RPS10)-ptxD- | LPESRSLYTVLRSPHIDKKSREQFSMLPKL |
| | eGFP fusion protein | VITHRVHDEILQLLAPHCELMTNQTDSTLT |
| | encoded by SEQ ID NO: | REEILRRCRDAQAMMAFMPDRVDADFLQAC |
| | 108 | PELRVVGCALKGFDNFDVDACTARGVWLTF |
| | | VPDLLTVPTAELAIGLAVGLGRHLRAADAF |
| | | VRSGEFQGWQPQFYGTGLDNATVGILGMGA |
| | | IGLAMADRLQGWGATLQYHEAKALDTQTEQ |
| | | RLGLRQVACSELFASSDFILLALPLNADTQ |
| | | HLVNAELLALVRPGALLVNPCRGSVVDEAA |
| | | VLAALERGQLGGYAADVFEMEDWARADRPR |
| | | LIDPALLAHPNTLFTPHIGSAVRAVRLEIE |
| | | RCAAQNIIQVLAGARPINAANRLPKAEPAA |
| | | CPVATMVSKGEELFTGVVPILVELDGDVNG |
| | | HKFSVSGEGEGDATYGKLTLKFICTTGKLP |
| | | VPWPTLVTTLTYGVQCFSRYPDHMKQHDFF |
| | | KSAMPEGYVQERTIFFKDDGNYKTRAEVKF |
| | | EGDTLVNRIELKGIDFKEDGNILGHKLEYN |
| | | YNSHNVYIMADKQKNGIKVNFKIRHNIEDG |
| | | SVQLADHYQQNTPIGDGPVLLPDNHYLSTQ |
| | | SALSKDPNEKRDQMVLLEFVTAAGITLGMD |
| | | ELYK |
| 110 | Nucleotide sequence of | ttctctgacattccatgtttccgaaacgga |
| | a region surrounding the | tcctataaaatatttc |
| | start codon of rice | |
| | mitochondrial NAD4L as | |
| | presented in FIG. 8 | |
| 111 | Amino acid sequence of | MDPIKYF |
| | the initial seven amino | |
| | acids of NAD4L | |
| | encoded by SEQ ID NO: | |
| | 110 | |
| 112 | Nucleotide sequence of | CCATCGTCTTTGTTAAAGTATGCTGCCTAA |
| | a region surrounding the | ACTC |
| | start codon of ptxD in | |
| | the pATP1-ptxD | |
| | expression unit as | |
| | presented in FIG. 8 | |
| 113 | Amino acid sequence of | MLPKL |
| | the initial seven amino | |
| | acids of ptxD encoded | |
| | by SEQ ID NO: 112 | |
| 114 | Nucleotide sequence of | CCATCGTCTTTGTTAAAGTCATTCCATGTT |
| | a region surrounding the | TCCGAAACGGATCCTCTGCCTAAACTC |
| | start codon of ptxD in | |
| | the pATP1-RNAed-ptxD | |
| | expression unit, in which | |
| | the start codon of ptxD | |
| | has been replaced with | |
| | the putative RNA | |
| | editing site of NAD4L, | |
| | as presented in FIG. 8 | |
| 115 | Nucleotide sequence of | CCAUCGUCUUUGUUAAAGUCAUUCCAUGUU |
| | a region surrounding the | UCCGAAAUGGAUCCUCUGCCUAAACUC |
| | start codon of the edited | |
| | mRNA obtained after | |
| | transcription and | |
| | subsequent RNA | |
| | processing of the | |
| | pATP1-RNAed-ptxD | |
| | transcript | |
| 116 | Amino acid sequence of | MDPLPKL |
| | the initial nine amino | |
| | acids of ptxD encoded | |
| | by the pATPI-RNAed- | |
| | ptxD edited mRNA | |
| | transcript | |
| 117 | Nucleotide sequence of | TGCATTAAGCTCAGGAATACGTTTATTTGC |
| | the DNA obtained by | TAATATGATGGCCGGTCATAGTTCAGTAAA |
| | PCR amplification of the | GATTTTAAGTGGGTTCGCTTGGACTATGCT |
| | Donor DNA integrated | ATTTCTGAATAATATTTTCTATTTCATAGG |
| | at the gRNA1 target site | AGATCTTGGTCCCTTATTTATAGTTCTAGC |
| | of rice mitochondrial | ATTAACCGGTCTGGAATTAGGTGTAGCTAT |
| | DNA, as presented in | ATTACAAGCTCATGTTTCTACGATCTCAAT |
| | FIG. 15A | TTGTATTTACTTGAATGATGCTATAAATCT |
| | | CCATCAAAATGAGTAATTTCATAATTGAAT |
| | | AAAAACGAGGAGCCGAAGATTTTAGGGGGC |
| | | GGGACAAACGCGGAAGTGTATTGCGTTACA |
| | | AAAAATGACAACTAGCATTTGTTTTTTCAT |
| | | TTCATGTTCGAATTCGTTTTTCGTTGGAAA |
| | | AACCAACGCCGACCCCAAACAAGTCTCTCC |
| | | AATATAAGGAGAGCGGAGCTTAAAA |
| 118 | Nucleotide sequence of | AGGAATACGTTTATTTGCTAATATGATGGC |
| | the DNA obtained by | CGGTCATAGTTCAGTAAAGATTTTAAGTGG |
| | PCR amplification of the | GTTCGCTTGGACTATGCTATTTCTGAATAA |
| | Donor DNA integrated | TATTTTCTATTTCATAGGAGATCTTGGTCC |
| | at the gRNA2 target site | CTTATTTATAGTTCTAGCATTAACCGGTCT |
| | of rice mitochondrial | GGAATTAGGTGTAGCTATATTACAAGCTCA |
| | DNA, as presented in | TGTTTCTACGATCTCAATTTGTATTTACTT |
| | FIG. 15B | GAATGATGCTATAAATCTCCATCAAAATGA |
| | | GTAATTTCATAATTGAATAAAAACGAGGAG |
| | | CCGAAGATTTTAGGGGGGGGACAAACGCGG |
| | | AAGTGTATTGCGTTACAAAAAATGACAACT |
| | | AGCATTTGTTTTTTCATTTCATGTTCGAAT |
| | | TCGTTTTTCGTTGGAAAAACCAACGCCGAC |
| | | CCCAAACAAGTCTCTCCAATATAAGGAGAG |
| | | CGGAGCT |
| 119 | Nucleotide sequence of | CATTCCATGTTTCCGAAAcGGATCCT |
| | the putative RNA | |
| | editing site of rice | |
| | mitochondrial NAD4L, | |
| | present in the RNAed- | |
| | ptxD-eGFP coding | |
| | regions of plasmids | |
| | pNAP246 and pNAP251 | |
| 120 | Amino acid sequence of | MANLVRWLFSTTRGTNGLPYFIFGVVVGGA |
| | the orf79 polypeptide | LLFALLKYQAPLYDPALMEKIIDHNIKAGH |
| | encoded by SEQ ID NO: | PIEVDYSWWGTSIRVVFPK |
| | 87 | |
| 121 | Nucleotide sequence of | cgaggtgtagcgcagtctggtcagcgcatc |
| | the multigene cassette | tgttttgggtacagagggccataggttcga |
| | encoding trnP-gRNA1- | atcctgtcaccttgaGTCTGGCCCCAAATT |
| | trnE-gRNA3-trnK. The | CTAATTTCTACTGTTGTAGATtagttctag |
| | three tRNA sequences | cattaaccggtcgtccctttcgtccagtgg |
| | are from rice | ttaggacatcgtcttttcatgtcgaagaca |
| | mitochondrial DNA | cgggttcgattcccgtaagggataGTCTGG |
| | | CCCCAAATTCTAATTTCTACTGTTGTAGAT |
| | | cggatatagaaacattaaagcattccagct |
| | | tatttgatacccacttcaagtttctatcaa |
| | | accatgtctttttcttcgaacgtcaatctc |
| | | gtag |
| 122 | Nucleotide sequence of | ATGCTGCCTAAACTCGTTATAACTCACCGA |
| | the ptxD coding region | GTACATGATGAGATCCTGCAACTGCTGGCG |
| | codon optimized for | CCACATTGCGAGCTGATGACCAACCAAACC |
| | expression in rice | GACAGCACACTGACACGCGAGGAAATTCTG |
| | mitochondria | CGTCGATGTCGTGATGCTCAAGCGATGATG |
| | | GCGTTCATGCCCGATCGAGTCGATGCAGAC |
| | | TTTCTTCAAGCCTGCCCTGAGCTGCGTGTA |
| | | GTCGGCTGCGCGCTCAAGGGCTTCGACAAT |
| | | TTCGATGTGGACGCCTGTACTGCCCGTGGG |
| | | GTCTGGCTGACCTTCGTGCCTGATCTGTTG |
| | | ACAGTCCCAACTGCCGAGCTGGCGATCGGA |
| | | CTGGCGGTGGGGCTGGGGCGACATCTGCGA |
| | | GCAGCAGATGCGTTCGTCCGTTCTGGCGAG |
| | | TTCCAAGGCTGGCAACCACAATTCTATGGC |
| | | ACAGGGCTGGATAACGCTACAGTCGGCATC |
| | | CTTGGCATGGGCGCCATCGGACTGGCCATG |
| | | GCTGATCGATTGCAAGGATGGGGCGCGACC |
| | | CTGCAATATCACGAGGCGAAGGCTCTGGAT |
| | | ACACAAACCGAGCAACGACTCGGCCTGCGT |
| | | CAAGTGGCGTGCAGCGAACTCTTCGCCAGC |
| | | TCGGACTTCATCCTGCTGGCGCTTCCCTTG |
| | | AATGCCGATACCCAACATCTGGTCAACGCC |
| | | GAGCTGCTTGCCCTCGTACGACCAGGCGCT |
| | | CTGCTTGTAAACCCCTGTCGTGGTTCGGTA |
| | | GTGGATGAAGCCGCCGTGCTCGCGGCGCTT |
| | | GAGCGAGGCCAACTCGGCGGGTATGCGGCG |
| | | GATGTATTCGAAATGGAAGACTGGGCTCGT |
| | | GCGGACCGACCACGACTGATCGATCCTGCG |
| | | CTGCTCGCGCATCCTAATACACTGTTCACT |
| | | CCACATATAGGGTCGGCAGTGCGTGCGGTG |
| | | CGTCTGGAGATTGAACGTTGTGCAGCGCAA |
| | | AACATCATCCAAGTATTGGCAGGTGCGCGT |
| | | CCAATCAACGCTGCGAACCGTCTGCCCAAG |
| | | GCCGAGCCTGCCGCATGTTGA |
| 123 | Amino acid sequence of | MAAKIRIVMKSFMSQANKVEGVIPYAQKVG |
| | the fusion protein | LPESRSLYTVLRSPHIDKKSREQFSMLPKL |
| | containing the ptxD | VITHRVHDEILQLLAPHCELMTNQTDSTLT |
| | protein fused with the | REEILRRCRDAQAMMAFMPDRVDADFLQAC |
| | mitochondrial targeting | PELRVVGCALKGFDNFDVDACTARGVWLTF |
| | sequence of the rps10 | VPDLLTVPTAELAIGLAVGLGRHLRAADAF |
| | gene (At5g47030) | VRSGEFQGWQPQFYGTGLDNATVGILGMGA |
| | | IGLAMADRLQGWGATLQYHEAKALDTQTEQ |
| | | RLGLRQVACSELFASSDFILLALPLNADTQ |
| | | HLVNAELLALVRPGALLVNPCRGSVVDEAA |
| | | VLAALERGQLGGYAADVFEMEDWARADRPR |
| | | LIDPALLAHPNTLFTPHIGSAVRAVRLEIE |
| | | RCAAQNIIQVLAGARPINAANRLPKAEPAA |
| | | C |
| 124 | Nucleotide sequence of | GGCCCAAATTCAATTGTATATGAGCTCATA |
| | the 7478 bp Donor DNA | TACAAGACCTCACTAGTAAGGAAGGCACTT |
| | from pNAP420 | GCTGCCGGAGTTCAACAGGCAAATATAAGA |
| | | AAAGAAGTCCTGTTCACTTCATCATCTGTG |
| | | GGTTGTACTGCTTGAAGGTTCTTCTGAGGG |
| | | GTAGAATTTGAATTCCTTCTTTGCTTGTGA |
| | | GATAACCATTTCCAGAAACTCATATATAGA |
| | | GAGCGGGTATCGGTGAAAATGGATCTTACC |
| | | AGGAGTGGCATTGAATAGGCAGGCTCTGGG |
| | | ATGTAATCTCACTCAAGAGGTCATTTGTTG |
| | | GCCCCGCCTTCACTAGACTAGAGTTTTAGG |
| | | ATAGGTTGGGGAACCTATACGTCAAGCCCC |
| | | TACGAAGATTGAGAAAAATCGATGCACATA |
| | | AGCCATCCGAAACCAGTATTGGAAAGTGTT |
| | | CAGTTTCGTTTTCCATTCTGAAATGTTCAT |
| | | AGTAGTATAGTATGTTTTCCGTTGGGTCGA |
| | | CGCCATGTGATCGCTACTAAAGATAGAGTT |
| | | TCCTTGGAAAAACCGAGGCCAGTTGAGATC |
| | | AGTCTCCCTTTCTAGGAGCAGAGCTTAAAA |
| | | AGATGGGAAATTCCAATGAATTTCGATCAC |
| | | AATCATGTGGTAATAATGGGTTTGAATCAG |
| | | AGAGACTCGATCTGGAAACTCCTCAATGAT |
| | | TATAACGTGAACTCGTTGAAGAGAAGGAGA |
| | | CAAGCAGAAATAGACGCTTTTTTTGAACCA |
| | | TTIGAGAGGGCGCAGCGTATCCGTTTCAAT |
| | | AACTGGCAGAACGGAATAGAGTTGTTAGAT |
| | | GGGGCTGAATGGAGGAACGGCGATATAGTT |
| | | ATCCCTGGAGGCGGCGGACCAGTAATTTCA |
| | | AGCCCCTTGGATCAATTTTTCATTGATCCA |
| | | TTATTTGGTCTTGATATGGGTAACTTTTAT |
| | | TTATCATTCACAAATGAATCCTTGTCTATG |
| | | GCGGTAACTGTCGTTTTGGTGCCATCTTTA |
| | | TTTGGAGTTGTTACGAAAAAGGGCGGGGGA |
| | | AAGTCAGTGCCAAATGCATGGCAATCCTTG |
| | | GTAGAGCTTATTTATGATTTCGTGCTGAAC |
| | | CTGGTAAACGAACAAATAGGTGGAAATGTT |
| | | AAACAAAAGTTTTTCCCTCGCATCTCGGTC |
| | | ACTTTTACTTTTTCGTTATTTCGTAATCCC |
| | | CAGGGTATGATACCCTTTAGCTTCACAGTG |
| | | ACAAGTCATTTTCTCATTACTTTGGCTCTT |
| | | TCATTTTCCATTTTTATAGGCATTACGATC |
| | | GTTGGATTTCAAAGACATGGGCTTCATTTT |
| | | TTTAGCTTCTTATTACCAGCGGGAGTCCCA |
| | | CTGCCATTAGCACCTTTTTTAGTACTCCTT |
| | | GAGCTAATCTCTCATTGTTTTCGTGCATTA |
| | | AGCTCAGGAATACGTTTATTTGCTAATATG |
| | | ATGGCCGGTCATAGTTCAGTAAAGATTTTA |
| | | AGTGGGTTCGCTTGGACTATGCTATTTCTG |
| | | AATAATATTTTCTATTTCATAGGAGATCTT |
| | | GGTCCCTTATTTATTGTATTGGCTCTTACT |
| | | GGATTGGAATTAGGTGTAGCTATATTACAA |
| | | GCTCATGTTTCTACGATCTCAATTTGTATT |
| | | TACTTGAATGATGCTATAAATCTCCATCAA |
| | | AATGAGTAATTTCATAATTGAATAAAAACG |
| | | AGGAGCCGAAGATTTTAGGGGGGGGACAAA |
| | | CGCGGAAGTGTATTGCGTTACAAAAAATGA |
| | | CAACTAGCATTTGTTTTTTCATTTCATGTT |
| | | CGAATTCGTTTTTCGTTGGAAAAACCAACG |
| | | CCGACCCCAAACAAGTCTCTCCAATATAAG |
| | | GAGAGCGGAGCTTAAAAATATTATTTTATT |
| | | GTGCTATGGCAAATCTGGTCCGATGGCTCT |
| | | TCTCCACTACCCGAGGGACTAACGGTCTTC |
| | | CATATTTCATCTTCGGTGTCGTTGTAGGAG |
| | | GCGCCCTGTTGTTTGCTTTGCTAAAGTATC |
| | | AGGCCCCTCTGTACGACCCGGCTTTAATGG |
| | | AAAAAATCATAGATCATAATATAAAAGCCG |
| | | GGCACCCTATAGAGGTTGACTATTCGTGGT |
| | | GGGGCACCTCTATTCGTGTAGTCTTTCCTA |
| | | AGTAAGAAAGACAGGACAGTGGTGGTTTGC |
| | | TCATACTTTCATTACAAAACCATACTATGG |
| | | AATTAGGGATAACAGGGTAATAATCACAAG |
| | | TGAGAACCACAGGTAGCAATAGGTATTACA |
| | | GAAATTTCCTCGAGTCTGCTTGAAAGCCTG |
| | | CAGAGTCCAATTTTGAGTATTTTCAGTTAG |
| | | AATCTAGAGTCAGCCTATTCAGTTCTTAGC |
| | | CCTTAAGGGTAAGGCAGGGGGTAATATGGA |
| | | TAGTCTCTGTCCCTGTATTCACATTCCACC |
| | | TTCAACAAAGTGTTGATTTCCCGTAAAGCT |
| | | AACTGTAGTCCTTTAAGTAAGTAGATATCT |
| | | TAGGCAAGTTAGCAATCTCGTTATATTACC |
| | | AAGGCCTTCCCTTCTATTGTAGAAAGAGTT |
| | | CTCAGCCATCTAATTGCAGTGCCAGTTGCC |
| | | AGCTATCCAGTTTCATTTGAAGTTGCTGGG |
| | | GGTCCAAACGAGCTAGTTGCTTTTATTCGT |
| | | CCTATAAGTCCTTCCACAAGCGAGTCAATA |
| | | GGGTGCTGGCTAGTTGTAGTTGTTGGCGTG |
| | | CCTTTCCTTTCATCTTGAATATTAATAAAT |
| | | ATTTGGATAAATTACTTTAGAATAAGAAGT |
| | | TCATGTTTTAATACGACTCACTATAGTAAG |
| | | TAATACGAATCCATACTAGGAAAATGAAAA |
| | | TGTGAGTCCTAGGCACTGGAATTGGTTCTC |
| | | TTCTCCCTAATCCCTATAAGCCAGAAAGGG |
| | | TAATAGGCTTCAGTGTAAGCATTTCCTTCA |
| | | AGCAAGTCATCTCAAGTTTTAAATTCTAGA |
| | | GAATAGCTCCGATCAACCCATTTTAGTTTG |
| | | GTTCTGCAATTCATTCGCATAAATGAAAAA |
| | | AAAAGCGAGATGTGCACGAAAGAAGATCAT |
| | | AGTTCAGCTTTAAAATGGTGGTGTCCCTGT |
| | | GTTAGTAAGTGGTTGAAATAGCTCATGGGA |
| | | GTGTCTGCCCCATTCGATAATGGCATTTAT |
| | | GATCTAGTGGAGTGAGTGATTGTGTGGTGT |
| | | TCAGTCTAAGGCTTTTTGAAAAGCGGATTT |
| | | CTCCCTTCTCTCATCCATCGTCTTTGTTAA |
| | | AGTGAATTTCTCTGACATTCCATGTTTCCG |
| | | AAACGGATCCTATACTGCCTAAACTCGTTA |
| | | TAACTCACCGAGTACATGATGAGATCCTGC |
| | | AACTGCTGGCGCCACATTGCGAGCTGATGA |
| | | CCAACCAAACCGACAGCACACTGACACGCG |
| | | AGGAAATTCTGCGTCGATGTCGTGATGCTC |
| | | AAGCGATGATGGCGTTCATGCCCGATCGAG |
| | | TCGATGCAGACTTTCTTCAAGCCTGCCCTG |
| | | AGCTGCGTGTAGTCGGCTGCGCGCTCAAGG |
| | | GCTTCGACAATTTCGATGTGGACGCCTGTA |
| | | CTGCCCGTGGGGTCTGGCTGACCTTCGTGC |
| | | CTGATCTGTTGACAGTCCCAACTGCCGAGC |
| | | TGGCGATCGGACTGGCGGTGGGGCTGGGGC |
| | | GACATCTGCGAGCAGCAGATGCGTTCGTCC |
| | | GTTCTGGCGAGTTCCAAGGCTGGCAACCAC |
| | | AATTCTATGGCACAGGGCTGGATAACGCTA |
| | | CAGTCGGCATCCTTGGCATGGGCGCCATCG |
| | | GACTGGCCATGGCTGATCGATTGCAAGGAT |
| | | GGGGCGCGACCCTGCAATATCACGAGGCGA |
| | | AGGCTCTGGATACACAAACCGAGCAACGAC |
| | | TCGGCCTGCGTCAAGTGGCGTGCAGCGAAC |
| | | TCTTCGCCAGCTCGGACTTCATCCTGCTGG |
| | | CGCTTCCCTTGAATGCCGATACCCAACATC |
| | | TGGTCAACGCCGAGCTGCTTGCCCTCGTAC |
| | | GACCAGGCGCTCTGCTTGTAAACCCCTGTC |
| | | GTGGTTCGGTAGTGGATGAAGCCGCCGTGC |
| | | TCGCGGCGCTTGAGCGAGGCCAACTCGGCG |
| | | GGTATGCGGCGGATGTATTCGAAATGGAAG |
| | | ACTGGGCTCGTGCGGACCGACCACGACTGA |
| | | TCGATCCTGCGCTGCTCGCGCATCCTAATA |
| | | CACTGTTCACTCCACATATAGGGTCGGCAG |
| | | TGCGTGCGGTGCGTCTGGAGATTGAACGTT |
| | | GTGCAGCGCAAAACATCATCCAAGTATTGG |
| | | CAGGTGCGCGTCCAATCAACGCTGCGAACC |
| | | GTCTGCCCAAGGCCGAGCCTGCCGCATGTC |
| | | CAGTTGCTACTATGGTGAGTAAGGGAGAGG |
| | | AGCTGTTCACCGGGGTGGTGCCTATCCTGG |
| | | TCGAGCTGGATGGTGATGTAAACGGTCATA |
| | | AATTCAGTGTGTCCGGTGAAGGTGAAGGTG |
| | | ATGCCACCTATGGTAAGCTGACCCTTAAGT |
| | | TCATCTGTACCACCGGAAAGCTGCCTGTGC |
| | | CTTGGCCTACCCTCGTGACCACCCTGACAT |
| | | ATGGAGTGCAATGTTTCAGTCGTTATCCTG |
| | | ATCATATGAAGCAACATGATTTCTTTAAAT |
| | | CCGCCATGCCTGAAGGTTATGTCCAAGAGC |
| | | GTACCATATTCTTTAAAGATGATGGTAACT |
| | | ATAAGACCCGTGCCGAGGTGAAGTTCGAGG |
| | | GTGATACCCTGGTGAACCGTATTGAGCTTA |
| | | AGGGTATCGATTTCAAGGAGGATGGAAACA |
| | | TCCTGGGGCATAAGCTGGAGTATAACTATA |
| | | ACAGTCATAACGTCTATATCATGGCCGATA |
| | | AGCAAAAGAACGGTATCAAGGTGAACTTCA |
| | | AGATCCGTCATAATATCGAAGATGGAAGTG |
| | | TGCAACTCGCCGATCATTATCAACAAAACA |
| | | CCCCTATCGGTGATGGTCCTGTGCTGCTGC |
| | | CTGATAACCATTATCTGAGTACCCAATCCG |
| | | CCCTGAGTAAAGATCCTAACGAGAAGCGTG |
| | | ATCAAATGGTACTGCTTGAGTTCGTTACCG |
| | | CCGCCGGGATCACTCTCGGTATGGATGAGC |
| | | TGTATAAGTAATGAGGTACCAAGCGATCGC |
| | | AATAATACGACTCACTATAGACCTAGGAAA |
| | | AGATCTAGACGAGGTGTAGCGCAGTCTGGT |
| | | CAGCGCATCTGTTTTGGGTACAGAGGGCCA |
| | | TAGGTTCGAATCCTGTCACCTTGAGTCTGG |
| | | CCCCAAATTCTAATTTCTACTGTTGTAGAT |
| | | TAGTTCTAGCATTAACCGGTCGTCCCTTTC |
| | | GTCCAGTGGTTAGGACATCGTCTTTTCATG |
| | | TCGAAGACACGGGTTCGATTCCCGTAAGGG |
| | | ATAGTCTGGCCCCAAATTCTAATTTCTACT |
| | | GTTGTAGATGTCACATTTCTACCGGTGCAC |
| | | ATTCCAGCTTATTTGATACCCACTTCAAGT |
| | | TTCTATCAAACCATGTCTTTTTCTTCGAAC |
| | | GTCAATCTCGTAGTTCTTCCGAACTCAACT |
| | | CCGGTTTTAGAGCTAGAAATAGCAAGTTAA |
| | | AATAAGGCTAGTCCGTTATCAACTTGAAAA |
| | | AGTGGCACCGAGTCGGTGCTGGGGAAATAG |
| | | CTCAGTTGGTTAGAGTGCTGGTCTGTCACG |
| | | CCAGAAGTCGCGGGTTCGAACCCCGTTTTC |
| | | CCCGATCTGGGATCTGATATGTGGGTTTTA |
| | | GAGCTAGAAATAGCAAGTTAAAATAAGGCT |
| | | AGTCCGTTATCAACTTGAAAAAGTGGCACC |
| | | GAGTCGGTGCTGGGAGAGTGGCCGAGCGGT |
| | | CAAAAGCGACAGACTGTAAATCTGTTGAAG |
| | | GTTTTCTACGTAGGTTCGAATCCTGCCTCT |
| | | CCCAGAATAATACGACTCACTATAGAATAA |
| | | TAGAGGGCTTATAGTTTAATTGGTTGAAAC |
| | | GTACCGCTCATAACGGTGATATTGTAGGTT |
| | | CGAGCCCTACTAAGCCCAGTCTGGCCCCAA |
| | | ATTCTAATTTCTACTGTTGTAGATAATCGG |
| | | CATGTACTATGGAATGGAGGTATGGCTGAG |
| | | TGGCTTAAGGCATTGGTTTGCTAAATCGAC |
| | | ATACAAGAAGATTGTATCATGGGTTCGAAT |
| | | CCCATTTCCTCCGGTCTGGCCCCAAATTCT |
| | | AATTTCTACTGTTGTAGATGATATGCAAGC |
| | | ACGAGATTCCTGGAGTATAGCCAAGTGGTA |
| | | AGGCATCGGTTTTTGGTACCGGCATGCAAA |
| | | GGTTCGAATCCTTTTACTCCAGAACCCCTT |
| | | GGGGCCTCTAAACGGGTCTTGAGGGGTTTT |
| | | TTGGAAAGATCTAAAAAGCTTAAGCGGCCG |
| | | CAAAAACCCCTTGGGGCCTCTAAACGGGTC |
| | | TTGAGGGGTTTTTTGTAGATAGACCTTTTT |
| | | ATTTTTCGTCATTCGATCACGAAAAGGCCG |
| | | GCCAAACCTCGAGGTAATTAAAGCGGCCGA |
| | | AATTAGCTAGCAAATAAGCATGCAATCACA |
| | | AGTGAGAACCACAGGTAGCAATAGGTATTA |
| | | CAGTAGGGATAACAGGGTAATGAATTCGAA |
| | | ACCATCTTCTCACTCTGACCCCCACATATC |
| | | AGATCCCAGATGCATAGGAAAAGCGGTATC |
| | | AAGAATAGTAGTATAAAGAAAGATAGTACA |
| | | GTACTCAAGTAAATGAATTCGCCTAAGGAT |
| | | CGATGGAAAGATCAAGGTCCCCGTGAAAAA |
| | | GTAGATACTAGATCGATATGATACTCTCAT |
| | | CTCTGGAGTAACTTCTTCCATTATGCTGAT |
| | | CTCTAGGTCCGTTCCATCATCATCGTAATA |
| | | GTATGGTCCCAGGTGTCCGAGCTATAGATC |
| | | AAGATCATATCCAGTCACATTTCTACCGGT |
| | | GCACTTCTCATGAAATAATTCCCTTTCCAA |
| | | GGAAAGGAAAACAAGAACTCGAATACTCGT |
| | | AATAGCGATCCCGATCCACCTACTTTTTTC |
| | | TATTCTTTGATTCGAAACGTGCTAAAGCAC |
| | | AAGCCATTTTTATGCATGGGGCATAAGAGT |
| | | GGACAATCTATGTTATCGAAGGAAGTAAAT |
| | | AACAACACTTCAGCGTTTAGGTCTACCTTC |
| | | AGTAAACCAATAGTTTTGCAGCATTGGAAT |
| | | TTGAGTTGGCCAGGTAAGGTCCTCTAAAAA |
| | | GAAAAGAAGAAACTACTTAGAATAGATAAA |
| | | TGCCATTGGTTTTCTCGTACTATACGATCT |
| | | TTTTTTGTTTTGTTTTTTGGCCATGATTGT |
| | | GCTGCTCCTGTGAAGGCTAGTGGGAAAGCT |
| | | CACCGTTCGTTGTGATGAGTGGGGGCCTTG |
| | | TATCTGTATTCGGATCAGCTCCTTAACAGA |
| | | GTTTCCTGCTTGAACCCTGGCTGGGAGCTG |
| | | GGAGAGGTGTCCCACTACAGGTGCAAATAA |
| | | ACCATTTGACCTTACAGGGGAAAGGAAACA |
| | | AACCACTCAATAATCGGTAGAAATTCCTCC |
| | | TACTGAACAGCTTTCCTTTTCTCGCCTTAA |
| | | CTACTACTTCAAAGCAAGGCGGAATATCAC |
| | | GGGATAGGAATGAAAGAACTTCTTACTCAA |
| | | CTTTCTAGCTATATAAAAATAGTTAGCAAT |
| | | ATGAAACGAGTAACTTAAGCCCTAGTAAAA |
| | | GGCTACTCTTTGAATCCCCTCTTTAAGGCA |
| | | TATAAAATTAGTACTCTTCCTGAGCTAGCT |
| | | TAAGCATATCTTGAGCGAGTGAGTTGTATT |
| | | TCCCTCCATCAAGTTCTAAGCGATCAAATA |
| | | AGGTCCTTGCTCTCGAGCCAATGCCAATAC |
| | | CAATAGAGAGGGTCTAAACGAAGGATTCAA |
| | | AGGCGCG |
| 125 | Nucleotide sequence of | GGCCCAAATTCAATTGTATATGAGCTCATA |
| | the 7477 bp Donor DNA | TACAAGACCTCACTAGTAAGGAAGGCACTT |
| | from pNAP422 | GCTGCCGGAGTTCAACAGGCAAATATAAGA |
| | | AAAGAAGTCCTGTTCACTTCATCATCTGTG |
| | | GGTTGTACTGCTTGAAGGTTCTTCTGAGGG |
| | | GTAGAATTTGAATTCCTTCTTTGCTTGTGA |
| | | GATAACCATTTCCAGAAACTCATATATAGA |
| | | GAGCGGGTATCGGTGAAAATGGATCTTACC |
| | | AGGAGTGGCATTGAATAGGCAGGCTCTGGG |
| | | ATGTAATCTCACTCAAGAGGTCATTTGTTG |
| | | GCCCCGCCTTCACTAGACTAGAGTTTTAGG |
| | | ATAGGTTGGGGAACCTATACGTCAAGCCCC |
| | | TACGAAGATTGAGAAAAATCGATGCACATA |
| | | AGCCATCCGAAACCAGTATTGGAAAGTGTT |
| | | CAGTTTCGTTTTCCATTCTGAAATGTTCAT |
| | | AGTAGTATAGTATGTTTTCCGTTGGGTCGA |
| | | CGCCATGTGATCGCTACTAAAGATAGAGTT |
| | | TCCTTGGAAAAACCGAGGCCAGTTGAGATC |
| | | AGTCTCCCTTTCTAGGAGCAGAGCTTAAAA |
| | | AGATGGGAAATTCCAATGAATTTCGATCAC |
| | | AATCATGTGGTAATAATGGGTTTGAATCAG |
| | | AGAGACTCGATCTGGAAACTCCTCAATGAT |
| | | TATAACGTGAACTCGTTGAAGAGAAGGAGA |
| | | CAAGCAGAAATAGACGCTTTTTTTGAACCA |
| | | TTIGAGAGGGCGCAGCGTATCCGTTTCAAT |
| | | AACTGGCAGAACGGAATAGAGTTGTTAGAT |
| | | GGGGCTGAATGGAGGAACGGCGATATAGTT |
| | | ATCCCTGGAGGCGGCGGACCAGTAATTTCA |
| | | AGCCCCTTGGATCAATTTTTCATTGATCCA |
| | | TTATTTGGTCTTGATATGGGTAACTTTTAT |
| | | TTATCATTCACAAATGAATCCTTGTCTATG |
| | | GCGGTAACTGTCGTTTTGGTGCCATCTTTA |
| | | TTTGGAGTTGTTACGAAAAAGGGCGGGGGA |
| | | AAGTCAGTGCCAAATGCATGGCAATCCTTG |
| | | GTAGAGCTTATTTATGATTTCGTGCTGAAC |
| | | CTGGTAAACGAACAAATAGGTGGAAATGTT |
| | | AAACAAAAGTTTTTCCCTCGCATCTCGGTC |
| | | ACTTTTACTTTTTCGTTATTTCGTAATCCC |
| | | CAGGGTATGATACCCTTTAGCTTCACAGTG |
| | | ACAAGTCATTTTCTCATTACTTTGGCTCTT |
| | | TCATTTTCCATTTTTATAGGCATTACGATC |
| | | GTTGGATTTCAAAGACATGGGCTTCATTTT |
| | | TTTAGCTTCTTATTACCAGCGGGAGTCCCA |
| | | CTGCCATTAGCACCTTTTTTAGTACTCCTT |
| | | GAGCTAATCTCTCATTGTTTTCGTGCATTA |
| | | AGCTCAGGAATACGTTTATTTGCTAATATG |
| | | ATGGCCGGTCATAGTTCAGTAAAGATTTTA |
| | | AGTGGGTTCGCTTGGACTATGCTATTTCTG |
| | | AATAATATTTTCTATTTCATAGGAGATCTT |
| | | GGTCCCTTATTTATTGTATTGGCTCTTACT |
| | | GGATTGGAATTAGGTGTAGCTATATTACAA |
| | | GCTCATGTTTCTACGATCTCAATTTGTATT |
| | | TACTTGAATGATGCTATAAATCTCCATCAA |
| | | AATGAGTAATTTCATAATTGAATAAAAACG |
| | | AGGAGCCGAAGATTTTAGGGGGCGGGACAA |
| | | ACGCGGAAGTGTATTGCGTTACAAAAAATG |
| | | ACAACTAGCATTTGTTTTTTCATTTCATGT |
| | | TCGAATTCGTTTTTCGTTGGAAAAACCAAC |
| | | GCCGACCCCAAACAAGTCTCTCCAATATAA |
| | | GGAGAGCGGAGCTTAAAAATATTATTTTAT |
| | | TGTGCTATGGCAAATCTGGTCCGATGGCTC |
| | | TTCTCCACTACCCGAGGGACTAACGGTCTT |
| | | CCATATTTCATCTTCGGTGTCGTTGTAGGA |
| | | GGCGCCCTGTTGTTTGCTTTGCTAAAGTAT |
| | | CAGGCCCCTCTGTACGACCCGGCTTTAATG |
| | | GAAAAAATCATAGATCATAATATAAAAGCC |
| | | GGGCACCCTATAGAGGTTGACTATTCGTGG |
| | | TGGGGCACCTCTATTCGTGTAGTCTTTCCT |
| | | AAGTAAGAAAGACAGGACAGTGGTGGTTTG |
| | | CTCATACTTTCATTACAAAACCATACTATG |
| | | GAATTAGGGATAACAGGGTAATAATCACAA |
| | | GTGAGAACCACAGGTAGCAATAGGTATTAC |
| | | AGAAATTTCCTCGAGTCTGCTTGAAAGCCT |
| | | GCAGAGTCCAATTTTGAGTATTTTCAGTTA |
| | | GAATCTAGAGTCAGCCTATTCAGTTCTTAG |
| | | CCCTTAAGGGTAAGGCAGGGGGTAATATGG |
| | | ATAGTCTCTGTCCCTGTATTCACATTCCAC |
| | | CTTCAACAAAGTGTTGATTTCCCGTAAAGC |
| | | TAACTGTAGTCCTTTAAGTAAGTAGATATC |
| | | TTAGGCAAGTTAGCAATCTCGTTATATTAC |
| | | CAAGGCCTTCCCTTCTATTGTAGAAAGAGT |
| | | TCTCAGCCATCTAATTGCAGTGCCAGTTGC |
| | | CAGCTATCCAGTTTCATTTGAAGTTGCTGG |
| | | GGGTCCAAACGAGCTAGTTGCTTTTATTCG |
| | | TCCTATAAGTCCTTCCACAAGCGAGTCAAT |
| | | AGGGTGCTGGCTAGTTGTAGTTGTTGGCGT |
| | | GCCTTTCCTTTCATCTTGAATATTAATAAA |
| | | TATTTGGATAAATTACTTTAGAATAAGAAG |
| | | TTCATGTTTTAATACGACTCACTATAGTAA |
| | | GTAATACGAATCCATACTAGGAAAATGAAA |
| | | ATGTGAGTCCTAGGCACTGGAATTGGTTCT |
| | | CTTCTCCCTAATCCCTATAAGCCAGAAAGG |
| | | GTAATAGGCTTCAGTGTAAGCATTTCCTTC |
| | | AAGCAAGTCATCTCAAGTTTTAAATTCTAG |
| | | AGAATAGCTCCGATCAACCCATTTTAGTTT |
| | | GGTTCTGCAATTCATTCGCATAAATGAAAA |
| | | AAAAAGCGAGATGTGCACGAAAGAAGATCA |
| | | TAGTTCAGCTTTAAAATGGTGGTGTCCCTG |
| | | TGTTAGTAAGTGGTTGAAATAGCTCATGGG |
| | | AGTGTCTGCCCCATTCGATAATGGCATTTA |
| | | TGATCTAGTGGAGTGAGTGATTGTGTGGTG |
| | | TTCAGTCTAAGGCTTTTTGAAAAGCGGATT |
| | | TCTCCCTTCTCTCATCCATCGTCTTTGTTA |
| | | AAGTTGAACAGTCACTCACTTTTGACAGTT |
| | | ATACGATTCCAGAACTGCCTAAACTCGTTA |
| | | TAACTCACCGAGTACATGATGAGATCCTGC |
| | | AACTGCTGGCGCCACATTGCGAGCTGATGA |
| | | CCAACCAAACCGACAGCACACTGACACGCG |
| | | AGGAAATTCTGCGTCGATGTCGTGATGCTC |
| | | AAGCGATGATGGCGTTCATGCCCGATCGAG |
| | | TCGATGCAGACTTTCTTCAAGCCTGCCCTG |
| | | AGCTGCGTGTAGTCGGCTGCGCGCTCAAGG |
| | | GCTTCGACAATTTCGATGTGGACGCCTGTA |
| | | CTGCCCGTGGGGTCTGGCTGACCTTCGTGC |
| | | CTGATCTGTTGACAGTCCCAACTGCCGAGC |
| | | TGGCGATCGGACTGGCGGTGGGGCTGGGGC |
| | | GACATCTGCGAGCAGCAGATGCGTTCGTCC |
| | | GTTCTGGCGAGTTCCAAGGCTGGCAACCAC |
| | | AATTCTATGGCACAGGGCTGGATAACGCTA |
| | | CAGTCGGCATCCTTGGCATGGGCGCCATCG |
| | | GACTGGCCATGGCTGATCGATTGCAAGGAT |
| | | GGGGCGCGACCCTGCAATATCACGAGGCGA |
| | | AGGCTCTGGATACACAAACCGAGCAACGAC |
| | | TCGGCCTGCGTCAAGTGGCGTGCAGCGAAC |
| | | TCTTCGCCAGCTCGGACTTCATCCTGCTGG |
| | | CGCTTCCCTTGAATGCCGATACCCAACATC |
| | | TGGTCAACGCCGAGCTGCTTGCCCTCGTAC |
| | | GACCAGGCGCTCTGCTTGTAAACCCCTGTC |
| | | GTGGTTCGGTAGTGGATGAAGCCGCCGTGC |
| | | TCGCGGCGCTTGAGCGAGGCCAACTCGGCG |
| | | GGTATGCGGCGGATGTATTCGAAATGGAAG |
| | | ACTGGGCTCGTGCGGACCGACCACGACTGA |
| | | TCGATCCTGCGCTGCTCGCGCATCCTAATA |
| | | CACTGTTCACTCCACATATAGGGTCGGCAG |
| | | TGCGTGCGGTGCGTCTGGAGATTGAACGTT |
| | | GTGCAGCGCAAAACATCATCCAAGTATTGG |
| | | CAGGTGCGCGTCCAATCAACGCTGCGAACC |
| | | GTCTGCCCAAGGCCGAGCCTGCCGCATGTC |
| | | CAGTTGCTACTATGGTGAGTAAGGGAGAGG |
| | | AGCTGTTCACCGGGGTGGTGCCTATCCTGG |
| | | TCGAGCTGGATGGTGATGTAAACGGTCATA |
| | | AATTCAGTGTGTCCGGTGAAGGTGAAGGTG |
| | | ATGCCACCTATGGTAAGCTGACCCTTAAGT |
| | | TCATCTGTACCACCGGAAAGCTGCCTGTGC |
| | | CTTGGCCTACCCTCGTGACCACCCTGACAT |
| | | ATGGAGTGCAATGTTTCAGTCGTTATCCTG |
| | | ATCATATGAAGCAACATGATTTCTTTAAAT |
| | | CCGCCATGCCTGAAGGTTATGTCCAAGAGC |
| | | GTACCATATTCTTTAAAGATGATGGTAACT |
| | | ATAAGACCCGTGCCGAGGTGAAGTTCGAGG |
| | | GTGATACCCTGGTGAACCGTATTGAGCTTA |
| | | AGGGTATCGATTTCAAGGAGGATGGAAACA |
| | | TCCTGGGGCATAAGCTGGAGTATAACTATA |
| | | ACAGTCATAACGTCTATATCATGGCCGATA |
| | | AGCAAAAGAACGGTATCAAGGTGAACTTCA |
| | | AGATCCGTCATAATATCGAAGATGGAAGTG |
| | | TGCAACTCGCCGATCATTATCAACAAAACA |
| | | CCCCTATCGGTGATGGTCCTGTGCTGCTGC |
| | | CTGATAACCATTATCTGAGTACCCAATCCG |
| | | CCCTGAGTAAAGATCCTAACGAGAAGCGTG |
| | | ATCAAATGGTACTGCTTGAGTTCGTTACCG |
| | | CCGCCGGGATCACTCTCGGTATGGATGAGC |
| | | TGTATAAGTAATGAGGTACCAAGCGATCGC |
| | | AATAATACGACTCACTATAGACCTAGGAAA |
| | | AGATCTAGACGAGGTGTAGCGCAGTCTGGT |
| | | CAGCGCATCTGTTTTGGGTACAGAGGGCCA |
| | | TAGGTTCGAATCCTGTCACCTTGAGTCTGG |
| | | CCCCAAATTCTAATTTCTACTGTTGTAGAT |
| | | TAGTTCTAGCATTAACCGGTCGTCCCTTTC |
| | | GTCCAGTGGTTAGGACATCGTCTTTTCATG |
| | | TCGAAGACACGGGTTCGATTCCCGTAAGGG |
| | | ATAGTCTGGCCCCAAATTCTAATTTCTACT |
| | | GTTGTAGATGTCACATTTCTACCGGTGCAC |
| | | ATTCCAGCTTATTTGATACCCACTTCAAGT |
| | | TTCTATCAAACCATGTCTTTTTCTTCGAAC |
| | | GTCAATCTCGTAGTTCTTCCGAACTCAACT |
| | | CCGGTTTTAGAGCTAGAAATAGCAAGTTAA |
| | | AATAAGGCTAGTCCGTTATCAACTTGAAAA |
| | | AGTGGCACCGAGTCGGTGCTGGGGAAATAG |
| | | CTCAGTTGGTTAGAGTGCTGGTCTGTCACG |
| | | CCAGAAGTCGCGGGTTCGAACCCCGTTTTC |
| | | CCCGATCTGGGATCTGATATGTGGGTTTTA |
| | | GAGCTAGAAATAGCAAGTTAAAATAAGGCT |
| | | AGTCCGTTATCAACTTGAAAAAGTGGCACC |
| | | GAGTCGGTGCTGGGAGAGTGGCCGAGCGGT |
| | | CAAAAGCGACAGACTGTAAATCTGTTGAAG |
| | | GTTTTCTACGTAGGTTCGAATCCTGCCTCT |
| | | CCCAGAATAATACGACTCACTATAGAATAA |
| | | TAGAGGGCTTATAGTTTAATTGGTTGAAAC |
| | | GTACCGCTCATAACGGTGATATTGTAGGTT |
| | | CGAGCCCTACTAAGCCCAGTCTGGCCCCAA |
| | | ATTCTAATTTCTACTGTTGTAGATAATCGG |
| | | CATGTACTATGGAATGGAGGTATGGCTGAG |
| | | TGGCTTAAGGCATTGGTTTGCTAAATCGAC |
| | | ATACAAGAAGATTGTATCATGGGTTCGAAT |
| | | CCCATTTCCTCCGGTCTGGCCCCAAATTCT |
| | | AATTTCTACTGTTGTAGATGATATGCAAGC |
| | | ACGAGATTCCTGGAGTATAGCCAAGTGGTA |
| | | AGGCATCGGTTTTTGGTACCGGCATGCAAA |
| | | GGTTCGAATCCTTTTACTCCAGAACCCCTT |
| | | GGGGCCTCTAAACGGGTCTTGAGGGGTTTT |
| | | TTGGAAAGATCTAAAAAGCTTAAGCGGCCG |
| | | CAAAAACCCCTTGGGGCCTCTAAACGGGTC |
| | | TTGAGGGGTTTTTTGTAGATAGACCTTTTT |
| | | ATTTTTCGTCATTCGATCACGAAAAGGCCG |
| | | GCCAAACCTCGAGGTAATTAAAGCGGCCGA |
| | | AATTAGCTAGCAAATAAGCATGCAATCACA |
| | | AGTGAGAACCACAGGTAGCAATAGGTATTA |
| | | CAGTAGGGATAACAGGGTAATGAATTCGAA |
| | | ACCATCTTCTCACTCTGACCCCCACATATC |
| | | AGATCCCAGATGCATAGGAAAAGCGGTATC |
| | | AAGAATAGTAGTATAAAGAAAGATAGTACA |
| | | GTACTCAAGTAAATGAATTCGCCTAAGGAT |
| | | CGATGGAAAGATCAAGGTCCCCGTGAAAAA |
| | | GTAGATACTAGATCGATATGATACTCTCAT |
| | | CTCTGGAGTAACTTCTTCCATTATGCTGAT |
| | | CTCTAGGTCCGTTCCATCATCATCGTAATA |
| | | GTATGGTCCCAGGTGTCCGAGCTATAGATC |
| | | AAGATCATATCCAGTCACATTTCTACCGGT |
| | | GCACTTCTCATGAAATAATTCCCTTTCCAA |
| | | GGAAAGGAAAACAAGAACTCGAATACTCGT |
| | | AATAGCGATCCCGATCCACCTACTTTTTTC |
| | | TATTCTTTGATTCGAAACGTGCTAAAGCAC |
| | | AAGCCATTTTTATGCATGGGGCATAAGAGT |
| | | GGACAATCTATGTTATCGAAGGAAGTAAAT |
| | | AACAACACTTCAGCGTTTAGGTCTACCTTC |
| | | AGTAAACCAATAGTTTTGCAGCATTGGAAT |
| | | TTGAGTTGGCCAGGTAAGGTCCTCTAAAAA |
| | | GAAAAGAAGAAACTACTTAGAATAGATAAA |
| | | TGCCATTGGTTTTCTCGTACTATACGATCT |
| | | TTTTTTGTTTTGTTTTTTGGCCATGATTGT |
| | | GCTGCTCCTGTGAAGGCTAGTGGGAAAGCT |
| | | CACCGTTCGTTGTGATGAGTGGGGGCCTTG |
| | | TATCTGTATTCGGATCAGCTCCTTAACAGA |
| | | GTTTCCTGCTTGAACCCTGGCTGGGAGCTG |
| | | GGAGAGGTGTCCCACTACAGGTGCAAATAA |
| | | ACCATTTGACCTTACAGGGGAAAGGAAACA |
| | | AACCACTCAATAATCGGTAGAAATTCCTCC |
| | | TACTGAACAGCTTTCCTTTTCTCGCCTTAA |
| | | CTACTACTTCAAAGCAAGGCGGAATATCAC |
| | | GGGATAGGAATGAAAGAACTTCTTACTCAA |
| | | CTTTCTAGCTATATAAAAATAGTTAGCAAT |
| | | ATGAAACGAGTAACTTAAGCCCTAGTAAAA |
| | | GGCTACTCTTTGAATCCCCTCTTTAAGGCA |
| | | TATAAAATTAGTACTCTTCCTGAGCTAGCT |
| | | TAAGCATATCTTGAGCGAGTGAGTTGTATT |
| | | TCCCTCCATCAAGTTCTAAGCGATCAAATA |
| | | AGGTCCTTGCTCTCGAGCCAATGCCAATAC |
| | | CAATAGAGAGGGTCTAAACGAAGGATTCAA |
| | | AGGCGCG |
| 126 | Nucleotide sequence of | GAATTTCTCTGACATTCCATGTTTCCGAAA |
| | the longer version of the | cGGATCCT |
| | RNA editing site found | |
| | at the initiation codon of | |
| | the rice mitochondrial | |
| | nad4L gene | |
| 127 | Nucleotide sequence of | GTAGGGCTTTCTGAGGAGTAAGCCTAATTC |
| | the 5HRA PCR primer | CGTTAATGCAG |
| | specific to wild-type | |
| | mtDNA for | |
| | amplification of the 5′ | |
| | homologous junction | |
| 128 | Nucleotide sequence of | GAGAGACTTGTTTGGGGTCGGCGTTGG |
| | the ORFB PCR primer | |
| | specific to the Donor | |
| | DNA for amplification | |
| | of the 5′ homologous | |
| | junction | |
| 129 | Nucleotide sequence of | ACCACAGGTAGCAATAGGTATTACAGTAGG |
| | the 420A PCR primer | GATAACAG |
| | specific to the Donor | |
| | DNA for amplification | |
| | of the 3′ homologous | |
| | junction | |
| 130 | Nucleotide sequence of | AGTGCTCAGAATAATCCAGGTCGCTCGACG |
| | the 3HRA specific to | |
| | wild-type mtDNA for | |
| | amplification of the 3′ | |
| | homologous junction | |
| 131 | Nucleotide sequence of | TGAACAGTCACTCACTTTTGACAGTTATAc |
| | the cox2 RNA editing | GATTCCAGAA |
| | site present in pNAP422 | |
| 132 | Nucleotide sequence of | GAGAGAAGATGACCCAGATCATGTTCG |
| | the PCR primer OsAct1- | |
| | F2 for amplification of | |
| | the rice Actin1 sequence | |
| 133 | Nucleotide sequence of | CTGGCAGTATCAAGCTCCTGTTCATAA |
| | the PCR primer OsAct1- | |
| | R2 for amplification of | |
| | the rice Actin1 sequence | |
| 134 | Nucleotide sequence of | GTCTGCCCCATTCGATAATGGCA |
| | the OsATP1-PRO-FP1 | |
| | PCR primer for | |
| | amplification of the | |
| | cDNA of the mOsPtxD | |
| | transcripts | |
| 135 | Nucleotide sequence of | TCCACATCGAAATTGTCGAAGCCCTT |
| | the mOsPtxD-RP1 PCR | |
| | primer for amplification | |
| | of the cDNA of the | |
| | mOsPtxD transcripts | |

### Definitions

In some embodiments, the meaning of abbreviations can be as follows: “sec” can mean second(s), “min” can mean minute(s), “h” can mean hour(s), “d” can mean day(s), “μL” can mean microliter(s), “ml” can mean milliliter(s), “L” can mean liter(s), “μM” can mean micromolar, “mM” can mean millimolar, “M” can mean molar, “mmol” can mean millimole(s), “μmole” can mean micromole(s), “g” can mean gram(s), “μg” can mean microgram(s), “ng” can mean nanogram(s), “U” can mean unit(s), “nt” can mean nucleotide(s); “bp” can mean base pair(s), “kb” can mean kilobase(s) and “kbp” can mean kilobase pair(s).

In some embodiments, “transgenic” can refer to any cell, cell line, callus, tissue, organism part or whole organism (e.g., plant), the genome of which has been edited or altered by the presence of a heterologous nucleic acid, such as a recombinant DNA construct. In some embodiments, transgenic events can include those created by sexual crosses or asexual propagation. In some embodiments, the term “transgenic” may not encompass an edited genome or alteration of a genome (e.g., chromosomal or extra-chromosomal) by breeding methods or by naturally occurring events such as random cross-fertilization, non-recombinant viral infection, non-recombinant bacterial transformation, non-recombinant transposition, or spontaneous mutation. In some embodiments, the term “transgenic” may encompass an edited genome or alteration of a genome (e.g., chromosomal or extra-chromosomal) by breeding methods or by naturally occurring events such as random cross-fertilization, non-recombinant viral infection, non-recombinant bacterial transformation, non-recombinant transposition, or spontaneous mutation.

In some embodiments, “genome”, for example, of a cell or whole organism can encompass chromosomal DNA found within a nucleus (nuclear DNA), and organellar DNA (e.g., mitochondrial DNA, plastid DNA) found within subcellular components of a cell. Methods and compositions of a disclosure can be used for editing of a nuclear genome, organellar genome (e.g., mitochondria, chloroplasts), or both.

In some embodiments, the terms “full complement” and “full-length complement” can be used interchangeably herein, and can refer to a complement of a given nucleotide sequence. In some aspects, a complement and a nucleotide sequence can comprise a same number of nucleotides. In some aspects, a complement and a nucleotide sequence can comprise 100% complementary. In some embodiments, a complement and a nucleotide sequence can differ in a number of nucleotides. In some embodiments, complementarity (e.g., between a complement and a nucleotide sequence) can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%. In some embodiments, complementarity (e.g., between a complement and a nucleotide sequence) can be at most about 10%, at most about 15%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 91%, at most about 92%, at most about 93%, at most about 94%, at most about 95%, at most about 96%, at most about 97%, at most about 98%, at most about 99%, or 100%.

In some embodiments, “polynucleotide”, “nucleic acid”, “nucleic acid sequence”, “nucleotide sequence”, or “nucleic acid fragment”, which can be used interchangeably, can refer to a polymer of a nucleic acid (e.g., RNA, DNA, or both, and analogs thereof) that can be single-stranded or double-stranded (or both single-stranded and double-stranded), optionally containing synthetic, non-natural or altered nucleotide bases. In some embodiments, nucleotides (e.g., in their 5′-monophosphate form) can be referred to by a single letter designation as follows (for RNA or DNA, respectively): “A” for adenylate or deoxyadenylate, “C” for cytidylate or deoxycytidylate, “G” for guanylate or deoxyguanylate, “U” for uridylate, “T” for deoxythymidylate, “R” for purine-based nucleotides (A or G), “Y” for pyrimidine-based nucleotides (C or T), “K” for G or T, “H” for A or C or T, “I” for inosine, and “N” for any nucleotide. In some embodiments, a polynucleotide can be linear or circular.

In some embodiments, “polypeptide”, “peptide”, “amino acid sequence” and “protein”, which can be used interchangeably herein, can refer to a polymer of amino acid residues. In some embodiments, these terms can apply to amino acid polymers in which one or more amino acid residue can be, for example, an artificial chemical analogue of a corresponding naturally occurring amino acid and/or to naturally occurring amino acid polymers. In some embodiments, the terms “polypeptide”, “peptide”, “amino acid sequence”, and “protein” can be inclusive of modifications including, but not limited to, glycosylation, lipid attachment, sulfation, gamma-carboxylation of glutamic acid residues, hydroxylation and ADP-ribosylation.

In some embodiments, a “functional fragment” of a polynucleotide or polypeptide can refer to any subset of contiguous nucleotides or contiguous amino acids, respectively, in which an original (e.g., wild type) activity (or substantially similar activity) of a polynucleotide or polypeptide can be retained. In some embodiments, the terms “functional fragment”, “functional subfragment”, “fragment that is functionally equivalent”, “subfragment that is functionally equivalent”, “functionally equivalent fragment”, “a biologically active fragment” and “functionally equivalent subfragment” can be used interchangeably herein.

In some embodiments, the terms “functional variant”, “variant that is functionally equivalent” and “functionally equivalent variant” can be used interchangeably herein. In some embodiments, in the context of a polynucleotide or a polypeptide, these terms can refer to a variant of the nucleic acid sequence or the amino acid sequence, respectively, in which the original activity (or substantially similar activity) of the polynucleotide or polypeptide can be retained. In some embodiments, fragments and variants can be obtained via methods such as site-directed mutagenesis and synthetic construction.

In some embodiments, an activity of a functional fragment or functional variant can be, for example, about: 100%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, 20%, 15%, 10%, or less than 10% of that of an original (e.g., wild type) activity.

In some embodiments, an “RNA transcript” can refer to a product resulting from an RNA polymerase-catalyzed transcription of a DNA sequence. In some embodiments, when an RNA transcript is a perfect complementary copy of a DNA sequence, it can be referred to as a primary transcript. In some embodiments, an RNA transcript can be referred to as a mature RNA, for example, when it is an RNA sequence derived from post-transcriptional processing of a primary transcript.

In some embodiments, a “messenger RNA” or “mRNA” can refer to an RNA that is without introns and that can be translated into protein by a cell.

In some embodiments, “sense” RNA can refer to an RNA transcript that includes an mRNA. In some embodiments, sense RNA can be translated into protein within a cell or in vitro.

In some embodiments, “antisense RNA” can refer to an RNA transcript that can be complementary to all or part of a target RNA (e.g., a primary transcript or mRNA). In some embodiments, antisense RNA can be used to block expression of a target gene. In some embodiments, a complementarity of an antisense RNA may be with any part of a specific gene transcript, i.e., at a 5′ non-coding sequence, 3′ non-coding sequence, introns, or a coding sequence. In some embodiments, “functional RNA” can refer to antisense RNA, ribozyme RNA, or other RNA that may not be translated but yet can have an effect on cellular processes. In some embodiments, the terms “complement” and “reverse complement” can be used interchangeably herein, for example, with respect to mRNA transcripts and can be used to define the antisense RNA of a message.

In some embodiments, “cDNA” can refer to a DNA that can be complementary to and synthesized from a mRNA template using a reverse transcriptase enzyme. In some embodiments, a cDNA can be single-stranded or converted into a double-stranded form using a Klenow fragment of DNA polymerase I.

In some embodiments, a “coding region” can refer to a portion of a messenger RNA (or a corresponding portion of another nucleic acid molecule such as a DNA molecule) which can encode a protein or polypeptide. In some embodiments, a “non-coding region” can refer to a portion of a messenger RNA or other nucleic acid molecule that is not a coding region, including but not limited to, for example, a promoter region, a 5′ untranslated region (“UTR”), a 3′ UTR, an intron and a terminator. In some embodiments, the terms “coding region” and “coding sequence” can be used interchangeably herein. In some embodiments, the terms “non-coding region” and “non-coding sequence” can be used interchangeably herein.

In some embodiments, “coding sequence” can be abbreviated “CDS”. In some embodiments, “Open reading frame” can be abbreviated “ORF”.

In some embodiments, “gene” can refer to a nucleic acid fragment that can express a functional molecule such as, but not limited to, a specific protein, including: introns, exons, regulatory sequences preceding (5′ non-coding sequences) and following (3′ non-coding sequences) a coding sequence. In some embodiments, “Native gene” can refer to a gene as found in nature, for example, with its own regulatory sequences.

In some embodiments, a “mutated gene” can be a gene that has been altered relative to a corresponding naturally occurring gene; e.g., through human intervention. In some embodiments, such a “mutated gene” can have a sequence that differs from a sequence of a corresponding non-mutated gene by at least one nucleotide addition, deletion, or substitution. In some embodiments, a mutated gene can comprise an alteration that results from a polynucleotide guided polypeptide system as disclosed herein. In some embodiments, a mutated organism can be an organism comprising a mutated gene; e.g., a mutated plant with an organellar genome comprising a mutated gene. In some embodiments, the terms “mutated gene” and “mutant gene” can be used interchangeably herein.

In some embodiments, a “silent mutation” can refer to a mutated sequence that has a same functionality as a wild-type sequence; e.g., replacement of a codon in a protein-coding region with a synonymous codon that can encode a same amino acid.

As used herein, a “targeted mutation” can be a DNA modification made at or near a specific target site in a genome. In some embodiments, a targeted mutation may be as small as a single nucleotide change in a native gene. In some embodiments, a targeted mutation may involve a larger DNA modification such as an insertion of one or more heterologous DNAs, e.g., a heterologous regulatory element, a heterologous protein-coding sequence, or an expression cassette coding for a heterologous protein or functional RNA. In some embodiments, a targeted mutation may also involve a change in a sequence of a target site.

In some embodiments, the term “SDN” can refer to “site-directed nuclease”. In some embodiments, an SDN-induced mutation can include; an induction of site-specific random mutations; an induction of mutations in a predefined sequence of a particular gene; a replacement or an insertion of an entire gene; or any combination thereof. In some embodiments, SDN-induced mutations can be referred to as SDN-1, SDN-2 and SDN-3, respectively.

In some embodiments, a “codon-modified gene” or “codon-preferred gene” or “codon-optimized gene” can be a gene having its frequency of codon usage designed to mimic a frequency of preferred codon usage of a host cell in a compartment of interest. In some embodiments, a compartment of interest can comprise a nucleus, a mitochondrion, a chloroplast, or any combination thereof.

In some embodiments, a “mature” protein can refer to a post-translationally processed polypeptide; for example, one from which any pre- or pro-peptides present in a primary translation product have been removed.

In some embodiments, a “precursor” protein can refer to a primary product of translation of an mRNA; for example, with pre- and pro-peptides still present. In some embodiments, pre- and pro-peptides may, for example, comprise intracellular localization signals.

In some embodiments, “isolated” can refer to materials, such as nucleic acid molecules, proteins, and cells that may be substantially free or otherwise removed from components that normally accompany or interact with materials in a naturally occurring environment. In some embodiments, isolated polynucleotides can be purified from a host cell in which they can naturally occur. In some embodiments, nucleic acid purification methods can be used to obtain isolated polynucleotides. In some embodiments, isolated polynucleotides can include, for example, recombinant polynucleotides and chemically synthesized polynucleotides.

In some embodiments, “heterologous”, for example, with respect to sequence, can mean a sequence that originates from a foreign species, or, if from the same species, is substantially modified from its native form in composition and/or genomic locus by deliberate human intervention. In some embodiments, the terms “heterologous nucleotide sequence”, “heterologous sequence”, “heterologous nucleic acid fragment”, and “heterologous nucleic acid sequence” can be used interchangeably herein.

In some embodiments, “recombinant” can refer to an artificial combination of two or more otherwise separated segments of sequence, e.g., by chemical synthesis or by a manipulation of isolated segments of nucleic acids by genetic engineering techniques. In some embodiments, “Recombinant” can also include reference to a cell or vector, for example, that has been modified by an introduction of a heterologous nucleic acid or a cell derived from a cell so modified.

In some embodiments, a “recombinant DNA construct” can refer to a combination of nucleic acid fragments that may not normally be found together in nature. In some embodiments, a recombinant DNA construct may comprise, for example, regulatory sequences and coding sequences that are derived from different sources, or regulatory sequences and coding sequences derived from the same source. In some embodiments, sequences in a recombinant DNA construct can be arranged in a manner different than that normally found in nature. In some embodiments, the terms “recombinant DNA construct”, “recombinant DNA molecule”, “recombinant construct”, “DNA construct” and “construct” can be used interchangeably herein. In some embodiments, a recombinant DNA construct may be any of the following non-limiting examples: single-stranded, double-stranded, or both single-stranded and double-stranded; linear or circular; DNA, RNA, or a combination of DNA and RNA; a plasmid DNA, a viral DNA, a viral RNA, or a viroid RNA.

In some embodiments, “expression” can refer to a production of a functional product. For example, expression of a nucleic acid fragment may refer to transcription of the nucleic acid fragment (e.g., transcription resulting in mRNA or functional RNA) and/or translation of mRNA into a precursor or mature protein.

In some embodiments, an “expression cassette” can refer to a construct containing, for example, a polynucleotide, a regulatory element(s), and a polynucleotide that allow for expression of a polynucleotide in a host. In some embodiments, the terms “expression cassette” and “expression construct” can be used interchangeably herein.

In some embodiments, the terms “entry clone” and “entry vector” can be used interchangeably herein.

In some embodiments, “regulatory sequences” can refer to nucleotide sequences, for example, located upstream (e.g., 5′ non-coding sequences), within (e.g., in introns), or downstream (e.g., 3′ non-coding sequences) of a coding sequence. In some embodiments, regulatory sequences can influence, for example, the transcription, RNA processing or stability, or translation of the associated coding sequence.

In some embodiments, regulatory sequences may include, but are not limited to, promoters, translation leader sequences, 5′ untranslated sequences, 3′ untranslated sequences, introns, polyadenylation target sequences, RNA processing sites, effector binding sites, and stem-loop structures. In some embodiments, a regulatory sequence may act in “cis” or “trans”. In some embodiments, the nucleic acid molecule regulated by a regulatory sequence may not necessarily have to encode a functional peptide or polypeptide, e.g., the regulatory sequence can modulate the expression of a short interfering RNA or an antisense RNA. In some embodiments, the terms “regulatory sequence” and “regulatory element” can be used interchangeably herein.

In some embodiments, “promoter” can refer to a nucleic acid fragment that can control transcription of another nucleic acid fragment. In some embodiments, a promoter can include a core promoter (also known as minimal promoter) sequence. In some embodiments, a core promoter can be a minimal sequence for direct transcription initiation. In some embodiments, a core promoter can optionally include enhancers or other regulatory elements. In some embodiments, promoters may be derived in their entirety from a native gene, or be composed of different elements derived from different promoters found in nature, or even comprise synthetic DNA segments. Different promoters may direct the expression of a gene in different tissues or cell types, or at different stages of development, or in response to different environmental conditions.

In some embodiments, a “promoter functional in a plant” can be a promoter that can control transcription in plant cells. In some embodiments, a promoter can be from any suitable origin, which can include plant cells and non-plant cells.

In some embodiments, a “tissue-specific promoter” and “tissue-preferred promoter” can be used interchangeably and can refer to a promoter that can be expressed predominantly in one tissue, one organ or one cell type. In some embodiments, a tissue-specific promoter may not be necessarily exclusive in one tissue, one organ or one cell type. In some embodiments, a Root-preferred promoter can include, for example, the following: soybean root-specific glutamine synthase gene; cytosolic glutamine synthase (GS); root-specific control element in the GRP 1.8 gene of French bean; root-specific promoter of A. tumefaciens mannopine synthase (MAS); root-specific promoters isolated from Parasponia andersonii and Trema tomentosa; A. rhizogenes rolC and rolD root-inducing genes; Agrobacterium wound-induced TR1′ and TR2′ genes; VfENOD-GRP3 gene promoter; and rolB promoter. In some embodiments, a Seed-preferred promoter can include a seed-specific promoter active during seed development, a seed-germinating promoter active during seed germination, or any combination thereof. In some embodiments, a seed-preferred promoter can include CimI (cytokinin-induced message); cZ19B1 (maize 19 kDa zein); milps (myo-inositol-1-phosphate synthase); END1; and END2, or any combination thereof. In some embodiments, for a dicot, a seed-preferred promoter can include; bean β-phaseolin; napin; β-conglycinin; soybean lectin; cruciferin; and any combination thereof. In some embodiments, for monocots, a seed-preferred promoter can include maize 15 kDa zein; 22 kDa zein; 27 kDa gamma zein; waxy; shrunken 1; shrunken 2; globulin 1; oleosin; nud; Zea mays-Rootmet2 promoter, or any combination thereof. In some embodiments, a leaf-preferred promoter can include a plant rbcS promoter, such as a soybean rbcS promoter, a maize rbcS promoter; a Zea mays PEPC1 promoter, or any combination thereof.

In some embodiments, a “developmentally regulated promoter” can refer to a promoter whose activity can be determined by developmental events.

In some embodiments, an “inducible promoter” can refer to a promoter that selectively expresses an operably linked DNA sequence in response to a presence of an endogenous or exogenous stimulus, for example by a chemical compound (e.g., a chemical inducer) or in response to an environmental, hormonal, chemical, and/or developmental signal. In some embodiments, an Inducible or regulated promoter can include, for example, promoters regulated by light, heat, stress, flooding or drought, phytohormones, wounding, or chemicals such as ethanol, jasmonate, salicylic acid, or safeners. In some embodiments, a pathogen-inducible promoter that can be induced following infection by a pathogen can include, those regulating expression of PR proteins, SAR proteins, beta-1,3-glucanase, chitinase, or any combination thereof. In some embodiments, a stress-inducible promoter can include a plant RAB17 promoter, such as a maize RAB17 promoter. In some embodiments, a chemical-inducible promoter can include, a maize ln2-2 promoter, an activated by benzene sulfonamide herbicide safeners; a maize GST promoter, an activated by hydrophobic electrophilic compound used as pre-emergent herbicides; a tobacco PR-1a promoter, activated by salicylic acid, or any combination thereof. In some embodiments, a chemical-regulated promoter can include a steroid-responsive promoter, for example, a glucocorticoid-inducible promoter, a tetracycline-inducible and a tetracycline-repressible promoter.

In some embodiments, a “constitutive promoter” can refer to promoters active in all or most tissues or cell types of an organism at all or most developing stages. In some embodiments, a promoter classified as “constitutive” (e.g. ubiquitin), some variation in absolute levels of expression can exist among different tissues or stages. In some embodiments, the term “constitutive promoter” or “tissue-independent promoter” can be used interchangeably herein. In some embodiments, constitutive promoters include the following: the core promoter of the Rsyn7 promoter; the core CaMV 35S promoter; plant actin promoter, such as a rice actin promoter and a maize actin promoter; plant ubiquitin promoter, such as a maize ubiquitin promoter and a soybean ubiquitin promoter; pEMU; MAS promoter; ALS promoter; plant GOS2 promoter, such as a maize GOS2 promoter; soybean GM-EF1 A2 promoter; plant U6 polymerase III promoter, such as a maize U6 polymerase III promoter and a soybean U6 polymerase III promoter (GM-U6-9.1 and GM-U6-13.1); and any combination thereof.

In some embodiments, an enhancer element can be any nucleic acid molecule that increases transcription of a nucleic acid molecule when functionally linked to a promoter regardless of its relative position. In some embodiments, an enhancer may be an innate element of the promoter or a heterologous element inserted to enhance the level or tissue-specificity of a promoter.

In some embodiments, a repressor (also sometimes called herein silencer) can be defined as any nucleic acid molecule which inhibits the transcription when functionally linked to a promoter regardless of relative position.

In some embodiments, a “translation leader sequence” can refer to a polynucleotide sequence located between the promoter sequence of a gene and the coding sequence. In some embodiments, the translation leader sequence can be present in the fully processed mRNA upstream of the translation start sequence. In some embodiments, the translation leader sequence may affect processing of the primary transcript to mRNA, mRNA stability or translation efficiency.

In some embodiments, a “transcription terminator”, “termination sequence”, or “terminator” can refer to DNA sequences that, when operably linked to the 3′ end of a polynucleotide sequence that is to be expressed, can terminate transcription from the polynucleotide sequence. In some embodiments, a transcription termination can refer to the process by which RNA synthesis by RNA polymerase can be stopped and both the RNA and the enzyme are released from the DNA template.

In some embodiments, “operably linked” can refer to the association of fragments in a single fragment (e.g., a polynucleotide or polypeptide), or in a single complex, so that the function of one can be regulated by the other. In some embodiments, a linkage may be covalent or non-covalent. In some embodiments, with respect to nucleic acid fragments, a promoter can be operably linked with a nucleic acid fragment if the promoter can regulate the transcription of that nucleic acid fragment. In some embodiments, with respect to a polypeptide, an organelle targeting peptide can be operably linked with a polypeptide if the organelle targeting peptide can transport that polypeptide into the relevant organelle. In some embodiments, with respect to a complex, a guide RNA can be operably linked to a Cas polypeptide if the guide RNA/Cas polypeptide complex can cleave a target sequence as directed by the guide RNA.

In some embodiments, a “phenotype” can refer to the detectable characteristics of a cell or organism.

In some embodiments, the term “introduced” can mean providing a polynucleic acid (e.g., expression construct) or protein into a cell. In some embodiments, “introduced” can include reference to the incorporation of a nucleic acid into a eukaryotic or prokaryotic cell, for example, where the nucleic acid may be incorporated into the genome of the cell. In some embodiments, “introduced” can include reference to the transient provision of a nucleic acid or protein to the cell. In some embodiments, “introduced” can include reference to stable or transient gene editing method. In some embodiments, “introduced” can include reference to stable or transient transformation methods. Introduced can include sexually crossing. In some embodiments, “introduced”, for example, in the context of inserting a nucleic acid fragment (e.g., a recombinant DNA construct) into a cell, can include “transfection” or “transformation” or “transduction”. In some embodiments, “introduced” can include reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

In some embodiments, an “edited mitochondrial genome” may comprise introduction of (i) a replacement of at least one nucleotide, (ii) a substitution of at least one nucleotide, (iii) a deletion of at least one nucleotide (iv) an insertion of at least one nucleotide or (v) any combination of (i)-(iv). In some embodiments, a cell may comprise an edited mitochondrial genome with at least one nucleotide replacement, substitution, deletion, or insertion. In some embodiments, a cell may comprise a transformed mitochondrion, wherein the transformed mitochondrial comprises the edited mitochondrial genome.

In some embodiments, a “transformed cell” can be any cell which a nucleic acid fragment (e.g., a recombinant DNA construct) has been introduced or edited.

In some embodiments, “transformation” as used herein can refer to a stable transformation. In some embodiments, a transformation can refer to transient transformation.

In some embodiments, “stable transformation” can refer to an introduction of a nucleic acid fragment into a genome of a host organism resulting in genetically stable inheritance. In some embodiments, once stably transformed, the nucleic acid fragment can be stably integrated in the genome of the host organism and any subsequent generation.

In some embodiments, a “transient transformation” can refer to the introduction of a nucleic acid fragment into the nucleus, or DNA-containing organelle, thereby editing or modifying a host organism nucleus or organelle genomes resulting in gene expression without genetically stable inheritance.

In some embodiments, host organisms containing the transformed nucleic acid fragments can be referred to as “transgenic” organisms.

In some embodiments, a “transformation cassette” can refer to a construct having elements that facilitates transformation of a particular host cell. In some embodiments, the terms “transformation cassette” and “transformation construct” can be used interchangeably herein.

In some embodiments, “homoplasmic” can refer to a eukaryotic cell in which the copies of mitochondrial DNA are all identical. In some embodiments, “heteroplasmic” can refer to a eukaryotic cell in which the copies of mitochondrial DNA are not all identical.

In some embodiments, an “allele” can be one of several alternative forms of a gene occupying a given locus on a chromosome. In some embodiments, when the alleles present at a given locus on a pair of homologous chromosomes in a diploid plant are the same that plant can be homozygous at that locus. In some embodiments, if the alleles present at a given locus on a pair of homologous chromosomes in a diploid plant differ, that plant can be heterozygous at that locus. In some embodiments, if a transgene is present on one of a pair of homologous chromosomes in a diploid plant that plant can be hemizygous at that locus.

In some embodiments, the terms “organelle-specific” and “organelle-preferred” can be used interchangeably, and when used to describe a regulatory element (e.g., an organelle-specific promoter), refer to a regulatory element that is functional within a given cell (e.g., a plant cell) predominantly but not necessarily exclusively in an organelle (e.g., a mitochondrion, a plastid).

In some embodiments, an organelle-specific regulatory domain may be derived from an organellar polynucleotide of interest (e.g., a mitochondrial polynucleotide, a plastid polynucleotide). In some embodiments, an organelle-specific regulatory domain may comprise all or part of the nucleic acid sequence of an organellar polynucleotide of interest. In some embodiments, the organelle-specific regulatory domain may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the organellar polynucleotide of interest.

In some embodiments, the terms “mitochondrial-specific” and “mitochondrial-preferred” can be used interchangeably, and when used to describe a regulatory element (e.g., a mitochondrial-specific promoter), refer to a regulatory element that is functional within a given cell (e.g., a plant cell) predominantly but not necessarily exclusively in mitochondria.

In some embodiments, the terms “plastid-specific” and “plastid-preferred” can be used interchangeably, and when used to describe a regulatory element (e.g., a plastid-specific promoter), refer to a regulatory element that is functional within a given cell (e.g., a plant cell) predominantly but not necessarily exclusively in plastids.

In some embodiments, the terms “chloroplast-specific” and “chloroplast-preferred” can be used interchangeably, and when used to describe a regulatory element (e.g., a chloroplast-specific promoter), refer to a regulatory element that is functional within a given cell (e.g., a plant cell) predominantly but not necessarily exclusively in chloroplasts.

In some embodiments, the terms “mitochondrial genome” and “genome of a mitochondrion” can be used interchangeably and refer to the nucleic acid sequences present within endogenous mitochondrial genetic elements. In some embodiments, the mitochondrial genome may be edited by the addition of a sequence (e.g., a heterologous sequence) into an endogenous mitochondrial genetic element. In some embodiments, an autonomously replicating heterologous episomal element (e.g., a plasmid DNA) introduced into a mitochondrion is considered to be an independent genetic element and is not considered to be part of the mitochondrial genome.

In some embodiments, the terms “plastid genome”, “chloroplast genome”, “genome of a plastid” and “genome of a chloroplast” can be used interchangeably and refer to a nucleic acid sequence present within endogenous plastid genetic elements. In some embodiments, a plastid genome may be edited by the addition of a sequence (e.g., a heterologous sequence) into an endogenous plastid genetic element. In some embodiments, an autonomously replicating heterologous episomal element (e.g., a plasmid DNA) introduced into a plastid is considered to be an independent genetic element and is not considered to be part of the plastid genome.

In some embodiments, a “chloroplast transit peptide” can be an amino acid sequence that can direct a protein to the chloroplast or other plastid types present in the cell. In some embodiments, a chloroplast transit peptide can be translated in conjunction with the protein in the cell in which the protein can be made. In some embodiments, the terms “chloroplast transit peptide”, “plastid transit peptide”, “chloroplast targeting peptide” and “plastid targeting peptide” can be used interchangeably herein. “Chloroplast transit sequence” can refer to a nucleotide sequence that can encode a chloroplast transit peptide.

In some embodiments, a “signal peptide” can be an amino acid sequence that can direct a protein to the secretory system. The signal peptide can be translated in conjunction with a protein. For example, if the protein is to be directed to a vacuole, a vacuolar targeting signal (supra) can further be added, or if to an endoplasmic reticulum, an endoplasmic reticulum retention signal (supra) may be added. If a protein is to be directed to the nucleus, any signal peptide present can be removed and a nuclear localization signal can be included.

In some embodiments, a “mitochondrial targeting peptide” can be an amino acid sequence which can direct a precursor protein into the mitochondria. In some embodiments, the terms “mitochondrial targeting peptide”, “mitochondrial signal peptide” and “mitochondrial transit peptide” can be used interchangeably herein.

In some embodiments, an “organelle targeting polynucleotide” can be a nucleotide sequence which can direct import of the polynucleotide into an organelle. In some embodiments, the terms “organelle targeting polynucleotide”, “organelle targeting nucleic acid” and “organelle targeting nucleic acid sequence” can be used interchangeably herein. In some embodiments, an organelle targeting polynucleotide may be directed to, for example, the plastid (“plastid targeting polynucleotide”) or the mitochondria (“mitochondria targeting polynucleotide”). In some embodiments, a polynucleotide can be RNA (“organelle targeting RNA”), DNA (“organelle targeting DNA) or a combination of RNA and DNA.

In some embodiments, an organelle targeting RNA directed to the plastid can be termed a “plastid targeting RNA”. In some embodiments, the terms “plastid targeting RNA”, “chloroplast targeting RNA” and “transit RNA” are used interchangeably herein. In some embodiments, an organelle targeting RNA directed to the mitochondria can be termed a “mitochondria targeting RNA”.

In some embodiments, RNAs can be imported into mitochondria. In some embodiments, one such mitochondrial targeting RNA can be the yeast tRNALys. In some embodiments, yeast tRNALys and its variants can be imported into human mitochondria. In some embodiments, another RNA that can be imported into mitochondria can be 5S rRNA. In some embodiments, 5S rRNA can function as a vector for delivering heterologous RNA sequences into, for example, mitochondria (e.g., human). In some embodiments, RNAs can be used with the compositions and methods of the disclosure for example, for targeting to an organelle (e.g., the mitochondria).

In some embodiments, RNAs can be imported into plastids. In some embodiments, plastid targeting RNAs that can mediate import of attached heterologous RNA can include vd-5′UTR (e.g., viroid-derived ncRNA sequence acting as 5′UTR and eIF4E1 mRNA. In some embodiments, RNAs can be used with the compositions and methods of the disclosure for targeting to an organelle (e.g., the plastid).

In some embodiments, as used herein, “fusion” can refer to a protein and/or nucleic acid comprising one or more non-native sequences (e.g., moieties). In some embodiments, any of the molecules described herein (e.g., nucleic acids, proteins, polypeptides, polynucleic acid, Cas protein, guide polynucleotide) can be engineered as fusions. In some embodiments, a fusion can comprise one or more of the same non-native sequences. In some embodiments, a fusion can comprise one or more of different non-native sequences. In some embodiments, a fusion can be a chimera. In some embodiments, a fusion can comprise a nucleic acid affinity tag. In some embodiments, a fusion can comprise a barcode. In some embodiments, a fusion can comprise a peptide affinity tag. In some embodiments, a fusion can provide for subcellular localization of the site-directed polypeptide. In some embodiments, a fusion can provide a non-native sequence (e.g., affinity tag) that can be used to track or purify. In some embodiments, a fusion can be a small molecule such as biotin or a dye such as alexa fluor dyes, Cyanine3 dye, Cyanine5 dye, or any combination thereof.

In some embodiments, a fusion can refer to any protein with a functional effect. In some embodiments, a fusion protein can comprise deaminase activity, cytidine deaminase activity (US Patent Publication No. US20150166980, herein incorporated by reference), adenine deaminase activity (US Patent Publication No. US20180073012, herein incorporated by reference), uracil glycosylase inhibitor activity (US Patent Publication No. US20170121693, herein incorporated by reference), methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodeling activity, protease activity, oxidoreductase activity, transferase activity, hydrolase activity, lyase activity, isomerase activity, synthase activity, synthetase activity, or demyristoylation activity. In some embodiments, an effector protein can modify a genomic locus. In some embodiments, a fusion protein can be a fusion in a Cas protein. In some embodiments, a Cas protein can be a modified form that has nickase activity or that has no substantial nucleic acid-cleaving activity. In some embodiments, a fusion protein can be a non-native sequence in a Cas protein.

In some embodiments, as used herein, a “nucleic acid” can refer to a polynucleotide sequence, or fragment thereof. In some embodiments, a nucleic acid can comprise nucleotides. In some embodiments, a nucleic acid can be exogenous or endogenous to a cell. In some embodiments, a nucleic acid can exist in a cell-free environment. In some embodiments, a nucleic acid can be a gene or fragment thereof. In some embodiments, a nucleic acid can be DNA. In some embodiments, a nucleic acid can be RNA. In some embodiments, a nucleic acid can comprise one or more analogs (e.g. altered backbone, sugar, or nucleobase). In some embodiments, non-limiting examples of analogs can include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine.

In some embodiments, “silencing,” as used herein with respect to the target gene, can refer to the suppression of levels of mRNA or protein/enzyme expressed by the target gene, and/or the level of the enzyme activity or protein functionality. In some embodiments, the terms “suppression”, “suppressing” and “silencing”, which can be used interchangeably herein, can include lowering, reducing, declining, decreasing, inhibiting, eliminating or preventing. In some embodiments, “Silencing” or “gene silencing” can occur by any suitable mechanism. In some embodiments, non-limiting examples of silencing can include antisense, cosuppression, viral-suppression, hairpin suppression, stem-loop suppression, RNAi-based approaches, small RNA-based approaches, and any combination thereof.

In some embodiments, suppression of gene expression can also be achieved by, for example, use of artificial miRNA precursors, ribozyme constructs and gene disruption. In some embodiments, a modified plant miRNA precursor may be used, wherein the precursor has been modified, for example, to replace the miRNA encoding region with a sequence designed to produce a miRNA directed to the nucleotide sequence of interest. In some embodiments, a gene disruption may be achieved by use of transposable elements or by use of chemical agents that cause site-specific mutations.

### Sequence Identity, Similarity, and Variation

In some embodiments, a sequence alignment and percent identity or similarity calculation may be determined using a variety of comparison methods designed to detect homologous sequences including, but not limited to, the MEGALIGN™ program of the LASERGENE™ bioinformatics computing suite (DNASTAR™ Inc., Madison, Wl). In some embodiments, where sequence analysis software is used for analysis, results of an analysis can be based on “default values” of a program referenced. In some embodiments, as used herein “default values” can mean any set of values or parameters that originally load with the software when first initialized.

In some embodiments, “Clustal V method of alignment” can correspond to an alignment method labeled Clustal V and, for example, found in a MEGALIGN™ program of a LASERGENE™ bioinformatics computing suite (DNASTAR™ Inc., Madison, Wl). In some embodiments, for multiple alignments, default values can correspond to GAP PENALTY=10 and GAP LENGTH PENALTY=10. In some embodiments, default parameters for pairwise alignments and calculation of percent identity of protein sequences using the Clustal method can be, for example, KTUPLE=1, GAP PENALTY=3, WINDOW=5 and DIAGONALS SAVED=5. In some embodiments, for nucleic acids these parameters can be for example KTUPLE=2, GAP PENALTY=5, WINDOW=4 and DIAGONALS SAVED=4. In some embodiments, after alignment of sequences using the Clustal V program, “percent identity” and “divergence” values can be obtained by viewing the “sequence distances” table in the same program.

In some embodiments, the “Clustal W method of alignment” can correspond to the alignment method labeled Clustal W and, for example, found in the MEGALIGN™ v6.1 program of the LASERGENE™ bioinformatics computing suite (DNASTAR™ Inc., Madison, Wl). In some embodiments, default parameters for multiple alignment can correspond to for example: GAP PENALTY=10, GAP LENGTH PENALTY=0.2, Delay Divergence Sequences=30%, DNA Transition Weight=0.5, Protein Weight Matrix=Gonnet Series, DNA Weight Matrix=IUB. In some embodiments, after alignment of the sequences using the Clustal W program, “percent identity” values can be obtained by viewing the “sequence distances” table in the same program.

In some embodiments, sequence identity/similarity values can also be obtained using GAP Version 10 (GCG, Accelrys™, San Diego, CA) using for example the following parameters: % identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix. In some embodiments, GAP can use an algorithm to find an alignment of two complete sequences that can maximize the number of matches and minimizes the number of gaps. In some embodiments, GAP can consider all possible alignments and gap positions. In some embodiments, GAP can create the alignment with the largest number of matched bases and the fewest gaps, using, for example, a gap creation penalty and a gap extension penalty in units of matched bases.

In some embodiments, “BLAST” can be a searching algorithm provided by the National Center for Biotechnology Information (NCBI) that can be used to find regions of similarity between biological sequences. In some embodiments, BLAST can compare nucleotide or protein sequences to sequence databases. In some embodiments, BLAST can calculate the statistical significance of matches to identify sequences having sufficient similarity to a query sequence such that the similarity may not be predicted to have occurred randomly. In some embodiments, BLAST can report the identified sequences and their local alignment to the query sequence.

In some embodiments, the term “conserved domain” or “motif” can mean a set of amino acids conserved at specific positions along an aligned sequence of evolutionarily related proteins. In some embodiments, while amino acids at other positions can vary between homologous proteins, amino acids that are highly conserved at specific positions can indicate, for example, amino acids that are essential to the structure, the stability, or the activity of a protein.

In some embodiments, conserved domains or motifs can be identified by their high degree of conservation in aligned sequences of a family of protein homologues. In some embodiments, conserved domains can be used as identifiers, or “signatures”, for example, to determine if a protein with a newly determined sequence belongs to a previously identified protein family.

In some embodiments, polynucleotide and polypeptide sequences, variants thereof, and the structural relationships of these sequences can be described by the terms “homology”, “homologous”, “substantially identical”, “substantially similar” and “corresponding substantially” which are used interchangeably herein. In some embodiments, these can refer to polypeptide or nucleic acid fragments wherein changes in one or more amino acids or nucleotide bases may not affect the function of the molecule, such as the ability to mediate gene expression or to produce a certain phenotype. In some embodiments, these terms can also refer to modification(s) of nucleic acid fragments that may not substantially alter the functional properties of the resulting nucleic acid fragment relative to the initial, unmodified fragment. In some embodiments, these modifications can include deletion, replacement substitution, and/or insertion of one or more nucleotides in the nucleic acid fragment.

In some embodiments, substantially similar nucleic acid sequences encompassed may be defined by their ability to hybridize (for example, under moderately stringent conditions, e.g., 0.5×SSC, 0.1% SDS, 60° C.) with the sequences exemplified herein, or to any portion of the nucleotide sequences disclosed herein. In some embodiments, substantially similar nucleic acid sequences can be functionally equivalent to any of the nucleic acid sequences disclosed herein. In some embodiments, stringency conditions can be adjusted to screen for moderately similar fragments, such as homologous sequences from distantly related organisms, to highly similar fragments, such as genes that duplicate functional enzymes from closely related organisms. In some embodiments, post-hybridization washes can determine stringency conditions.

In some embodiments, the term “selectively hybridizes” can include reference to hybridization, for example under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. In some embodiments, selectively hybridizing sequences can have, for example, about at least 80% sequence identity, or 90% sequence identity, up to and including 100% sequence identity (i.e., fully complementary) with each other.

In some embodiments, the term “stringent conditions” or “stringent hybridization conditions” can include reference to conditions under which a probe can selectively hybridize to its target sequence in an in vitro hybridization assay. In some embodiments, stringent conditions can be sequence-dependent. In some embodiments, stringent conditions can be different in different circumstances. In some embodiments, by controlling the stringency of the hybridization and/or washing conditions, target sequences can be identified which are 100% complementary to the probe (homologous probing).

In some embodiments, stringency conditions can be adjusted to allow some mismatching in sequences so that lower degrees of similarity are detected (heterologous probing). In some embodiments, a probe can be less than about 1000 nucleotides in length, optionally less than 500 nucleotides in length.

In some embodiments, stringent conditions can comprise those in which a salt concentration is less than about 1.5 M Na ion. In some embodiments, stringent conditions can comprise those in which a salt concentration is less than about 0.01 to 1.0 M Na ion concentration (or other salt(s)) at pH 7.0 to 8.3. In some embodiments, stringent conditions can comprise a temperature of about 30° C. for short probes (e.g., 10 to 50 nucleotides). In some embodiments, stringent conditions can comprise a temperature of at least about 60° C. for long probes (e.g., greater than 50 nucleotides). In some embodiments, stringent conditions can also be achieved with the addition of destabilizing agents such as formamide. In some embodiments, exemplary low stringency conditions can include hybridization with a buffer solution of, for example, 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. In some embodiments, exemplary moderate stringency conditions can include hybridization in 40 to 45% formamide, 1 M NaCl, 1% SDS at 37° C., and a wash in 0.5× to 1×SSC at 55 to 60° C. In some embodiments, exemplary high stringency conditions can include hybridization in, for example, 50% formamide, 1 M NaCl, 1% SDS at 37° C., and a wash in 0.1×SSC at 60 to 65° C.

In some embodiments, “sequence identity” or “identity” in the context of nucleic acid or polypeptide sequences can refer to the nucleic acid bases or amino acid residues in two sequences that are the same when aligned for maximum correspondence over a specified comparison window.

In some embodiments, the term “percentage of sequence identity” can refer to a value determined by comparing two optimally aligned sequences over a comparison window. In some embodiments, a portion of the polynucleotide or polypeptide sequence in the comparison window may comprise additions or deletions (i.e., gaps) as compared to the reference sequence (which may or may not comprise additions or deletions) for optimal alignment of the two sequences. In some embodiments, a percentage can be calculated by, for example, determining a number of positions at which an identical nucleic acid base or amino acid residue occurs in both sequences to yield a number of matched positions, dividing a number of matched positions by a total number of positions in a window of comparison and multiplying the results by 100 to yield the percentage of sequence identity. In some embodiments, percent sequence identities can include, but are not limited to, 50%, 55%, 60%, 65%, 70%, 75%, 80%, 85%, 90% or 95%, or any percentage from 50% to 100%. In some embodiments, sequence identity can include an integer percentage from 50% to 100%. In some embodiments, these identities can be determined using any of the programs described herein.

In some embodiments, sequence identity can be useful in identifying polypeptides from other species or modified naturally or synthetically wherein such polypeptides have the same or similar function or activity. In some embodiments, percent identities can include, but are not limited to, 50%, 55%, 60%, 65%, 70%, 75%, 80%, 85%, 90% or 95%. In some embodiments, sequence identity (e.g., amino acid sequence identity) can include an integer percentage from 50% to 100%. In some embodiments, sequence (e.g., amino acid) identity can include, for example, about: 50%, 51%, 52%, 53%, 54%, 55%, 56%, 57%, 5⁸%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

### Definitions, Traits, and Processes Relevant to Plants

In some embodiments, “plant” can include reference to whole plants, plant organs, plant tissues, plant propagules, seeds and plant cells and progeny of same. In some embodiments, plant cells include, without limitation, cells from seeds, suspension cultures, embryos, meristematic regions, callus tissue, leaves, roots, shoots, gametophytes, sporophytes, pollen, and microspores.

In some embodiments, a “propagule” can include products of meiosis and/or mitosis able to propagate a new plant. In some embodiments, a propagule can include seeds, spores and parts of a plant that can serve as a means of vegetative reproduction, such as corms, tubers, offsets, or runners. In some embodiments, a propagule can include grafts where one portion of a plant can be grafted to another portion of a different plant (even one of a different species) to create a living organism. In some embodiments, a propagule can include plants and seeds produced by cloning or by bringing together meiotic products, or allowing meiotic products to come together to form an embryo or fertilized egg (naturally or with human intervention).

In some embodiments, a “progeny” can comprise any subsequent generation of a plant.

In some embodiments, the terms “monocot” and “monocotyledonous plant” can be used interchangeably herein. In some embodiments, a monocot can include the Gramineae.

In some embodiments, the terms “dicot” and “dicotyledonous plant” can be used interchangeably herein. In some embodiments, a dicot can include, for example, the following families: Brassicaceae, Leguminosae, and Solanaceae.

In some embodiments, “transgenic plant” can include reference to a plant which can comprise within its genome a heterologous polynucleotide. In some embodiments, a heterologous polynucleotide can be stably integrated within a genome (e.g., nuclear, plastid, mitochondrial) such that a polynucleotide can be passed on to successive generations. In some embodiments, a heterologous polynucleotide can be integrated into a genome alone or as part of a recombinant DNA construct.

In some embodiments, a “transgenic plant” can include reference to plants which can comprise more than one heterologous polynucleotide within their genome. In some embodiments, each heterologous polynucleotide can confer a different trait to a transgenic plant.

In some embodiments, multiple traits can be introduced into crop plants, and can be referred to as a gene stacking approach. In some embodiments, gene stacking can be used, for example, for development of genetically improved germplasm. In some embodiments, multiple genes conferring different characteristics of interest can be introduced into a plant. In some embodiments, gene stacking can be accomplished by many means including but not limited to co-transformation, retransformation, and crossing lines with different transgenes. In some embodiments, as used herein, the term “stacked” can include having multiple traits present in the same plant (e.g., both traits are incorporated into the nuclear genome, one trait is incorporated into the nuclear genome and one trait is incorporated into the genome of an organelle, or both traits are incorporated into the genome of an organelle).

In some embodiments, the term “crossed” or “cross” or “crossing” in the context ofthe disclosure can mean the fusion of gametes (e.g., via pollination) to produce progeny (e.g., cells, seeds, or plants). In some embodiments, the term can encompass both sexual crosses (e.g., the pollination of one plant by another) and selfing (e.g., self-pollination; when the pollen and ovule are from the same plant or genetically identical plants).

In some embodiments, the term “maternal inheritance” can refer to the transmission of traits that can be solely dependent on properties of the genome of the female gamete.

In some embodiments, the term “paternal inheritance” can refer to the transmission of traits that are solely dependent on properties of the genome of the male gamete.

In some embodiments, the term “introgression” can refer to the transmission of a desired allele of a genetic locus from one genetic background to another. In some embodiments, introgression of a desired allele at a specified locus can be transmitted to at least one progeny plant via a sexual cross between two parent plants, where at least one of the parent plants has the desired allele within its genome. In some embodiments, transmission of an allele can occur by recombination between two donor genomes, e.g., in a fused protoplast, where at least one of the donor protoplasts has the desired allele in its genome. In some embodiments, a desired allele can be, e.g., a transgene or a selected allele of a marker or QTL.

In some embodiments, “a plant-optimized nucleotide sequence” can be a nucleotide sequence that has been optimized for increased expression in plants, particularly for increased expression in a given plant or in one or more plants of interest. In some embodiments, a plant-optimized nucleotide sequence can be synthesized by modifying a nucleotide sequence encoding a protein by using plant-preferred codons for improved expression. In some embodiments, a host-preferred codon usage can be utilized for codon optimization. In some embodiments, a frequency of codon usage can be designed to mimic the frequency of preferred codon usage of a host cell in a compartment of interest, e.g., a nucleus, a mitochondrion or a chloroplast.

In some embodiments, plant-preferred genes can be synthesized. In some embodiments, additional sequence modifications can enhance gene expression in a plant host. In some embodiments, these can include, for example, elimination of any of the following: one or more sequences encoding spurious polyadenylation signals, one or more exon-intron splice site signals, one or more transposon-like repeats, and sequences that can be deleterious to gene expression. In some embodiments, a G-C content of a sequence may be adjusted, for example, to levels average for a given plant host, as calculated by reference to genes expressed in a host plant cell. In some embodiments, when possible, a sequence can be modified to avoid one or more predicted hairpin secondary mRNA structures. In some embodiments, “a plant-optimized nucleotide sequence” of a present disclosure can comprise one or more of such sequence modifications.

In some embodiments, a “trait” can refer to, for example, a physiological, morphological, biochemical, or physical characteristic of a plant or particular plant material or cell. In some instances, a characteristic can be visible to a human eye, such as seed or plant size, or can be measured by biochemical techniques, such as detecting a protein, starch, or oil content of seed or leaves, or by observation of a metabolic or physiological process, e.g. by measuring tolerance to water deprivation or particular salt or sugar concentrations, or by an observation of an expression level of a gene or genes, or by agricultural observations such as osmotic stress tolerance or yield.

In some embodiments, an “Agronomic characteristic” can be a measurable parameter including but not limited to, abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress.

### Herbicide Resistance in Plants

In some embodiments, an “herbicide resistance protein” or a protein resulting from expression of an “herbicide resistance-encoding nucleic acid molecule” can include proteins that can confer upon a cell the ability to tolerate a higher concentration of an herbicide, for example, compared with cells that do not express the protein. In some embodiments, the terms “herbicide resistance protein”, “herbicide resistant protein”, “herbicide tolerance protein” and “herbicide tolerant protein” may be used interchangeably herein.

In some embodiments, an herbicide resistance protein or a protein resulting from expression of a herbicide resistance-encoding nucleic acid molecule can include proteins that can confer upon a cell an ability to tolerate a concentration of a herbicide for a longer period of time than cells that do not express a protein. In some embodiments, herbicide resistance traits may be introduced into plants by, for example, genes coding for resistance to herbicides. In some embodiments, genes coding for resistance to herbicides include, for example, the following: genes that act to convey tolerance to inhibitors of acetolactate synthase (ALS), such as the sulfonylurea-type herbicides; genes (e.g., the bar gene, the pat gene) that act to convey tolerance to inhibitors of glutamine synthase, such as phosphinothricin or basta; genes that act to convey tolerance to inhibitors of the EPSP synthase gene, such as glyphosate; genes that act to convey tolerance to inhibitors of HPPD; genes that act to convey tolerance to inhibitors of an acetyl coenzyme A carboxylase (ACCase); and genes that act to convey tolerance to inhibitors of protoporphyrinogen oxidase (PPO or PROTOX).

In some embodiments, genes useful for conferring herbicide resistance in plants can include genes that encode herbicide resistance proteins. In some embodiments, herbicide resistance proteins can include herbicide tolerant versions of: an acetyl coenzyme A carboxylase (ACCase); a 4-hydroxyphenylpyruvate dioxygenase (HPPD); a sulfonylurea-tolerant acetolactate synthase (ALS); an imidazolinone-tolerant acetolactate synthase (ALS); a glyphosate-tolerant 5-enolpyruvylshikimate-3-phosphate synthase (EPSPS); a glyphosate-tolerant glyphosate oxidoreductase (GOX); a glyphosate N-acetyltransferase (GAT); a phosphinothricin acetyl transferase (PAT); a protoporphyrinogen oxidase (PPO or PROTOX); an auxin enzyme or receptor; a P450 polypeptide, or any combination thereof.

In some embodiments, as used herein, “Hydroxyphenylpyruvate dioxygenase” and “HPPD”, “4-hydroxy phenyl pyruvate (or pyruvic acid) dioxygenase (4-HPPD)” and “p-hydroxy phenyl pyruvate (or pyruvic acid) dioxygenase (p-OHPP)” can be synonymous and can refer to a non-heme iron-dependent oxygenase that catalyzes the conversion of 4-hydroxyphenylpyruvate to homogentisate. In some embodiments, in organisms that degrade tyrosine, a reaction catalyzed by HPPD can be a second step in a pathway. In some embodiments, in plants, formation of homogentisate can be necessary for the synthesis of plastoquinone, which can serve as a redox cofactor, and tocopherol. In some embodiments, a polynucleotide molecule encoding a herbicide tolerant hydroxyphenylpyruvate dioxygenase (HPPD) can provide tolerance to HPPD inhibitors.

In some embodiments, as used herein, an “HPPD inhibitor” can comprise any compound or combinations of compounds which can decrease an ability of HPPD to catalyze a conversion of 4-hydroxyphenylpyruvate to homogentisate. In specific embodiments, an HPPD inhibitor can comprise an herbicidal inhibitor of HPPD. In some embodiments, non-limiting examples of HPPD inhibitors include, triketones (such as, mesotrione, sulcotrione, topramezone, and tembotrione); isoxazoles (such as, pyrasulfotole and isoxaflutole); pyrazoles (such as, benzofenap, pyrazoxyfen, and pyrazolynate); and benzobicyclon. In some embodiments, agriculturally acceptable salts of various inhibitors can include salts (e.g., cations or anions) for a formation of salts for agricultural or horticultural use.

In some embodiments, an “ALS inhibitor-tolerant polypeptide” can comprise any polypeptide which when expressed in a plant can confer tolerance to at least one acetolactate synthase (ALS) inhibitor.

In some embodiments, ALS inhibitors can include, for example, sulfonylurea, imidazolinone, triazolopyrimidines, pryimidinyoxy(thio)benzoates, and/or sulfonylaminocarbonyltriazolinone herbicides.

In some embodiments, ALS mutations can fall into different classes with regard to tolerance to, for example, sulfonylureas, imidazolinones, triazolopyrimidines, and pyrimidinyl(thio)benzoates. In some embodiments, ALS mutations can include mutations having one or more of the following characteristics: (1) broad tolerance to all four of these groups (e.g., sulfonylureas, imidazolinones, triazolopyrimidines, and pyrimidinyl(thio)benzoates); (2) tolerance to imidazolinones and pyrimidinyl(thio)benzoates; (3) tolerance to sulfonylureas and triazolopyrimidines; and (4) tolerance to sulfonylureas and imidazolinones.

In some embodiments, polynucleotide molecules encoding proteins involved in herbicide resistance can include a polynucleotide molecule encoding a herbicide tolerant 5-enolpymvylshikimate-3-phosphate synthase (EPSPS) for example, for imparting glyphosate tolerance.

In some embodiments, glyphosate tolerance can also be obtained by expression of polynucleotide molecules encoding a glyphosate oxidoreductase (GOX) or a glyphosate-N-acetyl transferase (GAT).

In some embodiments, polynucleotides encoding an exogenous phosphinothricin acetyltransferase can be used for herbicide resistance. In some embodiments, plants containing an exogenous phosphinothricin acetyltransferase can exhibit improved tolerance to glufosinate herbicides, which can inhibit, for example, the enzyme glutamine synthase.

In some embodiments, polynucleotides encoding proteins with altered protoporphyrinogen oxidase (PPO or PROTOX) activity can be used for herbicide resistance. In some embodiments, plants containing such polynucleotides can exhibit improved tolerance to any of a variety of herbicides which can target, for example, the PPO enzyme (also referred to as “PPO inhibitors” or “PROTOX inhibitors”).

In some embodiments, dicamba monooxygenase can be used for providing dicamba tolerance.

In some embodiments, a polynucleotide molecule encoding AAD12 or encoding AAD1 can be used for providing resistance to, for example, auxin herbicides.

In some embodiments, a P450-encoding polynucleotide can be used for conferring herbicide resistance. In some embodiments, a P450-encoding sequence can provide tolerance to HPPD inhibitors by, for example, metabolism of the herbicide. Such sequences include, but are not limited to, the NSF1 gene.

### Resistance to Plant Pests

In some embodiments, a “plant pest” can mean any living stage of an entity that can directly or indirectly injure, cause damage to, or cause disease in any plant or plant product. In some embodiments, a plant pest can include a protozoan, a nonhuman animal, a parasitic plant, a bacterium, a fungus, a virus, a viroid, an infectious agent, a pathogen, or any article similar to or allied thereof.

In some embodiments, a plant pest invertebrate can comprise a pest nematode, a pest mollusk, a pest insect, or any combination thereof. In some embodiments, a pest mollusk can comprise a slug, a snail, or a combination thereof. In some embodiments, a plant pathogen can comprise a fungi, a nematode, or a combination thereof.

In some embodiments, a plant pathogen can be a eukaryotic plant pathogen. In some embodiments, a plant pathogen can include for example, a fungal pathogen, such as a phytopathogenic fungus.

In some embodiments, a target gene of interest (e.g., for gene silencing) can include any coding or non-coding sequence from any species (including, but not limited to, eukaryotes such as fungi; plants, including monocots and dicots, such as crop plants, ornamental plants, and non-domesticated or wild plants; invertebrates such as arthropods, annelids, nematodes, and mollusks; and vertebrates such as amphibians, fish, birds, and mammals). In some embodiments, non-limiting examples of a non-coding sequence (e.g., that can be expressed by a gene expression element such as a regulatory sequence) can include, 5′ untranslated regions, promoters, enhancers, or other non-coding transcriptional regions, 3′ untranslated regions, terminators, introns, microRNAs, microRNA precursor DNA sequences, small interfering RNAs, RNA components of ribosomes or ribozymes, small nucleolar RNAs, and other non-coding RNAs, or any combination thereof. In some embodiments, a gene of interest can include, translatable (coding) sequence, such as genes encoding transcription factors and genes encoding enzymes involved in a biosynthesis or catabolism of molecules of interest (such as amino acids, fatty acids and other lipids, sugars and other carbohydrates, biological polymers, and secondary metabolites including alkaloids, terpenoids, polyketides, non-ribosomal peptides, and secondary metabolites of mixed biosynthetic origin).

In some embodiments, a target gene (e.g., for gene silencing) can be an essential gene of a plant pest or plant pathogen. In some embodiments, essential genes can include genes that can be required for development of a pest or pathogen to a fertile reproductive adult. In some embodiments, essential genes can include genes that, when silenced or suppressed, can result in a death of an organism (e.g., as an adult or at any developmental stage, including gametes) or in an organism's inability to successfully reproduce (e.g., sterility in a male or female parent or lethality to a zygote, embryo, or larva).

In some embodiments, a plant can be transformed (e.g., in a nucleus, an organelle, or both) with an expression cassette encoding, for example, a dsRNA, a siRNA or a miRNA. The dsRNA, siRNA, or miRNA can suppress (e.g., expression of) at least one (e.g., at least 1, at least 2, at least 3, at least 4, at least 5, at least 6, at least 7, at least 8, at least 9, or at least 10) target genes present in a plant pest. In some embodiments, a dsRNA, siRNA, or miRNA can suppress, for example, 1, 2, 3, 4, 5, 6, 7, 8, 9, 10, 15, 20 or more target genes of a plant pest. In some embodiments, suppression of a target gene present in a plant pest can provide complete or nearly complete protection from a plant pest. In some embodiments, “complete protection” can mean that no (e.g., substantial) damage can be caused to a plant by a plant pest.

In some embodiments, resistance to pests in plants can be achieved by, for example, transgenic control. In some embodiments, in-plant transgenic control of, for example, insect pests, can be achieved through, for example, plant expression of crystal (Cry) delta endotoxin genes and/or Vegetative Insecticidal Proteins (VIP) such as from Bacillus thuringiensis. In some embodiments, non-limiting examples of Cry toxins include, for example, the 60 main groups of “Cry” toxins (e.g., Cry1-Cry59) and VIP toxins. In some embodiments, cry toxins can include subgroups of Cry toxins, for example, Cry1a.

In some embodiments, an expression cassette for use in transformation (e.g., into an organelle) may be constructed using, for example, a Cry sequence. In some embodiments, a Cry sequence can include, for example, a wild-type (e.g., native) nucleic acid sequence encoding at least one protein selected from a group consisting of: Cry1Ac, Cyt1Aa, Cry1Ab, Cry2Aa, Cry1I, Cry1C, Cry1D, Cry1E, Cry1Be, Cry1Fa and Vip3A. In some embodiments, a Cry sequence can include, for example, a modified (e.g., truncated or fusion) nucleic acid sequence encoding at least one protein selected from a group consisting of: Cry1Ac, Cyt1Aa, Cry1Ab, Cry2Aa, Cry1I, Cry1C, Cry1D, Cry1E, Cry1Be, Cry1Fa and Vip3A. In some embodiments, a modified sequence can comprise a truncated nucleic acid sequence. In some embodiments, a modified sequence can encode a modified protein fragment. In some embodiments, a truncated protein fragment can retain insecticidal activity. In some embodiments, a nucleic acid sequence can encode a full-length, or modified (e.g., truncated) protein. In some embodiments, a modified protein can be codon-optimized for an organelle of interest.

### Genome Modification

Disclosed herein in some embodiments, are compositions and methods that can be used, for genome modification of a target sequence in a genome (e.g., a nucleus, a plastid, or a mitochondrial genome) of an organism or cell (e.g., a plant or plant cell), for selecting the modified organism or cell, for gene editing, and for inserting a donor polynucleotide into the genome (e.g., a nucleus, a plastid, or a mitochondrial genome) of an organism or cell. In some embodiments, methods disclosed herein can employ a polynucleotide guided polypeptide system; e.g., a guide polynucleotide/Cas protein system. In some embodiments, a Cas protein can be guided by a guide polynucleotide to recognize a target polynucleic acid. In some embodiments, a Cas protein can introduce a single strand or double strand break at a specific target site into a genome of a cell. In some embodiments, a guide polynucleotide/Cas polypeptide system can provide for an effective system for modifying target sites within a genome of a plant, plant cell or seed.

In some embodiments, a variety of methods can be employed to further modify a target site to introduce a donor polynucleotide of interest. In some embodiments, a nucleotide sequence to be edited (e.g., a nucleotide sequence of interest) can be located within or outside a target site that can be recognized by a polynucleotide guided polypeptide.

Also disclosed herein are methods and compositions employing a polynucleotide guided polypeptide system for modification of multiple target sites within a genome of an organelle. Modification of multiple target sites within a genome of an organelle can facilitate a creation of a homoplasmic transformation event.

### Polynucleotide Guided Polypeptide Systems

In some embodiments, a polynucleotide-guided polypeptide can be a polypeptide that can bind to a target nucleic acid. In some embodiments, a polynucleotide-guided polypeptide can be a nuclease (e.g., a CRISPR nuclease). In some embodiments, a polynucleotide-guided polypeptide can be an endonuclease, a modified version thereof, and a biologically active fragment thereof. In some embodiments, a polynucleotide-guided polypeptide can be a Cas protein, a modified version thereof, and a biologically active fragment thereof. In some embodiments, a polynucleotide-guided polypeptide can be a MAD protein, a modified version thereof, and a biologically active fragment thereof. In some embodiments, a polynucleotide-guided polypeptide can be an Argonaute protein, a modified version thereof, and a biologically active fragment thereof. In some embodiments, a polynucleotide guided polypeptide can form a complex with a guide polynucleotide. In some embodiments, a polynucleotide guided polypeptide can be directed to a target nucleic acid by a guide polynucleotide. In some embodiments, a polynucleotide guided polypeptide can complex with a guide polynucleotide to recognize a target nucleic acid. In some embodiments, a polynucleotide guided polypeptide can introduce a single strand or double strand break at a specific target site (e.g., the genome of a cell).

In some embodiments, a polynucleotide guided polypeptide can be a Cas protein of a CRISPR/Cas system. In some embodiments, a Cas protein can be a Class 1 or a Class 2 Cas protein. In some embodiments, a Cas protein can be a Type I, Type II, Type III, Type IV, Type V, or Type VI Cas protein.

In some embodiments, a non-limiting examples of Cas proteins include c2c1, C2c2, c2c3, Cas1, Cas1B, Cas2, Cas3, Cas4, Cas5, Cas5e (CasD), Cas6, Cas6e, Cas6f, Cas7, Cas8a, Cas8a1, Cas8a2, Cas8b, Cas8c, Cas9 (Csn1 or Csx12), Cas10, Cas10d, Cas10, Cas10d, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmrl, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cul966, and homologs or modified versions thereof.

In some embodiments, a Cas protein may be from any suitable organism. In some embodiments, a suitable organism can comprise Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some embodiments, an organism can comprise Streptococcus pyogenes (S. pyogenes).

In some embodiments, a Cas protein can comprise a Cas9 protein. In some embodiments, a Cas9 protein can comprise a Cas9 sequences listed in SEQ ID NOs: 462, 474, 489, 494, 499, 505, and 518 of WO2007/025097 and incorporated herein by reference. In some embodiments, a Cas9 protein can unwind a DNA duplex in close proximity of a genomic target site. In some embodiments, a Cas9 protein can cleave both DNA strands upon recognition of a target sequence by a guide polynucleic acid. In some embodiments, a Cas9 endonuclease can cleave only if a correct protospacer-adjacent motif (PAM) is approximately oriented at a 3′ end of a target sequence. In some embodiments, a Mutagenesis of Streptococcus pyogenes Cas9 catalytic domains can produce “nicking” enzymes (Cas9n) that can induce single-strand nicks rather than double-strand breaks.

In some embodiments, a polynucleotide guided polypeptide can be a MAD polypeptide, e.g., a MAD2 (SEQ ID NO: 2) or a MAD7 polypeptide (SEQ ID NO: 3), with amino acid sequence corresponding to SEQ ID NO:2 and SEQ ID NO:7 of U.S. Pat. No. 9,982,279, respectively (herein incorporated by reference). In some embodiments, a MAD7 can be a Class 2 Type V-A CRISPR-Cas system isolated from Eubacterium rectale and re-engineered by INSCRIPTA™ (Boulder, CO). In some embodiments, analogous to Cas9, MAD7 can be an RNA-guided nuclease with a diverse protein structure, mechanism of action, and a demonstrated gene editing activity in E. coli and yeast cells. In some embodiments, similar to Acidaminococcus sp. Cas12a, MAD7 does not require a tracrRNA and prefers T-rich PAMs (TTTV and CTTV).

In some embodiments, a polynucleotide guided polypeptide may be an Argonaute protein such as Natronobacterium gregoryi Argonaute (“NgAgo”). In some embodiments, an Argonaute protein can be a DNA-guided endonuclease. In some embodiments, an Argonaute protein can bind a guide DNA such as a 5′-phosphorylated single-stranded guide DNA (gDNA) of for example, 24 nucleotides. In some embodiments, an Argonaute protein can create a site-specific target nucleic acid (e.g., DNA) break (e.g., double-stranded breaks) when loaded with a gDNA. In some embodiments, an Argonaute protein/gDNA system may not require a protospacer-adjacent motif (PAM) for recognition of a target nucleic acid.

In some embodiments, a polynucleotide guided polypeptide as used herein can be a wildtype or a modified form of a polynucleotide guided polypeptide. In some embodiments, a polynucleotide guided polypeptide can be an active variant, an inactive variant, or a fragment of a wild type or modified polynucleotide guided polypeptide. In some embodiments, a polynucleotide guided polypeptide can comprise an amino acid change such as a deletion, replacement, insertion, substitution, variant, mutation, fusion, chimera, or any combination thereof relative to a wild-type version of a polynucleotide guided polypeptide. In some embodiments, a polynucleotide guided polypeptide can be a polypeptide with at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type exemplary polynucleotide guided polypeptide (e.g., Cas9 from S. pyogenes). In some embodiments, a polynucleotide guided polypeptide can be a polypeptide with at most about 5%, 10%, 20%, 30%, 40%, 50%, 60%, 70%, 80%, 90%, 100% sequence identity and/or sequence similarity to a wild type exemplary polynucleotide guided polypeptide. In some embodiments, variants or fragments can comprise at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type or modified polynucleotide guided polypeptide or a portion thereof. In some embodiments, variants or fragments can be targeted to a nucleic acid locus in complex with a guide nucleic acid while lacking nucleic acid cleavage activity.

In some embodiments, a polynucleotide guided endonuclease can be a fusion protein. In some embodiments, a polynucleotide guided endonuclease can be fused to a cleavage domain, an epigenetic modification domain, a transcriptional activation domain, or a transcriptional repressor domain. In some embodiments, a non-limiting example of a suitable fusion partner can include a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity, or any combination thereof.

In some embodiments, a polynucleotide guided endonuclease can also be fused to a heterologous polypeptide providing increased or decreased stability. In some embodiments, a fused domain or heterologous polypeptide can be located at an N-terminus, a C-terminus, or internally within a polynucleotide guided endonuclease.

In some embodiments, a nucleic acid encoding a polynucleotide guided endonuclease (e.g., Cas endonuclease, Cas9 endonuclease, MAD polypeptide, MAD7 polypeptide), can be codon optimized for efficient translation into protein in a particular cell, organelle (e.g., nucleus, plastid or mitochondrion), or organism (e.g., wheat or rice).

In some embodiments, a nucleic acid encoding a polynucleotide guided endonuclease can be stably integrated in a genome (nuclear, mitochondrial, plastid) of a cell. In some embodiments, a nucleic acid encoding a polynucleotide guided polypeptide can be operably linked to a regulatory sequence active in a cell. In some embodiments, a nucleic acid encoding a polynucleotide guided polypeptide can be in an expression construct. In some embodiments, an expression construct can include any regulatory sequence that can direct expression of a nucleic acid sequence of interest (promoter, terminator, RNA-editing site).

In some embodiments, an expression construct can include any nucleic acid sequence that encodes a peptide capable of targeting a protein into an organelle of interest (e.g., into a nucleus, mitochondrion, or plastid).

In some embodiments, a polynucleotide guided polypeptide coding sequence can be modified to use codons preferred by a target organism, e.g., a plant, maize or soybean (nuclear, mitochondrial or plastid) codon-optimized sequence. In some embodiments, a sequence that encodes a polynucleotide guided polypeptide can be operably linked to one or more sequences encoding nuclear localization signals; e.g., to a SV40 nuclear targeting signal upstream of a polynucleotide guided polypeptide coding region and a bipartite VirD2 nuclear localization signal downstream of the polynucleotide guided polypeptide coding region. In some embodiments, a sequence that encodes a polynucleotide guided polypeptide can be operably linked to one or more sequences encoding chloroplast or mitochondrial localization signals, i.e., a chloroplast transit sequence or a mitochondrial targeting sequence.

In some embodiments, a polynucleotide guided polypeptide (e.g., Cas polypeptide, Cas9 polypeptide, MAD polypeptide, MAD7 polypeptide), can be provided in any form. In some embodiments, a polynucleotide guided polypeptide can be provided in a form of a protein, such as a polynucleotide guided polypeptide alone or complexed with a guide nucleic acid. In some embodiments, a polynucleotide guided polypeptide can be provided in a form of a nucleic acid encoding a polynucleotide guided polypeptide, such as an RNA (e.g., messenger RNA (mRNA)) or DNA.

In some embodiments, a polynucleotide guided polypeptide can be a polypeptide moiety (e.g., a chimeric polypeptide) that can form a programmable nucleoprotein molecular complex with a specificity conferring nucleic acid (SCNA). In some embodiments, a programmable nucleoprotein molecular complex can assemble in-vivo, in a target cell, or in an organelle. In some embodiments, a programmable nucleoprotein molecular complex can interact with a predetermined target nucleic acid sequence. In some embodiments, a programmable nucleoprotein molecular complex may comprise a polynucleotide molecule encoding a chimeric polypeptide. In some embodiments, a chimeric polypeptide can comprise a functional domain that can modify a target nucleic acid site. In some embodiments, a functional domain can be devoid of a specific nucleic acid binding site. In some embodiments, a chimeric polypeptide can comprise a linking domain that can interact with a SCNA. In some embodiments, a linking domain can be devoid of a specific target nucleic acid binding site. In some embodiments, a SCNA can comprise a nucleotide sequence complementary to a region of a target nucleic acid flanking a target site. In some embodiments, a SCNA can comprise a recognition region that can specifically attach to a linking domain of a chimeric polypeptide. In some embodiments, assembly of a chimeric polypeptide and an SCNA within a target cell can form a functional nucleoprotein complex. In some embodiments, a nucleoprotein complex can specifically modify a target nucleic acid at a target site.

In some embodiments, a polynucleotide guided endonuclease gene can be a full-length polynucleotide guided endonuclease (e.g., Cas endonuclease, Cas9 endonuclease, MAD polypeptide, MAD7 polypeptide), or any functional fragment or functional variant thereof.

Disclosed herein in some embodiments are compositions and methods comprising use of an endonuclease. In some embodiments, an endonuclease can be an enzyme that cleave a phosphodiester bond within a polynucleotide chain. In some embodiments, an endonuclease can comprise restriction endonucleases that cleave DNA at specific sites without damaging bases. In some embodiments, restriction endonucleases can include Type I, Type II, Type III, and Type IV endonucleases, which can further include subtypes. In some embodiments, Type I and Type III systems, both a methylase and restriction activity can be contained in a single complex. In some embodiments, an endonuclease can also include meganucleases, also known as homing endonucleases (HEases). In some embodiments, a meganuclease can bind and cut at a specific recognition site, which can be about 18 bp or more. In some embodiments, a meganuclease can be classified into four families based on conserved sequence motifs. In some embodiments, a meganuclease family can comprise LAGLIDADG (SEQ ID NO: 4), GIY-YIG, H-N-H, and His-Cys box families. In some embodiments, motifs can participate in a coordination of metal ions and hydrolysis of phosphodiester bonds. In some embodiments, HEases can have long recognition sites and can tolerate sequence polymorphisms in their DNA substrates. In some embodiments, a naming convention for a meganuclease can be similar to a convention for other restriction endonuclease.

In some embodiments, a meganuclease can also be characterized by prefix F-, I-, or PI- for enzymes encoded by free-standing ORFs, introns, and inteins, respectively. In some embodiments, one step in a recombination process can involve polynucleotide cleavage at or near a recognition site. In some embodiments, a cleaving activity can be used to produce a double-strand break. In some embodiments, a recombinase can be from an Integrase or Resolvase family.

In some embodiments, compositions and methods of a disclosure can use Transcription activator-like effector nucleases (TALENs; TAL effector nucleases). In some embodiments, TALENs can be a class of sequence-specific nucleases. In some embodiments, TALENs can be used to cleave (e.g., double-strand breaks) at specific target sequences (e.g., in a genome of a plant or other organism). In some embodiments, TALENs can be created by fusing a native or engineered transcription activator-like (TAL) effector, or functional part thereof, to the catalytic domain of an endonuclease, such as, for example, FokI. In some embodiments, a unique, modular TAL effector DNA binding domain can allow for a design of proteins with potentially any given DNA recognition specificity.

Disclosed herein in some embodiments, are compositions and methods comprising use of zinc finger nucleases (ZFNs). In some embodiments, ZFNs can be engineered cleavage (e.g., double-strand break) inducing agents comprised of a zinc finger DNA binding domain and a double-strand-break-inducing agent domain. In some embodiments, recognition site specificity can be conferred by a zinc finger domain, which can comprise two, three, or four zinc fingers, for example having a C2H2 structure.

In some embodiments, a Zinc finger domain can be amenable for designing polypeptides which specifically bind a selected polynucleotide recognition sequence. In some embodiments, a ZFN can consist of an engineered DNA-binding zinc finger domain linked to a non-specific endonuclease domain, for example, a nuclease domain from a Type IIS endonuclease such as FokI. In some embodiments, additional functionalities can be fused to a zinc-finger binding domain, including transcriptional activator domains, transcription repressor domains, and methylases. In some examples, a dimerization of nuclease domain may be required for cleavage activity. In some embodiments, each zinc finger can recognize, for example, three consecutive base pairs in a target DNA. In some embodiments, a 3-finger domain can recognize a sequence of 9 contiguous nucleotides, with a dimerization requirement of a nuclease, two sets of zinc finger triplets can be used to bind an 18 nucleotide recognition sequence.

### Guide Polynucleic Acid

In some embodiments, bacteria and archaea can have evolved adaptive immune defenses termed clustered regularly interspaced short palindromic repeats (CRISPR)/CRISPR-associated (Cas) systems that can use short RNA to direct degradation of foreign nucleic acids. In some embodiments, a type II CRISPR/Cas system from bacteria can employ a crRNA and tracrRNA to guide a Cas polypeptide to a nucleic acid target. In some embodiments, a crRNA (CRISPR RNA) can contain a region complementary to one strand of a double strand DNA target. In some embodiments, a crRNA can base pair with a tracrRNA (trans-activating CRISPR RNA) to form a RNA duplex that can direct a Cas polypeptide to recognize and optionally cleave a DNA target.

In some embodiments, as used herein, the term “guide polynucleotide”, can refer to a polynucleotide sequence that can form a complex with a polynucleotide guided polypeptide (e.g., a Cas protein, a MAD protein). In some embodiments, a guide polynucleotide can direct a polynucleotide guided polypeptide to recognize and optionally cleave (or nick) a DNA target site. In some embodiments, the terms “guide polynucleotide” and “guide polynucleic acid” can be used interchangeably herein. In some embodiments, a guide polynucleotide can be comprised of a single molecule (unimolecular) or two molecules (bimolecular). In some embodiments, a guide polynucleotide sequence can be an RNA sequence, a DNA sequence, or a combination thereof (an RNA-DNA combination sequence). In some embodiments, a guide polynucleotide that solely can comprise ribonucleic acids can also be referred to as a “guide RNA” (gRNA). In some embodiments, a guide polynucleic acid can be a guide RNA.

In some embodiments, the term “single guide RNA” (sgRNA) can refer to a synthetic fusion of two RNA molecules, for example, a crRNA (CRISPR RNA) comprising a variable targeting domain, and a tracrRNA. In some embodiments, a guide RNA can comprise a variable targeting domain (or VT domain) of 12 to 30 nucleotide sequences and an RNA fragment that can interact with a Cas protein.

In some embodiments, a guide polynucleotide can be bimolecular (i.e., two molecules; also referred to as “double molecule”, “dual” or “duplex” guide polynucleotide) comprising, for example, a first molecule having a nucleotide sequence domain (referred to as Variable Targeting domain or VT domain) that is complementary to a nucleotide sequence in a target polynucleic acid (e.g., target DNA) and a second molecule having a nucleotide sequence domain (referred to as Cas endonuclease recognition domain or CER domain) that interacts with a Cas polypeptide.

In some embodiments, complementarity between a guide polynucleic acid (e.g., the VT domain, spacer region) and a target polynucleic acid (e.g., protospacer) can be perfect, substantial, or sufficient. In some embodiments, perfect complementarity between two nucleic acids can mean that two nucleic acids can form a duplex in which every base in a duplex can be bonded to a complementary base by Watson-Crick pairing. In some embodiments, substantial or sufficient complementarity can mean that a sequence in one strand may not be completely and/or perfectly complementary to a sequence in an opposing strand, but that sufficient bonding occurs between bases on the two strands to form a stable hybrid complex in a set of hybridization conditions (e.g., salt concentration and temperature).

In some embodiments, the term “variable targeting domain” or “VT domain” can be used interchangeably herein and can refer to a nucleotide sequence that can be present in a guide polynucleotide. In some embodiments, a VT domain can be complementary to one strand of a double stranded DNA target site. In some embodiments, a percent complementation between a first nucleotide sequence domain (VT domain) and a target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. In some embodiments, a variable target domain can be 12, 13, 14, 15, 16, 17, 18, 19, 20, 21, 22, 23, 24, 25, 26, 27, 28, 29 or 30 nucleotides in length. In some embodiments, a variable target domain can comprise at least 17 nucleotides that are complementary to at least 17 nucleotides of a target polynucleic acid. In some embodiments, a variable targeting domain can comprise a contiguous stretch of nucleotides that are complementary to a target polynucleic acid. In some embodiments, nucleotides of a guide polynucleic acid that are complementary to a target polynucleic acid can be non-contiguous. In some embodiments, a variable targeting domain can comprise a contiguous stretch of 12 to 30 nucleotides. In some embodiments, a variable targeting domain can be composed of a DNA sequence, an RNA sequence, a modified DNA sequence, a modified RNA sequence, or any combination thereof.

In some embodiments, a nucleotide sequence linking a crNucleotide and the tracrNucleotide of a single guide polynucleotide can comprise an RNA sequence, a DNA sequence, or an RNA-DNA combination sequence. In some embodiments, a nucleotide sequence linking a crNucleotide and a tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In some embodiments, a nucleotide sequence linking a crNucleotide and a tracrNucleotide of a single guide polynucleotide can comprise a tetranucleotide loop sequence, such as, but not limiting to a GAAA tetranucleotide loop sequence.

In some embodiments, a guide polynucleic acid can be introduced into a plant cell via transformation of a recombinant DNA construct comprising a polynucleotide encoding a guide polynucleic acid operably linked to a promoter functional in a plant; e.g., a plant U6 polymerase III promoter, a CaMV 35S polymerase II promoter, a mitochondrial promoter, a plastid promoter.

In some embodiments, a plurality of guide polynucleic acids can be multiplexed to target multiple target nucleic acids. For example, 2, 3, 4, 5, 6, 7, 8, 9, 10, or more than 10 target nucleic acids can be targeted simultaneously or iteratively.

### Target Sites for Genome Modification

In some embodiments, the terms “target site”, “target sequence”, “target polynucleotide”, “target polynucleic acid”, “target locus”, “genomic target site”, “genomic target sequence”, and “genomic target locus” can be used interchangeably herein. In some embodiments, a target polynucleic acid can refer to a polynucleotide sequence in a genome (e.g., a plastid or a mitochondrial genome). In some embodiments, a genome can be part of a plant cell. In some embodiments, a target polynucleic acid can refer to a site (e.g., in a genome) recognized by a guide polynucleic acid. In some embodiments, a target polynucleic acid can refer to a site (e.g., in a genome) at which a single-strand or double-strand break can be induced (e.g., by a Cas polypeptide). In some embodiments, a target site can be an endogenous site in a genome. In some embodiments, a target site can be heterologous to an organism and thereby not be naturally occurring in a genome. In some embodiments, a target site can be found in a heterologous genomic location compared to where it occurs in nature. In some embodiments, as used herein, the terms “endogenous target sequence” and “native target sequence” can be used interchangeably herein and can refer to a target sequence that can be endogenous or native to a genome of an organism. In some embodiments, endogenous target sequence can occur at an endogenous or native position of a target sequence in a genome of an organism.

In some embodiments, a target polynucleic acid can be DNA, RNA, or both. In some embodiments, a target polynucleic acid can be DNA (e.g., target DNA). In some embodiments, a target polynucleic acid can be genomic DNA. In some embodiments, a target polynucleic acid can be nuclear DNA, mitochondrial DNA, plastid DNA, or any combination thereof.

In some embodiments, the terms “artificial target site” and “artificial target sequence” can be used interchangeably herein and can refer to a target sequence that has been introduced into a genome of a plant. In some embodiments, such an artificial target sequence can be identical in sequence to an endogenous or native target sequence in a genome of an organism but may be located in a different position (i.e., a non-endogenous or non-native position) in a genome of an organism.

In some embodiments, an “altered target site”, “altered target sequence”, “modified target site”, “modified target sequence” can be used interchangeably herein and can refer to a target sequence as disclosed herein that can comprise at least one alteration when compared to a non-altered target sequence. In some embodiments, such “alterations” can include, for example: (i) replacement of at least one nucleotide, (ii) a substitution of at least one nucleotide, (iii) a deletion of at least one nucleotide, (iv) an insertion of at least one nucleotide, or (v) any combination of (i)-(iv).

In some embodiments, a length of a target site can vary and can include, for example, target sites that are at least 12, 13, 14, 15, 16, 17, 18, 19, 20, 21, 22, 23, 24, 25, 26, 27, 28, 29, 30 or more nucleotides in length. In some embodiments, a target site can be palindromic. In some embodiments, a palindromic sequence can comprise a sequence that on one strand reads the same in the opposite direction on the complementary strand. In some embodiments, a nick/cleavage site can be within a target sequence. In some embodiments, a nick/cleavage site can be outside of a target sequence. In some embodiments, a cleavage could occur at nucleotide positions immediately opposite each other to produce a blunt end cut or, in other cases, incisions could be staggered to produce single-stranded overhangs, also called “sticky ends”, which can be either 5′ overhangs, or 3′ overhangs.

In some embodiments, a target nucleic acid sequence can be 5′ or 3′ of a PAM. In some embodiments, a target nucleic acid sequence can be, for example, 16, 17, 18, 19, 20, 21, 22, or 23 bases immediately 5′ of the first nucleotide of the PAM. In some embodiments, a target nucleic acid sequence can be, for example, 16, 17, 18, 19, 20, 21, 22, or 23 bases immediately 3′ of a last nucleotide of a PAM. In some embodiments, a target nucleic acid sequence can be 20 bases immediately 5′ of a first nucleotide of a PAM. In some embodiments, a target nucleic acid sequence can be 20 bases immediately 3′ of a last nucleotide of a PAM.

In some embodiments, a site-specific cleavage of a target nucleic acid by a polynucleotide guided polypeptide (e.g., Cas protein) can occur at locations determined by base-pairing complementarity between a guide nucleic acid and a target nucleic acid. In some embodiments, a site-specific cleavage of a target nucleic acid by a polynucleotide guided polypeptide (e.g., Cas protein) can occur at locations determined by a protospacer adjacent motif (PAM). In some embodiments, a cleavage site of Cas (e.g., Cas9) can be about 1 to about 25, or about 2 to about 5, or about 19 to about 23 base pairs (e.g., 3 base pairs) upstream or downstream of a PAM sequence. In some embodiments, a cleavage site of a Cas (e.g., Cas9) can be 3 base pairs upstream of a PAM sequence. In some embodiments, a cleavage site of a Cas (e.g., Cpf1) can be 19 bases on a (+) strand and 23 bases on a (−) strand, producing a 5′ overhang 5 nt in length. In some cases, a cleavage can produce blunt ends. In some cases, a cleavage can produce staggered or sticky ends with 5′ overhangs. In some cases, a cleavage can produce staggered or sticky ends with 3′ overhangs.

In some embodiments, different organisms can comprise different PAM sequences. In some embodiments, different Cas proteins can recognize different PAM sequences. In some embodiments, in S. pyogenes, a PAM can be a sequence in a target nucleic acid that can comprise a sequence 5′-NRR-3′, where R can be either A or G, where N can be any nucleotide and N can be immediately 3′ of a target nucleic acid sequence targeted by a spacer sequence. In some embodiments, a PAM sequence of S. pyogenes Cas9 (SpyCas9) can be 5′-NGG-3′, where N can be any DNA nucleotide and can be immediately 3′ of a CRISPR recognition sequence of a non-complementary strand of a target DNA. In some embodiments, a PAM of Cpf1 can be 5′-TTN-3′, where N can be any DNA nucleotide and can be immediately 5′ of the CRISPR recognition sequence.

In some embodiments, a consensus PAM sequence for various MAD polypeptides has been determined (U.S. Pat. No. 9,982,279). In some embodiments, a consensus PAM for MAD1-MAD8, and MAD10-MAD12 was determined to be TTTN. In some embodiments, a consensus PAM for MAD9 was determined to be NNG. In some embodiments, a consensus PAM for MAD13-MAD15 was determined to be TTN. In some embodiments, a consensus PAM for MAD16-MAD18 was determined to be TA. In some embodiments, a consensus PAM for MAD19-MAD20 was determined to be TTCN.

In some embodiments, active variants of genomic target sites can also be used. In some embodiments, active variants can comprise at least 65%, 70%, 75%, 80%, 85%, 90%, 91%, 92%, 93%, 94%, 95%, 96%, 97%, 98%, 99% or more sequence identity to a given target site. In some embodiments, active variants can retain biological activity. In some embodiments, active variants can be recognized by a polynucleotide guided polypeptide (e.g., Cas protein). In some embodiments, active variants can be cleaved by a polynucleotide guided polypeptide (e.g., Cas protein). In some embodiments, assays can be used to measure a double-strand break of a target site by an endonuclease. In some embodiments, assays can measure an overall activity and/or specificity of an endonuclease on DNA substrates containing recognition sites (e.g., target sites, active variants).

### Methods for Integrating a Donor Polynucleotide

In some embodiments, a disclosure provides methods to obtain an organelle (e.g., mitochondrion or plastid) comprising a donor polynucleotide. In some embodiments, a method can employ homologous recombination to provide integration of a polynucleotide at a target site. In some embodiments, a homologous recombination can be enhanced by introducing a double-strand break (DSBs) at selected endonuclease target sites. In some embodiments, described herein is a use of a polynucleotide guided polypeptide system which can provide flexible genome cleavage specificity and can result in a high frequency of double-strand breaks at an organellar DNA target site. In some embodiments, a specific cleavage can enable efficient gene editing of a nucleotide sequence of interest. In some embodiments, a nucleotide sequence of interest to be edited can be located within or outside a target site recognized and/or cleaved by a polynucleotide guided polypeptide (e.g., a Cas polypeptide, a MAD polypeptide).

In some embodiments, a polynucleotide of interest can be provided to an organelle in a donor polynucleotide. In some embodiments, a donor polynucleotide can be a nucleic acid sequence (e.g., DNA, RNA, or both) that can be integrated into a target nucleic acid, for example, a genome of a mitochondrion or a plastid. In some embodiments, a donor polynucleotide can be inserted into a genome e.g., at a cleavage site of a polynucleotide guided polypeptide. In some embodiments, a donor polynucleotide can be inserted into a genome by homologous recombination. In some embodiments, a donor polynucleotide can comprise DNA and can be referred to as donor DNA.

In some embodiments, a donor polynucleotide of any suitable size can be integrated into a genome. In some embodiments, a donor polynucleotide integrated into a genome can be less than 1 kb, about 1 kb, about 1.5 kb, about 2 kb, about 2.5 kb, about 3 kb, about 3.5 kb, about 4 kb, about 4.5 kb, about 5 kb, about 5.5 kb, about 6 kb, about 6.5 kb, about 7 kb, about 7.5 kb, about 8 kb, about 8.5 kb, about 9 kb, about 9.5 kb, about 10 kb, about 10.5 kb, about 11 kb, about 11.5 kb, about 12 kb, about 12.5 kb, about 13 kb, about 13.5 kb, about 14 kb, about 14.5 kb, about 15 kb, about 16 kb, about 17 kb, about 18 kb, about 19 kb, about 20 kb, about 25 kb, about 30 kb, about 35 kb, about 40 kb, about 45 kb, about 50 kb, about 100 kb, about 150 kb, about 200 kb, about 250 kb, about 300 kb, about 350 kb, about 400 kb, about 450 kb, about 500 kb, or less than about 500 kilobases (kb) in length. In some embodiments, a donor polynucleotide integrated into a genome can be at least about 1 kb, about 1 kb, about 1.5 kb, about 2 kb, about 2.5 kb, about 3 kb, about 3.5 kb, about 4 kb, about 4.5 kb, about 5 kb, about 5.5 kb, about 6 kb, about 6.5 kb, about 7 kb, about 7.5 kb, about 8 kb, about 8.5 kb, about 9 kb, about 9.5 kb, about 10 kb, about 10.5 kb, about 11 kb, about 11.5 kb, about 12 kb, about 12.5 kb, about 13 kb, about 13.5 kb, about 14 kb, about 14.5 kb, about 15 kb, about 16 kb, about 17 kb, about 18 kb, about 19 kb, about 20 kb, about 25 kb, about 30 kb, about 35 kb, about 40 kb, about 45 kb, about 50 kb, about 100 kb, about 150 kb, about 200 kb, about 250 kb, about 300 kb, about 350 kb, about 400 kb, about 450 kb, about 500 kb, or less than about 500 kilobases (kb) in length. In some embodiments, a donor polynucleotide integrated into a genome can be up to about 1 kb, about 1 kb, about 1.5 kb, about 2 kb, about 2.5 kb, about 3 kb, about 3.5 kb, about 4 kb, about 4.5 kb, about 5 kb, about 5.5 kb, about 6 kb, about 6.5 kb, about 7 kb, about 7.5 kb, about 8 kb, about 8.5 kb, about 9 kb, about 9.5 kb, about 10 kb, about 10.5 kb, about 11 kb, about 11.5 kb, about 12 kb, about 12.5 kb, about 13 kb, about 13.5 kb, about 14 kb, about 14.5 kb, about 15 kb, about 16 kb, about 17 kb, about 18 kb, about 19 kb, about 20 kb, about 25 kb, about 30 kb, about 35 kb, about 40 kb, about 45 kb, about 50 kb, about 100 kb, about 150 kb, about 200 kb, about 250 kb, about 300 kb, about 350 kb, about 400 kb, about 450 kb, or up to about 500 kb in length.

In some embodiments, a donor polynucleotide can comprise a polynucleotide of interest, a polynucleotide modification template, a heterologous expression cassette, or any combination thereof. In some embodiments, the term “polynucleotide modification template” can refer to a polynucleotide that can comprise at least one nucleotide modification when compared to a nucleotide sequence to be edited. In some embodiments, a nucleotide modification can be at least one nucleotide substitution, replacement, addition, or deletion. In some embodiments, a minor genome modification created by use of a polynucleotide modification template can include creation of a mutant allele (e.g., antibiotic resistant rRNA gene) and removal of a target site for a polynucleotide guided polypeptide. In some embodiments, a donor polynucleotide (e.g. donor DNA) can be flanked by a first and a second region of homology. In some embodiments, a first and second region of homology of a donor polynucleotide (e.g. donor DNA) can share homology to a first and a second genomic region, respectively, present in or flanking a target site (e.g., of an organellar genome).

In some embodiments, “Homology” can mean DNA sequences that are similar. In some embodiments, Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% homology or identity. In some embodiments, a “region of homology to a genomic region” can be a region of DNA that has a similar sequence to a given “genomic region” in an organellar genome. In some embodiments, a region of homology can be of any length that can be sufficient to promote homologous recombination at a cleaved target site. In some embodiments, a region of homology can comprise at least 5, 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that a region of homology can have sufficient homology to undergo homologous recombination with a corresponding genomic region. In some embodiments, a “Sufficient homology” can indicate that two polynucleotide sequences can have sufficient structural similarity to act as substrates for a homologous recombination reaction.

In some embodiments, a donor polynucleotide (e.g., donor DNA) may comprise an expression cassette (e.g., encoding a heterologous polynucleotide of interest). In some embodiments, a donor polynucleotide may comprise multiple expression cassettes. In some embodiments, an expression cassette may be a polycistronic expression cassette, e.g., where multiple protein-coding regions, functional RNAs, or a combination of both, are expressed under control of a single promoter.

In some embodiments, a “donor RNA” can be a corresponding RNA molecule that can comprise, for example, a same nucleic acid sequence as a donor DNA; i.e., with uridylate (“U”) in place of deoxythymidylate (“T”). In some embodiments, a “donor polynucleotide” may be either a donor DNA or a donor RNA, or a combination of DNA and RNA. In some embodiments, a donor polynucleotide may be either single-stranded or double-stranded.

In some embodiments, an alternative method for modification of an organellar genome can be a replacement of part or all of an organelle DNA with a “replacement DNA”. In some embodiments, an endogenous organellar DNA can be reduced or eliminated by use of site-specific endonucleases such as polynucleotide guided polypeptides (e.g., Cas polypeptide, Cas9 polypeptide, MAD polypeptide, MAD7 polypeptide). In some embodiments, at a same time or subsequently, a replacement DNA can be introduced. In some embodiments, the term “replacement DNA” can refer to fragments of organellar DNA or complete organellar DNA that can convey a new genotype and corresponding trait(s) when transformed into an organelle. In some embodiments, the terms “replacement DNA” and “replacement organellar DNA” can be used interchangeably herein. In some embodiments, in the case of organellar DNA fragments, they can be integrated into a remaining endogenous organellar DNA by homologous recombination. In a case of complete organellar DNA replacement, a replacement DNA can be isolated from cultivars, lines, sub species and other species which possess DNA compositions distinct from an endogenous organellar DNA of recipient cells. In some embodiments, a replacement DNA can also be partially and/or completely synthesized in vitro. In some embodiments, a replacement DNA can comprise both native and non-native sequences. In some embodiments, when replacement DNA is created in vitro, it can be a linear DNA with a repeat sequence at the ends. In some embodiments, a repeat sequence can be direct repeats or inverted repeats. In some embodiments, the ends can facilitate homologous recombination in vitro or in vivo to create circular DNA for replication of organellar DNA in cells. In some embodiments, a DNA created in vitro can also include exogenous DNA elements such as ones to allow selected amplification in bacterial cells. In some embodiments, a replacement DNA can comprise a DNA element functioning as a DNA replication origin in a recipient organelle. In some embodiments, a replacement DNA can comprise multiple DNA fragments that are capable of recombination within an organelle to result in a complete replacement DNA.

In some embodiments, a sequence functional as an origin of replication can be included with compositions (e.g., polynucleotides, constructs, cassettes) of the disclosure. Such sequences can include origin of replication for an organelle. In some embodiments, an origin of replication sequence can be a plastid origin of replication (e.g., plastid rRNA intergenic region) sequence. In some embodiments, an origin of replication sequence can be a mitochondrial origin of replication sequence.

In some embodiments, as used herein, a “genomic region” can refer to a segment of DNA in a genome of, for example, an organelle (e.g., a mitochondrion or a plastid). In some embodiments, a genomic region can be present on either side of a target site. In some embodiments, a genomic region can comprise a portion of a target site. In some embodiments, a genomic region can comprise at least 5, 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases. In some embodiments, a genomic region can comprise sufficient homology to undergo homologous recombination with a corresponding region of homology that is associated with a donor DNA.

In some embodiments, a donor polynucleotide, a polynucleotide of interest and/or trait can be stacked together in a complex trait locus. In some embodiments, a guide polynucleotide/polypeptide system can be used to generate double strand breaks and for stacking traits in a complex trait locus.

In some embodiments, two or more polynucleotides encoding RNA and/or proteins can be included in a cassette as a polycistronic unit. In some embodiments, a polynucleotide encoding an RNA can be expressed from separate cassettes.

In some embodiments, a guide polynucleotide/polypeptide system can be used for introducing one or more donor polynucleotides or one or more traits of interest into one or more target sites by providing one or more guide polynucleotides, one or more polynucleotide guided polypeptides (e.g., Cas polypeptides, MAD polypeptides), and optionally one or more donor polynucleotides (e.g., donor DNA) to a plant cell. In some embodiments, an organism can be produced from a cell that can comprise an alteration at said one or more target sites of an organellar DNA (e.g., mitochondrial DNA or plastid DNA), wherein an alteration can be selected from a group consisting of (i) replacement of at least one nucleotide, (ii) a substitution of at least one nucleotide, (iii) a deletion of at least one nucleotide, (iv) an insertion of at least one nucleotide, and (v) any combination of (i)-(iv).

In some embodiments, a structural similarity between a given genomic region and a corresponding region of homology flanking a donor polynucleotide (e.g. donor DNA) can be any degree of sequence identity that allows for homologous recombination to occur. In some embodiments, an amount of homology or sequence identity shared by a “region of homology” flanking a donor polynucleotide (e.g. donor DNA) and a “genomic region” of a plant genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination.

In some embodiments, a region of homology flanking a donor polynucleotide (e.g. donor DNA) can have homology to any sequence flanking a target site. While in some embodiments, regions of homology can share significant sequence homology to a genomic sequence immediately flanking a target site, the regions of homology can be designed to have sufficient homology to regions that may be further 5′ or 3′ to a target site. In still other embodiments, regions of homology can also have homology with a fragment of a target site along with downstream genomic regions. In one embodiment, a first region of homology further can comprise a first fragment of a target site and a second region of homology can comprise a second fragment of a target site, wherein a first and second fragments are dissimilar.

In some embodiments, as used herein, “homologous recombination” can refer to an exchange of DNA fragments between two DNA molecules at sites of homology. In some embodiments, a frequency of homologous recombination can be influenced by a number of factors. In some embodiments, a length of a region of homology can affect a frequency of homologous recombination events, for example, a longer a region of homology, can have a greater frequency of homologous recombination. In some embodiments, a length of a homology region needed to observe homologous recombination may vary among species.

In some embodiments, an intermolecular recombination can occur in mitochondria and in plastids, for example, plants with transformed mitochondrial DNA or transformed plastid DNA can arise through site-specific integration of foreign sequences by homologous recombination with a flanking sequence on a transformation vector.

In some embodiments, an intramolecular recombination between repeated sequences can generate, for example, inversions when repeats are palindromic or deletions when direct.

In some embodiments, endogenous mitochondrial or plastid sequences can be used to target insertions to achieve efficient foreign sequence integration by homologous recombination. In some embodiments, a positive correlation can be present between a rate of recombination and a length and/or degree of sequence homology.

In some embodiments, a minimum flanking sequence length for homologous recombination with an organellar genome can be influenced by an introduction of single-stranded or double-stranded breaks (or both) in an organellar genome, e.g., by polynucleotide guided polypeptide(s).

In some embodiments, an efficiency of a disclosed methods for genome engineering or modification can be at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100%.

In some embodiments, a method can comprise introducing into an organelle (e.g., a mitochondrion or a plastid) of a cell (e.g., a plant cell) a donor polynucleotide (e.g., a donor DNA), a guide polynucleic acid (or multiple guide polynucleic acids) and a polynucleotide guided polypeptide. In some embodiments, at least one single-strand or double-strand break can be introduced in a target site by a polynucleotide guided polypeptide, a first and second region of homology flanking a donor polynucleotide (e.g. donor DNA) can undergo homologous recombination with their corresponding genomic regions of homology resulting in exchange of DNA between the donor and the genome. In some embodiments, methods disclosed herein can result in an integration of a donor polynucleotide (e.g. donor DNA) into a single-strand or double-strand break(s) in a target site in an organellar genome, thereby altering an original target site and producing an altered genomic target site.

In some embodiments, a cell can be a eukaryotic cell. In some embodiments, a cell can comprise, a human cell, an animal cell, a non-human animal cell, a bacterial cell, a fungal cell, an insect cell, a plant cell, a protist cell, a yeast cell, an algal cell, or any combination thereof. In some embodiments, a cell can be a wheat cell, a maize cell, a rice cell, a barley cell, a sorghum cell, a rye cell, a canola cell, a broccoli cell, a cauliflower cell, and a soybean cell. In some embodiments, a cell can be part of an organism or a tissue. In some embodiments, an organism can comprise a plant, a transgenic plant, or parts thereof comprising a cell, a tissue, a propagation material, a seed, a pollen, a progeny, or any combination thereof produced by the methods described herein. In some embodiments, a cell can be an isolated and purified human cell. In some embodiments, the cell described herein can be an engineered non naturally occurring cell.

In some embodiments, a nucleotide to be edited can be located within or outside a target site recognized and cleaved by a polynucleotide guided polypeptide In some embodiments, at least one nucleotide modification may not be a modification at a target site recognized and cleaved by a polynucleotide guided polypeptide. In some embodiments, there can be at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the organellar DNA target site. In some embodiments, a nucleotide to be edited can be located both within and outside a target site (or multiple target sites) recognized and cleaved by a polynucleotide guided polypeptide.

In some embodiments, a donor polynucleotide can comprise a donor DNA. In some embodiments, a donor polynucleotide can be introduced by any suitable means. In some embodiments, a plant having a target site can be provided. In some embodiments, a donor polynucleotide (e.g. donor DNA) can be provided by any suitable transformation method including, for example, Agrobacterium-mediated transformation or biolistic particle bombardment. In some embodiments, a donor polynucleotide (e.g. donor DNA) may be present transiently in a cell or it can be introduced via a viral replicon. In some embodiments, in a presence of a guide polynucleotide (e.g., guide RNA), a polynucleotide guided polypeptide (e.g., Cas polypeptide, MAD polypeptide) and a target site, a donor polynucleotide (e.g. donor DNA) can be inserted into an organellar genome.

### Polynucleotides of Interest for Integration at a Target Site

In some embodiments, further provided are methods for identifying at least one plant cell comprising an organelle comprising a genome comprising a polynucleotide of interest integrated at a target site. In some embodiments, an organelle can comprise a mitochondrion, a plastid, or a combination thereof. In some embodiments, a donor polynucleotide can comprise a polynucleotide of interest. In some embodiments, a variety of methods can be used for identifying those plant cells with an insertion into a genome at or near to a target site without using a screenable marker phenotype. In some embodiments, a method can be viewed as directly analyzing a target sequence to detect any change in a target sequence, including but not limited to PCR methods, sequencing methods, nuclease digestion, Southern blots, and any combination thereof.

In some embodiments, a method can also comprise recovering a plant from a plant cell comprising a polynucleotide of interest integrated into its organellar genome. In some embodiments, a plant can be sterile or fertile.

In some embodiments, a polynucleotide or polypeptide of interest can comprise a herbicide-tolerance coding sequence, an insecticidal coding sequence, a nematocidal coding sequence, an antimicrobial coding sequence, an antifungal coding sequence, an antiviral coding sequence, an abiotic stress tolerance coding sequence, a biotic stress tolerance coding sequence, a sequence modifying a plant trait, or any combination thereof. In some embodiments, a plant trait can comprise yield, grain quality, nutrient content, starch quality and quantity, nitrogen fixation and/or utilization, and oil content and/or composition, or any combination thereof. In some embodiments, a polynucleotide of interest can include, a gene that improves crop yield, a polypeptide that improves a desirability of a crop, a gene encoding a protein conferring resistance to abiotic stress, such as drought, nitrogen, temperature, salinity, toxic metals or trace elements, or those conferring resistance to toxins such as pesticides and herbicides, or to biotic stress, such as attacks by fungi, viruses, bacteria, insects, and nematodes, and development of diseases associated with these organisms. In some embodiments, genes of interest can include, for example, those genes involved in information, such as zinc fingers, those involved in communication, such as kinases, and those involved in housekeeping, such as heat shock proteins. In some embodiments, a polynucleotide of interest can include a gene encoding an important trait for agronomics, insect resistance, disease resistance, herbicide resistance, fertility or sterility, grain characteristics, commercial products, or any combination thereof. In some embodiments, a gene of interest can include those involved in; oil, starch, carbohydrate, or nutrient metabolism; those affecting photosynthesis, photorespiration and ATP metabolism; or any combination thereof.

In some embodiments, commercial traits can also be obtained by expression of proteins encoded on a polynucleotide. In some embodiments, a commercial use of transformed plants can be a production of polymers and bioplastics. In some embodiments, polynucleotides of interest can include genes encoding proteins such as β-ketothiolase, PHBase (polyhydroxybutyrate synthase), and acetoacetyl-CoA reductase which can facilitate expression of polyhydroxyalkanoates (PHAs). In some embodiments, a commercial use can be expression of a gene or genes that can increase starch for ethanol production.

In some embodiments, a polynucleotide or polypeptide that can influence amino acid biosynthesis can include, for example, anthranilate synthase (AS; EC 4.1.3.27) which can catalyze a first reaction branching from an aromatic amino acid pathway to a biosynthesis of tryptophan in plants, fungi, and bacteria. In some embodiments, in plants, a chemical processes for a biosynthesis of tryptophan can be compartmentalized in a chloroplast. In some embodiments, additional donor sequences of interest can include Chorismate Pyruvate Lyase (CPL) which can refer to a gene encoding an enzyme which can catalyze a conversion of chorismate to pyruvate and pHBA. In some embodiments, a CPL gene can be from E. coli. In some embodiments, a CPL gene can bear GenBank accession number M96268.

In some embodiments, a polynucleotide sequence of interest can encode proteins involved in providing disease or pest resistance. In some embodiments, “disease resistance” or “pest resistance” can cause a plant to at least in part avoid a harmful symptom or outcome from a plant-pathogen interaction. In some embodiments, a pest resistance gene can encode resistance to a pest that has great yield drag. In some embodiments, a pest that has great yield drag can comprise rootworm, cutworm, European Corn Borer, or any combination thereof. In some embodiments, a disease resistance or insect resistance gene can comprise a lysozyme, a cecropin, or a combination thereof. In some embodiments, a disease resistance or insect resistance gene can provide antibacterial protection, antifungal protection, nematode protection, insect protection, or any combination thereof. In some embodiments, an antifungal resistance gene or protein can comprise a defensin, a glucanase, a chitinase or any combination thereof. In some embodiments, a nematode or insect protection gene or protein can comprise a Bacillus thuringiensis endotoxin, a protease inhibitor, a collagenase, a lectin, a glycosidase, or any combination thereof. In some embodiments, a gene encoding a disease resistance trait can include a detoxification gene. In some embodiments, a detoxification gene can comprise a fumonisin gene; an avirulence (avr) gene, a disease resistance (R) gene, or any combination thereof. In some embodiments, an insect resistance gene can encode resistance to pests that have great yield drag such as rootworm, cutworm, European Corn Borer, or any combination thereof. In some embodiments, an insect resistance gene can comprise a Bacillus thuringiensis (Bt) toxic protein gene.

In some embodiments, transgenes, recombinant DNA molecules, DNA sequences of interest, or donor polynucleotides can comprise one or more DNA sequences for gene silencing of a target gene. In some embodiments, a target gene can comprise a plant pest gene or a plant pathogen gene. In some embodiments, a method for gene silencing can comprise expression of a DNA sequence in a plant. In some embodiments, a method for gene silencing can comprise cosuppression, antisense suppression, double-stranded RNA (dsRNA) interference, hairpin RNA (hpRNA) interference, intron-containing hairpin RNA (ihpRNA) interference, transcriptional gene silencing, and microRNA (miRNA) interference.

In some embodiments, a fertile plant can be a plant that can produce viable male and female gametes and can be self-fertile. In some embodiments, a self-fertile plant can produce a progeny plant without a contribution from any other plant of a gamete and a genetic material contained therein. Also disclosed herein in some embodiments, are methods comprising a use of a plant that may not be self-fertile. In some embodiments, a plant may not produce male gametes, or female gametes, or both, that are viable or otherwise capable of fertilization. In some embodiments, as used herein, a “male-sterile plant” can be a plant that does not produce male gametes that are viable or otherwise capable of fertilization. In some embodiments, as used herein, a “female-sterile plant” can be a plant that does not produce female gametes that are viable or otherwise capable of fertilization. In some embodiments, male-sterile and female-sterile plants can be female-fertile and male-fertile, respectively. In some embodiments, a male-fertile (but female-sterile) plant can produce viable progeny when crossed with a female-fertile plant. In some embodiments, a female-fertile (but male-sterile) plant can produce viable progeny when crossed with a male-fertile plant. In some embodiments, in some crop species a use of hybrid plants has been shown to dramatically increase crop yield. In some embodiments, a hybrid crop system can require a male sterile line that can serve as a female parent to produce hybrid seed through fertilization with pollen donor plants. In some embodiments, a method to convey male sterility without manual or mechanical intervention can comprise a use of a cytoplasmic male sterility (CMS) gene. In some embodiments, a CMF gene can comprise a nucleic acid. In some embodiments, a CMF gene can comprise a heterologous nucleic acid. In some embodiments, a nucleic acid can comprise DNA, RNA, or a combination thereof. In some embodiments, a coding region, an open reading frame, or a combination thereof. In some embodiments, a CMS gene can be a maternally inherited trait conferred by a mitochondrial genome that results in a failure to produce functional pollen and/or male reproductive organs except in a presence of restorer-of-fertility (RF) genes. In some embodiments, a chimeric mitochondrial ORF can be found to lead to male sterility, producing unisex-female plants. In some embodiments, a creation of a chimeric CMS gene can be a consequence of the highly recombinogenic, repetitive nature of plant mitochondrial genomes. In some embodiments, methods described herein could be used to introduce custom-designed, CMS ORFs into mitochondria of various monocot species, dicot species, or a combination thereof. In some embodiments, a monocot species can comprise wheat, maize, rice, barley, sorghum, sugarcane, rye, canola, broccoli, cauliflower, or any combination thereof. In some embodiments, a dicot can comprise soybean, potato, tomato or any combination thereof. In some embodiments, a CMS ORF of a CMS gene can be encoded by a CMS coding region. In some embodiments, a CMS gene can comprise an orf79 gene from rice. In some embodiments, a CMS gene can comprise an orf256 gene from wheat. In some embodiments, a CMS gene can comprise T-urf13 from maize.

### Phosphite Selection of Transformed Cells

In some embodiments, an embryogenic callus culture of a plant can be initiated and maintained for 6-8 weeks. In some embodiments, the plant may be selected from the group consisting of: rice, wheat, maize, sorghum, barley, rye, canola, broccoli, cauliflower, and soybean. In some embodiments, the plant is rice. In some embodiments, three to four days prior to transformation, the cultures are transferred to fresh callus maintenance media including a standard medium or a modified medium with phosphorus (P) content from phosphite rather than the standard phosphate. In some embodiments, approximately four hours prior to transformation, calli are prepared for bombardment by plating tissue in a target zone on a same phosphite or phosphate-containing media supplemented with mannitol and sorbitol for osmotic protection.

In some embodiments, a plant callus (e.g., a rice callus) can be transformed with a ptxD expression cassette. In some embodiments, the ptxD expression cassette is a nuclear expression cassette. In some embodiments, the ptxD expression cassette is a mitochondrial expression cassette. In some embodiments, transformation is performed using a technique selected from the group consisting of: microinjection, meristem transformation, electroporation, Agrobacterium-mediated transformation, viral based gene transfer, transfection, vacuum infiltration, biolistic particle bombardment or any combination thereof. In some embodiments, transformation may be performed using biolistic particle bombardment. In some embodiments, a variation of a transformation condition can comprise varying particle size and amount. In some embodiments, a variation of a transformation condition can comprise varying the amount of DNA on the particle. In some embodiments, a variation in transformation condition can be the concentration of selective agent in the first selection after bombardment, or in subsequent selections. In some embodiments, the following steps can be followed for culture, selection, and regeneration:

After bombardment, a callus can be incubated in darkness for 16-20 hours at 26° C., then clumps approximately 1-3 mm in size can be subcultured to selective media supplemented with between 0.1 mM and 50 mM P from phosphite salts in place of phosphate salts, with or without casamino acids. In some embodiments, selective media are supplemented with 5 mM, 50 mM, or 100 mM P from phosphite salts in place of phosphate salts.

In some embodiments, microorganisms that have been transformed to express phosphite dehydrogenase or a biologically active fragment thereof can be cultured on phosphite media, wherein the phosphite media comprises phosphite concentration about 0.1 mM to about 150 mM.

In some embodiments, microorganisms that have been transformed to express phosphite dehydrogenase or a biologically active fragment thereof can be cultured on phosphite media, wherein the phosphite media comprises phosphite concentration about 0.1 mM to about 1 mM, about 0.1 mM to about 25 mM, about 0.1 mM to about 50 mM, about 0.1 mM to about 60 mM, about 0.1 mM to about 70 mM, about 0.1 mM to about 80 mM, about 0.1 mM to about 90 mM, about 0.1 mM to about 100 mM, about 0.1 mM to about 110 mM, about 0.1 mM to about 125 mM, about 0.1 mM to about 150 mM, about 1 mM to about 25 mM, about 1 mM to about 50 mM, about 1 mM to about 60 mM, about 1 mM to about 70 mM, about 1 mM to about 80 mM, about 1 mM to about 90 mM, about 1 mM to about 100 mM, about 1 mM to about 110 mM, about 1 mM to about 125 mM, about 1 mM to about 150 mM, about 25 mM to about 50 mM, about 25 mM to about 60 mM, about 25 mM to about 70 mM, about 25 mM to about 80 mM, about 25 mM to about 90 mM, about 25 mM to about 100 mM, about 25 mM to about 110 mM, about 25 mM to about 125 mM, about 25 mM to about 150 mM, about 50 mM to about 60 mM, about 50 mM to about 70 mM, about 50 mM to about 80 mM, about 50 mM to about 90 mM, about 50 mM to about 100 mM, about 50 mM to about 110 mM, about 50 mM to about 125 mM, about 50 mM to about 150 mM, about 60 mM to about 70 mM, about 60 mM to about 80 mM, about 60 mM to about 90 mM, about 60 mM to about 100 mM, about 60 mM to about 110 mM, about 60 mM to about 125 mM, about 60 mM to about 150 mM, about 70 mM to about 80 mM, about 70 mM to about 90 mM, about 70 mM to about 100 mM, about 70 mM to about 110 mM, about 70 mM to about 125 mM, about 70 mM to about 150 mM, about 80 mM to about 90 mM, about 80 mM to about 100 mM, about 80 mM to about 110 mM, about 80 mM to about 125 mM, about 80 mM to about 150 mM, about 90 mM to about 100 mM, about 90 mM to about 110 mM, about 90 mM to about 125 mM, about 90 mM to about 150 mM, about 100 mM to about 110 mM, about 100 mM to about 125 mM, about 100 mM to about 150 mM, about 110 mM to about 125 mM, about 110 mM to about 150 mM, or about 125 mM to about 150 mM phosphorus from phosphite salts. In some embodiments, microorganisms that have been transformed to express phosphite dehydrogenase or a biologically active fragment thereof can be cultured on phosphite media, wherein the phosphite media comprises phosphite concentration about 0.1 mM, about 1 mM, about 25 mM, about 50 mM, about 60 mM, about 70 mM, about 80 mM, about 90 mM, about 100 mM, about 110 mM, about 125 mM, or about 150 mM phosphorus from phosphite salts. In some embodiments, microorganisms that have been transformed to express phosphite dehydrogenase or a biologically active fragment thereof can be cultured on phosphite media, wherein the phosphite media comprises phosphite concentration at least about 0.1 mM, about 1 mM, about 25 mM, about 50 mM, about 60 mM, about 70 mM, about 80 mM, about 90 mM, about 100 mM, about 110 mM, or about 125 mM phosphorus from phosphite salts. In some embodiments, microorganisms that have been transformed to express phosphite dehydrogenase or a biologically active fragment thereof can be cultured on phosphite media, wherein the phosphite media comprises phosphite concentration at most about 1 mM, about 25 mM, about 50 mM, about 60 mM, about 70 mM, about 80 mM, about 90 mM, about 100 mM, about 110 mM, about 125 mM, or about 150 mM phosphorus from phosphite salts.

In some embodiments, calli on selective media can then be returned to dark incubation for 2-3 weeks. After 2-3 weeks of dark incubation, small white clumps approximately 1-3 mm in size can again be subcultured to fresh selective medium containing phosphite as a P source and incubated for approximately 2-4 weeks in a lighted plant growth chamber at 26-28° C. In some embodiments, one or more additional rounds of subculturing to fresh selection medium with 2-4 weeks of incubation in the light may be performed until the growth differential between callus clumps becomes apparent. In some embodiments, the phosphite level is increased to from 5 to 50 or from 50 to 100 mM P from phosphite at the second or later rounds of selection.

Vigorously growing calli (individual putative events) can then be transferred to individual plates of fresh selective medium containing phosphite at 5 to 100 mM P from phosphite as a P source, maintaining their individual identity.

At the end of this last 2-4-week selection period, calli representing putative ptxD transformation events and maintaining growth can be transferred to a Chu N6-based medium for embryo maturation, still substituting phosphite for phosphate P as a selective agent at concentrations in the range of 5-100 mM P, but removing growth regulator 2,4-D, and supplementing with 2.5 g/L phytagel in addition to 8 g/L agar.

Mature somatic embryos showing signs of normal maturation can be transferred to a germination medium, still substituting phosphite for phosphate P (in the range of 5-100 mM P) as selective agent. In some embodiments, this medium can be supplemented with growth regulators 0.2 mg/L naphthaleneacetic acid and 2 mg/L 6-benzylamino purine and 2.5 g/L Phytagel in addition to 8 g/L agar.

In some embodiments, these events can be grown in a continuous light growth environment at 26-28° C. for root and shoot formation. In some embodiments, these events can be grown in a 16 h/8 h light/dark growth chamber at 26-28° C. for root and shoot formation.

In some embodiments, plants showing both root and shoot development after the previous step may be transferred to pots containing an artificial potting medium and gently acclimatized to greenhouse conditions. The plants may be grown to maturity and seed production in a greenhouse.

### Alternative Dual Selection Process

In some embodiments, a ptxD expression cassette is linked to or co-transformed with a second selectable marker expression cassette. In some embodiments, the second selectable marker expression cassette is a 35S:HPT expression cassette conferring hygromycin B resistance, and a selection of nuclear transformation events can be facilitated with the use of a standard medium supplemented with 25-50 mg/L hygromycin B. In some embodiments, Hygromycin B can be added in place of, or in addition to, phosphite-containing selective medium. In some embodiments, variations in a timing of introduction of a phosphite selection in conjunction with hygromycin selection are tested to optimize recovery of a transformant expressing a ptxD gene.

### Screenable and Selectable Markers

In some embodiments, a donor polynucleotide can also be a phenotypic marker. In some embodiments, a phenotypic marker can be a screenable or a selectable marker that can include a visual screenable marker, a selectable marker, or a combination thereof. In some embodiments, a selectable marker can comprise a positive or negative selectable marker. In some embodiments, any phenotypic marker can be used. In some embodiments, a selectable or screenable marker can comprise a DNA segment that can allow one to identify or select for or against a molecule or a cell that contains it, e.g., under particular conditions. In some embodiments, a marker can encode an activity, such as, but not limited to, production of RNA, peptide, or protein, or can provide a binding site for RNA, peptides, proteins, inorganic and organic compounds or compositions and the like.

In some embodiments, an example of a selectable or screenable marker can include, but are not limited to, DNA segments that comprise restriction enzyme sites; DNA segments that encode products which provide resistance against otherwise toxic compounds including antibiotics, such as, spectinomycin, ampicillin, kanamycin, tetracycline, hygromycin; DNA segments that encode products which are otherwise lacking in a recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan fluorescent protein (CFP), yellow fluorescent protein (YFP), red fluorescent protein (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification, or any combination thereof.

In some embodiments, additional selectable markers can include polynucleotides that encode proteins that can confer resistance/tolerance to herbicidal compounds, such as glyphosate, sulfonylureas, glufosinate ammonium, bromoxynil, imidazolinones, and 2,4-dichlorophenoxyacetate (2,4-D). In some embodiments, a herbicide resistance protein can include a herbicide tolerant version of the following: an acetyl coenzyme A carboxylase (ACCase); a 4-hydroxphenylpyruvate dioxygenase (HPPD); a sulfonylurea-tolerant acetolactate synthase (ALS); an imidazolinone-tolerant acetolactate synthase (ALS); a glyphosate-tolerant 5-enolpyruvylshikimate-3-phosphate synthase (EPSPS); a glyphosate-tolerant glyphosate oxidoreductase (GOX); a glyphosate N-acetyltransferase (GAT); a phosphinothricin acetyl transferase (PAT); a protoporphyrinogen oxidase (PPO or PROTOX); an auxin enzyme or receptor; a P450 polypeptide, or any combination thereof. In some embodiments, non-limiting examples of genes useful for conferring herbicide resistance in plants can include genes that encode the above proteins. In some embodiments, a neomycin phosphotransferase II (nptII) gene can encode a protein to provide resistance to antibiotics kanamycin and geneticin and a hygromycin phosphotransferase (HPT) gene can encode a protein to provide resistance to hygromycin.

In some embodiments, a DNA transformation of organellar genomes can be performed, for example, in plastids and mitochondria. In some embodiments, a selectable marker gene can include, for example, photosynthesis (atpB, tscA, psaA/B, petB, petA, ycf3, rpoA, rbcL), antibiotic resistance (rrnS, rrnL, aadA, nptII, aphA-6), herbicide resistance (psbA, bar, AHAS (ALS), EPSPS, HPPD, sul) and metabolism (BADH, codA, ARG8, ASA2) genes. In some embodiments, a sul gene from bacteria can comprise herbicidal sulfonamide-insensitive dihydropteroate synthase activity and can be used as a selectable marker when a protein product is targeted to a plant mitochondria.

In some embodiments, a sequence encoding a marker can be incorporated into a genome of an organelle. In some embodiments, an incorporated sequence encoding a marker can be subsequently removed from a transformed organellar genome. In some embodiments, a removal of a sequence encoding a marker may be facilitated by a presence of direct repeats before and after a region encoding a marker. In some embodiments, removal of a sequence encoding a marker can occur via an endogenous homologous recombination system of an organelle or by use of a site-specific recombinase system such as cre-lox or a site-directed recombination method. In some embodiments, a site-directed recombination method can comprise FLP-FRT recombination.

In some embodiments, Caspase Activatable-GFP (CA-GFP) is a modified version of GFP in which fluorescence is completely quenched by appendage of a hydrophobic quenching peptide that tetramerizes GFP and prevents maturation of a chromophore. In some embodiments, a sequence of a CA-GFP protein can correspond to a GFP with a fusion of DEVDFQGPCNDSSDPLVVAASIIGILHLILWILDRL (SEQ ID NO: 5) at the carboxy terminus. In some embodiments, a caspase recognition sequence comprising the amino acids DEVD (SEQ ID NO: 6) can be present in CA-GFP between the fluorescence and the quenching domains. In some embodiments, GFP fluorescence can be fully restored in vivo by catalytic removal of a quenching peptide by cleavage with caspase. In some embodiments, a nucleic acid sequence encoding CA-GFP can be modified by replacement of a caspase recognition sequence with a mitochondrial RNA editing sequence. In some embodiments, an RNA editing sequence can be selected such that a C-to-U conversion results in creation of a stop codon in an mRNA. In some embodiments, expression of a nucleic acid sequence encoding a modified CA-GFP would result in quenching in a cytoplasm or in plastids but would produce fluorescence in mitochondria, thus providing a screenable marker. In some embodiments, a candidate RNA editing sequence for this purpose is present in a wheat mitochondrial cox2 gene at positions 449, 587 and 620 of a gene, where an A residue of an initiation codon is the first base. In some embodiments, a candidate RNA editing sequence for this purpose is present in a wheat mitochondrial cox2 gene at positions 449, 587 and 620 of a gene, where an A residue of an initiation codon is the first base can comprise SEQ ID NO: 7, SEQ ID NO: 8 and SEQ ID NO: 9, respectively.

Disclosed herein in some embodiments, are methods that can provide transformation efficiency into an organelle (e.g., mitochondria, plastids) of, for example, at least about: 1%, 2%, 5%, 10%, 20%, 30%, 40%, 50%, 60%, 70%, 80%, 90%, or 100% transformation efficiency.

### Phosphite Dehydrogenase as a Selectable Marker

In some embodiments, a phosphite dehydrogenase enzyme (PtxD; EC:1.20.1.1) or a biologically active fragment thereof can comprise a protein which exists in some bacteria and can comprise an enzyme which oxidizes phosphorous acid in an NAD+-dependent or NADP+-dependent manner to generate phosphate and NADH or NADPH. In some embodiments, the following reaction formula can correspond to a case where phosphorous acid is oxidized in an NAD+-dependent manner: H₂O+NAD⁺+HPO₃⁻²=H⁺+NADH+HPO₄⁻².

In some embodiments, a phosphite dehydrogenase or a biologically active fragment thereof can comprise a phosphonate dehydrogenase, a NAD-dependent phosphite dehydrogenase, a NAD:phosphite oxidoreductase, or any combination thereof. In some embodiments, a phosphite dehydrogenase or a biologically active fragment thereof can be inhibited by NaCl, NADH and sulfite.

In some embodiments, many organisms can typically utilize phosphate as a source of phosphorus to promote growth. In some organisms, phosphite can be detrimental to growth. In some embodiments, phosphite at low concentrations can be used to limit fungal growth in plants.

In some embodiments, a nuclear genome of yeast, algae and plants can be transformed with a PtxD gene and genetically modified organisms have been shown to utilize phosphite as a phosphorus source for growth. In some embodiments, a chloroplast genome of an alga, Chlamydomonas reinhardtii, has also been transformed with a PtxD gene and shown to convey an ability to grow on phosphite to an alga.

In some embodiments, a polynucleotide encoding a modified phosphite dehydrogenase enzyme or a biologically active fragment thereof is introduced into a cell. In some embodiments, a modified phosphite dehydrogenase enzyme or a biologically active fragment thereof can comprise a phosphite dehydrogenase enzyme or a biologically active fragment thereof operably linked to an organelle targeting peptide (e.g., a mitochondrial targeting peptide, or a plastid targeting peptide). In some embodiments, a polynucleotide can be stably integrated into a nuclear genome of a cell. In some embodiments, a polynucleotide can be transiently expressed in a nuclear genome of a cell.

In some embodiments, a polynucleotide encoding a phosphite dehydrogenase enzyme or a biologically active fragment thereof can be introduced into an organelle of a cell. In some embodiments, an organelle can comprise a mitochondrion, a plastid, or any combination thereof. In some embodiments, a polynucleotide can be stably integrated into a mitochondrial DNA or plastid DNA of a cell. In some embodiments, a polynucleotide can be operably linked to at least one regulatory sequence in a mitochondrion or plastid of a cell.

In some embodiments, a phosphite dehydrogenase enzyme or a biologically active fragment thereof can be of bacterial origin. In some embodiments, an enzyme can be a PtxD polypeptide (i.e., PtxD or PtxD-like), which can comprise any polypeptide that is capable of catalyzing oxidation of phosphite to phosphate and that is (a) at least 80%, 85%, 90%, 91%, 92%, 93%, 94%, 95%, 96%, 97%, 98%, 99% or 100% identical to PtxD (SEQ ID NO: 29; GenBank: AAC71709.1) of Pseudomonas stutzeri WM 88, (b) a derivative of PtxD of SEQ ID NO: 29, (c) a homolog (i.e., a paralog or ortholog) of PtxD (SEQ ID NO: 29) from the same or a different bacterial species, or (d) a derivative of (c).

In some embodiments, exemplary homologs of PtxD of Pseudomonas stutzeri may be provided by Herrera-Estrella et al. US Patent Application Publication No. 2013/0067975, herein incorporated by reference. In some embodiments, exemplary homologs of PtxD of Pseudomonas stutzeri may be provided by Acinetobacter radioresistens SK82 (SEQ ID NO: 48; GenBank EET83888.1); Alcaligenes faecalis (SEQ ID NO: 49; GenBank AAT12779.1); Cyanothece sp. CCY0110 (SEQ ID NO: 50; GenBank EAZ89932.1); Gallionella ferruginea (SEQ ID NO: 51; GenBank EES62080.1); Janthinobacterium sp. Marseille (SEQ ID NO: 52; GenBank ABR91484.1); Klebsiella pneumoniae (SEQ ID NO: 53; Genbank ABR80271.1); Marinobacter algicola (SEQ ID NO: 54; GenBank EDM49754.1); Methylobacterium extorquens (SEQ ID NO: 55; NCBI YP_003066079.1); Nostoc sp. PCC 7120 (SEQ ID NO: 56; GenBank BAB77417.1); Oxalobacter formigenes (SEQ ID NO: 57; NCBI ZP_04579760.1); Streptomyces sviceus (SEQ ID NO: 58; GenBank EDY59675.1); Thioalkalivibrio sp. HL-EbGR7 (SEQ ID NO: 59; GenBank ACL72000.1); and Xanthobacter flavus (SEQ ID NO: 60; GenBank ABG73582.1), among others. In some embodiments, a phosphite dehydrogenase or a biologically active fragment thereof can comprise an amino acid sequence with at least 50%, 60%, 70%, 75%, 80%, 85%, 90%, 91%, 92%, 93%, 94%, or 95%, 96%, 97%, 98%, 99% or 100% sequence identity to one or more of SEQ ID NOS: 29 and 48, 49, 50, 51, 52, 53, 54, 55, 56, 57, 58, 59, or 60.

In some embodiments, a derivative of PtxD of Pseudomonas stutzeri may provide, altered cofactor affinity, altered cofactor specificity, altered thermostability, or any combination thereof.

In some embodiments, a phosphite dehydrogenase enzyme or a biologically active fragment thereof can contain a sequence region with sequence similarity or identity to any one or any combination of the following consensus motifs: an NAD-binding motif having a consensus sequence of VGILGMGAIG (SEQ ID NO: 61); a conserved signature sequence for the D-isomer specific 2-hydroxyacid family with a consensus sequence of XPGALLVNPCRGSWD (SEQ ID NO: 62), where X is K or R, or a shorter consensus sequence within SEQ ID NO: 62 of RGSWD (SEQ ID NO: 63); and/or a motif that may enable hydrogenases to use phosphite as a substrate, with a general consensus of GWQPQFYGTGL (SEQ ID NO: 64), but that can be better defined as GWX₁PX₂X₃YX₄X₅GL (SEQ ID NO: 65), where X₁ is R, Q, T, or K, X₂ is A, V, Q, R, K, H, or E, X₃ is L or F, X₄ is G, F, or S, and X₅ is T, R, M, L, A, or S. Further aspects of consensus sequences found by comparison of PtxD and PtxD homologs are described in U.S. Patent Application Publication No. 2004/0091985, which is incorporated herein by reference. In some embodiments, a phosphite dehydrogenase enzyme or a biologically active fragment thereof may (or may not) be a NAD-dependent enzyme with high specificity for phosphite as a substrate (e.g., Km˜50 μM) and/or with a molecular weight of about 36 kilodaltons. In some embodiments, a dehydrogenase enzyme may, but is not required to, act as a homodimer, and/or have an optimum activity at 35° C. and/or a pH of about 7.25-7.75.

### Benefits of Organisms Transformed to Express Phosphite Dehydrogenase

The systems and methods described herein may utilize at least one, at least two, at least three, at least four, or at least five selectable or screenable markers. Commonly used selectable marker genes in plant may include, for example, those that confer resistance or resistance to antibiotics, such as kanamycin and paromomycin (nptII), hygromycin B (aph IV), streptomycin or spectinomycin. (aadA) and gentamicin (aac3 and aacC4), or those that impart resistance or resistance to herbicides such as glufosinate (bar or pat), dicamba (DMO) and glyphosate (aroA or EPSPS). In some cases, a screenable marker may provide an ability to visually screen transformants such as luciferase or green fluorescent protein (GFP), or genes expressing known uidA genes (GUS) or beta glucuronidase of various chromogenic substrates. In some embodiments, one or more selectable or screenable markers may be used at different growth stage of a cell, a tissue, a propagation material, a seed, a pollen, a progeny, or any combination thereof. For example, a cell may be co-transformed with a first selectable marker (e.g., a gene that confers resistance to the antibiotic hygromycin) and a second selectable maker (a phosphite dehydrogenase), and may grow in a presence of a first selective agent (hygromycin) and then subsequently in a presence of a second selective agent (e.g., phosphite) at different growth stage. The transformation may also be performed in the absence of selection during one or more stages or steps of development or regeneration of the transformed cell, tissue, propagation material, seed, pollen, progeny, or any combination thereof. In some embodiments, one or more selectable or screenable markers may be incorporated in different organelles (e.g., nucleus and mitochondrial genomes). In some embodiments, one or more selectable or screenable markers may be removed upon successful transformation.

In some embodiments, phosphorus may be used as a selective agent, since phosphorus, in oxidized form, can be incorporated into many biomolecules in a plant or fungal cell to provide genetic material, membranes, and molecular messengers, among others.

In some cases, inorganic phosphate (Pi) can be a primary source of phosphorus for plants. Although a phosphate-based fertilizer can offer a cheap and widely used approach to enhancing plant growth, a phosphate-based fertilizer can come from a non-renewable resource that has been projected to be depleted in the next seventy to one hundred years, or sooner if the usage rate increases faster than expected.

In some cases, a phosphate-based fertilizer common to modem agriculture generally cannot be used efficiently by cultivated plants, due to several important factors. In some cases, phosphate is highly reactive and can form insoluble complexes with many soil components, which reduces an amount of available phosphorus. In some cases, soil microorganisms can rapidly convert phosphate into organic molecules that generally cannot be metabolized efficiently by plants, which reduces an amount of available phosphorus further. In some embodiments, growth of weeds can be stimulated by phosphate-based fertilizers, which not only reduces an amount of available phosphorus still further but which also can encourage weeds to compete with cultivated plants for space and other nutrients. In some embodiments, losses due to a conversion of phosphate into inorganic and organic forms that are not readily available for plant uptake and utilization, and competition from weeds, implies a use of excessive amounts of phosphate fertilizer, which not only increases production costs but also causes severe ecological problems.

Described herein is utilization of phosphite (Phi), a reduced form of phosphate. Although phosphite can be transported into plants using a same transport system as phosphate and may accumulate in plant tissues for extended periods of time, there apparently are no reports of any enzymes in plants that can metabolize phosphite into phosphate as the primary source of phosphorus in plants. Even during phosphate starvation, phosphite cannot satisfy a phosphorus nutritional requirement of a plant. In spite of similarities to phosphate, phosphite can comprise a form of phosphorus that generally cannot be metabolized directly by plants, and thus is not a plant nutrient. Methods disclosed herein can allow a plant to use phosphite for growth when other sources of phosphorus are not available by introducing a phosphite dehydrogenase gene or a biologically active fragment thereof in transgenic plants or transgenic fungi. By selectively allow expression of the phosphite dehydrogenase gene in a plant of interest, the systems and methods described herein may provide various benefits to crop cultivation by allowing phosphite metabolism as its primary source of phosphorus (e.g., controlling weed).

In some embodiments, phosphite can promote plant growth indirectly. In some embodiments, phosphite can be used as an anti-fungal agent (a fungicide) on cultivated plants. In some embodiments, phosphite can be thought to prevent diseases caused by oomycetes (water molds) on such diverse plants as potato, tobacco, avocado, and papaya, among others. In some embodiments, phosphite can promote plant growth, not directly as a plant nutrient, but by protecting plants from fungal pathogens that would otherwise affect plant growth.

In some embodiments, a concentration of phosphite in contact with a plant can be a critical factor for phosphite effectiveness because too much phosphite can be toxic to plants. In some cases, phosphite can compete with phosphate for entry into plant cells, since phosphite may be transported into plants via a phosphate transport system. In some cases, phosphite toxicity may be due to (1) reduced assimilation of phosphate by plants, in combination with (2) an inability to use phosphite as a source of phosphorus by oxidation to phosphate, which causes phosphite accumulation in a plant. In some embodiments, phosphite may be sensed in plants as phosphate, which can prevent a plant from inducing a phosphorus salvage pathway that promotes plant survival under conditions of low phosphate. In some embodiments, phosphite toxicity can affect such diverse plants as Brassica nigra, Allium cepa (onion), Zea mays L. (corn), Arabidopsis thaliana, or any combination thereof. In some embodiments, an exposure of a plant to phosphite may need to be controlled very carefully. In some cases, a better system may be needed for exploiting the benefits of phosphite to plants while reducing its drawbacks.

Disclosed herein in some embodiments, are systems, including methods and compositions, for making and using transgenic plants and/or transgenic fungi that metabolize phosphite as a source of phosphorus for supporting growth and a selective marker while minimizing the use of antibiotic or herbicide.

In some embodiments, a polynucleotide encoding a phosphite dehydrogenase or a biologically active fragment thereof can be incorporated into a mitochondrial genome of a plant or a fungus.

In some embodiments, the method described herein may promote growth or cultivation of a plants and/or fungi of interest comprising the edited mitochondrial genome, while suppressing the growth of an undesired plant (e.g., weed) that does not comprise the edited mitochondrial genome. For example, a plurality of plants may be grown in a presence of phosphite, wherein at least one desired plant of the plurality of plants comprises a mitochondrion having a heterologous polynucleotide that encodes phosphite dehydrogenase or a biologically active fragment thereof and at least one undesired plant (e.g., weed) of the plurality of plants lacking a mitochondrion having a heterologous polynucleotide that encodes phosphite dehydrogenase or a biologically active fragment thereof. In some embodiments, the presence of phosphite is sufficient to selectively promote growth of the at least one desired plant of the plurality of plants, resulting in an increased growth of the at least one desired plant of the plurality of plants relative to undesired plants (e.g., weed) lacking phosphite dehydrogenase or a biologically active fragment thereof. In some embodiments, phosphite may be applied to the plant, the plurality of plants, soil adjacent to the plants or any combination thereof. In some embodiments, the phosphite is applied as a foliar fertilizer, a soil amendment, or any combination thereof. In some embodiments, the phosphite may be dissolved in water and applied to the plant, the plurality of plants, soil adjacent to the plants or any combination thereof.

In some embodiments, a plant and/or fungi comprising a mitochondrion having a heterologous polynucleotide that encodes phosphite dehydrogenase or a biologically active fragment thereof may have a significant increase in growth, phenotype, and physiology with better phosphorus build-up and lower phosphite accumulation compared to a plant lacking a mitochondrion having a heterologous polynucleotide that encodes phosphite dehydrogenase or a biologically active fragment thereof.

In some embodiments, a fungal cell can be applied to a seed form of plants, the plants themselves, soil in which the plants are or will be disposed, or a combination thereof. In some embodiments, a fungal cell can express a phosphite dehydrogenase enzyme or a biologically active fragment thereof from a chimeric gene and may belong to a species of Trichoderma.

In some embodiments, a plant can be associated with a plurality of fungal cells to form mycorrhizae. In some embodiments, a fungal cell can express a phosphite dehydrogenase enzyme or a biologically active fragment thereof from a chimeric gene. In some embodiments, a fungal cell can render a plant capable of growth on phosphite (and/or hypophosphite) as a phosphorus source by oxidizing phosphite to phosphate.

In some embodiments, microorganisms (e.g., yeast, algae) may be grown on an industrial scale to produce desirable chemicals and/or biomolecules. In some cases, maintaining growth in a sterile environment can be a challenge. In some embodiments, microorganisms that have been transformed to express phosphite dehydrogenase or a biologically active fragment thereof can be cultured on phosphite media, which can inhibit a growth of non-transformed organisms. In some embodiments, a yeast that has undergone nuclear transformation with expression cassettes for phosphite dehydrogenase or a biologically active fragment thereof can grow on phosphite as a phosphorus source. In some embodiments, microorganisms transformed to express phosphite dehydrogenase or a biologically active fragment thereof in a mitochondria may provide an additional avenue for avoiding contamination by undesirable organisms.

### Methods Utilizing a Two Component RNA Guide and Polynucleotide Guided Polypeptide System

In some embodiments, a polynucleotide guided polypeptide system described herein can be especially useful for genome engineering in circumstances where endonuclease off-target cutting can be toxic to a targeted cell. In some embodiments, a polynucleotide guided polypeptide system described herein, a constant component, a polynucleotide encoding an organelle targeted polynucleotide guided polypeptide, can be stably integrated into a nuclear genome of a cell. In some embodiments, a polynucleotide encoding an organelle targeted polynucleotide guided polypeptide can be transiently expressed in a nuclear genome of a cell. In some embodiments, a polynucleotide can encode a modified polynucleotide guided polypeptide comprising an enzymatically active polynucleotide guided polypeptide (e.g., Cas polypeptide, a MAD polypeptide) fused to an organellar transport sequence (e.g., a mitochondrial targeting peptide or a chloroplast targeting peptide). In some embodiments, an expression of a polynucleotide encoding a modified polynucleotide guided polypeptide can be under control of a promoter. In some embodiments, a promoter can be a constitutive promoter, a tissue-specific promoter, or an inducible promoter, e.g., a temperature-inducible, stress-inducible, developmental stage inducible, or chemically inducible promoter. In some cases, in the absence of a variable component (e.g., a guide RNA or crRNA), a polynucleotide guided polypeptide may not cut a target nucleic acid. In an absence of a variable component (e.g., a guide RNA or crRNA) a presence of a polynucleotide guided polypeptide in a cell (e.g., a plant cell) may have little or no consequence. In some embodiments, a polynucleotide guided polypeptide system can be used to create and/or maintain a cell line or transgenic organism capable of efficient expression of a polynucleotide guided polypeptide. Expression of a polynucleotide guided polypeptide in a cell line or transgenic organism may have little or no consequence to cell viability.

In some embodiments, in order to induce cutting at desired genomic sites to achieve targeted genetic modifications, guide polynucleotides (e.g., guide RNAs or crRNAs) can be introduced by a variety of methods into cells containing a stably-integrated and expressed expression cassette for a polynucleotide guided polypeptide. In some embodiments, a guide polynucleotide (e.g., guide RNAs or crRNAs) can be chemically or enzymatically synthesized and introduced into a polynucleotide guided polypeptide expressing cells via direct delivery methods such a particle bombardment or electroporation. In some embodiments, a guide polynucleic acid can be fused to an RNA molecule that allows for transport into an organelle. In some embodiments, a guide polynucleic acid can be fused to an RNA molecule that allows for binding to a protein that facilitates transport into an organelle. In some embodiments, a guide polynucleic acid can be transported into an organelle by association with a modified polynucleotide guided polypeptide comprising an enzymatically active polynucleotide guided polypeptide fused to an organellar transport sequence.

In some embodiments, a gene can efficiently express a guide polynucleotide in a target cell. In some embodiments a guide polynucleotide can comprise a guide RNAs, a crRNAs, or a combination thereof. In some embodiments a gene that can efficiently express a guide polynucleotide in a target cell can be synthesized chemically, enzymatically or in a biological system. In some embodiments, a gene that can efficiently express a guide polynucleotide in a target cell can be introduced into a polynucleotide guided polypeptide expressing cell, via direct delivery methods, biological delivery methods, or a combination thereof. In some embodiments, a direct delivery method can comprise a particle bombardment, an electroporation, a vacuum infiltration, or any combination thereof. In some embodiments, a biological delivery method can comprise an Agrobacterium-mediated DNA delivery method.

In some embodiments, a method for altering a genome of an organelle can comprise: introducing into an organelle a first polynucleotide encoding at least one guide polynucleic acid. In some embodiments, at least one guide polynucleic acid can direct a polynucleotide guided polypeptide to cleave at least one target sequence present in an organelle genome. In some embodiments, a guide polynucleic acid can comprise a guide RNA. In some embodiments, a polynucleotide guided polypeptide can comprise a Cas polypeptide, a Cas9 polypeptide or a combination thereof. In some embodiments, a method can further comprise introducing into an organelle a second polynucleotide. In some embodiments, a second polynucleotide can encode a polynucleotide guided polypeptide. In some embodiments, a polynucleotide guided polypeptide, when associated with a guide polynucleic acid can cleave at least one target sequence. In some embodiments, a method can further comprise introducing into an organelle a third polynucleotide encoding at least one homologous organelle DNA sequence. In some embodiments, at least one homologous organelle DNA can be of sufficient size for homologous recombination. In some embodiments, integration of at least one homologous organelle DNA sequence into an organelle genome can result in removal of at least one target sequence. In some embodiments, an organelle can comprise a mitochondrion, a plastid, or a combination thereof.

Disclosed herein in some embodiments, are methods for selecting a plant comprising an altered organellar genome. In some embodiments, a method can be used to identify those cells having an altered genome at or near a target site without using a screenable or selectable marker phenotype. In some embodiments, a method can comprise directly analyzing a target sequence to detect any change in a target sequence, including but not limited to PCR methods, sequencing methods, nuclease digestion, Southern blots, and any combination thereof.

In some embodiments, sufficient homology or sequence identity can indicate that two polynucleotide sequences can have sufficient structural similarity to act as substrates for a homologous recombination reaction. In some embodiments, a structural similarity can include an overall length of each polynucleotide fragment, a sequence similarity of each polynucleotide, or a combination thereof. In some embodiments, a sequence similarity can be described by a percent sequence identity over a whole length of multiple sequences, by conserved regions comprising localized similarities such as contiguous nucleotides having 100% sequence identity, by percent sequence identity over a portion of a length of multiple sequences, or any combination thereof.

In some embodiments, an amount of homology or sequence identity shared by a target and a donor polynucleotide can vary. For example, a length of sequence homology can be at least about 20 bp, at least about 50 bp, at least about 100 bp, at least about 150 bp, at least about 250 bp, at least about 300 bp, at least about 400 bp, at least about 500 bp, at least about 600 bp, at least about 700 bp, at least about 800 bp, at least about 900 bp, at least about 1000 bp, at least about 1250 bp, at least about 1500 bp, at least about 1750 bp, at least about 2000 bp, at least about 2.5 kb, at least about 3 kb, at least about 4 kb, at least about 5 kb, at least about 6 kb, at least about 7 kb, at least about 8 kb, at least about 9 kb, or at least about 10 kb. In some embodiments, an amount of homology can also be described by a percent sequence identity over a full aligned length of two polynucleotides which can include a percent sequence identity of at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. In some embodiments, sufficient homology can include any combination of polynucleotide length, global percent sequence identity, conserved regions of contiguous nucleotides, local percent sequence identity, or any combination thereof. In some embodiments, a sufficient homology can be described as a region of 75-150 bp having at least 80% sequence identity to a region of a target locus. In some embodiments, a sufficient homology can also be described by a predicted ability of two polynucleotides to specifically hybridize under high stringency conditions.

In some embodiments, a plant cell having an introduced sequence can be grown or regenerated into a plant. In some embodiments, a plant can then be grown, and either pollinated with a same transformed strain or with a different transformed or untransformed strain, and a resulting progeny having a desired characteristic and/or comprising an introduced polynucleotide or polypeptide identified. In some embodiments, two or more generations can be grown to ensure that a polynucleotide can be stably maintained and inherited, and seeds harvested.

In some embodiments, any plant can be used. In some embodiments, a plant can comprise a monocot, or a dicot plant. In some embodiments, a monocot plant can comprise a corn (Zea mays), a rice (Oryza sativa), a rye (Secale cereale), a sorghum (Sorghum bicolor, Sorghum vulgare), a millet (e.g., pearl millet (Pennisetum glaucum), a proso millet (Panicum miliaceum), a foxtail millet (Setaria italica), a finger millet (Eleusine coracana)), a maize, a wheat (Triticum aestivum), a sugarcane (Saccharum spp.), an oat (Avena), a barley (Hordeum), a switchgrass (Panicum virgatum), a pineapple (Ananas comosus), a banana (Musa spp.), a palm, an ornamental, a turfgrass, another grass, or any combination thereof. In some embodiments, a dicot plant can comprise a soybean (Glycine max), a canola (Brassica napus and B. campestris), an alfalfa (Medicago sativa), a tobacco (Nicotiana tabacum), an Arabidopsis (Arabidopsis thaliana), a sunflower (Helianthus annuus), a cotton (Gossypium arboreum), a peanut (Arachis hypogaea), a tomato (Solanum lycopersicum), a potato (Solanum tuberosum), or any combination thereof.

In some embodiments, after creating a designed change in an organellar DNA, a next step can be to maintain an edited organellar DNA in a pool of unmodified organellar DNA and to shift a balance among organellar DNA to favor a maintenance of genome edited organellar DNA. In some embodiments, this can be achieved by reducing an amplification of unmodified organellar DNA. In some embodiments, guide polynucleic acids can be designed for multiple target sites in an unmodified organelle genome. In some embodiments, a donor polynucleotide can comprise a donor DNA. In some embodiments, a donor polynucleotide can be designed such that a target site has been altered to no longer be recognized by a relevant polynucleotide guided polypeptide system. In some embodiments, an expression of a polynucleotide guided polypeptides can result in an introduction of single-strand or double-strand breaks into an unmodified organellar DNA and can thereby increase a proportion of modified genomes. In some embodiments, a cell can be pretreated with relevant polynucleotide guided polypeptide systems to introduce cleavages in organellar DNA. In some embodiments, a pretreatment can reduce a number of organelle DNA molecules available for homologous recombination.

In some embodiments, a cell may be selected that is homoplasmic for an altered genome of an organelle. In some embodiments, a cell may be selected that comprises a plurality of mitochondrial genomes, wherein at least 10%-100% of the plurality of mitochondrial genomes comprise the edited mitochondrial genome. In some embodiments, the selected cell may comprise a plurality of mitochondrial genomes that is about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100% of the plurality of mitochondrial genomes comprise the edited mitochondrial genome. In some embodiments, the selected cell may comprise a plurality of mitochondrial genomes that is about 10%, about 20%, about 30%, about 40%, about 50%, about 60%, about 70%, about 80%, about 90%, or about 100% of the plurality of mitochondrial genomes comprise the edited mitochondrial genome. In some embodiments, the selected cell may comprise a plurality of mitochondrial genomes that is at least about 10%, about 20%, about 30%, about 40%, about 50%, about 60%, about 70%, about 80%, or about 90% of the plurality of mitochondrial genomes comprise the edited mitochondrial genome. In some embodiments, the selected cell may comprise a plurality of mitochondrial genomes that is at most about 20%, about 30%, about 40%, about 50%, about 60%, about 70%, about 80%, about 90%, or about 100% of the plurality of mitochondrial genomes comprise the edited mitochondrial genome. In some embodiments, an organelle can comprise a nucleus, a mitochondrion, a plastid, or a combination thereof.

In some embodiments, a method can comprise use of a single guide RNA (sgRNA). In some embodiments, a variable targeting domain can be fused to a polynucleotide that contains a tracrRNA sequence. In some embodiments, a method can comprise use of a duplex guide RNA. In some embodiments, a variable targeting domain and a tracrRNA sequence can be present on separate RNA molecules. In some embodiments, the terms “duplex guide RNA” and “dual guide RNA” can be used interchangeably.

In some embodiments, an expression level of a protein, an RNA, or a combination thereof can be higher when transformed into a plastid or mitochondrion as compared with that in a nucleus. In some embodiments, a protein and/or an RNA expression level can be at least about: 1%, 5%, 10%, 20%, 30%, 40%, 50%, 60%, 70%, 80%, or 90% higher with transformation of plastid or mitochondrial DNA as compared with a nuclear DNA transformation. In some embodiments, an expression stability of a protein, a transcript, or a combination thereof can be higher with a plastid or a mitochondrial transformation as compared with a nuclear transformation.

### Methods for Delivery

In some embodiments, any suitable delivery method can be used for introducing a composition and molecule disclosure herein into a host cell or organelle. In some embodiments, an organelle can comprise a mitochondrion, a plastid, or a combination thereof. In some embodiments, a host cell can comprise a yeast cell, a plant cell, or a combination thereof. In some embodiments, a composition can comprise a Cas protein, a polynucleotide-guided polypeptide, a guide polynucleic acid, a donor polynucleotide, a nucleic acid encoding a compositions, or any combination thereof. In some embodiments, a composition can be delivered simultaneously or temporally separated. In some embodiments, a choice of method of genetic modification can be dependent on a type of cell being transformed, a circumstance under which a transformation is taking place, or a combination thereof. In some embodiments, a circumstance under which a transformation is taking place can be in vitro, ex vivo, in vivo, in planta, or any combination thereof.

In some embodiments, a delivery method or transformation can include, a viral or bacteriophage infection, a transfection, a conjugation, a protoplast fusion, a lipofection, an electroporation, a calcium phosphate precipitation, a polyethyleneimine (PEI)-mediated transfection, a DEAE-dextran mediated transfection, a liposome-mediated transfection, a particle gun technology, a calcium phosphate precipitation, a direct micro injection, a nanoparticle-mediated nucleic acid delivery, a lipid nanoparticle, lipid-based vectors, polymeric vectors, polyethylenimine, poly(L-lysine), a vacuum infiltration, or any combination thereof.

In some embodiments, a DNA transformation can comprise a yeast nuclear genome transformation. In some embodiments, a DNA transformation can be facilitated by a development of shuttle vectors that can replicate in E. coli and yeast as autonomous plasmids. In some embodiments, a vector system can include low-copy-number plasmids and integrative DNA through homologous recombination.

In some embodiments, disclosed herein are methods comprising delivering a polynucleotide as described herein, a vector as described herein, a transcript thereof, a protein translated therefrom, or any combination thereof to a host cell or organelle. In some embodiments, disclosed herein is a cell produced by a method disclosed herein, an organism produced by a method disclosed herein, an organelles comprising or produced from a cell disclosed herein, or any combination thereof. In some embodiments, an organism can comprise an animal, a plant, a fungi, or a combination thereof. In some embodiments, a polynucleotide guided polypeptide in combination with, and optionally complexed with, a guide sequence can be delivered to a cell or an organelle.

In some embodiments, a method to introduce nucleic acids can comprise viral based gene transfer methods, non-viral based gene transfer methods, or a combination thereof. In some embodiments, a method can be used to administer a nucleic acid encoding a compositions of a disclosure to a cell in culture, or in a host organism. In some embodiments, a non-viral vector delivery system can include a DNA plasmid, an RNA, a naked nucleic acid, a nucleic acid complexed with a delivery vehicle, or any combination thereof. In some embodiments, a delivery vehicle can comprise a liposome. In some embodiments, an RNA can comprise a transcript of a vector described herein. In some embodiments, a viral vector delivery system can include a DNA virus, an RNA virus, or a combination thereof. In some embodiments, a viral vector delivery system can have either episomal or integrated genomes after delivery to a cell. In some embodiments, a viral vector based system for gene transfer can comprise a retrovirus, a lentivirus, an adenovirus, an adeno-associated virus, a herpes simplex virus, or any combination thereof.

In some embodiments, an adenoviral-based system can be used. In some embodiments, an adenoviral-based system can lead to a transient expression of a transgene. In some embodiments, an adenoviral based vector can have a high transduction efficiency in cells and may not require cell division. In some embodiments, a high titer, high levels of expression, or a combination thereof can be obtained with an adenoviral based vector. In some embodiments, an adeno-associated virus (“AAV”) vector can be used to transduce a cell with a target nucleic acid. In some embodiments, a vector can be used transduce a cell with a target nucleic acid for an in vitro production of nucleic acids and peptides, for in vivo and ex vivo gene therapy procedures, or any combination thereof.

In some embodiments, a cell transfected with one or more vectors described herein can be used to establish a new cell line comprising one or more vector-derived sequences. In some embodiments, a cell can be transiently transfected with a composition disclosed herein. In some embodiments, transient transfection can comprise transient transfection of one or more vectors, transfection with RNA, or a combination thereof. In some embodiments, a transiently transfected cell can be modified through an activity of a CRISPR complex. In some embodiments, a cell modified through an activity of a CRISPR complex can be used to establish a new cell line comprising cells containing a modification but lacking any other exogenous sequence.

In some embodiments, a composition disclosed herein can be provided as an RNA. In some embodiments, a composition disclosed herein can be produced by direct chemical synthesis or may be transcribed in vitro from a DNA. In some embodiments, a composition disclosed herein can be synthesized in vitro using an RNA polymerase enzyme. In some embodiments, an RNA polymerase enzyme can comprise a T7 polymerase, a T3 polymerase, an SP6 polymerase, or any combination thereof.

In some embodiments, an RNA can directly contact a target polynucleic acid. In some embodiments, a target polynucleic acid can comprise a target DNA. In some embodiments, a target polynucleic acid can be introduced into a cell using any suitable technique for introducing nucleic acid into a cell. In some embodiments, a suitable technique for introducing a nucleic acid into a cell can comprise a microinjection, an electroporation, a transfection, or any combination thereof.

In some embodiments, a nucleotide encoding a guide nucleic acid can comprise DNA or RNA. In some embodiments, a polynucleotide guided polypeptide can comprise DNA, RNA, or a combination thereof. In some embodiments, a nucleotide encoding a guide nucleic acid and a polynucleotide guided polypeptide can be provided to a cell using a suitable transfection technique. In some embodiments, a nucleic acid encoding a composition of a disclosure can be provided on a vector or a cassette. In some embodiments, a vector or a cassette can comprise a DNA vector. In some embodiments, a vector can comprise a plasmid, a cosmid, a minicircle, a phage, a virus, or any combination thereof. In some embodiments, a vector can transfer a nucleic acid into a target cell. In some embodiments, a vector comprising a nucleic acid can be maintained episomally. In some embodiments, a vector comprising a nucleic acid can comprise a plasmid, a minicircle DNA, a virus, or any combination thereof. In some embodiments, a virus can comprise a cytomegalovirus, an adenovirus, or a combination thereof. In some embodiments, a vector comprising a nucleic acid can be integrated into a target cell genome, through homologous recombination or random integration, e.g. retrovirus-derived vectors such as MMLV, HIV-1, and ALV.

In some embodiments, a polynucleotide guided polypeptide can be provided to cells as a polypeptide. In some embodiments, a protein can be fused to a polypeptide domain that increases solubility of a product. In some embodiments, a domain can be linked to a polypeptide through a defined protease cleavage site, e.g. a TEV sequence, which can be cleaved by a TEV protease. In some embodiments, a linker can comprise a flexible sequence. In some embodiments, a flexible sequence can comprise from 1 to 10 glycine residues.

In some embodiments, a composition as disclosed herein can be operably linked (e.g., covalently or non-covalently) to a polypeptide permeant domain to promote uptake by a cell or an organelle. In some embodiments, a polynucleotide composition can comprise a DNA, an RNA, or a combination thereof. In some embodiments, a disclosure can be associated with a peptide-based polynucleotide carrier that can comprise two functional units: a polynucleotide-binding domain (e.g., a polycationic KH repeat domain) and a polypeptide permeant domain.

In some embodiments, a number of polypeptide permeant domains can be used in a non-integrating polypeptide as disclosed herein, including a peptide, a peptidomimetic, a non-peptide carrier, and any combination thereof. In some embodiments, the terms “permeant peptide”, “cell penetrating peptide”, “CPP”, “protein transduction domain” and “PTD” can be used interchangeably herein. In some embodiments, a permeant peptide can be derived from a third alpha helix of Drosophila melanogaster transcription factor Antennapaedia, referred to as penetratin, which can comprise an amino acid sequence RQIKIWFQNRRMKWKK (SEQ ID NO: 10). In some embodiments, a CPP can comprise an amino acid sequence of any one of SEQ ID NO: 10, 11, 12, 13, 14, 15, 16, 17, 18, 19, 20, 21, 22, 23, 24, 25, 26, 27, or any combination thereof. In some embodiments, a CPP can comprise at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NO: 10, 11, 12, 13, 14, 15, 16, 17, 18, 19, 20, 21, 22, 23, 24, 25, 26, or 27. In some embodiments, a permeant peptide can comprise an HIV-1 tat basic region amino acid sequence, which can include, for example, amino acids 49-57 of a naturally-occurring tat protein. In some embodiments, a permeant domain can include a poly-arginine motif. In some embodiments, a poly-arginine motif can comprise a region of amino acids 34-56 of an HIV-1 rev protein, a nona-arginine, an octa-arginine, or any combination thereof. In some embodiments, a nona-arginine (R9) sequence can be used. In some embodiments, other cell penetrating peptides can include: Pep-1, MPG, gamma-ZEIN, Transportan, MAP, Pept 1, Pept 2, IVV-14, Ig(v), Amphiphilic model peptide, pVEC, HRSV, Bp100 TAT2 or any combination thereof. In some embodiments, a composition as disclosed herein can be fused to a combination of a polypeptide permeant domain. In some embodiments, a site at which a fusion can be made can be selected in order to optimize a biological activity, secretion or binding characteristics of a polypeptide.

In some embodiments, a polynucleotide composition can comprise a DNA, an RNA, or any combination thereof. In some embodiments, a polynucleotide composition disclosed herein can be associated with a peptide-based polynucleotide carrier that can comprise an organellar targeting signal. In some embodiments, for organelle-specific delivery, a peptide-based polynucleotide carrier can comprise two functional units: a polynucleotide-binding domain (e.g., a polycationic KH repeat domain) and an organelle-targeting peptide (e.g., a chloroplast transit peptide, a mitochondrial targeting peptide).

Disclosed herein are compositions that can be prepared by in vitro synthesis. In some embodiments, various commercial synthetic apparatuses can be used. In some embodiments, by using synthesizers, naturally occurring amino acids can be substituted with unnatural amino acids. In some embodiments, a particular sequence and a manner of preparation can be determined by convenience, economics, and purity required.

In some embodiments, where two or more different targeting complexes can be provided to a cell (e.g., two different guide nucleic acids that are complementary to different sequences within a same or different target DNA), a complex can be provided simultaneously (e.g., as two polypeptides and/or nucleic acids). In some embodiments, two or more different targeting complexes can be provided consecutively, e.g. a targeting complex being provided first, followed by a second targeting complex, or vice versa.

In some embodiments, in cases in which a targeting complex and a donor DNA can be provided to a cell, a targeting complex and donor DNA can be provided simultaneously. In some embodiments, a targeting complex and a donor DNA can be provided consecutively, e.g., a targeting complex(es) being provided first, followed by a donor DNA, or vice versa.

### Bioreactor

In some embodiments, a cell, a plant, a transgenic seed, a progeny plant, or a transgenic plant comprising one or more exogeneous polynucleotides in edited mitochondria genome described herein may be grown in a temperature-controlled incubator and/or in a greenhouse. In some cases, the temperature-controlled incubator and/or greenhouse is further configured to control a light-dark cycle. In some embodiment, a cell, a plant, a transgenic seed, a progeny plant, or a transgenic plant can be grown in darkness for predetermined duration in predetermined temperature. In some embodiments, a cell, a plant, a transgenic seed, a progeny plant, or a transgenic plant can be grown in darkness for 16-20 hours at 26° C. In some embodiments, a plant, a transgenic seed, a progeny plant, or a transgenic plant can be grown in a continuous light growth environment at 26-28° C. for root and shoot formation. In some embodiments, a plant, a transgenic seed, a progeny plant, or a transgenic plant can be grown in a 16 h/8 h light/dark growth chamber at 26-28° C. for root and shoot formation. In some embodiments, a progeny plant or a transgenic plant showing both root and shoot development may be transferred to pots containing an artificial potting medium and gently acclimatized to greenhouse conditions. In some embodiments, a plant, a transgenic seed, a progeny plant, or a transgenic plant can be grown in a field. In some embodiments, a field may be treated with phosphite.

### Compositions and Kits

Also provided herein are compositions that include any of the polynucleotides, polypeptides, vectors, or reagents (e.g., phosphite) described herein. Any of the compositions can include any of the polynucleotides, polypeptides, vectors, or reagent described herein and one or more (e.g., 1, 2, 3, 4, or 5) acceptable carriers or diluents. In some embodiments, the kit can include a cell, a tissue, a propagation material, a seed, a pollen, a progeny, or any combination described herein.

In some embodiments, any of the compositions described herein can include one or more buffers (e.g., a neutral-buffered saline, a phosphate-buffered saline (PBS)), one or more growth regulators (e.g., naphthaleneacetic acid, 6-benzylamino purine, phytagel), and one or more medium (e.g., germination medium, growth medium, maturation medium, phosphite medium).

In some embodiments, any of the compositions described herein can further include one or more (e.g., 1, 2, 3, 4, or 5) agents that promote the entry of any of the vectors or nucleic acids described herein into a cell (e.g., a plant cell).

In some embodiments, any of the vectors or nucleic acids described herein can be formulated using natural and/or synthetic polymers. Non-limiting examples of polymers that can be included in any of the pharmaceutical compositions described herein can include, but are not limited to: poloxamer, chitosan, dendrimers and poly(lactic-co-glycolic acid) (PLGA) polymers.

Also provided are kits that include any of the compositions described herein that include any of the polynucleotides, any of the polypeptides, any, or any of the vectors described herein.

In some embodiments, the kit can include instructions for performing any of the methods described herein.

### Specific Embodiments

Embodiment 1. A cell comprising a transformed mitochondrion, wherein the transformed mitochondrion comprises an exogenous polynucleotide encoding a phosphite dehydrogenase enzyme, wherein the cell produces the exogenous phosphite dehydrogenase and wherein the cell can grow in a medium wherein phosphite is present.

Embodiment 2. The cell of embodiment 1, wherein the cell can grow when phosphite is present as a primary phosphorus source and wherein phosphate is present at less than 3 mg/liter in the medium.

Embodiment 3. The cell of embodiment 1 or embodiment 2, wherein the cell is homoplasmic for the transformed mitochondrion.

Embodiment 4. The cell of any one of embodiments 1-3, wherein the phosphite dehydrogenase enzyme comprises an amino acid sequence with at least 95% sequence identity to SEQ ID NO: 29.

Embodiment 5. The cell of any one of embodiments 1-4, wherein the cell is selected from the group consisting of: a yeast cell, an algal cell, a plant cell, an insect cell, a non-human animal cell, an isolated and purified human cell, a mammalian tissue culture cell, and any combination thereof.

Embodiment 6. The cell of embodiment 5, wherein the cell is a plant cell.

Embodiment 7. The plant cell of embodiment 6, wherein the plant cell is selected from the group consisting of: a wheat cell, a maize cell, a rice cell, a barley cell, a sorghum cell, a rye cell, and a soybean cell.

Embodiment 8. A plant comprising the plant cell of embodiment 6 or embodiment 7.

Embodiment 9. A method for transforming a mitochondrion, the method comprising: (a) introducing into a cell a first polynucleotide encoding a phosphite dehydrogenase enzyme; (b) growing the cell under conditions in which the phosphite dehydrogenase enzyme is produced; (c) growing the cell in a medium wherein phosphite is present; and (d) selecting a cell comprising a transformed mitochondrion, wherein the transformed mitochondrion comprises a second polynucleotide.

Embodiment 10. The method of embodiment 9, wherein phosphite is present as a primary phosphorus source, further wherein phosphate is present at less than 3 mg/liter.

Embodiment 11. The method of embodiment 9 or embodiment 10, wherein the medium comprises between 0.1 and 50 mM phosphorus from phosphite salts.

Embodiment 12. The method of embodiment 11, wherein the medium comprises phosphite salts present at a concentration range selected from the group consisting of: 0.1-0.25 mM, 0.25-0.5 mM, 0.5-0.75 mM, 0.75-1.0 mM, 1.0-2.5 mM, 2.5-5.0 mM, 5.0-7.5 mM, 7.5-10 mM, 10-15 mM, 15-20 mM, 20-25 mM, 25-30 mM, 30-35 mM, 35-40 mM, 40-45 mM, and 45-50 mM.

Embodiment 13. The method of any one of embodiments 9-12, wherein step (a) further comprises introducing into the mitochondrion of the cell a Donor DNA, wherein the Donor DNA comprises: (a) a second polynucleotide encoding a polypeptide or a functional RNA, or both, wherein the polypeptide and the functional RNA are heterologous to the mitochondrion; (b) a third polynucleotide at one end; and (c) a fourth polynucleotide at the other end; wherein the third and the fourth polynucleotides each comprise a sequence capable of homologous recombination with an endogenous mitochondrial DNA sequence, wherein homologous recombination of all or part of the third polynucleotide, the fourth polynucleotide, or both the third polynucleotide and the fourth polynucleotide, with the endogenous mitochondrial DNA sequence results in integration of the second polynucleotide into the endogenous mitochondrial DNA sequence; and wherein step (d) further comprised selecting a cell with an altered mitochondrial genome, wherein the altered mitochondrial genome comprises the second polynucleotide.

Embodiment 14. The method of embodiment 13, wherein the Donor DNA further comprises the first polynucleotide, and further wherein the altered mitochondrial genome comprises both the first polynucleotide and the second polynucleotide.

Embodiment 15. The method of embodiment 13 or embodiment 14, wherein the sequence capable of homologous recombination in the third polynucleotide has a size of 25-75 nucleotides, 25-100 nucleotides, 25-150 nucleotides, 25-200 nucleotides, 25-300 nucleotides, 25-400 nucleotides, 25-500 nucleotides, 25-1000 nucleotides, 25-1500 nucleotides, or 25-2000 nucleotides.

Embodiment 16. The method of embodiment 15, wherein the sequence capable of homologous recombination in the fourth polynucleotide has a size of 25-75 nucleotides, 25-100 nucleotides, 25-150 nucleotides, 25-200 nucleotides, 25-300 nucleotides, 25-400 nucleotides, 25-500 nucleotides, 25-1000 nucleotides, 25-1500 nucleotides, or 25-2000 nucleotides.

Embodiment 17. The method of any one of embodiments 13-16, wherein the method further comprises: (f) selecting a cell that is homoplasmic for the altered mitochondrial genome.

Embodiment 18. The method of any one of embodiments 13-17, wherein the first polynucleotide, the second polynucleotide, the third polynucleotide and the fourth polynucleotide are all introduced into the mitochondrion as components of a single recombinant DNA construct.

Embodiment 19. The method of any one of embodiments 9-18, wherein the cell is selected from the group consisting of: a yeast cell, an algal cell, a plant cell, an insect cell, a non-human animal cell, an isolated and purified human cell, and a mammalian tissue culture cell.

Embodiment 20. The method of embodiment 19, wherein the cell is a plant cell.

Embodiment 21. The method of embodiment 20, wherein the plant cell is selected from the group consisting of: a wheat cell, a maize cell, a rice cell, a barley cell, a sorghum cell, a rye cell, and a soybean cell.

Embodiment 22. The method of embodiment 20, wherein the second polynucleotide comprises a cytoplasmic male sterility (CMS) coding region.

Embodiment 23. The method of embodiment 22, wherein plant cell is a rice cell, and further wherein the CMS coding region is orf79.

Embodiment 24. The method of embodiment 22, wherein plant cell is a wheat cell, and further wherein the CMS coding region is orf256.

Embodiment 25. The method of any one of embodiments 13-24, wherein at least one selected from the group consisting of: the first polynucleotide, the second polynucleotide, the third polynucleotide, the fourth polynucleotide, and any combination thereof, is introduced into the cell via microinjection, meristem transformation, electroporation, Agrobacterium-mediated transformation, viral based gene transfer, transfection, vacuum infiltration, biolistic particle bombardment or any combination thereof.

Embodiment 26. The method of any one of embodiments 13-25, wherein at least one selected from the group consisting of: the first polynucleotide, the second polynucleotide, the third polynucleotide, the fourth polynucleotide, and any combination thereof, is introduced into the cell as a peptide-polynucleotide complex, wherein the peptide-polynucleotide complex comprises at least one peptide.

Embodiment 27. The method of embodiment 26, wherein the at least one peptide of the peptide-polynucleotide complex comprises at least one selected from the group consisting of: a cell penetrating peptide (CPP), an organellar targeting peptide, a mitochondrial targeting peptide, a histidine-rich peptide, a lysine-rich peptide, and any combination thereof.

Embodiment 28. The method of any one of embodiments 13-27, wherein the method further comprises: (a) introducing into the mitochondrion of the cell a recombinant DNA construct comprising the following: (i) a first additional polynucleotide encoding at least one guide RNA, wherein the at least one guide RNA directs a polynucleotide guided polypeptide to cleave at least one target sequence present in an organelle genome; and (ii) a second additional polynucleotide encoding a polynucleotide guided polypeptide, wherein the polynucleotide guided polypeptide, when associated with the guide RNA, cleaves the at least one target sequence.

Embodiment 29. The method of any one of embodiments 13-27, wherein the method further comprises: (a) introducing into a nucleus of the cell: (i) a first additional polynucleotide encoding a modified polynucleotide guided polypeptide, wherein the modified polynucleotide guided polypeptide comprises a polynucleotide guided polypeptide operably linked to a mitochondrial targeting peptide, wherein the polynucleotide guided polypeptide when associated with a guide RNA, cleaves at least one target sequence present in the mitochondrial genome; and (ii) a second additional polynucleotide encoding at least one guide RNA, wherein the at least one guide RNA directs the polynucleotide guided polypeptide to cleave the at least one target sequence present in the mitochondrial genome.

Embodiment 30. The method of any one of embodiments 13-27, wherein the method further comprises: (a) introducing into a nucleus of the cell: (i) a first additional polynucleotide encoding a modified polynucleotide guided polypeptide, wherein the modified polynucleotide guided polypeptide comprises a polynucleotide guided polypeptide operably linked to a mitochondrial targeting peptide, wherein the polynucleotide guided polypeptide when associated with a guide RNA, cleaves at least one target sequence present in the mitochondrial genome; and (b) introducing into the mitochondrion of the cell: (i) a second additional polynucleotide encoding at least one guide RNA, wherein the at least one guide RNA directs the polynucleotide guided polypeptide to cleave the at least one target sequence present in the mitochondrial genome.

Embodiment 31. The method of any one of embodiments 28-30, wherein the polynucleotide guided polypeptide is at least one selected from the group consisting of: a Cas9 protein, a MAD2 protein, a MAD7 protein, a CRISPR nuclease, a nuclease domain of a Cas protein, a Cpf1 protein, an Argonaute, modified versions thereof, and any combination thereof.

Embodiment 32. The method of any one of embodiments 28-31, wherein homologous recombination of all or part of the third polynucleotide, or all or part of the fourth polynucleotide, or both, with the endogenous mitochondrial DNA sequence results in an altered mitochondrial genome lacking the at least one target sequence.

Embodiment 33. The method of any one of embodiments 13-32, wherein the method further comprises: (a) introducing into a nucleus of the cell: (i) a first additional polynucleotide encoding a modified site-directed nuclease, wherein the modified site-directed nuclease comprises a site-directed nuclease operably linked to a mitochondrial targeting peptide, wherein the site-directed nuclease cleaves at least one target sequence present in the mitochondrial genome.

Embodiment 34. The method of embodiment 33, wherein the site-directed nuclease is at least one selected from the group consisting of: a TALENS, a Zinc-Finger Nuclease, a Meganuclease, a restriction enzyme, and any combination thereof.

Embodiment 35. The method of any one of embodiments 9-34, wherein the method further comprises: (a) introducing into a nucleus of the cell: (i) a first additional polynucleotide encoding a selectable marker polypeptide that provides tolerance to a selective agent; and (b) selecting a cell that grows in the presence of the selective agent.

Embodiment 36. The method of embodiment 35, wherein the cell is grown simultaneously in the presence of the selective agent and in the presence of phosphite as the primary phosphorus source, wherein phosphate is present at less than 3 mg/liter.

Embodiment 37. The method of embodiment 35, wherein the cell is grown sequentially first in the presence of the selective agent and subsequently in the presence of phosphite as the primary phosphorus source, wherein phosphate is present at less than 3 mg/liter.

Embodiment 38. The method of any one of embodiments 35-37, wherein the selectable marker polypeptide is hygromycin phosphotransferase (HPT) and the selective agent is hygromycin.

Embodiment 39. The method of any one of embodiments 9-38, wherein the first polynucleotide encoding phosphite dehydrogenase enzyme further comprises a T7 RNA polymerase promoter, wherein expression of the phosphite dehydrogenase enzyme is under control of the T7 RNA polymerase promoter, and further wherein the method further comprises: (a) introducing into a nucleus of the cell: (i) a first additional polynucleotide encoding a modified T7 RNA polymerase, wherein the modified T7 RNA polymerase comprises a T7 RNA polymerase operably linked to a mitochondrial targeting peptide.

Embodiment 40. The method of embodiment 39, wherein the mitochondrial targeting peptide is encoded by SEQ ID NO: 38.

Embodiment 41. The method of any one of embodiments 39-40, wherein the first polynucleotide encoding a phosphite dehydrogenase enzyme further comprises SEQ ID NO: 44 or SEQ ID NO: 45, wherein expression of the phosphite dehydrogenase enzyme is under control of SEQ ID NO: 44 or SEQ ID NO: 45.

Embodiment 42. The method of any one of embodiments 9-41, wherein the first polynucleotide encoding a phosphite dehydrogenase enzyme further comprises a sequence encoding a mitochondrial RNA editing site, wherein the mitochondrial RNA editing site provides an AUG start codon in vivo.

Embodiment 43. The method of embodiment 42, wherein the sequence encoding the mitochondrial RNA editing site is SEQ ID NO: 46.

Embodiment 44. The method of embodiment 42, wherein the first polynucleotide encoding the phosphite dehydrogenase enzyme and the sequence encoding the mitochondrial RNA editing site comprises SEQ ID NO: 47.

Embodiment 45. A cell produced by the method of any one of embodiments 9-44, wherein the cell comprises a yeast cell, an algal cell, a plant cell, an insect cell, a non-human animal cell, an isolated and purified human cell, or a mammalian tissue culture cell.

Embodiment 46. The cell of embodiment 45, wherein the cell is a plant cell.

Embodiment 47. A plant, seed, root, stem, leaf, flower, or fruit produced from the plant cell of embodiment 46, wherein the plant, seed, root, stem, leaf, flower, or fruit comprises the altered mitochondrial genome.

Embodiment 48. A method of controlling weeds, the method comprising: growing a plurality of plants in the presence of phosphite, wherein at least one plant expresses in its mitochondria a heterologous polynucleotide that encodes a phosphite dehydrogenase enzyme and at least one plant does not express said enzyme, further wherein the plurality of plants are grown in the presence of sufficient phosphite to selectively promote the growth of the at least one plant expressing in its mitochondria the heterologous polynucleotide that encodes the phosphite dehydrogenase enzyme resulting in its increased growth relative to the at least one plant lacking said enzyme.

Embodiment 49. The method of embodiment 48, further comprising a step of applying phosphite to the plant, to soil adjacent to the plant, or to both.

Embodiment 50. The method of embodiment 49, wherein the phosphite is applied as a foliar fertilizer.

Embodiment 51. The method of embodiment 49, wherein the phosphite is applied as a soil amendment.

Embodiment 52. The method of any one of embodiments 48-51, wherein the at least one plant expressing in its mitochondria the heterologous polynucleotide that encodes the phosphite dehydrogenase enzyme is selected from the group consisting of: wheat, maize, rice, barley, sorghum, rye, sugarcane, potato, tomato, and soybean.

Embodiment 53. The method of any one of embodiments 48-52, wherein the at least one plant lacking said enzyme is a weed.

Embodiment 54. The method of any one of embodiments 48-53, wherein the phosphite dehydrogenase enzyme comprises an amino acid sequence with at least 95% sequence identity to SEQ ID NO: 29.

Embodiment 55. The method of embodiment 54, wherein the phosphite dehydrogenase enzyme comprises an amino acid sequence selected from the group consisting of: SEQ ID NO: 29, SEQ ID NO: 53, and SEQ ID NO: 59.

Embodiment 56. A method for transforming a cell, the method comprising: (a) introducing into the cell a first polynucleotide encoding a modified phosphite dehydrogenase enzyme, wherein the modified phosphite dehydrogenase enzyme comprises a phosphite dehydrogenase enzyme operably linked to a mitochondrial targeting peptide; (b) growing the cell under conditions in which the modified phosphite dehydrogenase enzyme is produced; (c) growing the cell in a medium wherein phosphite is present; and (d) selecting a cell comprising an altered nuclear genome, wherein the altered nuclear genome comprises a second polynucleotide.

Embodiment 57. The method of embodiment 56, wherein phosphite is present as a primary phosphorus source and further wherein phosphate is present at less than 3 mg/liter.

Embodiment 58. The method of embodiment 57, wherein the medium comprises between 0.1 and 20 mM phosphorus from phosphite salts.

Embodiment 59. The method of embodiment 58, wherein the medium comprises phosphite salts present at a concentration range selected from the group consisting of: 0.1-0.25 mM, 0.25-0.5 mM, 0.5-0.75 mM, 0.75-1.0 mM, 1.0-2.5 mM, 2.5-5.0 mM, 5.0-7.5 mM, 7.5-10 mM, 10-15 mM, 15-20 mM, 20-25 mM, 25-30 mM, 30-35 mM, 35-40 mM, 40-45 mM, and 45-50 mM.

Embodiment 60. The method of any one of embodiments 56-59, wherein the cell is selected from the group consisting of: a yeast cell, an algal cell, a plant cell, an insect cell, a non-human animal cell, an isolated and purified human cell, and a mammalian tissue culture cell.

Embodiment 61. The method of embodiment 60, wherein the cell is a plant cell.

Embodiment 62. The method of embodiment 61, wherein the plant cell is selected from the group consisting of: a wheat cell, a maize cell, a rice cell, a barley cell, a sorghum cell, a rye cell, and a soybean cell.

Embodiment 63. The method of embodiment 57, wherein the second polynucleotide is exogenous to the cell.

Embodiment 64. The method of embodiment 57, wherein the second polynucleotide comprises a cytoplasmic male sterility (CMS) coding region.

Embodiment 65. The method of embodiment 64, wherein the cell is a plant cell.

Embodiment 66. The method of embodiment 65, wherein the plant cell is a rice cell, and wherein the CMS coding region is orf79.

Embodiment 67. The method of embodiment 65, wherein the plant cell is a wheat cell, and wherein the CMS coding region is orf256.

### EXAMPLES

The present disclosure is further defined in the following Examples, in which parts and percentages are by weight and degrees are Celsius, unless otherwise stated. It should be understood that these Examples, while indicating embodiments, are given by way of illustration only. From the above discussion and these Examples, the essential characteristics of this disclosure can be ascertained, and without departing from the spirit and scope thereof, various changes and modifications of the disclosure can be envisioned to adapt it to various usages and conditions. Such modifications are also intended to fall within the scope of the appended claims.

### Example 1

### Vectors for Phosphite Selection of Mitochondrial Transformants in Plants

Plants are not known to use phosphite as a source of phosphorus for growth. Based on that fact, a bacterial PtxD gene, or a biologically active fragment thereof, can be used to confer an ability to metabolize phosphite in plants by expressing a gene in a nucleus or in chloroplasts. In this example a gene is used as a marker to select mitochondrial transformants. In one example, a selectable marker is used in a major crop plant, rice.

A PtxD, or a biologically active fragment thereof, coding region from Pseudomonas stutzeri, encoded in a PTX operon (accession number AF061070), can be optimized for codons to have good expression in rice mitochondria. Based on a codon usage of rice mitochondrial genes, a following codon that can be used less frequently can be changed to other synonymous codons that can be used more frequently: CCG, ACG, UAC, CAC, CAG, CGC and CGG.

In some embodiments, a PtxD, or a biologically active fragment thereof, CDS optimized for rice mitochondria can be at least 80%, 85%, 90%, 91%, 92%, 93%, 94%, 95%, 96%, 97%, 98%, 99% or 100% identical to SEQ ID NO: 28. In some embodiments, a PtxD CDS optimized for rice mitochondria (mOsPtxD) can consist of SEQ ID NO: 28.

In some embodiments, a nucleotide changed for codon optimization are shown in lower case (TABLE 1).

In some embodiments, a corresponding amino acid sequence of mOsPtxD can comprise SEQ ID NO: 29. In some embodiments, a mitochondrial-specific expression of mOsPtxD, can use a putative promoter sequence of an ATP1 gene that can be encoded in a rice mitochondrial DNA (accession number NC_011033). In some embodiments, an ATP1 promoter sequence can be presented in SEQ ID NO: 30.

In some embodiments, a designed expression cassette for mOsPtxD also contains a terminator region of an ATP1 gene. In some embodiments, a sequence of an ATP1 terminator can be presented in SEQ ID NO: 31.

In some embodiments, a DNA of an expression cassette can be synthesized with an addition of multiple cloning sites at each end. In some embodiments, a 5′ end can comprise SEQ ID NO: 32. In some embodiments, a 3′ end can comprise SEQ ID NO: 33.

In some embodiments, a synthesized DNA can be digested with a PspOMI and a MfeI restriction digest enzymes and cloned into a PspOMI/EcoRI cloning site of a pNAP76 vector.

In some embodiments, a construct pNAP76 (SEQ ID NO: 34) can consist of the following elements in a pBR322 vector: a pCOB1::eGFP::COB1 Ter (eGFP expression cassette under a control of a COB1 promoter and a terminator of rice mitochondria), a B4 autonomous sequence of a rice mitochondria, or any combination thereof. In some embodiments, a resulting construct with an mOsPtxD expression cassette can be transformed into rice calli using a biolistic transformation method as described in Example 5.

### Example 2

Co-Transformation of the mPtxD Mitochondrial Construct with a Nuclear Construct Encoding an Additional Selectable Marker

In some embodiments, mitochondrial transformants are known to occur less frequently than nuclear transformants using biolistic methods as shown in yeast. In some embodiments, to obtain mitochondrial transformants in plants efficiently, we perform a pre-selection and/or a simultaneous selection of nuclear transformants of DNA that is co-transformed with a mitochondrial construct and allow nuclear expression of a selectable marker gene. In this example, a gene that confers resistance to the antibiotic hygromycin (HPT, hygromycin phosphotransferase gene) can be used. In some embodiments, an HPT protein-coding sequence is presented in SEQ ID NO: 35.

In some embodiments, to express HPT in a nucleus, a CaMV 35S promoter can be used for strong constitutive expression. In some embodiments, a CaMV 35S promoter sequence can be presented in SEQ ID NO: 36.

In some embodiments, to terminate transcription of a transgene, a CaMV 3′ UTR can be used that can carry a poly(A) signal (SEQ ID NO: 37).

In some embodiments, a unique restriction site can be added to both ends of an HTP expression cassette and it can be synthesized in a cloning vector. In some embodiments, after amplifying a synthesized clone, DNA carrying an expression cassette can be released from a cloning vector. In some embodiments, a linearized DNA can be mixed with a DNA containing a mitochondrial mPtxD construct, which can be produced as described in Example 1, and can be transformed using a biolistic method as described in Example 5.

### Example 3

Co-Transformation of the mPtxD Mitochondrial Construct with a Nuclear Construct Encoding an Additional Selectable Marker and T7 RNA Polymerase to Enhance mPtxD Expression in Mitochondria

In some embodiments, a bacterial RNA polymerase and corresponding promoter can be used to enable high-level expression of a mitochondrial selectable marker gene for fast and efficient phosphite selection of cells transformed with a mitochondrial construct carrying an mPtxD gene. In some embodiments, high-level gene expression can be achieved in yeast. In some embodiments, a bacteriophage T7 RNA polymerase gene (accession #: M38308) can be used to achieve high-level gene expression. In some embodiments, a coding region for an amino terminal end of a polymerase can be fused with a coding region for a mitochondrial targeting sequence of an Arabidopsis gene, At5g47030, which can function in rice. In some embodiments, an MTS coding region of At5g47030 can comprise SEQ ID NO: 38.

In some embodiments, a maize ubiquitin 1 promoter can be used with a first intron (SEQ ID NO: 39) and a nos terminator (SEQ ID NO: 40) to confer high-level expression.

In some embodiments, an entire expression cassette for an MTS-T7 RNA polymerase gene comprise SEQ ID NO: 41, where a T7 RNA Polymerase CDS is immediately 3′ to an MTS coding region.

In some embodiments, an expression cassette can be synthesized and cloned into a construct that carries an HTP expression cassette as described above. In some embodiments, a DNA fragment containing both expression cassettes is used for co-transformation into rice cells together with a mitochondrial construct in which expression of mPtxD is under a control of a T7 RNA polymerase.

In some embodiments, a mitochondrial expression cassette can be created by inserting a promoter sequence (TAATACGACTCACTATAG; SEQ ID NO: 42) of a T7 RNA polymerase at a 5′ end of a known transcription start site of a ATP1 promoter, which is described in Example 1. There are three transcription start sites listed in a genome sequence at a GenBank (accession number NC_011033).

In some embodiments, a construct can comprise a T7 promoter inserted upstream of a first transcription start site, and a T7 terminator (SEQ ID NO: 43) can be inserted directly downstream of a stop codon. In some embodiments, an entire promoter sequence with a T7 promoter can comprise SEQ ID NO: 44.

In some embodiments, a construct can comprise a T7 promoter inserted upstream of a third transcription start site. In some embodiments, an entire promoter sequence with a T7 promoter can consist of SEQ ID NO: 45.

In some embodiments, an entire mitochondrial expression cassette for mPtxD can be synthesized as described in Example 1 and transformed into a rice cell using a biolistic method as described in Example 5.

### Example 4

### An Additional Method of Enabling Gene Expression Specific to Mitochondria in Plants

In some embodiments, a method to ensure mitochondrial-specific gene expression can comprise use of a regulatory element of gene expression endogenous to plant mitochondria. In some embodiments a regulatory element can comprise a promoter, a terminator. In some embodiments, a method to ensure mitochondrial-specific gene expression can comprise use of a natural RNA editing site present in a mitochondrion but not in other parts of a plant cell. In some embodiments, an RNA editing site can convert a defined C residue to a U residue of an RNA transcript. In some embodiments, an RNA editing site result in creating an AUG codon. In some embodiments, in rice, an RNA editing site can be annotated in a mitochondrial genome sequence (NC_011033). In some embodiments, an RNA editing site can be in a cox2 gene (at nucleotide position 214136), and can result in a change of a ACG codon to an AUG codon. In some embodiments, an RNA editing site can be specified by 16 nt upstream and 6 nt downstream. In some embodiments, the SEQ ID NO: 46 can be used to create an AUG translation initiation site on an mRNA, wherein an RNA editing site is shown in a lower case letter “c” (TABLE 1).

In some embodiments, this sequence can be fused with an ORF lacking an initiation codon of a PtxD gene, which can be optimized for a mitochondrial expression in rice as described in Example 1. In some embodiments, a resulting sequence can comprise SEQ ID NO: 47.

In some embodiments, a sequence can be further fused with a promoter and terminator sequences derived from an ATP1 gene in rice mitochondria as described in Example 1 to construct an expression cassette for mOsPtxD.

In some embodiments, while preferred embodiments of a present invention have been shown and described herein, such embodiments are provided by way of example only. Numerous variations, changes, and substitutions can be envisioned without departing from the disclosure herein. It should be understood that various alternatives to the embodiments of the disclosure described herein may be employed in practicing the methods and compositions described herein. It is intended that the following claims define the scope of the disclosure herein, and that methods and structures within the scope of these claims and their equivalents be covered thereby.

### Example 5

### Phosphite Selection of Transformed Cells

Embryogenic callus cultures of rice were initiated and maintained for a minimum of 4-6 weeks on a Chu-N6-based callus induction & maintenance medium supplemented with the plant growth regulator 2,4-D. Four days prior to transformation, callus cultures were subcultured to fresh N6-based callus maintenance medium, or a modified callus maintenance medium with all phosphorus (P) content from phosphite rather than the standard phosphate. Approximately four hours prior to transformation, calli were prepared for bombardment by plating tissue in the target zone on the same phosphite or phosphate-containing media supplemented with mannitol and sorbitol for osmotic protection.

Rice calli were transformed with ptxD expression constructs using biolistics (particle bombardment). Variations of transformation and culture conditions were performed, such as varying the basal medium from Chu N6 to Murashige and Skoog (MS) and varying the amount of gold per DNA prep between 1 and 3 mg/prep.

The following steps were followed for culture, selection and regeneration.

1. After bombardment, the callus was incubated in the dark for 16-20 hours at 26° C., then clumps approximately 1-3 mm in size were subcultured to a selective medium which was the callus maintenance medium supplemented with 5 mM P from phosphite salts in the place of phosphate salts and without casamino acids. In some experiments, 50 mM P from phosphite was used for the first selection. Calli on selective medium were returned to dark incubation for 2-3 weeks.

2. After 2-3 weeks of dark incubation, small (1-3 mm) clumps were again subcultured to fresh selective medium containing phosphite as P source and incubated for approximately 2-4 weeks in a lighted plant growth chamber with a 16 hr light-8 hr dark photoperiod, at a light intensity setting of 60 pmoles per square meter per second, at 26-28° C. In some experiments, at the second or later subculture, the phosphite concentration was increased from its initial level of 5 mM P to 50 mM P or from 50 mM P to 100 mM P from phosphite. A third subculture to fresh selection medium followed by 2-4 weeks of culturing in the lighted plant growth chamber was most often performed. In some experiments, the number of subcultures to fresh selection medium were as many as five, depending on the rate at which the events developed and became large enough to see clearly and isolate.

3. At the end of the third to fifth selection period, vigorously growing calli (individual putative events) were picked from the surrounding dying tissue and transferred to individual plates of fresh selective medium containing phosphite as P source, maintaining their individual identity. In some experiments, the phosphite level during individual event proliferation was 5, 50, or 100 mM P from phosphite, or some combination of these levels such as 5 then 50 mM, or 50 then 100 mM.

4. In some experiments, at the end of the last period of event proliferation, calli representing unique putative ptxD transformation events and still maintaining growth were transferred to a Chu N6-based medium for embryo maturation, still substituting phosphite for phosphate P as selective agent, but removing growth regulator 2,4-D, and supplementing with 2.5 g/L Phytagel as well as the standard 8 g/L agar. In some experiments, levels of P from phosphite were in the range of 5 to 50 mM P at this stage.

5. Mature somatic embryos showing signs of normal maturation in step 4 above were transferred to a Chu N6-based germination medium, still substituting phosphite for phosphate P as selective agent. In some experiments, levels of P from phosphite were again in the range of 5 to 50 mM P at this stage. This medium was supplemented with growth regulators 0.2 mg/L naphthaleneacetic acid and 2 mg/L 6-benzylamino purine, and 2.5 g/L Phytagel as well as the standard 8 g/l agar. Events at the maturation and germination stages were grown in a 16 hr/8 hr light/dark growth chamber at 26-28° C. at light intensity setting of 60 umoles per square meter per second.

Finally, plantlets showing both root and shoot development after step 5 were transferred to pots containing an artificial potting medium and moved to a greenhouse. For the first week after transplanting they were covered by a clear plastic humidity dome for acclimatization. They were then grown to maturity and seed production in a greenhouse.

### Alternative Dual Selection Process

Alternatively, when aptxD expression cassette was linked to or co-transformed with a 35S:HPT expression cassette conferring hygromycin B resistance, selection of nuclear transformation events were facilitated with the use of the phosphite selective media supplemented with 25-50 mg/L hygromycin B.

Variations in the timing of introduction of the hygromycin selection in conjunction with phosphite selection were performed for recovery of events expressing the ptxD gene. In some experiments, the first selection after bombardment was 25 mg/L hygromycin B, and subsequent selection levels were 50 mg/L hygromycin B. In other experiments the first selection after bombardment was 25 mg/L hygromycin B with 5 or 50 mM P from phosphite. In some experiments, the second selection after bombardment was 5, 50 or 100 mM P from phosphite with 50 mg/L hygromycin.

The steps and timeline for experiments with hygromycin selection alone or hygromycin selection in combination with phosphite selection were encompassed by the example given above for phosphite selection.

### Example 6

### PtxD Enzyme Targeted to the Mitochondria Enables Yeast Cells to Grow on Phosphite Medium

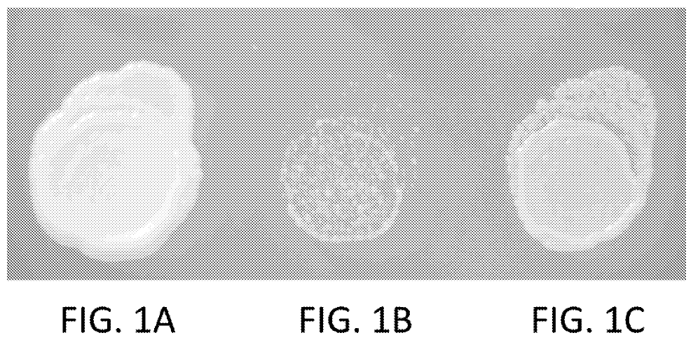

We designed a ptxD coding sequence (SEQ ID NO: 66) with codons optimized for good gene expression in the nucleus of yeast (Saccharomyces cerevisiae) without changing the amino acid composition, and had the corresponding DNA synthesized by an external vendor, GENEWIZ® (South Plainfield, NJ). We fused the yeast nuclear codon-optimized ptxD coding region with a sequence encoding the mitochondrial targeting sequence (MTS) of the yeast COX4 gene (SEQ ID NO: 67). This chimeric coding region (SEQ ID NO: 68) for the MTS-ptxD fusion protein was expressed using the strong constitutive promoter TEF1 (SEQ ID NO: 69) in the nucleus of yeast, using the pYES2 vector. The transformation of the resulting construct, pNY101, into the yeast strain CUY563, was performed using a yeast transformation kit (Frozen-EZ Yeast Transformation II Kit™ from the Zymo Research Corporation™) and selection on a single dropout formulation (without Uracil) of Synthetic Defined (SD) Yeast Media (URA dropout Medium™ MP Cat. No. 4813065). Then, transformants were transferred on the medium containing phosphite as a sole phosphorus source. For this monopotassium phosphite (Alfa Chemistry) was added to a synthetic defined broth containing 2% glucose without potassium phosphate and without uracil (Formedium CSM1202) to a final concentration of 7.34 mM. The transformants showed the ability to grow on the medium with phosphite as a sole phosphorus source (FIG. 1A), whereas the transformants with the empty vector, pYES2, did not (FIG. 1B).

### Example 7

### PtxD Enzyme Targeted to the Mitochondria Enables Rice Callus Cells to Grow on Phosphite Medium

In this Example, rice callus transformations were performed essentially as described in Example 5. We designed a ptxD coding sequence (SEQ ID NO: 70) with codons optimized for rice (Oryza sativa) nuclear expression and had the corresponding DNA synthesized (by GENEWIZ® South Plainfield, NJ).

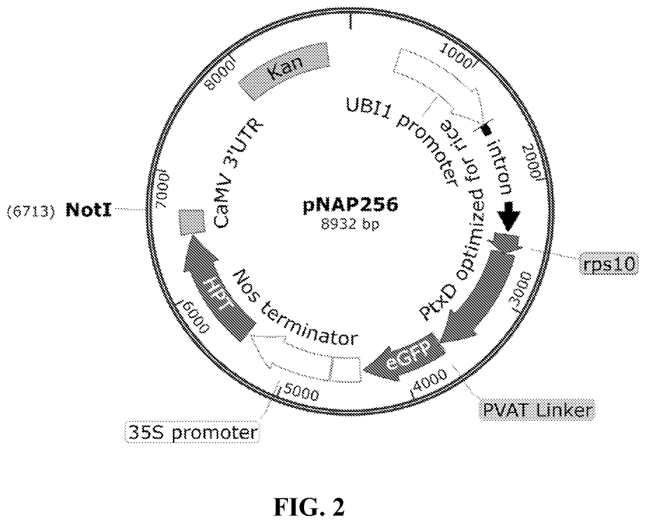

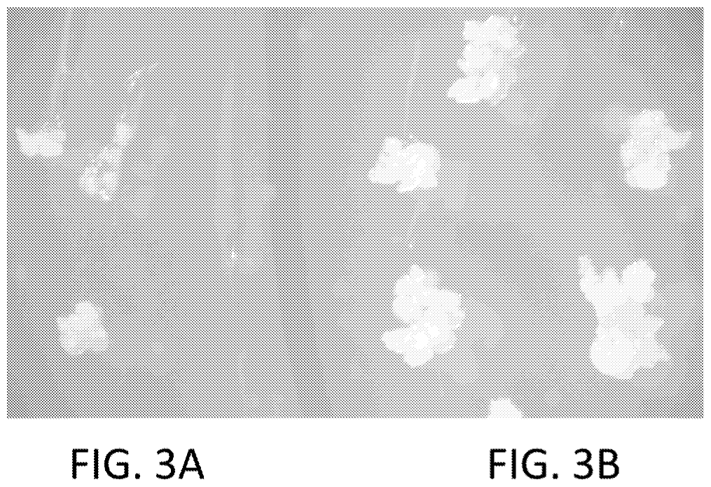

We used a plasmid DNA construct in which the codon-optimized ptxD coding region was fused with a sequence encoding a mitochondrial targeting sequence. In plasmid pNAP256 (FIG. 2) the codon-optimized ptxD coding region was fused with the MTS coding region (SEQ ID NO: 71) of the rice RPS10 gene, which encodes a mitochondrial ribosomal protein. In addition, the carboxyl end of the ptxD ORF was fused with the eGFP ORF by use of a sequence encoding a PVAT linker (SEQ ID NO: 72). In plasmid pNAP148, the codon-optimized ptxD coding region was fused with the MTS coding region (SEQ ID NO: 38) of the At5G47030 gene of Arabidopsis thaliana. In each plasmid, the chimeric coding region was expressed using the maize UBI promoter and its first intron (SEQ ID NO: 39) which provides strong constitutive expression in rice. After transformation of pNAP256 into rice callus cells using the biolistic method, we selected events that could grow on the medium with phosphite as the sole phosphorus source (FIG. 3A and FIG. 3B), whereas non-transformed rice callus cells did not show any selectable growth.

### Example 8

ptxD Gene Expressed in the Mitochondria Enables Yeast Cells to Grow on Phosphite Medium

We made construct pNY104 to transform yeast with mitochondrial plasmid DNAs carrying the ptxD gene. In yeast, we used the pHD6 plasmid backbone to clone and introduce the ptxD coding region that was codon-optimized for mitochondrial expression. For this the ARG8m in pHD6 was replaced by the ptxD coding region optimized for yeast mitochondrial expression by changing tryptophan codons to UGA, which is recognized as a stop codon in the cytoplasm but as a tryptophan codon in mitochondria. The optimized ptxD coding region (SEQ ID NO: 73) was put under control of the COX2 mitochondrial promoter (SEQ ID NO: 74) and COX2 mitochondrial terminator (SEQ ID NO: 75) and cloned into the backbone of pHD6. After transforming the plasmid into wild-type yeast cells (CUY563 strain), cells were selected on a medium containing phosphite as the sole phosphorus source, as described in Example 6 above. We obtained multiple transformants (FIG. 1C, pNY104) whereas transformation with a control plasmid without ptxD did not yield any positive colonies (FIG. 1B).

### Example 9

ptxD Gene Expressed in the Mitochondria Enables Rice Callus Cells to Grow on Phosphite Medium

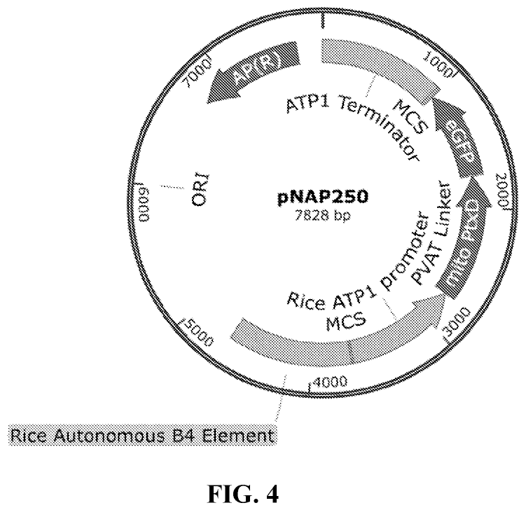

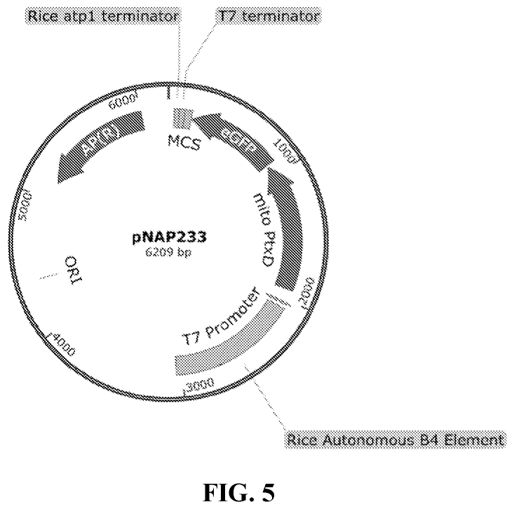

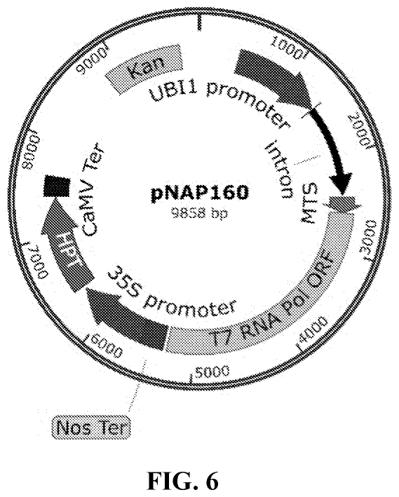

For the experiments in this Example, we designed two mitochondrial expression cassettes to have varying gene expression levels. The first expression cassette (FIG. 4, pNAP250) utilized the promoter elements of the rice mitochondrial ATP1 gene. The promoter of the rice ATP1 gene has been shown to have six transcriptional start sites. The 928 bp-long region upstream of ATG codon of the ATP1 gene (SEQ ID NO: 30) containing all six transcription start sites was chosen as a promoter. For termination of transcription in the first expression cassette, we cloned the 863 bp-long region downstream of the ATP1 stop codon (SEQ ID NO: 76). The sequence of the ATP1 gene region was based on the GenBank information of the mitochondrial DNA of rice Nipponbare (accession #: NC_011033). The second expression cassette (FIG. 5, pNAP233) had the T7 promoter sequence inserted upstream of the nearest transcription start site, which produced a synthetic promoter (SEQ ID NO: 77) with a length of only 139 bp. For the second expression cassette, the transcription termination region (SEQ ID NO: 78) consisted of the T7 terminator inserted upstream of a short AT-rich 40 bp sequence from the ATP1 terminator. To enhance transcription in mitochondria using the T7 promoter, we constructed nuclear expression vector pNAP160 (FIG. 6). Plasmid pNAP160 contains a sequence (SEQ ID NO: 79) encoding the bacterial T7 RNA polymerase fused to a mitochondrial targeting sequence of rice RPS10; this coding region is operably linked to a maize UBI promoter and intron, which produces strong constitutive expression in rice.

Plasmids pNAP250 and pNAP233 each have a sequence that encodes a fusion protein having a fluorescent reporter (eGFP) fused to the carboxyl end of a ptxD protein. Plasmid pNAP250 has a sequence (SEQ ID NO: 80) that encodes a fusion protein (SEQ ID NO: 81) in which the two enzymes are connected with a PVAT-linker (SEQ ID NO: 72). Plasmid pNAP233 has a sequence (SEQ ID NO: 82) that encodes a fusion protein (SEQ ID NO: 83) in which the two enzymes are connected with a GGGGS-linker (SEQ ID NO: 84). Since these fusions may compromise the function of ptxD as well as eGFP proteins, we first tested the two fusion proteins in yeast and confirmed that each fusion protein retained both enzymatic activities.

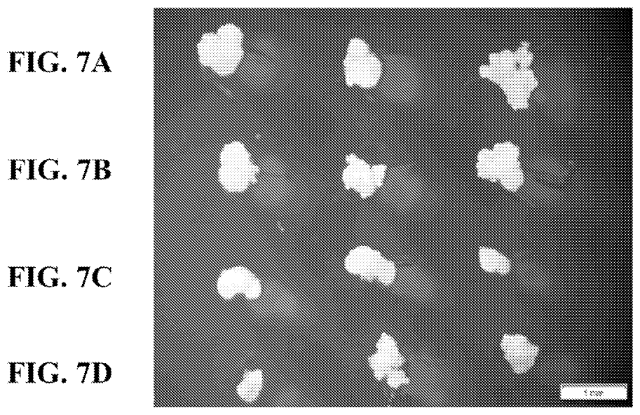

Transformations in this Example were performed by the biolistic microprojectile method essentially as described in Example 5. Plasmid DNA for mitochondrial transformation was co-bombarded with another DNA that allowed selection of nuclear transformation using a hygromycin resistant gene (HPT). As we expected the frequency of mitochondrial transformation to be significantly less than that of nuclear transformation, we planned to enrich for mitochondrial transformants by selecting mitochondrial transformants among cells that also received a nuclear selection marker. The double selection was performed by using hygromycin-containing media that had phosphite as the sole source of phosphorus. The constructs were transformed alongside a negative control (no mitochondrial expression plasmid but with a nuclear expression plasmid for an HPT gene). We observed that several independent rice calli grew on the medium with a double selection (FIG. 7A, pNAP250; FIG. 7C, pNAP233). No growth was observed among the negative control samples. PCR analysis of several positive events showed the presence of not only the ptxD gene but also mitochondrial plasmid DNA.

The expression cassettes for mitochondrial transformation were cloned into the pBR322 plasmid as done for yeast Edit Plasmids, and those for nuclear transformation were cloned into pUC-GW-Kan (GENEWIZ® vector).

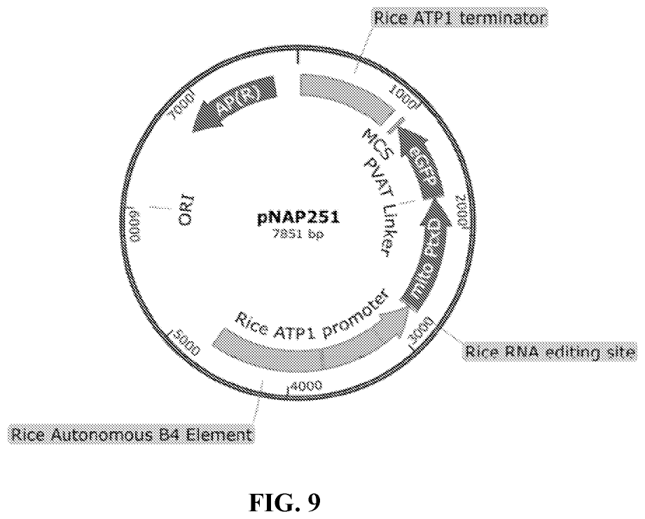

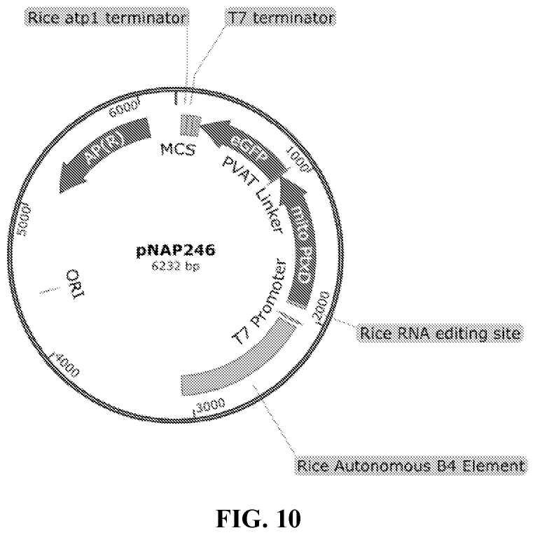

For the rice experiment, callus cells were grown for several weeks after biolistic transformation before fluorescence analysis using a confocal microscope. Our initial findings were that wild-type rice callus cells without any DNA transformation exhibited significant fluorescence that overlapped with the eGFP emission spectrum. Due to this recalcitrant issue, we decided to confirm mitochondrial transformation by adding an element for natural RNA editing in the ptxD mRNA encoded in our mitochondrial plasmids. As for the natural RNA editing of mRNA, extensive studies have been reported in the literature. Those studies showed that plant mitochondria have significant mRNA editing activities, which are found to a very limited extent in chloroplasts and not at all in the nucleus. The RNA editing sites are known to be specific to certain sequences. No pattern associated with the RNA editing sites has been discovered. One study with isolated wheat mitochondria showed that 16 nt upstream and 6 nt downstream of the editing sites were sufficient to induce the correct mRNA editing. Based on that finding, we designed vectors to create an AUG translational start codon in the mRNA of the selectable marker gene, the ptxD gene, using the RNA editing site for the rice mitochondrial gene, NAD4L (FIG. 8). Without RNA editing, i.e., plasmid DNA not transformed into mitochondria but into the nucleus or chloroplasts, the codon will remain as ACG on the mRNA transcript and therefore no ptxD protein will be produced. Plasmid pNAP251 (FIG. 9) is similar to pNAP250 (first expression unit) but has the RNA-editing site inserted. Likewise, plasmid pNAP246 (FIG. 10) is similar to pNAP233 (second expression unit) but has the RNA-editing site added. The pNAP251 and pNAP246 plasmids each have a sequence (SEQ ID NO: 85) that encodes a RNAed-ptxD-eGFP fusion protein (SEQ ID NO: 86) in which the ptxD and eGFP enzymes are connected with a PVAT-linker (SEQ ID NO: 72).

After biolistic transformation, transformed events with plasmids pNAP251 and pNAP246 (that each have the NAD4L RNA-editing element) were selected on hygromycin-containing media that had phosphite as the sole phosphorus source. The RNA-editing element and promoters we tested all produced rice calli with similar growth behavior (FIG. 7B, pNAP251; FIG. 7D, pNAP246), showing that these elements were functional and efficacious, i.e., plasmids were transformed into mitochondria.

### Example 10

Donor DNA Incorporated into the Rice Mitochondria Genome

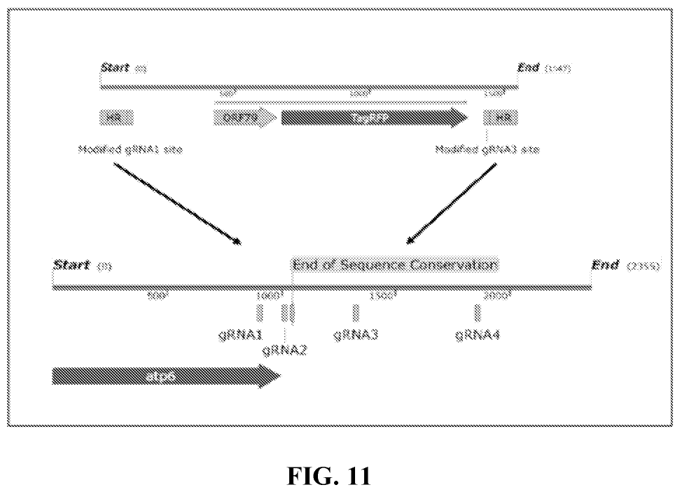

Mitochondrial transformation with ptxD using phosphite selection was deployed for gene editing of mitochondrial DNA in rice. The target of gene editing was the site of the rice CMS gene, orf79 (SEQ ID NO: 87), which is the region downstream of mitochondrial ATP6 gene. The orf79 is only present in the rice CMS line Boro II Taichung and is not present in wild-type rice mitochondria. The experiment was designed to insert the orf79 gene directly downstream of the ATP6 gene as it is found in the mitochondria of the rice CMS line Boro II Taichung. We chose the MAD7 site-specific nuclease, which belongs to the Cas12 class, as the CRISPR enzyme for this experiment. We chose two pairs of guide RNAs (gRNA1 & gRNA3; and gRNA2 & gRNA4) that were unique to mitochondrial DNA of the Nipponbare rice cultivar (FIG. 11). Each gRNA had the target sequence fused with crRNA, which is required for guide RNA function, and was present directly downstream of the ptxD-eGFP coding region in the Edit Plasmids as mentioned above. Each gRNA coding sequence was flanked by tRNA coding sequences to aid in subsequent RNA processing of the polycistronic transcript. Donor DNAs SEQ ID NO: 119 and SEQ ID NO: 120, corresponding to cleavage sites created by the gRNA1 & gRNA3 pair and the gRNA2 & gRNA4 pair, respectively, were synthesized to have ends homologous to the genomic sequence flanking the target sites. Each homologous region (labelled as HR in FIG. 11) had a length of 100 or 106 bp adjacent to the gRNA site. The short length was designed to prevent homologous recombination without CRISPR cleavages at the target sites as shown in our yeast mitochondrial editing experiments (WO 2019/040645 A1). The target sequences of gRNAs in the Donor DNAs were modified such that they would not be targets of CRISPR, i.e., gene edited mitochondrial DNA would be stable in the presence of MAD7 and gRNAs.

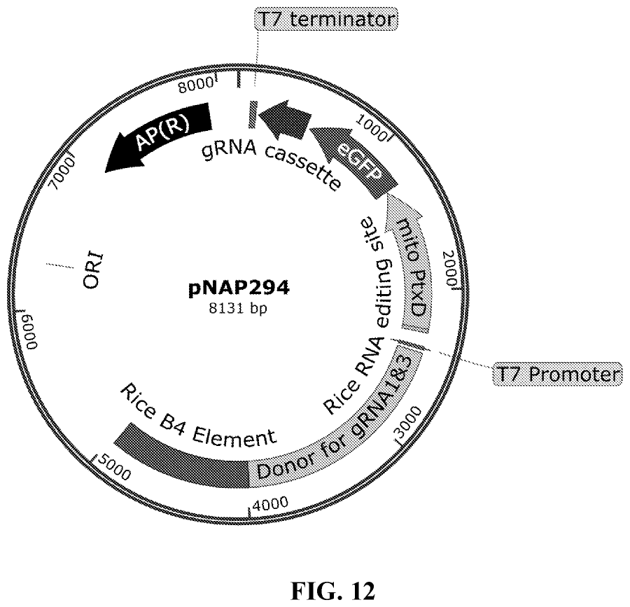

A map of a representative Edit Plasmid (pNAP294) is shown in FIG. 12. In pNAP294, 3′ to the ptxD-eGFP coding region is the 334-bp coding region (SEQ ID NO: 121) for the multigene cassette encoding trnP-gRNA1-trnE-gRNA3-trnK.

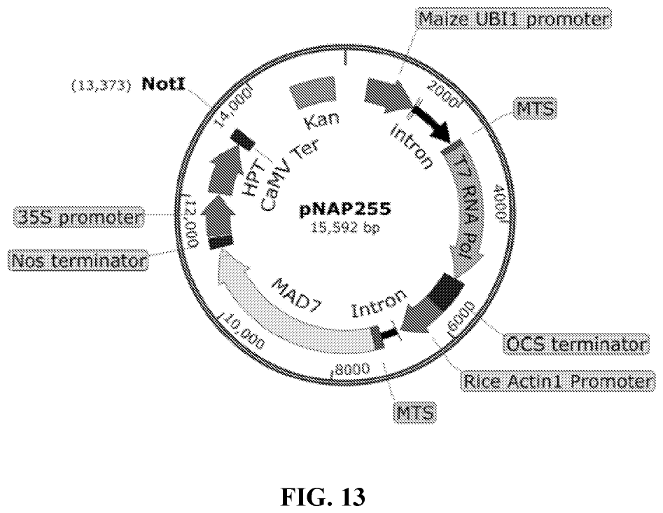

The pNAP255 construct (FIG. 13) has a sequence (SEQ ID NO: 88) that encodes a fusion protein (SEQ ID NO: 89) in which the MAD7 enzyme is fused at the amino terminus with a mitochondrial targeting sequence (SEQ ID NO: 90) of the rice RPS10 protein and expressed in the nucleus by the maize UBI promoter. To provide T7 RNA polymerase in mitochondria, the nuclear construct also has a sequence (SEQ ID NO: 38) encoding a fusion protein (SEQ ID NO: 91) in which the T7 RNA polymerase is fused at the amino terminus with the MTS (SEQ ID NO: 92) of the At5G47030 gene of Arabidopsis thaliana. Edit Plasmids containing the Donor DNAs and also having the T7 promoter for ptxD-eGFP and gRNA expression were transformed together with the pNAP255 construct (FIG. 13).

Rice callus tissue was transformed with these constructs essentially as described in Example 5 using the biolistic method and transformed events were selected on corresponding media over two months. Gene editing events were analyzed by PCR reactions that amplified the junction regions of the Donor DNA integration. We observed the integration of Donor DNA with varying frequencies. The most frequent integration was observed at the gRNA1 site (SEQ ID NO: 93) when the guide RNA was expressed under the T7 promoter (10 out of 15 independent transformation events; FIG. 14). The next most frequent integration was observed at the gRNA2 site (SEQ ID NO: 94) (2 out of 30 independent events examined). No integration was detected at the gRNA3 (SEQ ID NO: 95) or gRNA4 (SEQ ID NO: 96) sites among 60 events examined, or for control events without MAD7 expression among 45 events examined. The integrations at the gRNA1 and gRNA2 sites were further confirmed by sequencing of the PCR fragments of multiple events (FIG. 15). In all cases, the junction fragments contained the sequences as predicted from the precise integration of Donor DNA near the cleavage sites induced by MAD7 site-specific nuclease. One feature that was not expected from our prior experience with Cas9-induced recombination in yeast mitochondria is that wild-type gRNA sequences were conserved after the integration despite Donor DNAs containing modified sequence at the gRNA sites, e.g., to prevent subsequent cleavages through CRISPR. This unexpected result may be explained by the difference in nuclease function between MAD7 and Cas9. MAD7 produces nicks rather than blunt-end double-strand breaks at gRNA sites.

### Example 11

ptxD Gene Expressed in the Mitochondria Enables Rice Callus Cells to Grow on Phosphite Medium

Three other sets of transformation experiments were performed using the ptxD gene as a selectable marker for mitochondrial transformation. In contrast to Example 9, in these experiments the ptxD protein was not fused to the eGFP protein. Also, in these experiments the ptxD coding region did not contain a mitochondrial RNA editing site.

In the first set of experiments, rice callus cells were transformed with the construct (pNAP163) in which the ptxD coding region (SEQ ID NO: 122) was codon optimized for expression in rice mitochondria and was linked to the rice mitochondrial ATP1 promoter (SEQ ID NO: 30). This construct was co-transformed with a nuclear construct (pNAP152) that has the coding region of the hygromycin resistant gene expressed under a 35S promoter. pNAP163 and pNAP152 were constructed as described in earlier Examples.

In the second set, rice callus cells were transformed with the construct (pNAP164) that was designed to express the ptxD coding region optimized for rice mitochondria (SEQ ID NO: 122) under the hybrid promoter (SEQ ID NO: 44) comprising the rice mitochondrial promoter derived of the ATP1 gene in which the T7 promoter was embedded to enhance expression. This construct was co-transformed with a nuclear construct (pNAP160) that carried the hygromycin resistant gene expressed under 35S promoter as well as the T7 polymerase gene fused with a mitochondrial targeting sequence expressed under maize Ubiquitin promoter. Plasmids pNAP164 and pNAP160 were constructed as described in earlier Examples.

The third set was a control, in which rice callus cells were transformed with the construct (pNAP149) that encodes a fusion protein (SEQ ID NO: 123) containing the ptxD protein fused with the mitochondrial targeting peptide of the rps10 gene (At5g47030). The coding region for this fusion protein was expressed under the maize Ubiquitin promoter. The plasmid also contained the coding sequence for hygromycin phosphotransferase expressed under a 35S promoter.

In each of the above three cases, resistant cell lines were obtained that were able to grow in the presence of phosphite as the source of phosphorus in the media.

### Example 12

Donor DNA Containing the ptxD Selectable Marker Incorporated into the Rice Mitochondria Genome

Two other sets of transformation experiments were performed using the ptxD gene as a selectable marker for mitochondrial transformation and gene editing. In contrast to Example 10, in these experiments the expression unit for the ptxD:eGFP fusion protein was present on the Donor DNA. In the first set of experiments, rice callus cells were transformed with two polynucleotides at the same time. One polynucleotide was the gel-purified Donor DNA fragment derived from pNAP420 (SEQ ID NO: BB124) or the gel-purified Donor DNA from pNAP421 (SEQ ID NO: BB125). The Donor DNA was designed to integrate into the mitochondrial ATP6 gene of the Japonica rice cultivar Nipponbare. The 7.5 kb-long, linear Donor DNA had five segments arranged in the following configuration: [1.4 kb of 5′ homologous region spanning over the ATP6 gene]-[CMS orf79 gene]-[mOsPtxD-eGFP expression cassette with the ATP1 and T7 promoters and terminators]-[gRNA expression cassette driven by T7 promoter]-[0.9 kb of 3′ homologous region downstream of the ATP6 gene]. Two gRNAs were designed to cleave at internal sites of the 5′-HR and 3′-HR regions, respectively, in the presence of the MAD7 enzyme. The Donor DNAs from pNAP420 and pNAP422 only differ in the RNA editing sequence used to initiate translation of mOsPtxD. In pNAP420, the region containing the ATG translation initiation codon of mOsPtxD was replaced with a sequence containing a natural RNA editing site found at the initiation codon of the rice mitochondrial nad4L gene (where the RNA editing site is shown with a lower-case letter “c” in TABLE 1): SEQ ID NO: 126.

This sequence we used was longer than the deduced RNA editing recognition sites, which were shown to be 23 nt long (Chouty et al., 2004; DOI: 10.1093/nar/gkh969).

In pNAP422, the region containing the ATG translation initiation codon of mOsPtxD was replaced with a sequence containing a natural RNA editing site found at the initiation codon of the rice mitochondrial cox2 gene (where the RNA editing site is shown with a lower-case letter “c” in TABLE 1): SEQ ID NO: 131.

To express the MAD7 nuclease we used pNAP255 (Example 10; FIG. 13), which had the following three expression cassettes: 1) coding region for MTS-T7 polymerase under control of the maize Ubiquitin promoter; 2) coding region for MTS-MAD7 under control of the rice Actin 1 promoter; and 3) coding region for hygromycin phosphotransferase (HPT) under control of the 35S promoter. Transformation experiments with Donor DNA and pNAP255 were performed by biolistic method as described in Example 5 with selection on phosphite medium containing hygromycin.

In the second set of transformation experiments, rice callus cells were transformed exactly the same as the first set but without pNAP255, i.e., no MAD7 expression as a control, and selection was done on phosphite medium without hygromycin.

Transformation resulted in multiple independent events in all experiments including the control.

To assay for integration of the Donor DNA into the target ATP6 region, we designed primers to amplify the junction regions by PCR. For the 5′ junction region, the following two primers were designed:

5HRA (specific to wild-type mtDNA, no priming site in the Donor DNA): SEQ ID NO: 127

ORFB (specific to the Donor DNA, no priming site in wild-type mtDNA): SEQ ID NO: 128

For the 3′ junction region, the following primers were designed:
420A (specific to the Donor DNA, no priming site in wild-type mtDNA): SEQ ID NO: 129
3HRA (specific to wild-type mtDNA, no priming site in the Donor DNA): SEQ ID NO: 130

For the PCR experiments, crude DNA fractions were isolated from callus samples (several mg/sample) by heating to 100° C. for 20 min in the presence of 0.02N NaOH and 1 mM EDTA, subsequent phenol/chloroform extraction and ethanol precipitation. DNA was resuspended in TE. Approximately 100 ug DNA/sample were used for PCR reactions. PCR reactions were performed by use of LongAmp Taq (NEW ENGLAND BIOLABS®) following the manufacture's protocol. The PCR conditions were as follows:

5′ junction amplification: 95° C. for 30 sec-95° C. for 15 sec-65° C. for 3 min (repeat steps 2 &3 for 35 times)-65° C. for 10 min. 3′ junction amplification: 95° C. for 30 sec-95° C. for 15 sec-63° C. for 30 sec-65° C. for 2 min (repeat steps 2, 3 & 4 for 35 times)-65° C. for 10 min.

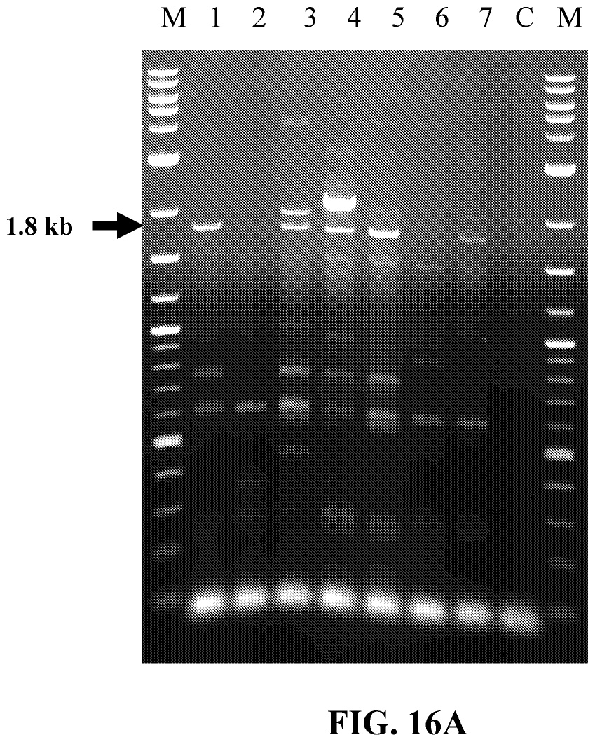

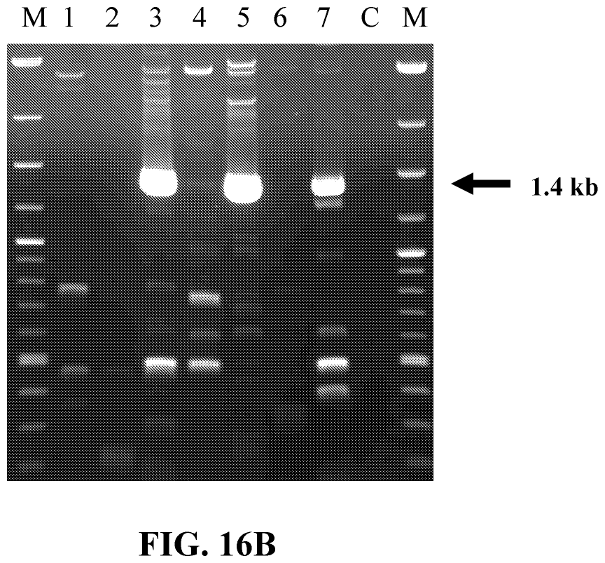

In summary, out of 19 independent events derived from the first set of experiments (Donor DNA+MAD7), 7 events were shown to carry both the 5′ and 3′ junction regions. Examples of PCR fragments corresponding to the 1.8 kb 5′ junction fragment and the 1.4 kb 3′ junction fragment are shown in FIG. 16A and FIG. 16B, respectively. PCR bands were isolated from the gel and sequenced directly. The sequences matched what were expected from the correct integration of Donor DNA. On the other hand, 8 independent events derived from the control experiments (without MAD7) did not produce any junction fragments. These data demonstrated that selection using the mOsPtxD gene resulted in the correct delivery of Donor DNA into rice mitochondria, and integration was facilitated by use of the CRISPR system.

### Example 13

### Analysis of In Vivo RNA Editing

To evaluate the efficiency of RNA editing sites that were designed to express ptxD protein only in mitochondria, we analyzed the ptxD transcripts from events transformed by three different constructs. Event #1 was derived from the co-transformation of pNAP420 (mitochondrial) and pNAP255 (nuclear) constructs, event #2 was derived from pNAP391 (mitochondrial) and pNAP199 (nuclear) constructs, and event #3 from pNAP422 (mitochondrial) and pNAP255 (nuclear) constructs. As for mitochondrial constructs, we transformed Donor DNA fragments of corresponding constructs, which were targeted to the ATP6 region with the homologous regions at their both ends same as the construct described above, as well as harboring the mOsPtxD selectable marker gene. RNA editing sites from the rice mitochondrial nad4L and cox2 genes were used to express mOsPtxD protein in rice mitochondria. The following three constructs carried the indicated sequences for creation of an AUG translation start codon by means of RNA editing (the RNA edited nucleotide is shown as a lower-case letter in TABLE 1; promoters for RNA expression are also indicated): pNAP391: nad4L_short (SEQ ID NO: 119), expressed under the ATP1 promoter;
pNAP420: nad4L_long (SEQ ID NO: 126), expressed under the ATP1+T7 promoter; and
pNAP422: cox2 (SEQ ID NO: 131), expressed under the ATP1+T7 promoter.

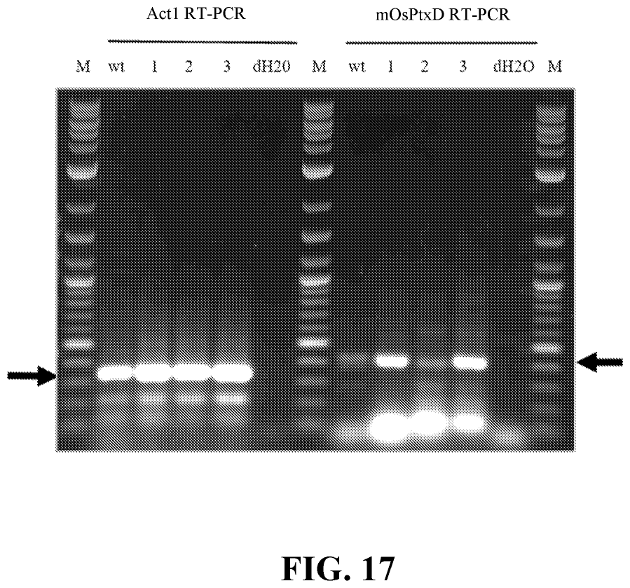

Total RNA was isolated from each event along with a wild-type callus as control using the RNeasy Plant Mini Kit (Cat No. 74904; QIAGEN®). To eliminate DNA contamination, RNA samples were treated with RNase-free DNase I and extracted through phenol and chloroform before precipitation in ethanol. Resuspended total RNA samples (5 ug each) were subjected to the first-strand DNA synthesis by using hexamer oligo nucleotides in 5′ RACE Protocol using the Template Switching RT Enzyme Mix (NEW ENGLAND BIOLABS® #M0466). Aliquots of cDNA were subjected to PCR to amplify the transcribed region of the rice Actin1 gene using primers OsAct1-F2: 5′-GAGAGAAGATGACCCAGATCATGTTCG-3′ (SEQ ID NO: 132) and OsAct1-R2: 5′-CTGGCAGTATCAAGCTCCTGTTCATAA-3′ (SEQ ID NO: 133). The genomic region contained an intron. Consequently, any genomic DNA contamination would have produced a 460 bp PCR product while the amplification from nuclear Act1 mRNA is expected to produce a 346 bp product. The control PCR reaction with Act1 primers produced the expected 346 bp mRNA product without any 460 bp genomic DNA product, showing the purity of our RNA samples (FIG. 17). The mOsPtxD transcripts were then amplified by using the first strand cDNA as template, OsATP1-PRO-FP1: 5′-GTCTGCCCCATTCGATAATGGCA-3′ (SEQ ID NO: 134) and mOsPtxD-RP1: 5′-TCCACATCGAAATTGTCGAAGCCCTT-3′ (SEQ ID NO: 135) primers and Q5-HI fidelity Taq polymerase (NEW ENGLAND BIOLABS®). The expected product with a length of 417 bp was amplified from the event samples with higher amounts for events #1 and 3 than for event #2 (FIG. 17), which corresponded to the presence or absence of the T7 promoter. This confirmed that the mOsPtxD gene was well expressed in mitochondria of these events. The mOsPtxD bands were isolated from the gel and subjected to the deep sequencing, which was contracted to AZENTA LIFE SCIENCES®.

Approximately, a half million reads were obtained from each sample. We analyzed the frequency of sequences with the desired RNA editing and the results are summarized below:
Event #1 (nad4L with 38 nt): 80 reads with RNA editing out of 459,959 reads (174 ppm);
Event #2 (nad4L with 26 nt): 37 reads with RNA editing out of 554,671 reads (66 ppm);
Event #3 (cox2 with 40 nt): 51 reads with RNA editing out of 600,272 reads (85 ppm).

In each case, the frequency of RNA editing was significantly less than what has been reported for these RNA editing sites at their native sites in the corresponding mitochondrial genes, nad4L and cox2, as detected by conventional sequencing of cDNA. Possibly the recognition sequence of each RNA editing site that we chose may have been suboptimal. Additionally, the recognition sequences may also be influenced by sequences present elsewhere in the mRNA. However, despite the low frequency of RNA editing, ptxD gene expression was sufficient to support the growth of callus cells on the selective medium.

What is claimed is:

1. A cell comprising an edited mitochondrial genome, wherein the edited mitochondrial genome comprises an exogenous polynucleotide encoding a phosphite dehydrogenase or a biologically active fragment thereof.

2. The cell of claim 1, wherein the cell is a eukaryotic cell.

3. The cell of any one of claims 1-2, wherein the eukaryotic cell is selected from the group consisting of a protist cell, a yeast cell, an algal cell, a plant cell, an insect cell, a non-human animal cell, an isolated and purified human cell, and a mammalian tissue culture cell.

4. The cell of claim 3, wherein the eukaryotic cell is a plant cell.

5. The cell of claim 3 or claim 4, wherein the plant cell is selected from the group consisting of: a wheat cell, a maize cell, a rice cell, a barley cell, a sorghum cell, a rye cell, a canola cell, a broccoli cell, a cauliflower cell, and a soybean cell.

6. The cell of any one of claims 1-5, wherein the edited mitochondrial genome comprises introduction of replacement, substitution, deletion or insertion of at least one nucleotide.

7. The cell of any one of claims 1-6, wherein the cell comprises a transformed mitochondrion, wherein the transformed mitochondrion comprises the edited mitochondrial genome.

8. The cell of any one of claims 1-7, wherein a nucleic acid sequence of the exogenous polynucleotide encoding the phosphite dehydrogenase or a biologically active fragment thereof comprises at least 80%, 85%, 90%, 95%, 96%, 97%, 98% or 99% sequence identity to SEQ ID NO: 28.

9. The cell of claim 8, wherein the nucleic acid sequence of the exogenous polynucleotide encoding the phosphite dehydrogenase or a biologically active fragment thereof comprises SEQ ID NO: 28.

10. The cell of any one of claims 1-9, wherein an amino acid sequence of the phosphite dehydrogenase or a biologically active fragment thereof encoded by the exogenous polynucleotide comprises at least 80%, 85%, 90%, 95%, 96%, 97%, 98% or 99% sequence identity to SEQ ID NO: 29, 48, 49, 50, 51, 52, 53, 54, 55, 56, 57, 58, 59, or 60.

11. The cell of claim 10, wherein the amino acid sequence of the phosphite dehydrogenase or a biologically active fragment thereof comprises SEQ ID NO: 29.

12. The cell of any one of claims 1-11, wherein a sequence encoding a start codon of the exogenous polynucleotide is replaced with a sequence encoding a mitochondrial RNA editing site.

13. The cell of claim 12, wherein the mitochondrial RNA editing site is from a mitochondrial nad4L gene or a mitochondrial cox2 gene.

14. The cell of any one of claims 12-13, wherein the sequence encoding the mitochondrial RNA editing site comprises SEQ ID NO: 46.

15. The cell of any one of claims 12-14, wherein the exogenous polynucleotide encoding the phosphite dehydrogenase or a biologically active fragment thereof comprises SEQ ID NO: 47.

16. The cell of any one of claims 1-15, wherein the edited mitochondrial genome further comprises a second polynucleotide encoding a polypeptide or a functional RNA, or both, wherein the polypeptide and the functional RNA are exogenous to the mitochondria.

17. The cell of claim 16, wherein the second polynucleotide comprises a cytoplasmic male sterility (CMS) coding region.

18. The cell of claim 17, wherein the CMS coding region is orf79.

19. The cell of claim 18, wherein the cell is a rice cell.

20. The cell of claim 17, wherein the CMS coding region is orf256 or is orf279.

21. The cell of claim 20, wherein the cell is a wheat cell.

22. The cell of any one of claims 1-21, wherein the cell further comprises a third exogenous polynucleotide in a nucleus of the cell, wherein the third exogenous polynucleotide encodes a selectable marker polypeptide that provides the cell with tolerance to a selective agent.

23. The cell of claim 22, wherein the selectable marker polypeptide is hygromycin phosphotransferase (HPT).

24. The cell of claim 23, wherein the selective agent is hygromycin.

25. The cell of any one of claims 1-24, wherein the cell comprises a plurality of mitochondrial genomes wherein at least 50%, 60%, 70%, 80%, 90%, or 100% of the plurality of mitochondrial genomes comprise the edited mitochondrial genome.

26. The cell of any one of claims 1-25, wherein the cell is homoplasmic for the edited mitochondrial genome.

27. The cell of any one of claims 1-26, wherein the cell expresses the phosphite dehydrogenase or the biologically active fragment thereof encoded by the exogenous polynucleotide.

28. The cell of any one of claims 1-27, wherein the cell grows in a medium wherein phosphite is present.

29. The cell of any one of claims 1-28, wherein the cell grows when phosphite is present as a primary phosphorus source and wherein phosphate is present at less than 3 mg/liter.

30. The cell of any one of claims 27-29, wherein the cell grows in a medium wherein the phosphite is present at 50 mM or greater.

31. The cell of any one of claims 27-29, wherein the cell grows in a medium wherein the phosphite is present at 100 mM or greater.

32. A transgenic plant or parts thereof comprising the cell of any one of claims 1-31.

33. The transgenic plant or parts thereof of claim 32 comprising a cell, a tissue, a propagation material, a seed, a pollen, a progeny, or any combination thereof.

34. The transgenic plant or parts thereof of claim 32 or 33, wherein the transgenic plant or parts thereof is grown in a temperature-controlled incubator.

35. The transgenic plant or parts thereof of claim 34, wherein the temperature-controlled incubator further comprises a light-dark cycle.

36. A method comprising introducing into a mitochondrion of a cell, a first polynucleotide encoding a first polypeptide, wherein the first polypeptide comprises a phosphite dehydrogenase or a biologically active fragment thereof.

37. The method of claim 36, wherein the method further comprises growing the cell under conditions in which the phosphite dehydrogenase or the biologically active fragment thereof is produced.

38. The method of claim 36 or 37, wherein the method further comprises growing the cell in a medium wherein a phosphite is present.

39. The method of any one of claims 36-38, wherein the method further comprises selecting a cell comprising an edited mitochondrial genome comprising the first polynucleotide.

40. The method of claim 39, wherein the method further comprises introducing into the mitochondrion of the cell a donor DNA, wherein the donor DNA comprises:
a. a second polynucleotide encoding a second polypeptide or a functional RNA, or both, wherein the second polypeptide and the functional RNA are exogenous to the mitochondrion;
b. a third polynucleotide at one end; and
c. a fourth polynucleotide at the other end;
wherein the third polynucleotide and the fourth polynucleotide each comprises a sequence capable of homologous recombination with an endogenous mitochondrial DNA sequence,
wherein homologous recombination of all or part of the third polynucleotide, the fourth polynucleotide, or both the third polynucleotide and the fourth polynucleotide, with the endogenous mitochondrial DNA sequence results in integration of the second polynucleotide into the endogenous mitochondrial DNA sequence; and
selecting a cell with the edited mitochondrial genome, wherein the edited mitochondrial genome comprises the second polynucleotide.

41. The method of claim 40, wherein the donor DNA further comprises the first polynucleotide.

42. The method of claim 40 or claim 41, wherein the edited mitochondrial genome comprises both the first polynucleotide and the second polynucleotide.

43. The method of any one of claims 40-42, wherein the second polynucleotide comprises a cytoplasmic male sterility (CMS) coding region.

44. The method of claim 43, wherein the CMS coding region comprises orf79.

45. The method of claim 44, wherein the orf79 is from a rice cell.

46. The method of claim 43, wherein the CMS coding region comprises orf256 or orf279.

47. The method of claim 46, wherein the orf256 or the orf279 is from a wheat cell.

48. The method of any one of claims 40-47, wherein the sequence capable of homologous recombination in the third polynucleotide has a size of 25-75 nucleotides, 25-100 nucleotides, 25-150 nucleotides, 25-200 nucleotides, 25-300 nucleotides, 25-400 nucleotides, 25-500 nucleotides, 25-1000 nucleotides, 25-1500 nucleotides, or 25-2000 nucleotides.

49. The method of any one of claims 40-48, wherein the sequence capable of homologous recombination in the fourth polynucleotide has a size of 25-75 nucleotides, 25-100 nucleotides, 25-150 nucleotides, 25-200 nucleotides, 25-300 nucleotides, 25-400 nucleotides, 25-500 nucleotides, 25-1000 nucleotides, 25-1500 nucleotides, or 25-2000 nucleotides.

50. The method of any one of claims 40-49, wherein the first polynucleotide, the second polynucleotide, the third polynucleotide and the fourth polynucleotide are all introduced into the mitochondrion as components of a single recombinant DNA construct.

51. The method of any one of claims 40-50, wherein at least one selected from the group consisting of: the first polynucleotide, the second polynucleotide, the third polynucleotide, the fourth polynucleotide, and any combination thereof, is introduced into the cell via microinjection, meristem transformation, electroporation, Agrobacterium-mediated transformation, viral based gene transfer, transfection, vacuum infiltration, biolistic particle bombardment or any combination thereof.

52. The method of any one of claims 40-51, wherein at least one selected from the group consisting of: the first polynucleotide, the second polynucleotide, the third polynucleotide, the fourth polynucleotide, and any combination thereof, is introduced into the cell as a peptide-polynucleotide complex, wherein the peptide-polynucleotide complex comprises at least one peptide.

53. The method of claim 52, wherein the at least one peptide of the peptide-polynucleotide complex comprises at least one selected from the group consisting of: a cell penetrating peptide (CPP), an organellar targeting peptide, a mitochondrial targeting peptide, a histidine-rich peptide, a lysine-rich peptide, and any combination thereof.

54. The method of any one of claims 36-53, wherein the method further comprises:
a. introducing into the mitochondrion of the cell a recombinant DNA construct comprising:
i. a first additional polynucleotide encoding at least one guide polynucleotide, wherein the at least one guide polynucleotide directs a polynucleotide guided polypeptide to cleave at least one target sequence present in an organelle genome; and
ii. a second additional polynucleotide encoding the polynucleotide guided polypeptide, wherein the polynucleotide guided polypeptide, when associated with the guide polynucleotide, cleaves the at least one target sequence.

55. The method of any one of claims 36-53, wherein the method further comprises:
a. introducing into a nucleus of the cell:
i. a first additional polynucleotide encoding a modified polynucleotide guided polypeptide, wherein the modified polynucleotide guided polypeptide comprises a polynucleotide guided polypeptide operably linked to a mitochondrial targeting peptide, wherein the polynucleotide guided polypeptide when associated with a guide RNA, cleaves at least one target sequence present in the mitochondrial genome; and
ii. a second additional polynucleotide encoding at least one guide RNA, wherein the at least one guide RNA directs the polynucleotide guided polypeptide to cleave the at least one target sequence present in the mitochondrial genome.

56. The method of any one of claims 36-53, wherein the method further comprises:
a. introducing into a nucleus of the cell:
i. a first additional polynucleotide encoding a modified polynucleotide guided polypeptide, wherein the modified polynucleotide guided polypeptide comprises a polynucleotide guided polypeptide operably linked to a mitochondrial targeting peptide, wherein the polynucleotide guided polypeptide when associated with a guide RNA, cleaves at least one target sequence present in the mitochondrial genome; and
b. introducing into the mitochondrion of the cell:
i. a second additional polynucleotide encoding at least one guide RNA, wherein the at least one guide RNA directs the polynucleotide guided polypeptide to cleave the at least one target sequence present in the mitochondrial genome.

57. The method of any one of claims 54-56, wherein the polynucleotide guided polypeptide is at least one selected from the group consisting of: a Cas9 protein, a Cas3 protein, a MAD2 protein, a MAD7 protein, a CRISPR nuclease, a nuclease domain of a Cas protein, a Cpf1 protein, an Argonaute, modified versions thereof, a biologically active fragment thereof, and any combination thereof.

58. The method of any one of claims 54-57, wherein homologous recombination of all or part of the third polynucleotide, or all or part of the fourth polynucleotide, or both, with endogenous mitochondrial DNA sequence results in an edited mitochondrial genome lacking the at least one target sequence.

59. The method of any one of claims 54-58, wherein the method further comprises:
a. introducing into a nucleus of the cell:
i. the second additional polynucleotide, wherein the second additional polynucleotide encodes a modified site-directed nuclease, wherein the modified site-directed nuclease comprises a site-directed nuclease operably linked to a mitochondrial targeting peptide, wherein the site-directed nuclease cleaves at least one target sequence present in the mitochondrial genome.

60. The method of claim 59, wherein the site-directed nuclease is at least one selected from the group consisting of: a TALEN, a Zinc-Finger Nuclease, a Meganuclease, a restriction enzyme, and any combination thereof.

61. The method of any one of claims 36-60, wherein the method further comprises:
a. introducing into a nucleus of the cell:
i. a third additional polynucleotide encoding a selectable marker polypeptide that provides tolerance to a selective agent; and
b. selecting a cell that grows in the presence of the selective agent.

62. The method of any one of claims 36-61, wherein the first polynucleotide encoding the phosphite dehydrogenase or a biologically active fragment thereof further comprises a T7 RNA polymerase promoter, wherein expression of the phosphite dehydrogenase or a biologically active fragment thereof is under control of the T7 RNA polymerase promoter.

63. The method of claim 62, further comprising:
a. introducing into a nucleus of the cell:
i. a fourth additional polynucleotide encoding a modified T7 RNA polymerase, wherein the modified T7 RNA polymerase comprises a T7 RNA polymerase operably linked to a mitochondrial targeting peptide.

64. The method of claim 63, wherein the mitochondrial targeting peptide is encoded by SEQ ID NO: 38.

65. The method of anyone of claims 36-64, wherein the phosphite dehydrogenase or a biologically active fragment thereof comprises an amino acid sequence with at least 70%, 80%, 85%, 90%, 91%, 92%, 93%, 94%, 95% or 99% sequence identity to SEQ ID NO: 29, 48, 49, 50, 51, 52, 53, 54, 55, 56, 57, 58, 59, or 60.

66. The method of any one of claims 36-65, wherein the first polynucleotide encoding the phosphite dehydrogenase or a biologically active fragment thereof further comprises SEQ ID NO: 44 or SEQ ID NO: 45.

67. The method of any one of claims 36-66, wherein a sequence encoding a start codon of the phosphite dehydrogenase or the biologically active fragment thereof is replaced with a sequence encoding a mitochondrial RNA editing site.

68. The method of claim 67, wherein the mitochondrial RNA editing site is from a mitochondrial nad4L gene or a mitochondrial cox2 gene.

69. The method of claim 67, wherein the sequence encoding the mitochondrial RNA editing site comprises SEQ ID NO: 46.

70. The method of any one of claims 36-67, wherein the first polynucleotide encoding the phosphite dehydrogenase or a biologically active fragment thereof comprises SEQ ID NO: 47.

71. The method of any one of claims 36-70, wherein the cell is grown simultaneously in a presence of a selective agent and in a presence of a phosphite as a primary phosphorus source, wherein phosphate is present at less than 3 mg/liter.

72. The method of any one of the claims 36-71, wherein the cell is grown sequentially first in a presence of a selective agent and subsequently in a presence of a phosphite as a primary phosphorus source, wherein phosphate is present at less than 3 mg/liter.

73. The method of any one of claims 61-72, wherein the selectable marker polypeptide is hygromycin phosphotransferase (HPT) and the selective agent is hygromycin.

74. The method of any one of claims 36-73, wherein the method further comprises removing the first polynucleotide encoding the phosphite dehydrogenase or a biologically active fragment thereof after inserting the second polynucleotide.

75. The method of any one of claims 36-74, wherein the method further comprises selecting a cell that comprises a plurality of mitochondrial genomes, wherein at least 50%, 60%, 70%, 80%, 90%, or 100% of the plurality of mitochondrial genomes comprise the edited mitochondrial genome.

76. The method of any one of claims 36-75, wherein the method further comprises selecting a cell that is homoplasmic for the edited mitochondrial genome.

77. A cell produced by the method of any one of claims 36-76, wherein the cell is a yeast cell, an algal cell, a plant cell, an insect cell, a non-human animal cell, an isolated and purified human cell, or a mammalian tissue culture cell.

78. The cell of claim 77, wherein the cell is a plant cell.

79. A plant, cell, tissue, propagation material, seed, root, leaf, flower, fruit, pollen, progeny, or part thereof, produced from the plant cell of claim 78, wherein the plant, cell, tissue, propagation material, seed, root, leaf, flower, fruit, pollen, progeny, or part thereof comprises the edited mitochondrial genome.

80. A method of controlling weeds, the method comprising:
a. growing a plurality of plants in a presence of a phosphite, wherein at least one plant of the plurality of plants comprises a mitochondrion having an exogenous polynucleotide that encodes phosphite dehydrogenase or a biologically active fragment thereof, wherein the presence of the phosphite is sufficient to selectively promote growth of the at least one plant of the plurality of plants, resulting in an increased growth of the at least one plant of the plurality of plants relative to plants lacking phosphite dehydrogenase or a biologically active fragment thereof.

81. The method of claim 80, further comprising applying the phosphite to the plant, the plurality of plants, soil adjacent to the plant, or any combination thereof.

82. The method of claim 81, wherein the phosphite is applied as a foliar fertilizer.

83. The method of claim 81, wherein the phosphite is applied as a soil amendment.

84. The method of any one of claims 80-83, wherein the at least one plant of the plurality of plants is selected from the group consisting of: wheat, maize, rice, barley, sorghum, rye, sugarcane, potato, tomato, canola, broccoli, cauliflower, and soybean.

85. The method of any one of claims 80-84, wherein a plant lacking phosphite dehydrogenase or a biologically active fragment thereof is a weed.

86. The method of any one of claims 80-85, wherein the phosphite dehydrogenase or a biologically active fragment thereof comprises an amino acid sequence with at least 80%, 85%, 90%, 91%, 92%, 93%, 94%, or 95% sequence identity to SEQ ID NO: 29, 48, 49, 50, 51, 52, 53, 54, 55, 56, 57, 58, 59, or 60.

87. The method of claim 86, wherein the phosphite dehydrogenase or a biologically active fragment thereof comprises an amino acid sequence of SEQ ID NO: 29.

88. A method of using the cell of any one of claims 1-31, or the method of any one of claims 36-78 for growing a plant.

89. A field or a greenhouse comprising the plant of claim 88.

90. A food product comprising the cell of any one of claims 1-31.

91. A field comprising the cell of any one of claims 1-31.

92. A kit comprising the cell of any one of claims 1-31 or the transgenic plant or parts thereof of anyone of claims 32-35.


SEQUENCE LISTING (135 sequences)

SEQ ID NO: 1 - protein, 1368 aa, Streptococcus pyogenes [sequence omitted]

SEQ ID NO: 2 - protein, 1334 aa, synthetic construct
MSSLTKFTNKYSKQLTIKNELIPVGKTLENIKENGLIDGDEQLNENYQKAKIIVDDFLRDFINKALNNTQIGNWRELADALNKEDEDNIEKLQDKIRGIIVSKFETFDLFSSYSIKKDEKIIDDDNDVEEEELDLGKKTSSFKYIFKKNLFKLVLPSYLKTTNQDKLKIISSFDNFSTYFRGFFENRKNIFTKKPISTSIAYRIVHDNFPKFLDNIRCFNVWQTECPQLIVKADNYLKSKNVIAKDKSLANYFTVGAYDYFLSQNGIDFYNNIIGGLPAFAGHEKIQGLNEFINQECQKDSELKSKLKNRHAFKMAVLFKQILSDREKSFVIDEFESDAQVIDAVKNFYAEQCKDNNVIFNLLNLIKNIAFLSDDELDGIFIEGKYLSSVSQKLYSDWSKLRNDIEDSANSKQGNKELAKKIKTNKGDVEKAISKYEFSLSELNSIVHDNTKFSDLLSCTLHKVASEKLVKVNEGDWPKHLKNNEEKQKIKEPLDALLEIYNTLLIFNCKSFNKNGNFYVDYDRCINELSSVVYLYNKTRNYCTKKPYNTDKFKLNFNSPQLGEGFSKSKENDCLTLLFKKDDNYYVGIIRKGAKINFDDTQAIADNTDNCIFKMNYFLLKDAKKFIPKCSIQLKEVKAHFKKSEDDYILSDKEKFASPLVIKKSTFLLATAHVKGKKGNIKKFQKEYSKENPTEYRNSLNEWIAFCKEFLKTYKAATIFDITTLKKAEEYADIVEFYKDVDNLCYKLEFCPIKTSFIENLIDNGDLYLFRINNKDFSSKSTGTKNLHTLYLQAIFDERNLNNPTIMLNGGAELFYRKESIEQKNRITHKAGSILVNKVCKDGTSLDDKIRNEIYQYENKFIDTLSDEAKKVLPNVIKKEATHDITKDKRFTSDKFFFHCPLTINYKEGDTKQFNNEVLSFLRGNPDINIIGIDRGERNLIYVTVINQKGEILDSVSFNTVTNKSSKIEQTVDYEEKLAVREKERIEAKRSWDSISKIATLKEGYLSAIVHEICLLMIKHNAIVVLENLNAGFKRIRGGLSEKSVYQKFEKMLINKLNYFVSKKESDWNKPSGLLNGLQLSDQFESFEKLGIQSGFIFYVPAAYTSKIDPTTGFANVLNLSKVRNVDAIKSFFSNFNEISYSKKEALFKFSFDLDSLSKKGFSSFVKFSKSKWNVYTFGERIIKPKNKQGYREDKRINLTFEMKKLLNEYKVSFDLENNLIPNLTSANLKDTFWKELFFIFKTTLQLRNSVTNGKEDVLISPVKNAKGEFFVSGTHNKTLPQDCDANGAYHIALKGLMILERNNLVREEKDTKKIMAISNVDWFEYVQKRRGVL

SEQ ID NO: 3 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 4 - protein, 9 aa, synthetic construct
LAGLIDADG

SEQ ID NO: 5 - protein, 36 aa, synthetic construct
DEVDFQGPCNDSSDPLVVAASIIGILHLILWILDRL

SEQ ID NO: 6 - protein, 4 aa, synthetic construct
DEVD

SEQ ID NO: 7 - RNA, 27 nt, Triticum aestivum
  source 1..27: mol_type = genomic RNA
acttttgacagttatacgattccagaa

SEQ ID NO: 8 - RNA, 27 nt, Triticum aestivum
  source 1..27: mol_type = genomic RNA
tgggctgtaccttcctcaggtgtcaaa

SEQ ID NO: 9 - RNA, 27 nt, Triticum aestivum
  source 1..27: mol_type = genomic RNA
gctgtacctggtcgttcaaatcttacc

SEQ ID NO: 10 - protein, 16 aa, Drosophila melanogaster
RQIKIWFQNRRMKWKK

SEQ ID NO: 11 - protein, 28 aa, synthetic construct
KHKHKHKHKHKHKHKHKHMFAFQYLLVM

SEQ ID NO: 12 - protein, 9 aa, Human immunodeficiency virus 1
RKKRRQRRR

SEQ ID NO: 13 - protein, 9 aa, synthetic construct
RRRRRRRRR

SEQ ID NO: 14 - protein, 21 aa, synthetic construct
KETWWETWWTEWSQPKKKRKV

SEQ ID NO: 15 - protein, 27 aa, synthetic construct
GALFLGFLGAAGSTMGAWSQPKKKRKV

SEQ ID NO: 16 - protein, 6 aa, synthetic construct
VRLPPP

SEQ ID NO: 17 - protein, 27 aa, synthetic construct
GWTLNSAGYLLGKINLKALAALAKKIL

SEQ ID NO: 18 - protein, 18 aa, synthetic construct
KLALKLALKALKAALKLA

SEQ ID NO: 19 - protein, 12 aa, synthetic construct
PLILLRLLRGQF

SEQ ID NO: 20 - protein, 12 aa, synthetic construct
PLIYLRLLRGQF

SEQ ID NO: 21 - protein, 15 aa, synthetic construct
KLWMRWYSPTTRRYG

SEQ ID NO: 22 - protein, 23 aa, synthetic construct
MGLGLHLLVLAAALQGAKKKRKV

SEQ ID NO: 23 - protein, 18 aa, synthetic construct
KLALKLALKALKAALKLA

SEQ ID NO: 24 - protein, 18 aa, Mus musculus
LLIILRRRIRKQAHAHSK

SEQ ID NO: 25 - protein, 10 aa, synthetic construct
RRIPNRRPRR

SEQ ID NO: 26 - protein, 11 aa, synthetic construct
KKLFKKILKYL

SEQ ID NO: 27 - protein, 18 aa, synthetic construct
RKKRRQRRRRKKRRQRRR

SEQ ID NO: 28 - DNA, 1011 bp, synthetic construct
  source 1..1011: mol_type = other DNA
atgctgcctaaactcgttataactcaccgagtacatgatgagatcctgcaactgctggcgccacattgcgagctgatgaccaaccaaaccgacagcacactgacacgcgaggaaattctgcgtcgatgtcgtgatgctcaagcgatgatggcgttcatgcccgatcgagtcgatgcagactttcttcaagcctgccctgagctgcgtgtagtcggctgcgcgctcaagggcttcgacaatttcgatgtggacgcctgtactgcccgtggggtctggctgaccttcgtgcctgatctgttgacagtcccaactgccgagctggcgatcggactggcggtggggctggggcgacatctgcgagcagcagatgcgttcgtccgttctggcgagttccaaggctggcaaccacaattctatggcacagggctggataacgctacagtcggcatccttggcatgggcgccatcggactggccatggctgatcgattgcaaggatggggcgcgaccctgcaatatcacgaggcgaaggctctggatacacaaaccgagcaacgactcggcctgcgtcaagtggcgtgcagcgaactcttcgccagctcggacttcatcctgctggcgcttcccttgaatgccgatacccaacatctggtcaacgccgagctgcttgccctcgtacgaccaggcgctctgcttgtaaacccctgtcgtggttcggtagtggatgaagccgccgtgctcgcggcgcttgagcgaggccaactcggcgggtatgcggcggatgtattcgaaatggaagactgggctcgtgcggaccgaccacgactgatcgatcctgcgctgctcgcgcatcctaatacactgttcactccacatatagggtcggcagtgcgtgcggtgcgtctggagattgaacgttgtgcagcgcaaaacatcatccaagtattggcaggtgcgcgtccaatcaacgctgcgaaccgtctgcccaaggccgagcctgccgcatgttga

SEQ ID NO: 29 - protein, 336 aa, Pseudomonas stutzeri
MLPKLVITHRVHDEILQLLAPHCELMTNQTDSTLTREEILRRCRDAQAMMAFMPDRVDADFLQACPELRVVGCALKGFDNFDVDACTARGVWLTFVPDLLTVPTAELAIGLAVGLGRHLRAADAFVRSGEFQGWQPQFYGTGLDNATVGILGMGAIGLAMADRLQGWGATLQYHEAKALDTQTEQRLGLRQVACSELFASSDFILLALPLNADTQHLVNAELLALVRPGALLVNPCRGSVVDEAAVLAALERGQLGGYAADVFEMEDWARADRPRLIDPALLAHPNTLFTPHIGSAVRAVRLEIERCAAQNIIQVLAGARPINAANRLPKAEPAAC

SEQ ID NO: 30 - DNA, 928 bp, Oryza sativa
  source 1..928: mol_type = genomic DNA
tctgcttgaaagcctgcagagtccaattttgagtattttcagttagaatctagagtcagcctattcagttcttagcccttaagggtaaggcagggggtaatatggatagtctctgtccctgtattcacattccaccttcaacaaagtgttgatttcccgtaaagctaactgtagtcctttaagtaagtagatatcttaggcaagttagcaatctcgttatattaccaaggccttcccttctattgtagaaagagttctcagccatctaattgcagtgccagttgccagctatccagtttcatttgaagttgctgggggtccaaacgagctagttgcttttattcgtcctataagtccttccacaagcgagtcaatagggtgctggctagttgtagttgttggcgtgcctttcctttcatcttgaatattaataaatatttggataaattactttagaataagaagttcatgttttatagactagtaagataactaactagttaagtaatacgaatccatactaggaaaatgaaaatgtgagtcctaggcactggaattggttctcttctccctaatccctataagccagaaagggtaataggcttcagtgtaagcatttccttcaagcaagtcatctcaagttttaaattctagagaatagctccgatcaacccattttagtttggttctgcaattcattcgcataaatgaaaaaaaaagcgagatgtgcacgaaagaagatcatagttcagctttaaaatggtggtgtccctgtgttagtaagtggttgaaatagctcatgggagtgtctgccccattcgataatggcatttatgatctagtggagtgagtgattgtgtggtgttcagtctaaggctttttgaaaagcggatttctcccttctctcatccatcgtctttgttaaagt

SEQ ID NO: 31 - DNA, 872 bp, Oryza sativa
  source 1..872: mol_type = genomic DNA
atagacctttttatttttcgtcattcgatcacgaaaacagggattctggaacggccaagaatcccagcggttgttcgggtcgaaaaaccgagaacaagacatgccacaaagtggcagatgaaggcaggggggagagcctagtcctcaacctcttcttccccaaaaggtagttatgaacgtgccaaacttattggatttattcttggaatgctcataaccacctttactctttttttcattctttactcagaggaagccatgccgtttggagaagagcaccaagtggggggagtgtggagtccccgaaagaggagctttctaaaggcaagagaaaagctccgatggagcccttggagctacagggaccaccaacccttcgcagcttggacgatttgattcttgtgccactcagccctgaggagggcgcctgctcgacccagtcaggtactactccgccgccggccccgagtaactctgcgggggtcggtgcagccctttcttctattccggagtgcataaacaaggatcctcaaaaagcgaaatcatttcgttaatggcatttcagaaatgagtcataggcgcctgtacaatgacagaatagagagtcctttttttccagaatgaatcattctattcaaatctcacaagttctctttacgcgtcttctaggggcattgttgaacgcaatctgcaggaacaagaaatgattctttcttattttgaaacagaattcaaaataaaggaggatttaattcggttgctttatgaaggccgacgccgtgccgatagatacgttatacacgaaacgaaaatagccagtacggtggacgcgttcctttccaaaaagggattatcaggagctcccagtgccg

SEQ ID NO: 32 - DNA, 33 bp, synthetic construct
  source 1..33: mol_type = other DNA
aaggtctcgaattcaatgggcccttagctcgag

SEQ ID NO: 33 - DNA, 37 bp, synthetic construct
  source 1..37: mol_type = other DNA
gagctcggtaccaaaggcgcgccaaacaattgtcgac

SEQ ID NO: 34 - DNA, 6541 bp, synthetic construct
  source 1..6541: mol_type = other DNA
ttcaatgggcccctgttttcgaagctatagcataggtttagtgaggtttaggtactttgacacctaaaccgatttgaaatgcgaaatatcgcatttctgtaagcataactagtattgtccctcgccgtgcacgcgcacgtgcacgcgcgggtgatgtgcgcatgcgtatgcgcacgtgatctattggtgcgcgcatgcgcacgcgttctattacgcgcgagcgcacacgcggatgcgtgtgcgcgtgtattccctcccttacttgaatgatccccccctcaaggggtatcatcccagtaagctcgggtctttcataggaagggagcaggcccccgctcccttccgtccatcagtgatttattcaaaacccgaaatcgagttgagttgaatcgggttcaagtcaactagatgaagggtttctttgtagtgagggaacgagttactgaacacagtgaaatatcacgttttgtcatgtcatgcacatgtgtctttccctcgatcccgaaccttgactggacgtataggtatgcggtatgccaatgacagttatcgaagctgccatcagttatgcatttatggattcgggtctagtgaatagggtatgcctaagcgcccacccgagattggactcgagggtgggtaagacccggagcgaggttgtccacgagcggaggcccctcgaagaggcgagcccggaaaccactcgtttttttagtacccaaaaccctagtgttttaaagtgagagggattctacctttggggaaggtaggattcctcggagcaaaggaaaactaatgttgaatgttttcgtggaagtgagataagtacttccttgggaagggagtacttatcggagtaaaggaaactgcggaggttcttgttgtaaggaggctagtcccgttgttaggaagagttagcggtgtttacaccggtgtcacgtgtacggggatacgtgtattgagaaaaaaggccgagaaggtcgagggggtcatcccattggccagactgagcatcaagccagccaagaagtaaaagctgagaaggagtgactcgcatgagtcaacacttactactcaggtccggtagagcaatctcaaattatcatatagaaatgttaatgttatgatttcggtattgatcaaaaggtgctgggaccttagggcatacattagtgccatgccctattgcggaacggtcgtatcctggtaacctagcccccgtaagagctctacctaatcgtcggggtagaaggctgtgcttattctcggcaaatagctaagtcgacaccccgagggagcaactcaactcttcgtagatcaaaacaagtgttcactggaaagtggatcaaagaaaaaaacttcttcgtttcgttggaaaaaccgacgccaatatcatattgactctctctcgtccaataagagtttccgagagttactttattcaaattctctcctttccaaagctccacaaggcaggcaaaaagagtaataggacaacaagcaatcttgtctttcatttatttggagttctttctttgttgagatggaaatcgacgttcttttgaaaagggctaggtagtttgcacgcaggcaaaacttcttcatgaaaggtaataaatagacttttttttcatgggtttcttaatgactagtcgttcgtttgaagccttaagaaaccggcagttttttttccgaatgaccttatttcgagaatcaactaaccgacaaatccgtagcccaggtgattcgctgcctccctctcgccaaaatgggatgaatcttctcatgcagcttttttcttgttcagggcgcagcgaagccaatttccatcaaggcaagggggtaaataagggggaagaggagttgtcacgatagaaaagagaaacttttgacagttatacgattccagaagtgagtaagggagaggagctgttcaccggggtggtgcctatcctggtcgagctggatggtgatgtaaacggtcataaattcagtgtgtccggtgaaggtgaaggtgatgccacctatggtaagctgacccttaagttcatctgtaccaccggaaagctgcctgtgccttggcctaccctcgtgaccaccctgacatatggagtgcaatgtttcagtcgttatcctgatcatatgaagcaacatgatttctttaaatccgccatgcctgaaggttatgtccaagagcgtaccatattctttaaagatgatggtaactataagacccgtgccgaggtgaagttcgagggtgataccctggtgaaccgtattgagcttaagggtatcgatttcaaggaggatggaaacatcctggggcataagctggagtataactataacagtcataacgtctatatcatggccgataagcaaaagaacggtatcaaggtgaacttcaagatccgtcataatatcgaagatggaagtgtgcaactcgccgatcattatcaacaaaacacccctatcggtgatggtcctgtgctgctgcctgataaccattatctgagtacccaatccgccctgagtaaagatcctaacgagaagcgtgatcaaatggtactgcttgagttcgttaccgccgccgggatcactctcggtatggatgagctgtataagtaatagacggatgagactgatcacacctgatcagtgatcaattctggcacaatgaatttacgagttattttacacaatgaatttacaagcagatgagtttgcaacggtagacctatctcctgaaaagagttcagtaaacaagggaacgaagcgaccgataacgtcccctcggggaggagtgttttggatccgtaaccatggctttgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 35 - DNA, 1026 bp, synthetic construct
  source 1..1026: mol_type = other DNA
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatag

SEQ ID NO: 36 - DNA, 1082 bp, synthetic construct
  source 1..1082: mol_type = other DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccggcgcgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagat

SEQ ID NO: 37 - DNA, 209 bp, synthetic construct
  source 1..209: mol_type = other DNA
gatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaatta

SEQ ID NO: 38 - DNA, 108 bp, Arabidopsis thaliana
  source 1..108: mol_type = genomic DNA
atgtttaaacaagcttctcgtctcctctcccgatctgtcgccgccgcatcttccaaatcggtgacgactcgtgccttttcaacggaacttccatcgacgctcgattcc

SEQ ID NO: 39 - DNA, 2015 bp, synthetic construct
  source 1..2015: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcc

SEQ ID NO: 40 - DNA, 253 bp, unidentified
  source 1..253: mol_type = unassigned DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 41 - DNA, 5064 bp, synthetic construct
  source 1..5064: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatgtttaaacaagcttctcgtctcctctcccgatctgtcgccgccgcatcttccaaatcggtgacgactcgtgccttttcaacggaacttccatcgacgctcgattccaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaagcatgctgaagcggccgggtaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 42 - DNA, 18 bp, unidentified
  source 1..18: mol_type = unassigned DNA
taatacgactcactatag

SEQ ID NO: 43 - DNA, 41 bp, unidentified
  source 1..41: mol_type = unassigned DNA
aaccccttggggcctctaaacgggtcttgaggggttttttg

SEQ ID NO: 44 - DNA, 919 bp, synthetic construct
  source 1..919: mol_type = other DNA
tctgcttgaaagcctgcagagtccaattttgagtattttcagttagaatctagagtcagcctattcagttcttagcccttaagggtaaggcagggggtaatatggatagtctctgtccctgtattcacattccaccttcaacaaagtgttgatttcccgtaaagctaactgtagtcctttaagtaagtagatatcttaggcaagttagcaatctcgttatattaccaaggccttcccttctattgtagaaagagttctcagccatctaattgcagtgccagttgccagctatccagtttcatttgaagttgctgggggtccaaacgagctagttgcttttattcgtcctataagtccttccacaagcgagtcaatagggtgctggctagttgtagttgttggcgtgcctttcctttcatcttgaatattaataaatatttggataaattactttagaataagaagttcatgttttaatacgactcactatagtaagtaatacgaatccatactaggaaaatgaaaatgtgagtcctaggcactggaattggttctcttctccctaatccctataagccagaaagggtaataggcttcagtgtaagcatttccttcaagcaagtcatctcaagttttaaattctagagaatagctccgatcaacccattttagtttggttctgcaattcattcgcataaatgaaaaaaaaagcgagatgtgcacgaaagaagatcatagttcagctttaaaatggtggtgtccctgtgttagtaagtggttgaaatagctcatgggagtgtctgccccattcgataatggcatttatgatctagtggagtgagtgattgtgtggtgttcagtctaaggctttttgaaaagcggatttctcccttctctcatccatcgtctttgttaaagt

SEQ ID NO: 45 - DNA, 946 bp, synthetic construct
  source 1..946: mol_type = other DNA
tctgcttgaaagcctgcagagtccaattttgagtattttcagttagaatctagagtcagcctattcagttcttagcccttaagggtaaggcagggggtaatatggatagtctctgtccctgtattcacattccaccttcaacaaagtgttgatttcccgtaaagctaactgtagtcctttaagtaagtagatatcttaggcaagttagcaatctcgttatattaccaaggccttcccttctattgtagaaagagttctcagccatctaattgcagtgccagttgccagctatccagtttcatttgaagttgctgggggtccaaacgagctagttgcttttattcgtcctataagtccttccacaagcgagtcaatagggtgctggctagttgtagttgttggcgtgcctttcctttcatcttgaatattaataaatatttggataaattactttagaataagaagttcatgttttatagactagtaagataactaactagttaagtaatacgaatccatactaggaaaatgaaaatgtgagtcctaggcactggaattggttctcttctccctaatccctataagccagaaagggtaataggcttcagtgtaagcatttccttcaagcaagtcatctcaagttttaaattctagagaatagctccgatcaacccattttagtttggttctgcaattcattcgcataaatgaaaaaaaaagcgagatgtgcacgaaagaagatcatagttcagctttaaaatggtggtgtccctgtgttagtaagtggttgaaatagctcatgggagtgtctgccccattcgataatggcataatacgactcactatagtttatgatctagtggagtgagtgattgtgtggtgttcagtctaaggctttttgaaaagcggatttctcccttctctcatccatcgtctttgttaaagt

SEQ ID NO: 46 - DNA, 27 bp, Triticum aestivum
  source 1..27: mol_type = genomic DNA
acttttgacagttatacgattccagaa

SEQ ID NO: 47 - DNA, 1035 bp, synthetic construct
  source 1..1035: mol_type = other DNA
acttttgacagttatacgattccagaactgcctaaactcgttataactcaccgagtacatgatgagatcctgcaactgctggcgccacattgcgagctgatgaccaaccaaaccgacagcacactgacacgcgaggaaattctgcgtcgatgtcgtgatgctcaagcgatgatggcgttcatgcccgatcgagtcgatgcagactttcttcaagcctgccctgagctgcgtgtagtcggctgcgcgctcaagggcttcgacaatttcgatgtggacgcctgtactgcccgtggggtctggctgaccttcgtgcctgatctgttgacagtcccaactgccgagctggcgatcggactggcggtggggctggggcgacatctgcgagcagcagatgcgttcgtccgttctggcgagttccaaggctggcaaccacaattctatggcacagggctggataacgctacagtcggcatccttggcatgggcgccatcggactggccatggctgatcgattgcaaggatggggcgcgaccctgcaatatcacgaggcgaaggctctggatacacaaaccgagcaacgactcggcctgcgtcaagtggcgtgcagcgaactcttcgccagctcggacttcatcctgctggcgcttcccttgaatgccgatacccaacatctggtcaacgccgagctgcttgccctcgtacgaccaggcgctctgcttgtaaacccctgtcgtggttcggtagtggatgaagccgccgtgctcgcggcgcttgagcgaggccaactcggcgggtatgcggcggatgtattcgaaatggaagactgggctcgtgcggaccgaccacgactgatcgatcctgcgctgctcgcgcatcctaatacactgttcactccacatatagggtcggcagtgcgtgcggtgcgtctggagattgaacgttgtgcagcgcaaaacatcatccaagtattggcaggtgcgcgtccaatcaacgctgcgaaccgtctgcccaaggccgagcctgccgcatgttga

SEQ ID NO: 48 - protein, 335 aa, Acinetobacter radioresistens
MKQKIVLTHWVHPEIIDYLQSVADVVPNMTRDTMSRAELLERAKDADALMVFMPDSIDDDFLASCPKLKIVSAALKGYDNFDVDACTRRGIWFSIVPDLLTIPTAELTIGLLLGLTRHLAEGDRRIRTHGFNGWRPELYGTGLTGRTLGIIGMGAVGRAIAKRLSSFDMRVLYCDDIALNQEQEKAWNARQVSLDELLSSSDFVVPMLPMTPQTLHLLNAETIGTMRTGSYLINACRGSVVDELAVAEALESGKLAGYAADVFELEEWIRVDRPTAIPQELLTNTAQTFFTPHLGSAVDDVRFEIEQLAANNILQALTGQRPSDAINNPILEGVN

SEQ ID NO: 49 - protein, 333 aa, Alcaligenes faecalis
MKPRIVTTHRIHPDTLALLETAAEVISNQSDSTMSREEVLLRTNDADGMMVFMPDSIDADFLSACPNLKVIGAALKGYDNFDVEACTRHGIWFTIVPDLLTSPTAELTIGLLLSITRNMLQGDNYIRSRQFNGWTPRFYGTGLTGKTAGIIGTGAVGRAVAKRLAAFDMQIQYTDPQPLPQESERAWNASRTSLDQLLATSDFIIPMLPMSSDTHHTINARALDRMKPGAYLVNACRGSIVDERAVVHALRTGHLGGYAADVFEMEEWARPDRPHSIPDELLDPALPTFFTPHLGSAVKSVRMEIEREAALSILEALQGRIPRGAVNHVGAGR

SEQ ID NO: 50 - protein, 335 aa, unidentified; identical to SEQ ID NO: 48

SEQ ID NO: 51 - protein, 335 aa, Gallionella ferruginea
MKPKIVITSWVHPQTLDMLRPHCDVVANETRERLSREEIIKRCSDAVAVMTFMPDSIDDAFLAECPQLRLVACALKGYDNYDVAACTRRGVRITNVPDLLTIPTAELTVGLLIGLTRKVLQGDRFVRSGQFTGWRPMLYGAGLTGRTLGIIGMGAVGRAIAARLQGYEMELLYTDPQPLPPELEARLGLRKVGLVQLLAESDYVVPMVPYTQDTLHMINAASLSIMKPGAYLVNTCRGSVVDEKAVADALDSGKLAGYAADVFELEEWMRPDRPESISERLLSNTELTLFTPHIGSAVDTVRLAIEMEAATNILQVLKGQIPQGAINHPLDKVAV

SEQ ID NO: 52 - protein, 336 aa, Janthinobacterium sp.
MKPKIVITHWVHPEIVEMLSSVAEVVTNDTLETLPREELLRRSKDADAVMAFMPDSVDDSFLAACPKLKIVFAALKGYDNFDVDACTKRGVWFGIVPDLLTVPTAELTVGLLLGLTRHVMAGDDHVRSGTFHGWRPKLYGAGLAGSTIGIIGMGRVGKAIAKRLSGFEMNAVYCDSVPLNPVDEQAWNARQVSFDELLTCSDFVVPMLPMTSDTFHLIDAHAISKMRRGSYLLNTSRGSVVDENAVVEALNQGHLAGYAADVFEMEEWARPDRPLTVPQALLNNRTQTLFTPHVGSGVKKVRLEIERYSAHSILQALAGQRPDGALNEPLKASVAA

SEQ ID NO: 53 - protein, 336 aa, Klebsiella pneumoniae
MLPKLVITHRVHDEILQLLAPHCELVTNQTDSTLTREEILRRCRDAQAMMAFMPDRVDADFLQACPELRVVGCALKGFDNFDVDACTARGVWLTFVPDLLTVPTAELAIGLAVGLGRHLRAADAFVRSGEFQGWQPQFYGTGLDNATVGILGMGAIGLAMAERLQGWGATLQYHEAKALDTQTEQRLGLRQVACSELFASSDFILLALPLNADTQHLVNAELLALVRPGALLVNPCRGSVVDEAAVLAALERGQLGGYAADVFEMEDWARADRPRLIDPALLAHPNTLFTPHIGSAVRAVRLEIERCAAQNIIQVLAGARPINAANRLPKAEPAAC

SEQ ID NO: 54 - protein, 336 aa, Marinobacter algicola
MKPRVVITHRVHDSILASLEPHCELITNQSAVTLPPDSVRARAATADAMMAFMPDRVSEEFLVACPDLKVIGAALKGFDNFDVDACTRHGVWLTFVPDLLTVPTAELTVGLTIGLIRQIRPADQFVRSGEFQGWQPQFYGLGIEGSTIGIVGMGAIGKAVATRLQGWGARVLYSQPESLPAAEEGALGLSRSELDDLLAESDIVILALALNEHTLHTLNADRLRQMKRGSFLINPCRGSVVDEAAVLQSLTYGHLSGYAADVFEMEDWARPDRPQRIDPALLAHPNTLFTAHTGSAVRDVRFAIELRAADNILQALRGHQPQDAVNSPLEPKGTVC

SEQ ID NO: 55 - protein, 339 aa, Methylobacterium extorquens
MRFKVVVTNPVFPETREILEGLCDVDINPGPEPWPAAEVRARCSDADALLAFMTDCVDAGFLEACPRLKVVACALKGWDNFDVEACTRSGIWLTAVPDLLTEPTAELAVGLAIGLCRNVVAGDRAVRAGFDGWRPRLYGSGLYGSVVGVAGMGKVGRAITRRLKGFGARELLYFDEQALPASAEAELGACRVSWDTLVGRSDVLILALPLTPDTRHMLDAAALAAASPGLRIVNAGRGSVVDEAAVAEALAEGRLGGYAADVFEMEDWALDDRPRRIAPGLLTVEDRTLFTPHLGSGVVDTRRRIEAAAAHNLLDALKGLVPADSINHPESLRGFDGAN

SEQ ID NO: 56 - protein, 332 aa, Nostoc sp.
MKPKVVITNWVHPEVIELLKPSCEVIANPSKEALSREEILQRAKDAEALMVFMPDTIDEAFLRECPKLKIIAAALKGYDNFDVAACTHRGIWFTIVPSLLSAPTAEITIGLLIGLGRQMLEGDRFIRTGKFTGWRPQFYSLGLANRTLGIVGMGALGKAIAGRLAGFEMQLLYSDPVALPPEQEATGNISRVPFETLIESSDFVVLVVPLQPATLHLINANTLAKMKPGSFLINPCRGSVVDEQAVCKALESGHLAGYAADVFEMEDWYRSDRPHNIPQPLLENTKQTFFTPHIGSAVDELRHNIALEAAQNILQALQGQKPQGAVNYLRES

SEQ ID NO: 57 - protein, 331 aa, Oxalobacter formigenes
MNKQKVVLTHWVHPEIVEMLQEKTDVVANLSRKTFTRDELLERAAAADALMAFMPDCIDEDFLKACPKLKVIGAALKGYDNFDVKACTERGVWLTIAPDLLTIPTAELTVGLVLAITRNMLEGDRHIRSGQFNGWRPELYGLGLHKRTAGIIGMGFVGKAVAERLKGFGMDILYADQSPLSQEEERELGLTRTGLPQLMHSSDVVIPLLPLTEQTFHLFDKDILGQMKQGSYLVNACRGSVVDEKAVVHSLKTGQLAGYAADVFEMEDWIRSDRPREIPQELLDNTAQTFFTPHLGSAVDEIRIEIERYCATSILQALAGDIPDGRVNDIR

SEQ ID NO: 58 - protein, 328 aa, Streptomyces sp.
MVTHWIHPEVVDYLRRFCDPVVPVETEVLGRRQCLELAADADALIMCMADRVDDDFLAQCPRLRVISTVVKGYDNFDAEACARRGVWLTVLPDLLTAPTAELAVTLAVALGRRIREGDALMRSGRYDGWRPVLYGTGLYRSRVGVVGMGRLGRAVARRLSGFEPSEVLYYDKQPLGASEERRLGVRAAGLEELMGRCQVVLSLLPLAMDTRHLIGSDAIAAARPGQLLVNVGRGSVVDEDAVAAALDCGPLGGYAADVFGCEDLTAPGHLREVPRRLLTHPRTLLTPHLGSAVDVIRRDMEIAAAHQVEQALSGRVPDHEVTAGLLRE

SEQ ID NO: 59 - protein, 336 aa, Thioalkalivibrio sp.
MLPKLVITHRVHDEILQLLAPHCELMTNQTDSTLPREEILRRCRDAQAMMAFMPDRVDADFLQACPELRVVGCALKGFDNFDVDACTARGVWLTFVPDLLTVPTAELAIGLAVGLGRHLRAADAFVRSGEFQGWQPQFYGTGLDNATVGILGMGAIGLAMADRLQGWGATLQYHEAKALDTQTEQRLGLRRVACSELFASSDFILLALPLNADTQHLVNAELLALVRPGALLVNPCRGSVVDEAAVLAALERGQLGGYAADVFEMEDWARADRPRLIDPALLTHPNTLFTPHIGSAVRAVRLEIERCAAQNIIQVLAGARPINAANRLPKAEPAAC

SEQ ID NO: 60 - protein, 331 aa, Xanthobacter flavus
MARKTIVTNWVHPEVLDLLSTRGPAEANTTREPWPRDEIIRRAHGADAMLAFMTDHVDAAFLDACPELKIVACALKGADNFDMEACRARKVAVTIVPDLLTAPTAELAVGLMITLGRNLLAGDRLIRERPFAGWRPVLYGTGLDGAEVGIVGMGAVGQAIAHRLRPFRCRLSYCDARPLSPAAEDAQGLLRRDLADLVARSDYLVLALPLTPASRHLIDAAALAGMKPGALLINPARGSLVDEAAVADALEAGHLGGYAADVFETEDWARPDRPAAIEARLLAHPRTVLTPHIGSAVDSVRRDIALAAARDILRHLDGLQQDPPSRDRSAA

SEQ ID NO: 61 - protein, 10 aa, synthetic construct
VGILGMGAIG

SEQ ID NO: 62 - protein, 16 aa, synthetic construct
  VARIANT 1: note = K or R
XPGALLVNPCRGSVVD

SEQ ID NO: 63 - protein, 6 aa, synthetic construct
RGSVVD

SEQ ID NO: 64 - protein, 11 aa, synthetic construct
GWQPQFYGTGL

SEQ ID NO: 65 - protein, 11 aa, synthetic construct
  VARIANT 3: note = R, Q, T, or K
  VARIANT 5: note = A, V, Q, R, K, H, or E
  VARIANT 6: note = L or F
  VARIANT 8: note = G, F, or S
  VARIANT 9: note = T, R, M, L, A, or S
GWXPXXYXXGL

SEQ ID NO: 66 - DNA, 1011 bp, synthetic construct
  source 1..1011: mol_type = other DNA
atgttgccaaagttggtcatcacccatagagtccatgatgaaatcttgcaattgttagctccacattgcgaattaatgactaatcaaactgattctacattaactagagaagaaatcttgagaagatgtagagatgctcaagctatgatggccttcatgccagatagagttgatgctgatttcttgcaagcatgtccagaattgagagtcgttggttgcgccttgaagggtttcgataatttcgatgtcgatgcttgtactgctagaggtgtctggttgacattcgtcccagatttgttgactgtcccaactgctgaattggctattggtttggccgtcggtcttggtagacatttgagagccgctgatgctttcgttagatctggtgagtttcaaggttggcaaccacaattctacggtactggtttggataacgccactgttggtatcttgggtatgggtgccatcggtttggctatggccgatagattgcaaggttggggtgctactttgcaataccatgaagctaaggccttggatacccaaactgaacaaagattaggtttgagacaagttgcttgttctgaactttttgcctcttcagatttcatcttgttggccttgcctttgaacgccgatactcaacatttggtcaatgccgaattattggccttggtcagaccaggtgccttgcttgtcaacccttgtagaggttctgttgttgatgaagctgccgttttggccgctcttgaaagaggtcaattaggtggttatgccgccgatgttttcgaaatggaggattgggctagagctgataggccaagattgatcgatccagctttgttagctcatcctaacaccttgttcactccacatatcggttctgctgttagagctgttagacttgaaattgagagatgcgccgcccagaacatcatccaagtcttggctggtgccagacctattaacgccgccaatagattgccaaaggctgaaccagctgcttgttaa

SEQ ID NO: 67 - DNA, 75 bp, Saccharomyces cerevisiae
  source 1..75: mol_type = genomic DNA
atgttatcattgagacaatctataagatttttcaagccagccacaagaactttgtgttcatctagatatttgtta

SEQ ID NO: 68 - DNA, 1086 bp, synthetic construct
  source 1..1086: mol_type = other DNA
atgttatcattgagacaatctataagatttttcaagccagccacaagaactttgtgttcatctagatatttgttaatgttgccaaagttggtcatcacccatagagtccatgatgaaatcttgcaattgttagctccacattgcgaattaatgactaatcaaactgattctacattaactagagaagaaatcttgagaagatgtagagatgctcaagctatgatggccttcatgccagatagagttgatgctgatttcttgcaagcatgtccagaattgagagtcgttggttgcgccttgaagggtttcgataatttcgatgtcgatgcttgtactgctagaggtgtctggttgacattcgtcccagatttgttgactgtcccaactgctgaattggctattggtttggccgtcggtcttggtagacatttgagagccgctgatgctttcgttagatctggtgagtttcaaggttggcaaccacaattctacggtactggtttggataacgccactgttggtatcttgggtatgggtgccatcggtttggctatggccgatagattgcaaggttggggtgctactttgcaataccatgaagctaaggccttggatacccaaactgaacaaagattaggtttgagacaagttgcttgttctgaactttttgcctcttcagatttcatcttgttggccttgcctttgaacgccgatactcaacatttggtcaatgccgaattattggccttggtcagaccaggtgccttgcttgtcaacccttgtagaggttctgttgttgatgaagctgccgttttggccgctcttgaaagaggtcaattaggtggttatgccgccgatgttttcgaaatggaggattgggctagagctgataggccaagattgatcgatccagctttgttagctcatcctaacaccttgttcactccacatatcggttctgctgttagagctgttagacttgaaattgagagatgcgccgcccagaacatcatccaagtcttggctggtgccagacctattaacgccgccaatagattgccaaaggctgaaccagctgcttgttaa

SEQ ID NO: 69 - DNA, 400 bp, Saccharomyces cerevisiae
  source 1..400: mol_type = genomic DNA
catagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaag

SEQ ID NO: 70 - DNA, 1011 bp, synthetic construct
  source 1..1011: mol_type = other DNA
atgctgccgaaactcgttatcactcaccgggtgcacgatgagatcctgcaactgctggcgccacattgcgagctgatgaccaaccagaccgacagcacgctgacgcgcgaggaaattctgcgccgctgccgcgatgctcaggcgatgatggcgttcatgcccgatcgggtcgatgcagactttcttcaagcctgccctgagctgcgcgtggtcggctgcgcgctcaagggcttcgacaatttcgatgtggacgcctgcactgcccgcggggtctggctgaccttcgtgcctgatctgttgacggtcccgactgccgagctggcgatcggactggcggtggggctggggcggcatctgcgggcagcagatgcgttcgtccgctctggcgagttccagggctggcaaccacagttctacggcacggggctggataacgctacggtcggcatccttggcatgggcgccatcggactggccatggctgatcgcttgcagggatggggcgcgaccctgcagtaccacgaggcgaaggctctggatacacaaaccgagcaacggctcggcctgcgccaggtggcgtgcagcgaactcttcgccagctcggacttcatcctgctggcgcttcccttgaatgccgatacccagcatctggtcaacgccgagctgcttgccctcgtgcggccgggcgctctgcttgtgaacccctgccgcggttcggtggtggatgaagccgccgtgctcgcggcgcttgagcggggccagctcggcgggtatgcggcggatgtgttcgaaatggaagactgggctcgcgcggaccggccgcggctgatcgatcctgcgctgctcgcgcatccgaatacgctgttcactccgcacatcgggtcggcagtgcgcgcggtgcgcctggagattgaacgctgcgcagcgcagaacatcatccaggtgttggcaggtgcgcgcccaatcaacgctgcgaaccgcctgcccaaggccgagcctgccgcatgctga

SEQ ID NO: 71 - DNA, 168 bp, synthetic construct
  source 1..168: mol_type = other DNA
atggccgccaagatccgcatcgtgatgaaatcttttatgagccaagctaacaaagttgaaggggttattccatacgcgcagaaggttggattgcctgaatcacgatccttgtataccgtgctacgatcgcctcacatcgacaagaagtcgagggagcagttctcgatg

SEQ ID NO: 72 - protein, 4 aa, synthetic construct
PVAT

SEQ ID NO: 73 - DNA, 1011 bp, synthetic construct
  source 1..1011: mol_type = other DNA
atgttaccaaaattagttattactcatagagtacatgatgaaattttacaattattagcaccacattgtgaattaatgactaaccaaactgatagtacattaacaagagaagaaattttaagaagatgtagagatgctcaagcaatgatggcattcatgcctgatagagttgatgcagatttcttacaagcttgtcctgaattaagagtagttggttgtgcattaaaaggtttcgataatttcgatgtagatgcttgtactgctagaggagtttgattaactttcgtacctgatttattaacagttccaactgctgaattagcaattggattagcagtaggattaggaagacatttaagagcagcagatgcattcgttagatctggtgaattccaaggttgacaaccacaattctatggtacaggattagataatgctacagttggtattttaggtatgggtgctattggattagctatggctgatagattacaaggatgaggtgcaactttacaatatcatgaagcaaaagctttagatacacaaactgaacaaagattaggtttaagacaagtagcatgtagtgaattattcgctagttcagatttcattttattagcattacctttaaatgctgatactcaacatttagttaatgctgaattattagctttagtaagaccaggtgctttattagtaaatccttgtagaggttcagtagtagatgaagctgctgtattagcagcattagaaagaggtcaattaggtggatatgcagcagatgtattcgaaatggaagattgagctagagcagatagaccaagattaattgatcctgcattattagcacatccaaatacattattcactccacatattggatcagcagtaagagcagtaagattagaaattgaaagatgtgcagcacaaaacattattcaagtattagcaggtgcaagaccaattaacgctgcaaacagattacctaaagctgaacctgctgcatgttaa

SEQ ID NO: 74 - DNA, 425 bp, Saccharomyces cerevisiae
  source 1..425: mol_type = genomic DNA
tattgtgttaccttatttataaaggtatgaagcaaaggtgttattatttattattattattattattattaatataatatatatatatatatatgatatgaatattattagttttcgggaagcgggaatcccgtaaggagtgagggaccctccctatactaagggagggggaccgaaccccgaaggagttttatttttagtattttataaaatatatatttatatgattaataatattatatatattatttataaaaataatatataattttaattatttttaataaaaaaaggtggggtttggtaatataatatttttattttatttataatatataataataaattataaataaattttaattaaaagtagtattaacatattataaatagacaaaagagtctaaaggttaagatttattaaa

SEQ ID NO: 75 - DNA, 102 bp, Saccharomyces cerevisiae
  source 1..102: mol_type = genomic DNA
ttaatatttttaattattaaaaataataataataataataattataataatattcttaaatataataaagatatagatttatattctattcaatcaccttat

SEQ ID NO: 76 - DNA, 863 bp, Oryza sativa
  source 1..863: mol_type = genomic DNA
atagacctttttatttttcgtcattcgatcacgaaaacagggattctggaacggccaagaatcccagcggttgttcgggtcgaaaaaccgagaacaagacatgccacaaagtggcagatgaaggcaggggggagagcctagtcctcaacctcttcttccccaaaaggtagttatgaacgtgccaaacttattggatttattcttggaatgctcataaccacctttactctttttttcattctttactcagaggaagccatgccgtttggagaagagcaccaagtggggggagtgtggagtccccgaaagaggagctttctaaaggcaagagaaaagctccgatggagcccttggagctacagggaccaccaacccttcgcagcttggacgatttgattcttgtgccactcagccctgaggagggcgcctgctcgacccagtcaggtactactccgccgccggccccgagtaactctgcgggggtcggtgcagccctttcttctattccggagtgcataaacaaggatcctcaaaaagcgaaatcatttcgttaatggcatttcagaaatgagtcataggcgcctgtacaatgacagaatagagagtcctttttttccagaatgaatcattctattcaaatctcacaagttctctttacgcgtcttctaggggcattgttgaacgcaatctgcaggaacaagaaatgattctttcttattttgaaacagaattcaaaataaaggaggatttaattcggttgctttatgaaggccgacgccgtgccgatagatacgttatacacgaaacgaaaatagccagtacggtggacgcgttcctttccaaaaagggattatcaggagctc

SEQ ID NO: 77 - DNA, 139 bp, synthetic construct
  source 1..139: mol_type = other DNA
gtctgccccattcgataatggcataatacgactcactatagtttatgatctagtggagtgagtgattgtgtggtgttcagtctaaggctttttgaaaagcggatttctcccttctctcatccatcgtctttgttaaagt

SEQ ID NO: 78 - DNA, 176 bp, synthetic construct
  source 1..176: mol_type = other DNA
ggtaccaagcgatcgcaaacctaggaaaagatctaaaaagcttaagcggccgcaaaaaccccttggggcctctaaacgggtcttgaggggttttttgtagatagacctttttatttttcgtcattcgatcacgaaaaggccggccaaacctcgaggaaaggtaccaaaggcgcgcc

SEQ ID NO: 79 - DNA, 2826 bp, synthetic construct
  source 1..2826: mol_type = other DNA
atggccgccaagatccgcatcgtgatgaaatcttttatgagccaagctaacaaagttgaaggggttattccatacgcgcagaaggttggattgcctgaatcacgatccttgtataccgtgctacgatcgcctcacatcgacaagaagtcgagggagcagttctcgatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaagcatgctga

SEQ ID NO: 80 - DNA, 1740 bp, synthetic construct
  source 1..1740: mol_type = other DNA
atgctgcctaaactcgttataactcaccgagtacatgatgagatcctgcaactgctggcgccacattgcgagctgatgaccaaccaaaccgacagcacactgacacgcgaggaaattctgcgtcgatgtcgtgatgctcaagcgatgatggcgttcatgcccgatcgagtcgatgcagactttcttcaagcctgccctgagctgcgtgtagtcggctgcgcgctcaagggcttcgacaatttcgatgtggacgcctgtactgcccgtggggtctggctgaccttcgtgcctgatctgttgacagtcccaactgccgagctggcgatcggactggcggtggggctggggcgacatctgcgagcagcagatgcgttcgtccgttctggcgagttccaaggctggcaaccacaattctatggcacagggctggataacgctacagtcggcatccttggcatgggcgccatcggactggccatggctgatcgattgcaaggatggggcgcgaccctgcaatatcacgaggcgaaggctctggatacacaaaccgagcaacgactcggcctgcgtcaagtggcgtgcagcgaactcttcgccagctcggacttcatcctgctggcgcttcccttgaatgccgatacccaacatctggtcaacgccgagctgcttgccctcgtacgaccaggcgctctgcttgtaaacccctgtcgtggttcggtagtggatgaagccgccgtgctcgcggcgcttgagcgaggccaactcggcgggtatgcggcggatgtattcgaaatggaagactgggctcgtgcggaccgaccacgactgatcgatcctgcgctgctcgcgcatcctaatacactgttcactccacatatagggtcggcagtgcgtgcggtgcgtctggagattgaacgttgtgcagcgcaaaacatcatccaagtattggcaggtgcgcgtccaatcaacgctgcgaaccgtctgcccaaggccgagcctgccgcatgtccagttgctactatggtgagtaagggagaggagctgttcaccggggtggtgcctatcctggtcgagctggatggtgatgtaaacggtcataaattcagtgtgtccggtgaaggtgaaggtgatgccacctatggtaagctgacccttaagttcatctgtaccaccggaaagctgcctgtgccttggcctaccctcgtgaccaccctgacatatggagtgcaatgtttcagtcgttatcctgatcatatgaagcaacatgatttctttaaatccgccatgcctgaaggttatgtccaagagcgtaccatattctttaaagatgatggtaactataagacccgtgccgaggtgaagttcgagggtgataccctggtgaaccgtattgagcttaagggtatcgatttcaaggaggatggaaacatcctggggcataagctggagtataactataacagtcataacgtctatatcatggccgataagcaaaagaacggtatcaaggtgaacttcaagatccgtcataatatcgaagatggaagtgtgcaactcgccgatcattatcaacaaaacacccctatcggtgatggtcctgtgctgctgcctgataaccattatctgagtacccaatccgccctgagtaaagatcctaacgagaagcgtgatcaaatggtactgcttgagttcgttaccgccgccgggatcactctcggtatggatgagctgtataagtaa

SEQ ID NO: 81 - protein, 579 aa, synthetic construct
MLPKLVITHRVHDEILQLLAPHCELMTNQTDSTLTREEILRRCRDAQAMMAFMPDRVDADFLQACPELRVVGCALKGFDNFDVDACTARGVWLTFVPDLLTVPTAELAIGLAVGLGRHLRAADAFVRSGEFQGWQPQFYGTGLDNATVGILGMGAIGLAMADRLQGWGATLQYHEAKALDTQTEQRLGLRQVACSELFASSDFILLALPLNADTQHLVNAELLALVRPGALLVNPCRGSVVDEAAVLAALERGQLGGYAADVFEMEDWARADRPRLIDPALLAHPNTLFTPHIGSAVRAVRLEIERCAAQNIIQVLAGARPINAANRLPKAEPAACPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDQMVLLEFVTAAGITLGMDELYK

SEQ ID NO: 82 - DNA, 1740 bp, synthetic construct
  source 1..1740: mol_type = other DNA
atgctgcctaaactcgttataactcaccgagtacatgatgagatcctgcaactgctggcgccacattgcgagctgatgaccaaccaaaccgacagcacactgacacgcgaggaaattctgcgtcgatgtcgtgatgctcaagcgatgatggcgttcatgcccgatcgagtcgatgcagactttcttcaagcctgccctgagctgcgtgtagtcggctgcgcgctcaagggcttcgacaatttcgatgtggacgcctgtactgcccgtggggtctggctgaccttcgtgcctgatctgttgacagtcccaactgccgagctggcgatcggactggcggtggggctggggcgacatctgcgagcagcagatgcgttcgtccgttctggcgagttccaaggctggcaaccacaattctatggcacagggctggataacgctacagtcggcatccttggcatgggcgccatcggactggccatggctgatcgattgcaaggatggggcgcgaccctgcaatatcacgaggcgaaggctctggatacacaaaccgagcaacgactcggcctgcgtcaagtggcgtgcagcgaactcttcgccagctcggacttcatcctgctggcgcttcccttgaatgccgatacccaacatctggtcaacgccgagctgcttgccctcgtacgaccaggcgctctgcttgtaaacccctgtcgtggttcggtagtggatgaagccgccgtgctcgcggcgcttgagcgaggccaactcggcgggtatgcggcggatgtattcgaaatggaagactgggctcgtgcggaccgaccacgactgatcgatcctgcgctgctcgcgcatcctaatacactgttcactccacatatagggtcggcagtgcgtgcggtgcgtctggagattgaacgttgtgcagcgcaaaacatcatccaagtattggcaggtgcgcgtccaatcaacgctgcgaaccgtctgcccaaggccgagcctgccgcatgtggaggtggaggttcagtgagtaagggagaggagctgttcaccggggtggtgcctatcctggtcgagctggatggtgatgtaaacggtcataaattcagtgtgtccggtgaaggtgaaggtgatgccacctatggtaagctgacccttaagttcatctgtaccaccggaaagctgcctgtgccttggcctaccctcgtgaccaccctgacatatggagtgcaatgtttcagtcgttatcctgatcatatgaagcaacatgatttctttaaatccgccatgcctgaaggttatgtccaagagcgtaccatattctttaaagatgatggtaactataagacccgtgccgaggtgaagttcgagggtgataccctggtgaaccgtattgagcttaagggtatcgatttcaaggaggatggaaacatcctggggcataagctggagtataactataacagtcataacgtctatatcatggccgataagcaaaagaacggtatcaaggtgaacttcaagatccgtcataatatcgaagatggaagtgtgcaactcgccgatcattatcaacaaaacacccctatcggtgatggtcctgtgctgctgcctgataaccattatctgagtacccaatccgccctgagtaaagatcctaacgagaagcgtgatcaaatggtactgcttgagttcgttaccgccgccgggatcactctcggtatggatgagctgtataagtaa

SEQ ID NO: 83 - protein, 579 aa, synthetic construct
MLPKLVITHRVHDEILQLLAPHCELMTNQTDSTLTREEILRRCRDAQAMMAFMPDRVDADFLQACPELRVVGCALKGFDNFDVDACTARGVWLTFVPDLLTVPTAELAIGLAVGLGRHLRAADAFVRSGEFQGWQPQFYGTGLDNATVGILGMGAIGLAMADRLQGWGATLQYHEAKALDTQTEQRLGLRQVACSELFASSDFILLALPLNADTQHLVNAELLALVRPGALLVNPCRGSVVDEAAVLAALERGQLGGYAADVFEMEDWARADRPRLIDPALLAHPNTLFTPHIGSAVRAVRLEIERCAAQNIIQVLAGARPINAANRLPKAEPAACGGGGSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDQMVLLEFVTAAGITLGMDELYK

SEQ ID NO: 84 - protein, 5 aa, synthetic construct
GGGGS

SEQ ID NO: 85 - DNA, 1763 bp, synthetic construct
  source 1..1763: mol_type = other DNA
cattccatgtttccgaaacggatcctctgcctaaactcgttataactcaccgagtacatgatgagatcctgcaactgctggcgccacattgcgagctgatgaccaaccaaaccgacagcacactgacacgcgaggaaattctgcgtcgatgtcgtgatgctcaagcgatgatggcgttcatgcccgatcgagtcgatgcagactttcttcaagcctgccctgagctgcgtgtagtcggctgcgcgctcaagggcttcgacaatttcgatgtggacgcctgtactgcccgtggggtctggctgaccttcgtgcctgatctgttgacagtcccaactgccgagctggcgatcggactggcggtggggctggggcgacatctgcgagcagcagatgcgttcgtccgttctggcgagttccaaggctggcaaccacaattctatggcacagggctggataacgctacagtcggcatccttggcatgggcgccatcggactggccatggctgatcgattgcaaggatggggcgcgaccctgcaatatcacgaggcgaaggctctggatacacaaaccgagcaacgactcggcctgcgtcaagtggcgtgcagcgaactcttcgccagctcggacttcatcctgctggcgcttcccttgaatgccgatacccaacatctggtcaacgccgagctgcttgccctcgtacgaccaggcgctctgcttgtaaacccctgtcgtggttcggtagtggatgaagccgccgtgctcgcggcgcttgagcgaggccaactcggcgggtatgcggcggatgtattcgaaatggaagactgggctcgtgcggaccgaccacgactgatcgatcctgcgctgctcgcgcatcctaatacactgttcactccacatatagggtcggcagtgcgtgcggtgcgtctggagattgaacgttgtgcagcgcaaaacatcatccaagtattggcaggtgcgcgtccaatcaacgctgcgaaccgtctgcccaaggccgagcctgccgcatgtccagttgctactatggtgagtaagggagaggagctgttcaccggggtggtgcctatcctggtcgagctggatggtgatgtaaacggtcataaattcagtgtgtccggtgaaggtgaaggtgatgccacctatggtaagctgacccttaagttcatctgtaccaccggaaagctgcctgtgccttggcctaccctcgtgaccaccctgacatatggagtgcaatgtttcagtcgttatcctgatcatatgaagcaacatgatttctttaaatccgccatgcctgaaggttatgtccaagagcgtaccatattctttaaagatgatggtaactataagacccgtgccgaggtgaagttcgagggtgataccctggtgaaccgtattgagcttaagggtatcgatttcaaggaggatggaaacatcctggggcataagctggagtataactataacagtcataacgtctatatcatggccgataagcaaaagaacggtatcaaggtgaacttcaagatccgtcataatatcgaagatggaagtgtgcaactcgccgatcattatcaacaaaacacccctatcggtgatggtcctgtgctgctgcctgataaccattatctgagtacccaatccgccctgagtaaagatcctaacgagaagcgtgatcaaatggtactgcttgagttcgttaccgccgccgggatcactctcggtatggatgagctgtataagtaa

SEQ ID NO: 86 - protein, 581 aa, synthetic construct
MDPLPKLVITHRVHDEILQLLAPHCELMTNQTDSTLTREEILRRCRDAQAMMAFMPDRVDADFLQACPELRVVGCALKGFDNFDVDACTARGVWLTFVPDLLTVPTAELAIGLAVGLGRHLRAADAFVRSGEFQGWQPQFYGTGLDNATVGILGMGAIGLAMADRLQGWGATLQYHEAKALDTQTEQRLGLRQVACSELFASSDFILLALPLNADTQHLVNAELLALVRPGALLVNPCRGSVVDEAAVLAALERGQLGGYAADVFEMEDWARADRPRLIDPALLAHPNTLFTPHIGSAVRAVRLEIERCAAQNIIQVLAGARPINAANRLPKAEPAACPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDQMVLLEFVTAAGITLGMDELYK

SEQ ID NO: 87 - DNA, 240 bp, Oryza sativa
  source 1..240: mol_type = genomic DNA
atggcaaatctggtccgatggctcttctccactacccgagggactaacggtcttccatatttcatcttcggtgtcgttgtaggaggcgccctgttgtttgctttgctaaagtatcaggcccctctgtacgacccggctttaatggaaaaaatcatagatcataatataaaagccgggcaccctatagaggttgactattcgtggtggggcacctctattcgtgtagtctttcctaagtaa

SEQ ID NO: 88 - DNA, 3957 bp, synthetic construct
  source 1..3957: mol_type = other DNA
atggccgccaagatccgcatcgtgatgaaatcttttatgagccaagctaacaaagttgaaggggttattccatacgcgcagaaggttggattgcctgaatcacgatccttgtataccgtgctacgatcgcctcacatcgacaagaagtcgagggagcagttctcgatgaacaacggtacaaataatttccaaaacttcatcgggatctcaagtttgcaaaagacactgcgtaatgctctgatccctactgaaaccactcaacaattcatcgtcaagaacggaataattaaagaagatgagttacgtggtgagaaccgacaaattctgaaagatatcatggatgattattatcgtggattcatctctgagactctgagttctattgatgatatagattggactagtctgttcgaaaagatggaaattcaactgaagaatggtgataataaagataccttgattaaggaacaaacagagtatcgtaaagcaatccataagaaatttgctaacgatgatcgatttaagaacatgtttagtgccaaactgattagtgatatattacctgagttcgtcatccataacaataattattcggcatcagagaaagaggaaaagacccaagtgataaagttgttctcgcgatttgcaactagtttcaaggattatttcaagaaccgtgcaaattgtttctcagcagatgatatttcatcaagtagttgtcatcgtatcgtcaacgataatgcagagatattcttctcaaatgcactggtctatcgtcgaatcgtaaagtcgctgagtaatgatgatatcaacaaaatttcgggtgatatgaaagattcattaaaagaaatgagtctggaagaaatatattcttatgagaagtatggtgaatttattactcaagaaggtattagtttctataatgatatctgtggtaaagtaaattcattcatgaacctgtattgtcaaaagaataaggaaaacaagaatttatataaacttcaaaaacttcataaacaaattctatgtattgcagatactagttatgaggtcccatataagtttgaaagtgatgaggaagtgtatcaatcagttaacggtttccttgataacattagtagtaaacatatagtcgaaagattacgtaaaatcggtgataactataacggttataacctggataaaatttatatcgtgtccaaattctatgagagtgttagtcaaaagacctatcgtgattgggaaacaattaataccgccctcgaaattcattataataatatcttgcctggtaacggtaaaagtaaagccgataaagtaaagaaagcagttaagaatgatttacaaaagtccatcaccgaaataaatgaactagtgtcaaactataagctgtgtagtgatgataacatcaaagcagagacttatatacatgagattagtcatatcttgaataactttgaagcacaagaattgaaatataatccagaaattcatctagttgaatccgagcttaaagcaagtgagcttaaaaacgtgctggatgtgatcatgaatgcctttcattggtgttcggtttttatgactgaggaacttgttgataaagataacaatttttatgcagaactggaggagatttatgatgaaatttatccagtaattagtctgtataacctggttcgtaactatgttacccaaaaaccatatagtactaagaagattaaattgaactttggaataccaacattagcagatggttggtcaaagtccaaagagtattctaataacgctatcatactgatgcgtgataatctgtattatctgggtatctttaatgcaaagaataaaccagataagaagattatcgagggtaatacatcagaaaataagggtgattataagaagatgatttataatttgctccctggtcctaacaaaatgatcccaaaagttttcttgagtagtaagacaggggtggaaacctataaaccaagtgcctatatcctagaggggtataaacaaaataaacatatcaagtcttcaaaagatttcgatatcactttctgtcatgatctgatcgattatttcaaaaactgtattgcaattcatcctgagtggaagaacttcgggtttgatttcagtgataccagtacttatgaagatatttccgggttctatcgtgaggtagagttacaaggttataagattgattggacatatattagtgaaaaggatattgatctgctgcaagaaaagggtcaactgtatctgttccaaatatataacaaagatttctcgaagaaatcaaccgggaatgataaccttcataccatgtatctgaaaaatcttttctcagaagaaaatcttaaggatatcgtcctgaaacttaacggtgaagctgaaatattcttcagaaagagtagtataaagaacccaatcattcataagaaaggttcgatattagtcaaccgtacctatgaagcagaagaaaaggatcaatttggtaacattcaaattgtgcgtaagaatattccagagaacatttatcaagagctgtataaatatttcaacgataagtcagataaagagctgtctgatgaagcagccaaactgaagaatgtagtgggacatcatgaggcagcaacaaatatagtcaaggattatcgttatacatatgataaatatttccttcatatgcctattacaatcaatttcaaagccaataaaactggtttcattaatgatagaatcttacaatatatcgctaaagaaaaggatttacatgttatcggtattgatcgaggtgagcgtaacctgatctatgtgtcagtgattgatacttgtggtaatatagttgaacaaaagagttttaacattgtaaacggttatgattatcaaataaaactgaaacaacaagagggtgctagacaaattgctcgaaaggaatggaaagaaattggtaagattaaagagatcaaagagggttatctgagtttagtaatccatgagatctctaagatggtaatcaaatataatgcaattatagcaatggaggatttgtcttatggtttcaagaaagggcgtttcaaggtcgaacgacaagtttatcaaaagttcgaaactatgctcatcaataaactcaactatctggtattcaaagatatttcgattaccgagaatggtggactcctgaaaggttatcaactgacatatattcctgataaacttaagaacgtgggtcatcaatgtggttgtattttctatgtgcctgctgcatatacaagtaagattgatccaaccaccggtttcgtgaatatcttcaagttcaaagatctgacagtggatgcaaagcgtgagttcattaagaagttcgattcaattcgttatgatagtgaaaagaatctgttctgtttcacattcgattataataacttcattacacaaaacacagtcatgagtaagtcatcgtggagtgtgtatacatatggtgtgcgtatcaaacgtcgatttgtgaacggtcgtttctcaaacgaaagtgataccattgatataaccaaagatatggagaagacattggaaatgacagatattaactggcgtgatggtcatgatcttcgtcaagatattatagattatgaaattgttcaacatatattcgaaattttccgtctaacagtgcaaatgcgtaactccttgtctgaactggaggatcgtgattatgatcgtctcatttcacctgtactgaacgaaaataacatattctatgatagtgcaaaggctggagatgcacttcctaaggatgccgatgcaaatggtgcatattgtattgcattaaaagggttatatgaaattaaacaaattaccgaaaattggaaagaagatggtaaattttcgcgtgataaactcaaaatcagtaataaagattggttcgatttcatccaaaataagcgttatctctaa

SEQ ID NO: 89 - protein, 1318 aa, synthetic construct
MAAKIRIVMKSFMSQANKVEGVIPYAQKVGLPESRSLYTVLRSPHIDKKSREQFSMNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 90 - protein, 56 aa, Oryza sativa
MAAKIRIVMKSFMSQANKVEGVIPYAQKVGLPESRSLYTVLRSPHIDKKSREQFSM

SEQ ID NO: 91 - protein, 938 aa, synthetic construct
MAAKIRIVMKSFMSQANKVEGVIPYAQKVGLPESRSLYTVLRSPHIDKKSREQFSMNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 92 - protein, 36 aa, Arabidopsis thaliana
MFKQASRLLSRSVAAASSKSVTTRAFSTELPSTLDS

SEQ ID NO: 93 - DNA, 25 bp, Oryza sativa
  source 1..25: mol_type = genomic DNA
tttatagttctagcattaaccggtc

SEQ ID NO: 94 - DNA, 25 bp, Oryza sativa
  source 1..25: mol_type = genomic DNA
tttattcaattatgaaattactcat

SEQ ID NO: 95 - DNA, 25 bp, Oryza sativa
  source 1..25: mol_type = genomic DNA
cttccggatatagaaacattaaagc

SEQ ID NO: 96 - DNA, 25 bp, Oryza sativa
  source 1..25: mol_type = genomic DNA
tttagtcacatttctaccggtgcac

SEQ ID NO: 97 - DNA, 25 bp, synthetic construct
  source 1..25: mol_type = other DNA
ttcattgtattggctcttactggat

SEQ ID NO: 98 - DNA, 16 bp, synthetic construct
  source 1..16: mol_type = other DNA
tttattcattactcat

SEQ ID NO: 99 - DNA, 25 bp, synthetic construct
  source 1..25: mol_type = other DNA
gtgacggatatagaaacattaaagc

SEQ ID NO: 100 - DNA, 57 bp, synthetic construct
  source 1..57: mol_type = other DNA
gtctggccccaaattctaatttctactgttgtagattagttctagcattaaccggtc

SEQ ID NO: 101 - DNA, 57 bp, synthetic construct
  source 1..57: mol_type = other DNA
gtctggccccaaattctaatttctactgttgtagatttcaattatgaaattactcat

SEQ ID NO: 102 - DNA, 57 bp, synthetic construct
  source 1..57: mol_type = other DNA
gtctggccccaaattctaatttctactgttgtagatcggatatagaaacattaaagc

SEQ ID NO: 103 - DNA, 57 bp, synthetic construct
  source 1..57: mol_type = other DNA
gtctggccccaaattctaatttctactgttgtagatgtcacatttctaccggtgcac

SEQ ID NO: 104 - DNA, 1554 bp, synthetic construct
  source 1..1554: mol_type = other DNA
gggcccgccggtcatagttcagtaaagattttaagtgggttcgcttggactatgctatttctgaataatattttctatttcataggagatcttggtcccttactttaatgtttctatatccgtggaattaggtgtagctatattacaagctcatgtttctacgatctcaatttgtatttacttgaatgatgctataaatctccatcaaaatgagtaatttcataattgaataaaaacgaggagccgaagattttagggggcgggacaaacgcggaagtgtattgcgttacaaaaaatgacaactagcatttgttttttcatttcatgttcgaattcgtttttcgttggaaaaaccaacgccgaccccaaacaagtctctccaatataaggagagcggagcttaaaaatattattttattgtgctatggcaaatctggtccgatggctcttctccactacccgagggactaacggtcttccatatttcatcttcggtgtcgttgtaggaggcgccctgttgtttgctttgctaaagtatcaggcccctctgtacgacccggctttaatggaaaaaatcatagatcataatataaaagccgggcaccctatagaggttgactattcgtggtggggcacctctattcgtgtagtctttcctaagggaggtggaggttcaagtgagctgattaaggagaacatgcatatgaagctgtatatggagggtaccgtgaacaaccatcatttcaagtgtacatccgagggtgaaggtaagccttatgagggtacccaaaccatgagaatcaaggtggtcgagggtggtcctctccctttcgctttcgatattctggctaccagtttcatgtatggtagtagaaccttcatcaaccatactcaaggtatccctgatttctttaagcaatccttccctgagggtttcacatgggagagagtcaccacatatgaagatgggggtgtgctgaccgctacccaagataccagtctccaagatggttgtctcatctataacgtcaagatcagaggggtgaacttcccatccaacggtcctgtgatgcaaaagaaaacactcggttgggaggccaacaccgagatgctgtatcctgctgatggtggtctggaaggtagaagtgatatggccctgaagctcgtgggtgggggtcatctgatctgtaacttcaagaccacatatagatccaagaaaccagctaagaacctcaagatgcctggtgtctattatgtggatcatagactggaaagaatcaaggaggccgataaagagacttatgtcgagcaacatgaggtggctgtggctagatattgtgatctccctagtaaactggggcataagtaagaaagacaggacagtggtggtttgctcatactttcattacaaaaccatactatggaatgctttaatgtttctatatccgtcactagggaatctcgtgcttgcatatctaaatctaagttttgagacagacctttcatgggttcaaagaaaagaagagtacgagtgggtgatgtgattgagggccc

SEQ ID NO: 105 - DNA, 1425 bp, synthetic construct
  source 1..1425: mol_type = other DNA
gggcccgcattaactggtctggaattaggtgtagctatattacaagctcatgtttctacgatctcaatttgtatttacttgaatgatgctataaatctccatcaaaatgagtaatgaataaaaacgaggagccgaagattttagggggcgggacaaacgcggaagtgtattgcgttacaaaaaatgacaactagcatttgttttttcatttcatgttcgaattcgtttttcgttggaaaaaccaacgccgaccccaaacaagtctctccaatataaggagagcggagcttaaaaatattattttattgtgctatggcaaatctggtccgatggctcttctccactacccgagggactaacggtcttccatatttcatcttcggtgtcgttgtaggaggcgccctgttgtttgctttgctaaagtatcaggcccctctgtacgacccggctttaatggaaaaaatcatagatcataatataaaagccgggcaccctatagaggttgactattcgtggtggggcacctctattcgtgtagtctttcctaagggaggtggaggttcaagtgagctgattaaggagaacatgcatatgaagctgtatatggagggtaccgtgaacaaccatcatttcaagtgtacatccgagggtgaaggtaagccttatgagggtacccaaaccatgagaatcaaggtggtcgagggtggtcctctccctttcgctttcgatattctggctaccagtttcatgtatggtagtagaaccttcatcaaccatactcaaggtatccctgatttctttaagcaatccttccctgagggtttcacatgggagagagtcaccacatatgaagatgggggtgtgctgaccgctacccaagataccagtctccaagatggttgtctcatctataacgtcaagatcagaggggtgaacttcccatccaacggtcctgtgatgcaaaagaaaacactcggttgggaggccaacaccgagatgctgtatcctgctgatggtggtctggaaggtagaagtgatatggccctgaagctcgtgggtgggggtcatctgatctgtaacttcaagaccacatatagatccaagaaaccagctaagaacctcaagatgcctggtgtctattatgtggatcatagactggaaagaatcaaggaggccgataaagagacttatgtcgagcaacatgaggtggctgtggctagatattgtgatctccctagtaaactggggcataagtaagaaagacaggacagtggtggtttgctcatactttcattacaaaaccatactatggaattcacttctcatgaaataattccctttccaaggaaaggaaaacaagaactcgaatactcgtaatagcgatcccgatccacctacttttttctattctttgattcggggccc

SEQ ID NO: 106 - DNA, 24 bp, Oryza sativa
  source 1..24: mol_type = genomic DNA
cgtgcattaagctcaggaatacgt

SEQ ID NO: 107 - DNA, 24 bp, Oryza sativa
  source 1..24: mol_type = genomic DNA
catcggaccagatttgccatagca

SEQ ID NO: 108 - DNA, 1905 bp, synthetic construct
  source 1..1905: mol_type = other DNA
atggccgccaagatccgcatcgtgatgaaatcttttatgagccaagctaacaaagttgaaggggttattccatacgcgcagaaggttggattgcctgaatcacgatccttgtataccgtgctacgatcgcctcacatcgacaagaagtcgagggagcagttctcgatgctgccgaaactcgttatcactcaccgggtgcacgatgagatcctgcaactgctggcgccacattgcgagctgatgaccaaccagaccgacagcacgctgacgcgcgaggaaattctgcgccgctgccgcgatgctcaggcgatgatggcgttcatgcccgatcgggtcgatgcagactttcttcaagcctgccctgagctgcgcgtggtcggctgcgcgctcaagggcttcgacaatttcgatgtggacgcctgcactgcccgcggggtctggctgaccttcgtgcctgatctgttgacggtcccgactgccgagctggcgatcggactggcggtggggctggggcggcatctgcgggcagcagatgcgttcgtccgctctggcgagttccagggctggcaaccacagttctacggcacggggctggataacgctacggtcggcatccttggcatgggcgccatcggactggccatggctgatcgcttgcagggatggggcgcgaccctgcagtaccacgaggcgaaggctctggatacacaaaccgagcaacggctcggcctgcgccaggtggcgtgcagcgaactcttcgccagctcggacttcatcctgctggcgcttcccttgaatgccgatacccagcatctggtcaacgccgagctgcttgccctcgtgcggccgggcgctctgcttgtgaacccctgccgcggttcggtggtggatgaagccgccgtgctcgcggcgcttgagcggggccagctcggcgggtatgcggcggatgtgttcgaaatggaagactgggctcgcgcggaccggccgcggctgatcgatcctgcgctgctcgcgcatccgaatacgctgttcactccgcacatcgggtcggcagtgcgcgcggtgcgcctggagattgaacgctgcgcagcgcagaacatcatccaggtgttggcaggtgcgcgcccaatcaacgctgcgaaccgcctgcccaaggccgagcctgccgcatgcccagttgctactatggtgagtaagggagaggagctgttcaccggggtggtgcctatcctggtcgagctggatggtgatgtaaacggtcataaattcagtgtgtccggtgaaggtgaaggtgatgccacctatggtaagctgacccttaagttcatctgtaccaccggaaagctgcctgtgccttggcctaccctcgtgaccaccctgacatatggagtgcaatgtttcagtcgttatcctgatcatatgaagcaacatgatttctttaaatccgccatgcctgaaggttatgtccaagagcgtaccatattctttaaagatgatggtaactataagacccgtgccgaggtgaagttcgagggtgataccctggtgaaccgtattgagcttaagggtatcgatttcaaggaggatggaaacatcctggggcataagctggagtataactataacagtcataacgtctatatcatggccgataagcaaaagaacggtatcaaggtgaacttcaagatccgtcataatatcgaagatggaagtgtgcaactcgccgatcattatcaacaaaacacccctatcggtgatggtcctgtgctgctgcctgataaccattatctgagtacccaatccgccctgagtaaagatcctaacgagaagcgtgatcaaatggtactgcttgagttcgttaccgccgccgggatcactctcggtatggatgagctgtataagtaa

SEQ ID NO: 109 - protein, 634 aa, synthetic construct
MAAKIRIVMKSFMSQANKVEGVIPYAQKVGLPESRSLYTVLRSPHIDKKSREQFSMLPKLVITHRVHDEILQLLAPHCELMTNQTDSTLTREEILRRCRDAQAMMAFMPDRVDADFLQACPELRVVGCALKGFDNFDVDACTARGVWLTFVPDLLTVPTAELAIGLAVGLGRHLRAADAFVRSGEFQGWQPQFYGTGLDNATVGILGMGAIGLAMADRLQGWGATLQYHEAKALDTQTEQRLGLRQVACSELFASSDFILLALPLNADTQHLVNAELLALVRPGALLVNPCRGSVVDEAAVLAALERGQLGGYAADVFEMEDWARADRPRLIDPALLAHPNTLFTPHIGSAVRAVRLEIERCAAQNIIQVLAGARPINAANRLPKAEPAACPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDQMVLLEFVTAAGITLGMDELYK

SEQ ID NO: 110 - DNA, 46 bp, Oryza sativa
  source 1..46: mol_type = genomic DNA
ttctctgacattccatgtttccgaaacggatcctataaaatatttc

SEQ ID NO: 111 - protein, 7 aa, Oryza sativa
MDPIKYF

SEQ ID NO: 112 - DNA, 34 bp, synthetic construct
  source 1..34: mol_type = other DNA
ccatcgtctttgttaaagtatgctgcctaaactc

SEQ ID NO: 113 - protein, 5 aa, Pseudomonas stutzeri
MLPKL

SEQ ID NO: 114 - DNA, 57 bp, synthetic construct
  source 1..57: mol_type = other DNA
ccatcgtctttgttaaagtcattccatgtttccgaaacggatcctctgcctaaactc

SEQ ID NO: 115 - RNA, 57 nt, synthetic construct
  source 1..57: mol_type = other RNA
ccatcgtctttgttaaagtcattccatgtttccgaaatggatcctctgcctaaactc

SEQ ID NO: 116 - protein, 7 aa, synthetic construct
MDPLPKL

SEQ ID NO: 117 - DNA, 445 bp, synthetic construct
  source 1..445: mol_type = other DNA
tgcattaagctcaggaatacgtttatttgctaatatgatggccggtcatagttcagtaaagattttaagtgggttcgcttggactatgctatttctgaataatattttctatttcataggagatcttggtcccttatttatagttctagcattaaccggtctggaattaggtgtagctatattacaagctcatgtttctacgatctcaatttgtatttacttgaatgatgctataaatctccatcaaaatgagtaatttcataattgaataaaaacgaggagccgaagattttagggggcgggacaaacgcggaagtgtattgcgttacaaaaaatgacaactagcatttgttttttcatttcatgttcgaattcgtttttcgttggaaaaaccaacgccgaccccaaacaagtctctccaatataaggagagcggagcttaaaa

SEQ ID NO: 118 - DNA, 428 bp, synthetic construct
  source 1..428: mol_type = other DNA
aggaatacgtttatttgctaatatgatggccggtcatagttcagtaaagattttaagtgggttcgcttggactatgctatttctgaataatattttctatttcataggagatcttggtcccttatttatagttctagcattaaccggtctggaattaggtgtagctatattacaagctcatgtttctacgatctcaatttgtatttacttgaatgatgctataaatctccatcaaaatgagtaatttcataattgaataaaaacgaggagccgaagattttagggggcgggacaaacgcggaagtgtattgcgttacaaaaaatgacaactagcatttgttttttcatttcatgttcgaattcgtttttcgttggaaaaaccaacgccgaccccaaacaagtctctccaatataaggagagcggagct

SEQ ID NO: 119 - DNA, 26 bp, Oryza sativa
  source 1..26: mol_type = genomic DNA
cattccatgtttccgaaacggatcct

SEQ ID NO: 120 - protein, 79 aa, Oryza sativa
MANLVRWLFSTTRGTNGLPYFIFGVVVGGALLFALLKYQAPLYDPALMEKIIDHNIKAGHPIEVDYSWWGTSIRVVFPK

SEQ ID NO: 121 - DNA, 334 bp, synthetic construct
  source 1..334: mol_type = other DNA
cgaggtgtagcgcagtctggtcagcgcatctgttttgggtacagagggccataggttcgaatcctgtcaccttgagtctggccccaaattctaatttctactgttgtagattagttctagcattaaccggtcgtccctttcgtccagtggttaggacatcgtcttttcatgtcgaagacacgggttcgattcccgtaagggatagtctggccccaaattctaatttctactgttgtagatcggatatagaaacattaaagcattccagcttatttgatacccacttcaagtttctatcaaaccatgtctttttcttcgaacgtcaatctcgtag

SEQ ID NO: 122 - DNA, 1011 bp, synthetic construct; identical to SEQ ID NO: 28
  source 1..1011: mol_type = other DNA

SEQ ID NO: 123 - protein, 391 aa, synthetic construct
MAAKIRIVMKSFMSQANKVEGVIPYAQKVGLPESRSLYTVLRSPHIDKKSREQFSMLPKLVITHRVHDEILQLLAPHCELMTNQTDSTLTREEILRRCRDAQAMMAFMPDRVDADFLQACPELRVVGCALKGFDNFDVDACTARGVWLTFVPDLLTVPTAELAIGLAVGLGRHLRAADAFVRSGEFQGWQPQFYGTGLDNATVGILGMGAIGLAMADRLQGWGATLQYHEAKALDTQTEQRLGLRQVACSELFASSDFILLALPLNADTQHLVNAELLALVRPGALLVNPCRGSVVDEAAVLAALERGQLGGYAADVFEMEDWARADRPRLIDPALLAHPNTLFTPHIGSAVRAVRLEIERCAAQNIIQVLAGARPINAANRLPKAEPAAC

SEQ ID NO: 124 - DNA, 7478 bp, synthetic construct
  source 1..7478: mol_type = other DNA
ggcccaaattcaattgtatatgagctcatatacaagacctcactagtaaggaaggcacttgctgccggagttcaacaggcaaatataagaaaagaagtcctgttcacttcatcatctgtgggttgtactgcttgaaggttcttctgaggggtagaatttgaattccttctttgcttgtgagataaccatttccagaaactcatatatagagagcgggtatcggtgaaaatggatcttaccaggagtggcattgaataggcaggctctgggatgtaatctcactcaagaggtcatttgttggccccgccttcactagactagagttttaggataggttggggaacctatacgtcaagcccctacgaagattgagaaaaatcgatgcacataagccatccgaaaccagtattggaaagtgttcagtttcgttttccattctgaaatgttcatagtagtatagtatgttttccgttgggtcgacgccatgtgatcgctactaaagatagagtttccttggaaaaaccgaggccagttgagatcagtctccctttctaggagcagagcttaaaaagatgggaaattccaatgaatttcgatcacaatcatgtggtaataatgggtttgaatcagagagactcgatctggaaactcctcaatgattataacgtgaactcgttgaagagaaggagacaagcagaaatagacgctttttttgaaccatttgagagggcgcagcgtatccgtttcaataactggcagaacggaatagagttgttagatggggctgaatggaggaacggcgatatagttatccctggaggcggcggaccagtaatttcaagccccttggatcaatttttcattgatccattatttggtcttgatatgggtaacttttatttatcattcacaaatgaatccttgtctatggcggtaactgtcgttttggtgccatctttatttggagttgttacgaaaaagggcgggggaaagtcagtgccaaatgcatggcaatccttggtagagcttatttatgatttcgtgctgaacctggtaaacgaacaaataggtggaaatgttaaacaaaagtttttccctcgcatctcggtcacttttactttttcgttatttcgtaatccccagggtatgataccctttagcttcacagtgacaagtcattttctcattactttggctctttcattttccatttttataggcattacgatcgttggatttcaaagacatgggcttcatttttttagcttcttattaccagcgggagtcccactgccattagcaccttttttagtactccttgagctaatctctcattgttttcgtgcattaagctcaggaatacgtttatttgctaatatgatggccggtcatagttcagtaaagattttaagtgggttcgcttggactatgctatttctgaataatattttctatttcataggagatcttggtcccttatttattgtattggctcttactggattggaattaggtgtagctatattacaagctcatgtttctacgatctcaatttgtatttacttgaatgatgctataaatctccatcaaaatgagtaatttcataattgaataaaaacgaggagccgaagattttagggggcgggacaaacgcggaagtgtattgcgttacaaaaaatgacaactagcatttgttttttcatttcatgttcgaattcgtttttcgttggaaaaaccaacgccgaccccaaacaagtctctccaatataaggagagcggagcttaaaaatattattttattgtgctatggcaaatctggtccgatggctcttctccactacccgagggactaacggtcttccatatttcatcttcggtgtcgttgtaggaggcgccctgttgtttgctttgctaaagtatcaggcccctctgtacgacccggctttaatggaaaaaatcatagatcataatataaaagccgggcaccctatagaggttgactattcgtggtggggcacctctattcgtgtagtctttcctaagtaagaaagacaggacagtggtggtttgctcatactttcattacaaaaccatactatggaattagggataacagggtaataatcacaagtgagaaccacaggtagcaataggtattacagaaatttcctcgagtctgcttgaaagcctgcagagtccaattttgagtattttcagttagaatctagagtcagcctattcagttcttagcccttaagggtaaggcagggggtaatatggatagtctctgtccctgtattcacattccaccttcaacaaagtgttgatttcccgtaaagctaactgtagtcctttaagtaagtagatatcttaggcaagttagcaatctcgttatattaccaaggccttcccttctattgtagaaagagttctcagccatctaattgcagtgccagttgccagctatccagtttcatttgaagttgctgggggtccaaacgagctagttgcttttattcgtcctataagtccttccacaagcgagtcaatagggtgctggctagttgtagttgttggcgtgcctttcctttcatcttgaatattaataaatatttggataaattactttagaataagaagttcatgttttaatacgactcactatagtaagtaatacgaatccatactaggaaaatgaaaatgtgagtcctaggcactggaattggttctcttctccctaatccctataagccagaaagggtaataggcttcagtgtaagcatttccttcaagcaagtcatctcaagttttaaattctagagaatagctccgatcaacccattttagtttggttctgcaattcattcgcataaatgaaaaaaaaagcgagatgtgcacgaaagaagatcatagttcagctttaaaatggtggtgtccctgtgttagtaagtggttgaaatagctcatgggagtgtctgccccattcgataatggcatttatgatctagtggagtgagtgattgtgtggtgttcagtctaaggctttttgaaaagcggatttctcccttctctcatccatcgtctttgttaaagtgaatttctctgacattccatgtttccgaaacggatcctatactgcctaaactcgttataactcaccgagtacatgatgagatcctgcaactgctggcgccacattgcgagctgatgaccaaccaaaccgacagcacactgacacgcgaggaaattctgcgtcgatgtcgtgatgctcaagcgatgatggcgttcatgcccgatcgagtcgatgcagactttcttcaagcctgccctgagctgcgtgtagtcggctgcgcgctcaagggcttcgacaatttcgatgtggacgcctgtactgcccgtggggtctggctgaccttcgtgcctgatctgttgacagtcccaactgccgagctggcgatcggactggcggtggggctggggcgacatctgcgagcagcagatgcgttcgtccgttctggcgagttccaaggctggcaaccacaattctatggcacagggctggataacgctacagtcggcatccttggcatgggcgccatcggactggccatggctgatcgattgcaaggatggggcgcgaccctgcaatatcacgaggcgaaggctctggatacacaaaccgagcaacgactcggcctgcgtcaagtggcgtgcagcgaactcttcgccagctcggacttcatcctgctggcgcttcccttgaatgccgatacccaacatctggtcaacgccgagctgcttgccctcgtacgaccaggcgctctgcttgtaaacccctgtcgtggttcggtagtggatgaagccgccgtgctcgcggcgcttgagcgaggccaactcggcgggtatgcggcggatgtattcgaaatggaagactgggctcgtgcggaccgaccacgactgatcgatcctgcgctgctcgcgcatcctaatacactgttcactccacatatagggtcggcagtgcgtgcggtgcgtctggagattgaacgttgtgcagcgcaaaacatcatccaagtattggcaggtgcgcgtccaatcaacgctgcgaaccgtctgcccaaggccgagcctgccgcatgtccagttgctactatggtgagtaagggagaggagctgttcaccggggtggtgcctatcctggtcgagctggatggtgatgtaaacggtcataaattcagtgtgtccggtgaaggtgaaggtgatgccacctatggtaagctgacccttaagttcatctgtaccaccggaaagctgcctgtgccttggcctaccctcgtgaccaccctgacatatggagtgcaatgtttcagtcgttatcctgatcatatgaagcaacatgatttctttaaatccgccatgcctgaaggttatgtccaagagcgtaccatattctttaaagatgatggtaactataagacccgtgccgaggtgaagttcgagggtgataccctggtgaaccgtattgagcttaagggtatcgatttcaaggaggatggaaacatcctggggcataagctggagtataactataacagtcataacgtctatatcatggccgataagcaaaagaacggtatcaaggtgaacttcaagatccgtcataatatcgaagatggaagtgtgcaactcgccgatcattatcaacaaaacacccctatcggtgatggtcctgtgctgctgcctgataaccattatctgagtacccaatccgccctgagtaaagatcctaacgagaagcgtgatcaaatggtactgcttgagttcgttaccgccgccgggatcactctcggtatggatgagctgtataagtaatgaggtaccaagcgatcgcaataatacgactcactatagacctaggaaaagatctagacgaggtgtagcgcagtctggtcagcgcatctgttttgggtacagagggccataggttcgaatcctgtcaccttgagtctggccccaaattctaatttctactgttgtagattagttctagcattaaccggtcgtccctttcgtccagtggttaggacatcgtcttttcatgtcgaagacacgggttcgattcccgtaagggatagtctggccccaaattctaatttctactgttgtagatgtcacatttctaccggtgcacattccagcttatttgatacccacttcaagtttctatcaaaccatgtctttttcttcgaacgtcaatctcgtagttcttccgaactcaactccggttttagagctagaaatagcaagttaaaataaggctagtccgttatcaacttgaaaaagtggcaccgagtcggtgctggggaaatagctcagttggttagagtgctggtctgtcacgccagaagtcgcgggttcgaaccccgttttccccgatctgggatctgatatgtgggttttagagctagaaatagcaagttaaaataaggctagtccgttatcaacttgaaaaagtggcaccgagtcggtgctgggagagtggccgagcggtcaaaagcgacagactgtaaatctgttgaaggttttctacgtaggttcgaatcctgcctctcccagaataatacgactcactatagaataatagagggcttatagtttaattggttgaaacgtaccgctcataacggtgatattgtaggttcgagccctactaagcccagtctggccccaaattctaatttctactgttgtagataatcggcatgtactatggaatggaggtatggctgagtggcttaaggcattggtttgctaaatcgacatacaagaagattgtatcatgggttcgaatcccatttcctccggtctggccccaaattctaatttctactgttgtagatgatatgcaagcacgagattcctggagtatagccaagtggtaaggcatcggtttttggtaccggcatgcaaaggttcgaatccttttactccagaaccccttggggcctctaaacgggtcttgaggggttttttggaaagatctaaaaagcttaagcggccgcaaaaaccccttggggcctctaaacgggtcttgaggggttttttgtagatagacctttttatttttcgtcattcgatcacgaaaaggccggccaaacctcgaggtaattaaagcggccgaaattagctagcaaataagcatgcaatcacaagtgagaaccacaggtagcaataggtattacagtagggataacagggtaatgaattcgaaaccatcttctcactctgacccccacatatcagatcccagatgcataggaaaagcggtatcaagaatagtagtataaagaaagatagtacagtactcaagtaaatgaattcgcctaaggatcgatggaaagatcaaggtccccgtgaaaaagtagatactagatcgatatgatactctcatctctggagtaacttcttccattatgctgatctctaggtccgttccatcatcatcgtaatagtatggtcccaggtgtccgagctatagatcaagatcatatccagtcacatttctaccggtgcacttctcatgaaataattccctttccaaggaaaggaaaacaagaactcgaatactcgtaatagcgatcccgatccacctacttttttctattctttgattcgaaacgtgctaaagcacaagccatttttatgcatggggcataagagtggacaatctatgttatcgaaggaagtaaataacaacacttcagcgtttaggtctaccttcagtaaaccaatagttttgcagcattggaatttgagttggccaggtaaggtcctctaaaaagaaaagaagaaactacttagaatagataaatgccattggttttctcgtactatacgatctttttttgttttgttttttggccatgattgtgctgctcctgtgaaggctagtgggaaagctcaccgttcgttgtgatgagtgggggccttgtatctgtattcggatcagctccttaacagagtttcctgcttgaaccctggctgggagctgggagaggtgtcccactacaggtgcaaataaaccatttgaccttacaggggaaaggaaacaaaccactcaataatcggtagaaattcctcctactgaacagctttccttttctcgccttaactactacttcaaagcaaggcggaatatcacgggataggaatgaaagaacttcttactcaactttctagctatataaaaatagttagcaatatgaaacgagtaacttaagccctagtaaaaggctactctttgaatcccctctttaaggcatataaaattagtactcttcctgagctagcttaagcatatcttgagcgagtgagttgtatttccctccatcaagttctaagcgatcaaataaggtccttgctctcgagccaatgccaataccaatagagagggtctaaacgaaggattcaaaggcgcg

SEQ ID NO: 125 - DNA, 7477 bp, synthetic construct
  source 1..7477: mol_type = other DNA
ggcccaaattcaattgtatatgagctcatatacaagacctcactagtaaggaaggcacttgctgccggagttcaacaggcaaatataagaaaagaagtcctgttcacttcatcatctgtgggttgtactgcttgaaggttcttctgaggggtagaatttgaattccttctttgcttgtgagataaccatttccagaaactcatatatagagagcgggtatcggtgaaaatggatcttaccaggagtggcattgaataggcaggctctgggatgtaatctcactcaagaggtcatttgttggccccgccttcactagactagagttttaggataggttggggaacctatacgtcaagcccctacgaagattgagaaaaatcgatgcacataagccatccgaaaccagtattggaaagtgttcagtttcgttttccattctgaaatgttcatagtagtatagtatgttttccgttgggtcgacgccatgtgatcgctactaaagatagagtttccttggaaaaaccgaggccagttgagatcagtctccctttctaggagcagagcttaaaaagatgggaaattccaatgaatttcgatcacaatcatgtggtaataatgggtttgaatcagagagactcgatctggaaactcctcaatgattataacgtgaactcgttgaagagaaggagacaagcagaaatagacgctttttttgaaccatttgagagggcgcagcgtatccgtttcaataactggcagaacggaatagagttgttagatggggctgaatggaggaacggcgatatagttatccctggaggcggcggaccagtaatttcaagccccttggatcaatttttcattgatccattatttggtcttgatatgggtaacttttatttatcattcacaaatgaatccttgtctatggcggtaactgtcgttttggtgccatctttatttggagttgttacgaaaaagggcgggggaaagtcagtgccaaatgcatggcaatccttggtagagcttatttatgatttcgtgctgaacctggtaaacgaacaaataggtggaaatgttaaacaaaagtttttccctcgcatctcggtcacttttactttttcgttatttcgtaatccccagggtatgataccctttagcttcacagtgacaagtcattttctcattactttggctctttcattttccatttttataggcattacgatcgttggatttcaaagacatgggcttcatttttttagcttcttattaccagcgggagtcccactgccattagcaccttttttagtactccttgagctaatctctcattgttttcgtgcattaagctcaggaatacgtttatttgctaatatgatggccggtcatagttcagtaaagattttaagtgggttcgcttggactatgctatttctgaataatattttctatttcataggagatcttggtcccttatttattgtattggctcttactggattggaattaggtgtagctatattacaagctcatgtttctacgatctcaatttgtatttacttgaatgatgctataaatctccatcaaaatgagtaatttcataattgaataaaaacgaggagccgaagattttagggggcgggacaaacgcggaagtgtattgcgttacaaaaaatgacaactagcatttgttttttcatttcatgttcgaattcgtttttcgttggaaaaaccaacgccgaccccaaacaagtctctccaatataaggagagcggagcttaaaaatattattttattgtgctatggcaaatctggtccgatggctcttctccactacccgagggactaacggtcttccatatttcatcttcggtgtcgttgtaggaggcgccctgttgtttgctttgctaaagtatcaggcccctctgtacgacccggctttaatggaaaaaatcatagatcataatataaaagccgggcaccctatagaggttgactattcgtggtggggcacctctattcgtgtagtctttcctaagtaagaaagacaggacagtggtggtttgctcatactttcattacaaaaccatactatggaattagggataacagggtaataatcacaagtgagaaccacaggtagcaataggtattacagaaatttcctcgagtctgcttgaaagcctgcagagtccaattttgagtattttcagttagaatctagagtcagcctattcagttcttagcccttaagggtaaggcagggggtaatatggatagtctctgtccctgtattcacattccaccttcaacaaagtgttgatttcccgtaaagctaactgtagtcctttaagtaagtagatatcttaggcaagttagcaatctcgttatattaccaaggccttcccttctattgtagaaagagttctcagccatctaattgcagtgccagttgccagctatccagtttcatttgaagttgctgggggtccaaacgagctagttgcttttattcgtcctataagtccttccacaagcgagtcaatagggtgctggctagttgtagttgttggcgtgcctttcctttcatcttgaatattaataaatatttggataaattactttagaataagaagttcatgttttaatacgactcactatagtaagtaatacgaatccatactaggaaaatgaaaatgtgagtcctaggcactggaattggttctcttctccctaatccctataagccagaaagggtaataggcttcagtgtaagcatttccttcaagcaagtcatctcaagttttaaattctagagaatagctccgatcaacccattttagtttggttctgcaattcattcgcataaatgaaaaaaaaagcgagatgtgcacgaaagaagatcatagttcagctttaaaatggtggtgtccctgtgttagtaagtggttgaaatagctcatgggagtgtctgccccattcgataatggcatttatgatctagtggagtgagtgattgtgtggtgttcagtctaaggctttttgaaaagcggatttctcccttctctcatccatcgtctttgttaaagttgaacagtcactcacttttgacagttatacgattccagaactgcctaaactcgttataactcaccgagtacatgatgagatcctgcaactgctggcgccacattgcgagctgatgaccaaccaaaccgacagcacactgacacgcgaggaaattctgcgtcgatgtcgtgatgctcaagcgatgatggcgttcatgcccgatcgagtcgatgcagactttcttcaagcctgccctgagctgcgtgtagtcggctgcgcgctcaagggcttcgacaatttcgatgtggacgcctgtactgcccgtggggtctggctgaccttcgtgcctgatctgttgacagtcccaactgccgagctggcgatcggactggcggtggggctggggcgacatctgcgagcagcagatgcgttcgtccgttctggcgagttccaaggctggcaaccacaattctatggcacagggctggataacgctacagtcggcatccttggcatgggcgccatcggactggccatggctgatcgattgcaaggatggggcgcgaccctgcaatatcacgaggcgaaggctctggatacacaaaccgagcaacgactcggcctgcgtcaagtggcgtgcagcgaactcttcgccagctcggacttcatcctgctggcgcttcccttgaatgccgatacccaacatctggtcaacgccgagctgcttgccctcgtacgaccaggcgctctgcttgtaaacccctgtcgtggttcggtagtggatgaagccgccgtgctcgcggcgcttgagcgaggccaactcggcgggtatgcggcggatgtattcgaaatggaagactgggctcgtgcggaccgaccacgactgatcgatcctgcgctgctcgcgcatcctaatacactgttcactccacatatagggtcggcagtgcgtgcggtgcgtctggagattgaacgttgtgcagcgcaaaacatcatccaagtattggcaggtgcgcgtccaatcaacgctgcgaaccgtctgcccaaggccgagcctgccgcatgtccagttgctactatggtgagtaagggagaggagctgttcaccggggtggtgcctatcctggtcgagctggatggtgatgtaaacggtcataaattcagtgtgtccggtgaaggtgaaggtgatgccacctatggtaagctgacccttaagttcatctgtaccaccggaaagctgcctgtgccttggcctaccctcgtgaccaccctgacatatggagtgcaatgtttcagtcgttatcctgatcatatgaagcaacatgatttctttaaatccgccatgcctgaaggttatgtccaagagcgtaccatattctttaaagatgatggtaactataagacccgtgccgaggtgaagttcgagggtgataccctggtgaaccgtattgagcttaagggtatcgatttcaaggaggatggaaacatcctggggcataagctggagtataactataacagtcataacgtctatatcatggccgataagcaaaagaacggtatcaaggtgaacttcaagatccgtcataatatcgaagatggaagtgtgcaactcgccgatcattatcaacaaaacacccctatcggtgatggtcctgtgctgctgcctgataaccattatctgagtacccaatccgccctgagtaaagatcctaacgagaagcgtgatcaaatggtactgcttgagttcgttaccgccgccgggatcactctcggtatggatgagctgtataagtaatgaggtaccaagcgatcgcaataatacgactcactatagacctaggaaaagatctagacgaggtgtagcgcagtctggtcagcgcatctgttttgggtacagagggccataggttcgaatcctgtcaccttgagtctggccccaaattctaatttctactgttgtagattagttctagcattaaccggtcgtccctttcgtccagtggttaggacatcgtcttttcatgtcgaagacacgggttcgattcccgtaagggatagtctggccccaaattctaatttctactgttgtagatgtcacatttctaccggtgcacattccagcttatttgatacccacttcaagtttctatcaaaccatgtctttttcttcgaacgtcaatctcgtagttcttccgaactcaactccggttttagagctagaaatagcaagttaaaataaggctagtccgttatcaacttgaaaaagtggcaccgagtcggtgctggggaaatagctcagttggttagagtgctggtctgtcacgccagaagtcgcgggttcgaaccccgttttccccgatctgggatctgatatgtgggttttagagctagaaatagcaagttaaaataaggctagtccgttatcaacttgaaaaagtggcaccgagtcggtgctgggagagtggccgagcggtcaaaagcgacagactgtaaatctgttgaaggttttctacgtaggttcgaatcctgcctctcccagaataatacgactcactatagaataatagagggcttatagtttaattggttgaaacgtaccgctcataacggtgatattgtaggttcgagccctactaagcccagtctggccccaaattctaatttctactgttgtagataatcggcatgtactatggaatggaggtatggctgagtggcttaaggcattggtttgctaaatcgacatacaagaagattgtatcatgggttcgaatcccatttcctccggtctggccccaaattctaatttctactgttgtagatgatatgcaagcacgagattcctggagtatagccaagtggtaaggcatcggtttttggtaccggcatgcaaaggttcgaatccttttactccagaaccccttggggcctctaaacgggtcttgaggggttttttggaaagatctaaaaagcttaagcggccgcaaaaaccccttggggcctctaaacgggtcttgaggggttttttgtagatagacctttttatttttcgtcattcgatcacgaaaaggccggccaaacctcgaggtaattaaagcggccgaaattagctagcaaataagcatgcaatcacaagtgagaaccacaggtagcaataggtattacagtagggataacagggtaatgaattcgaaaccatcttctcactctgacccccacatatcagatcccagatgcataggaaaagcggtatcaagaatagtagtataaagaaagatagtacagtactcaagtaaatgaattcgcctaaggatcgatggaaagatcaaggtccccgtgaaaaagtagatactagatcgatatgatactctcatctctggagtaacttcttccattatgctgatctctaggtccgttccatcatcatcgtaatagtatggtcccaggtgtccgagctatagatcaagatcatatccagtcacatttctaccggtgcacttctcatgaaataattccctttccaaggaaaggaaaacaagaactcgaatactcgtaatagcgatcccgatccacctacttttttctattctttgattcgaaacgtgctaaagcacaagccatttttatgcatggggcataagagtggacaatctatgttatcgaaggaagtaaataacaacacttcagcgtttaggtctaccttcagtaaaccaatagttttgcagcattggaatttgagttggccaggtaaggtcctctaaaaagaaaagaagaaactacttagaatagataaatgccattggttttctcgtactatacgatctttttttgttttgttttttggccatgattgtgctgctcctgtgaaggctagtgggaaagctcaccgttcgttgtgatgagtgggggccttgtatctgtattcggatcagctccttaacagagtttcctgcttgaaccctggctgggagctgggagaggtgtcccactacaggtgcaaataaaccatttgaccttacaggggaaaggaaacaaaccactcaataatcggtagaaattcctcctactgaacagctttccttttctcgccttaactactacttcaaagcaaggcggaatatcacgggataggaatgaaagaacttcttactcaactttctagctatataaaaatagttagcaatatgaaacgagtaacttaagccctagtaaaaggctactctttgaatcccctctttaaggcatataaaattagtactcttcctgagctagcttaagcatatcttgagcgagtgagttgtatttccctccatcaagttctaagcgatcaaataaggtccttgctctcgagccaatgccaataccaatagagagggtctaaacgaaggattcaaaggcgcg

SEQ ID NO: 126 - DNA, 38 bp, Oryza sativa
  source 1..38: mol_type = genomic DNA
gaatttctctgacattccatgtttccgaaacggatcct

SEQ ID NO: 127 - DNA, 41 bp, Oryza sativa
  source 1..41: mol_type = genomic DNA
gtagggctttctgaggagtaagcctaattccgttaatgcag

SEQ ID NO: 128 - DNA, 27 bp, Oryza sativa
  source 1..27: mol_type = genomic DNA
gagagacttgtttggggtcggcgttgg

SEQ ID NO: 129 - DNA, 38 bp, synthetic construct
  source 1..38: mol_type = other DNA
accacaggtagcaataggtattacagtagggataacag

SEQ ID NO: 130 - DNA, 30 bp, Oryza sativa
  source 1..30: mol_type = genomic DNA
agtgctcagaataatccaggtcgctcgacg

SEQ ID NO: 131 - DNA, 40 bp, Oryza sativa
  source 1..40: mol_type = genomic DNA
tgaacagtcactcacttttgacagttatacgattccagaa

SEQ ID NO: 132 - DNA, 27 bp, Oryza sativa
  source 1..27: mol_type = genomic DNA
gagagaagatgacccagatcatgttcg

SEQ ID NO: 133 - DNA, 27 bp, Oryza sativa
  source 1..27: mol_type = genomic DNA
ctggcagtatcaagctcctgttcataa

SEQ ID NO: 134 - DNA, 23 bp, Oryza sativa
  source 1..23: mol_type = genomic DNA
gtctgccccattcgataatggca

SEQ ID NO: 135 - DNA, 26 bp, Pseudomonas stutzeri
  source 1..26: mol_type = genomic DNA
tccacatcgaaattgtcgaagccctt